(19) United States
(12) Patent Application Publication
(10) Pub. No.: US 2015/0175980 A1
(43) Pub. Date: Jun. 25, 2015

(54) NOVEL CELL WALL DECONSTRUCTION ENZYMES OF SCYTALIDIUM THERMOPHILUM, MYRIOCOCCUM THERMOPHILUM, AND AUREOBASIDIUM PULLULANS, AND USES THEREOF

(71) Applicant: CONCORDIA UNIVERSITY, MONTREAL (CA)

(72) Inventors: Adrian Tsang, Montreal (CA); Justin Powlowski, Montreal (CA); Gregory Butler, Montreal (CA)

(21) Appl. No.: 14/405,602

(22) PCT Filed: Jun. 7, 2013
(86) PCT No.: PCT/CA2013/050434

§ 371 Date: Dec. 4, 2014

Related U.S. Application Data
(60) Provisional application No. 61/657,075, filed on Jun. 8, 2012.
(60) Provisional application No. 61/657,078, filed on Jun. 8, 2012.
(60) Provisional application No. 61/657,082, filed on Jun. 8, 2012.

(51) Int. Cl. C12N 9/18; C12N 9/24; A23K 1/165; C12N 9/90; C12N 9/88; A23L 1/30; C07K 16/40; C12N 9/42

(52) U.S. Cl. CPC C12N 9/18; C07K 16/40; C12N 9/248; C12N 9/2402; C12N 9/2437; C12N 9/2482; C12N 9/24; C12N 9/90; C12N 9/88; A23L 1/3014; A23K 1/1653; C12Y 302/01; C12Y 302/01055; C12Y 302/01004; C12Y 302/01037; C12Y 301/01072; C12Y 501/03003; A23V 2002/00

(57) ABSTRACT

The present invention relates to novel polypeptides and enzymes (e.g., thermostable proteins and enzymes) having activities relating to biomass processing and/or degradation (e.g., cell wall deconstruction), as well as polynucleotides, vectors, cells, compositions and tools relating to same, or functional variants thereof. More particularly, the present invention relates to secreted enzymes that may be isolated from the fungi, Scytalidium thermophilum strain CBS 625.91, Myriococcum thermophilum strain CBS 389.93, and Aureobasidium pullulans strain ATCC 62921. Uses thereof in various industrial processes such as in biofuels, food preparation, animal feed, pulp and paper, textiles, detergents, waste treatment and others are also disclosed.

### FIELD OF THE INVENTION

The present invention relates to novel polypeptides and enzymes having activities relating to biomass processing and/or degradation (e.g., cell wall deconstruction), as well as polynucleotides, vectors, cells, compositions and tools relating to same, or functional variants thereof. More particularly, the present invention relates to secreted enzymes that may be isolated from the fungi, Scytalidium thermophilum strain CBS 625.91, Myriococcum thermophilum strain CBS 389.93, and Aureobasidium pullulans strain ATCC 62921. Uses thereof in various industrial processes such as in biofuels, food preparation, animal feed, pulp and paper, textiles, detergents, waste treatment and others are also disclosed.

### SEQUENCE LISTING

This application contains a Sequence Listing in computer readable form entitled “Seq_Listing_SCYTH_MYRTH_AURPU.txt”, created Jun. 6, 2013 having a size of about 7.78 MB. The computer readable form is incorporated herein by reference.

### BACKGROUND OF THE INVENTION

Biomass-processing enzymes have a number of industrial applications such as in: the biofuel industry (e.g., improving ethanol yield and/or increasing the efficiency and economy of ethanol production); the food industry (e.g., production of cereal-based food products; the feed-enzyme industry (e.g., increasing the digestibility/absorption of nutrients); the pulp and paper industry (e.g., enhancing bleachability of pulp); the textile industry (e.g., treatment of cellulose-based fabrics); the waste treatment industry (e.g., de-colorization of synthetic dyes); the detergent industry (e.g., providing eco-friendly cleaning products); and the rubber industry (e.g., catalyzing the conversion of latex into foam rubber).

In particular, driven by the limited availability of fossil fuels, there is a growing interest in the biofuel industry for improving the conversion of biomass into second-generation biofuels. This process is heavily dependent on inexpensive and effective enzymes for the conversion of lignocellulose to ethanol. Cellulase enzyme cocktails involve the concerted action of endoglucanases, cellobiohydrolases (also known as exoglucanases), and beta-glucosidases. The current cost of cellulose-degrading enzymes is too high for bioethanol to compete economically with fossil fuels. Cost reduction may result from the discovery of cellulase enzymes with, for example, higher specific activity, lower production costs, and/or greater compatibility with processing conditions including temperature, pH and the presence of inhibitors in the biomass, or produced as the result of biomass pre-treatment.

Conversion of plant biomass to glucose may also be enhanced by supplementing cellulose cocktails with enzymes that degrade the other components of biomass, including hemicelluloses, pectins and lignins, and their linkages, thereby improving the accessibility of cellulose to the cellulase enzymes. Such enzymes include, without being limiting, to: xylanases, mannanases, arabinanases, esterases, glucuronidases, xyloglucanases and arabinofuranosidases for hemicelluloses; lignin peroxidases, manganese-dependent peroxidases, versatile peroxidases, and laccases for lignin; and pectate lyase, pectin lyase, polygalacturonase, pectin acetyl esterase, alpha-arabinofuranosidase, beta-galactosidase, galactanase, arabinanase, rhamnogalacturonase, rhamnogalacturonan lyase, and rhamnogalacturonan acetyl esterase, xylogalacturonosidase, xylogalacturonase, and rhamnogalacturonan lyase. Additionally, glycoside hydrolase family 61 (GH61) proteins have been shown to stimulate the activity of cellulase preparations.

These enzymes may also be useful for other purposes in processing biomass. For example, the lignin modifying enzymes may be used to alter the structure of lignin to produce novel materials, and hemicellulases may be employed to produce 5-carbon sugars from hemicelluloses, which may then be further converted to chemical products.

There is also a growing need for improved enzymes for food processing and feed applications. Cereal-based food products such as pasta, noodles and bread can be prepared from dough which is usually made from the basic ingredients (cereal) flour, water and optionally salt. As a result of a consumer-driven need to replace the chemical additives by more natural products, several enzymes have been developed with dough and/or cereal-based food product-improving properties, which are used in all possible combinations depending on the specific application conditions. Suitable enzymes include, for example, xylanase, starch degrading enzymes, oxidizing enzymes, fatty material splitting enzymes, protein degrading, and modifying or crosslinking enzymes. Many of these enzymes are also used for treating animal feed or animal feed additives, to make them more digestible or to improve their nutritional quality. Amylases are used for the conversion of plant starches to glucose. Pectin-active enzymes are used in fruit processing, for example to increase the yield of juices, and in fruit juice clarification, as well as in other food processing steps.

There is also a growing need for improved enzymes in other industries. In the pulp and paper industry, enzymes are used to make the bleaching process more effective and to reduce the use of oxidative chemicals. In the textile industry, enzymatic treatment is often used in place of (or in addition to) a bleaching treatment to achieve a “used” look of jeans, and can also improve the softness/feel of fabrics. When used in detergent compositions, enzymes can enhance cleaning ability or act as a softening agent. In the waste treatment industry, enzymes play an important role in changing the characteristics of the waste, for example, to become more amenable to further treatment and/or for bio-conversion to value-added products.

There is also a growing need for industrial enzymes and proteins that are “thermostable” in that they retain a level of their function or protein activity at temperatures about 50° C. These thermostable enzymes are highly desirable, for example, to be able to perform reactions at elevated temperatures to avoid or reduce contamination by microorganisms (e.g., bacteria).

There thus remains a need in the above-mentioned industries and others for biomass-processing enzymes, polynucleotides encoding same, and recombinant vectors and strains for expressing same.

The present description refers to a number of documents, the content of which is herein incorporated by reference in their entirety.

### SUMMARY OF THE INVENTION

In general, the present invention relates to soluble, secreted proteins relating to biomass processing and/or degradation (e.g., cell wall deconstruction) that may be isolated from the fungi, Scytalidium thermophilum strain CBS 625.91, Myriococcum thermophilum strain CBS 389.93, and Aureobasidium pullulans strain ATCC 62921, as well as polynucleotides, vectors, compositions, cells, antibodies, kits, products and uses associated with same. Briefly, these fungal strains were cultured in vitro and genomic DNA along with total RNA were isolated therefrom. These nucleic acids were then used to determine/assemble fungal genomic sequences and generate cDNA libraries. Bioinformatic tools were used to predict genes in the assembled genomic sequences, and those genes encoding proteins relating to biomass-degradation (e.g., cell wall deconstruction) were identified based on bioinformatics (e.g., the presence of conserved domains). Sequences predicted to encode proteins which are targeted to the mitochondria or bound to the cell wall were removed. cDNA clones comprising full-length sequences predicted to encode soluble, secreted proteins relating to biomass-degradation were fully sequenced and cloned into appropriate expression vectors for protein production and characterization. The full-length genomic, exonic, intronic, coding and polypeptide sequences are disclosed herein, along with corresponding putative (biological) functions and/or protein activities, where available.

The soluble, secreted, biomass degradation proteins of the present invention comprise a proteome which is referred to herein as the SSBD proteome of Scytalidium thermophilum strain CBS 625.91, Myriococcum thermophilum, or Aureobasidium pullulans.

Accordingly, in some aspects the present invention relates to an isolated polypeptide which is:
(a) a polypeptide comprising the amino acid sequence of any one of SEQ ID NOs: 571-855, 1468-1773, or 2548-2934
(b) a polypeptide comprising an amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% amino acid sequence identity to the polypeptide defined in (a);
(c) a polypeptide comprising an amino acid sequence encoded by the nucleic acid sequence of any one of SEQ ID NOs: 286-570, 1162-1467, or 2161-2547;
(d) a polypeptide comprising an amino acid sequence encoded by any one the exonic nucleic acid sequences corresponding to the positions as defined in Tables 2A-2C;
(e) a polypeptide comprising an amino acid sequence encoded by a polynucleotide molecule that hybridizes under medium-high stringency conditions, high stringency conditions, or very high stringency conditions with the full-length complement of a polynucleotide molecule comprising the nucleic acid sequence defined in (c) or (d);
(f) a polypeptide comprising an amino acid sequence encoded by a polynucleotide molecule having at least 60%, at least 65% at least 70%, at least 75%, at least 80%, at least 85%, at least 90%, at least 91%, at least 92%, at least 93%, at least 94%, at least 95%, at least 96%, at least 97%, at least 98%, or at least 99% nucleic acid sequence identity to a polynucleotide molecule comprising the nucleic acid sequence defined in (c) or (d);
(g) a functional variant of the polypeptide defined in (a) comprising a substitution, deletion, and/or insertion at one or more residues; or
(h) a functional fragment of the polypeptide of any one of (a) to (g).

In some embodiments, the above mentioned polypeptide has a corresponding function and/or protein activity according to Tables 1A-1C.

In some embodiments, the above mentioned polypeptide comprises or consists of the amino acid sequence of any one of SEQ ID NOs: 571-855, 1468-1773, or 2548-2934.

In some embodiments, the above mentioned polypeptide is a recombinant polypeptide.

In some embodiments, above mentioned polypeptide is obtainable from a fungus. In some embodiments, the fungus is from the genus Scytalidium, Myriococcum, or Aureobasidium. In some embodiments, the fungus is Scytalidium thermophilum, Myriococcum thermophilum, or Aureobasidium pullulans.

In some aspects, the present invention relates to an antibody that specifically binds to any one of the above mentioned polypeptides.

In some aspects, the present invention relates to an isolated polynucleotide molecule encoding any one of the above mentioned polypeptides.

In some aspects, the present invention relates to an isolated polynucleotide molecule which is:
(a) a polynucleotide molecule comprising a nucleic acid sequence encoding the polypeptide of any one of SEQ ID NOs: 571-855, 1468-1773, or 2548-2934;
(b) a polynucleotide molecule comprising the nucleic acid sequence of any one of SEQ ID NOs: 1-285, 856-1161, or 1774-2160;
(c) a polynucleotide molecule comprising the nucleic acid sequence of any one of SEQ ID NOs: SEQ ID NOs: 286-570, 1162-1467, or 2161-2547;
(d) a polynucleotide molecule comprising any one of the exonic nucleic acid sequences corresponding to the positions as defined in Tables 2A-2C;
(e) a polynucleotide molecule comprising a nucleic acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% nucleic acid sequence identity to any one of the polynucleotide molecules defined in (a) to (d); or
(f) a polynucleotide molecule that hybridizes under medium-high stringency conditions, high stringency conditions, or very high stringency conditions with the full-length complement of any one of the polynucleotide molecules defined in (a) to (e).

In some embodiments, the above mentioned polynucleotide molecule is obtainable from a fungus. In some embodiments, the fungus is from the genus Scytalidium, Myriococcum, or Aureobasidium. In some embodiments, the fungus is Scytalidium thermophilum, Myriococcum thermophilum, or Aureobasidium pullulans.

In some aspects, the present invention relates to a vector comprising any one of the above mentioned polynucleotide molecules. In some embodiments, the vector comprises a regulatory sequence operatively linked to the polynucleotide molecule for expression of same in a suitable host cell. In some embodiments, the suitable host cell is a bacterial cell; a fungal cell; or a filamentous fungal cell.

In some embodiments, the present invention relates to a recombinant host cell comprising any one of the above mentioned polynucleotide molecules or vectors. In some embodiments, the present invention relates to a polypeptide obtainable by expressing the above mentioned polynucleotide or vector in a suitable host cell. In some embodiments, the suitable host cell is a bacterial cell; a fungal cell; or a filamentous fungal cell.

In some aspects, the present invention relates to a composition comprising any one of the above mentioned polypeptides or the recombinant host cells. In some embodiments, the composition further comprising a suitable carrier. In some embodiments, the composition further comprises a substrate of the polypeptide. In some embodiments, the substrate is biomass.

In some aspects, the present invention relates to a method for producing any one of the above mentioned polypeptides, the method comprising: (a) culturing a strain comprising the above mentioned polynucleotide molecule or vector under conditions conducive for the production of the polypeptide; and (b) recovering the polypeptide. In some embodiments, the strain is a bacterial strain; a fungal strain; or a filamentous fungal strain.

In some aspects, the present invention relates to a method for producing any one of the above mentioned polypeptides, the method comprising: (a) culturing the above mentioned recombinant host cell under conditions conducive for the production of the polypeptide; and (b) recovering the polypeptide.

In some aspects, the present invention relates to a method for preparing a food product, the method comprising incorporating any one of the above mentioned polypeptides during preparation of the food product. In some embodiments, the food product is a bakery product.

In some aspects, the present invention relates to the use of the above mentioned polypeptide for the preparation or processing of a food product. In some embodiments, the food product is a bakery product.

In some aspects, the present invention relates to the use of any one of the above mentioned polypeptides for the preparation or processing of a food product. In some embodiments, the food product is a bakery product.

In some aspects, the present invention relates to the above mentioned polypeptide for use in the preparation or processing of a food product. In some embodiments, the food product is a bakery product.

In some aspects the present invention relates to the use of any one of the above mentioned polypeptides for the preparation of animal feed. In some aspects the present invention relates to the use of any one of the above mentioned polypeptides for increasing digestion or absorption of animal feed. In some aspects, the present invention relates to any one of the above mentioned polypeptides for use in the preparation of animal feed, or for increasing digestion or absorption of animal feed. In some embodiment, the animal feed is a cereal-based feed.

In some aspects the present invention relates to the use of any one of the above mentioned polypeptides for the production or processing of kraft pulp or paper. In some aspects the present invention relates to any one of the above mentioned polypeptides for the production or processing of kraft pulp or paper. In some embodiments, the processing comprises prebleaching and/or de-inking.

In some aspects the present invention relates to the use of any one of the above mentioned polypeptides for processing lignin. In some aspects the present invention relates to any one of the above mentioned polypeptides for processing lignin.

In some aspects the present invention relates to the use of any one of the above mentioned polypeptides for producing ethanol. In some aspects the present invention relates to any one of the above mentioned polypeptides for producing ethanol.

In some embodiments, the above mentioned uses are in conjunction with cellulose or a cellulase.

In some aspects the present invention relates to the use of any one of the above mentioned polypeptides for treating textiles or dyed textiles. In some aspects the present invention relates to any one of the above mentioned polypeptides for treating textiles or dyed textiles.

In some aspects the present invention relates to the use of any one of the above mentioned polypeptides for degrading biomass or pretreated biomass. In some aspects the present invention relates to any one of the above mentioned polypeptides for degrading biomass or pretreated biomass.

In some embodiments, the present invention relates to proteins and/or enzymes that are thermostable. In some embodiments, a polypeptide of the present invention retains a level of its function and/or protein activity at about 50° C., about 55° C., about 60° C., about 65° C., about 70° C., about 75° C., about 80° C., or about 95° C. In some embodiments, a polypeptide of the present invention retains a level of its function and/or protein activity between about 50° C. and about 95° C., between about 50° C. and about 90° C., between about 50° C. and about 85° C., between about 50° C. and about 80° C., between about 50° C. and about 75° C., between about 50° C. and about 70° C., or between about 50° C. and about 65° C. In some embodiments, a polypeptide of the present invention has optimal or maximal function and/or protein activity greater than 50° C., greater than 55° C., greater than 60° C., greater than 65° C., or greater than 70° C. In some embodiments, a polypeptide of the present invention has optimal or maximal function and/or protein activity between about 50° C. and about 95° C., between about 50° C. and about 90° C., between about 50° C. and about 85° C., between about 50° C. and about 80° C., between about 50° C. and about 75° C., between about 50° C. and about 70° C., or between about 50° C. and about 65° C.

Unless defined otherwise, the scientific and technological terms and nomenclature used herein have the same meaning as commonly understood by a person of ordinary skill to which this invention pertains. Commonly understood definitions of molecular biology terms can be found for example in Dictionary of Microbiology and Molecular Biology, 2nd ed. (Singleton et al., 1994, John Wiley & Sons, New York, N.Y.) or The Harper Collins Dictionary of Biology (Hale & Marham, 1991, Harper Perennial, New York, N.Y.), Rieger et al., Glossary of genetics: Classical and molecular, 5th edition, Springer-Verlag, New-York, 1991; Alberts et al., Molecular Biology of the Cell, 4th edition, Garland science, New-York, 2002; and, Lewin, Genes VII, Oxford University Press, New-York, 2000. Generally, the procedures of molecular biology methods and the like are common methods used in the art. Such standard techniques can be found in reference manuals such as for example Sambrook et al., (2000, Molecular Cloning—A Laboratory Manual, Third Edition, Cold Spring Harbor Laboratories); and Ausubel et al., (1994, Current Protocols in Molecular Biology, John Wiley & Sons, New-York).

Further objects and advantages of the present invention will be clear from the description that follows.

### DEFINITIONS

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

In the present description, a number of terms are extensively utilized. In order to provide a clear and consistent understanding of the specification and claims, including the scope to be given such terms, the following definitions are provided.

Nucleotide sequences are presented herein by single strand, in the 5′ to 3′ direction, from left to right, using the one-letter nucleotide symbols as commonly used in the art and in accordance with the recommendations of the IUPAC-IUB Biochemical Nomenclature Commission.

The use of the word “a” or “an” when used in conjunction with the term “comprising” in the claims and/or the specification may mean “one” but it is also consistent with the meaning of “one or more”, “at least one”, and “one or more than one”.

As used in the specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps.

The term “about” is used to indicate that a value includes the standard deviation of error for the device or method being employed to determine the value. In general, the terminology “about” is meant to designate a possible variation of up to 10%. Therefore, a variation of 1, 2, 3, 4, 5, 6, 7, 8, 9 and 10% of a value is included in the term “about”.

The term “DNA” or “RNA” molecule or sequence (as well as sometimes the term “oligonucleotide”) refers to a molecule comprised generally of the deoxyribonucleotides adenine (A), guanine (G), thymine (T) and/or cytosine (C). In “RNA”, T is replaced by uracil (U).

The present description refers to a number of routinely used recombinant DNA (rDNA) technology terms. Nevertheless, definitions of selected examples of such rDNA terms are provided for clarity and consistency.

As used herein, “polynucleotide” or “nucleic acid molecule” refers to a polymer of nucleotides and includes DNA (e.g., genomic DNA, cDNA), RNA molecules (e.g., mRNA), and chimeras thereof. The nucleic acid molecule can be obtained by cloning techniques or synthesized. DNA can be double-stranded or single-stranded (coding strand or non-coding strand [antisense]). Conventional deoxyribonucleic acid (DNA) and ribonucleic acid (RNA) are included in the terms “nucleic acid molecule” and “polynucleotide” as are analogs thereof (e.g., generated using nucleotide analogs, e.g., inosine or phosphorothioate nucleotides). Such nucleotide analogs can be used, for example, to prepare polynucleotides that have altered base-pairing abilities or increased resistance to nucleases. A nucleic acid backbone may comprise a variety of linkages known in the art, including one or more of sugar-phosphodiester linkages, peptide-nucleic acid bonds (referred to as “peptide nucleic acids” (PNA); Hydig-Hielsen et al., PCT Intl Pub. No. WO 95/32305), phosphorothioate linkages, methylphosphonate linkages or combinations thereof. Sugar moieties of the nucleic acid may be ribose or deoxyribose, or similar compounds having known substitutions, e.g., 2′ methoxy substitutions (containing a 2′-O-methylribofuranosyl moiety; see PCT No. WO 98/02582) and/or 2′ halide substitutions. Nitrogenous bases may be conventional bases (A, G, C, T, U), known analogs thereof (e.g., inosine or others; see “The Biochemistry of the Nucleic Acids 5-36”, Adams et al., ed., 11th ed., 1992), or known derivatives of purine or pyrimidine bases (see, Cook, PCT Intl Pub. No. WO 93/13121) or “abasic” residues in which the backbone includes no nitrogenous base for one or more residues (Arnold et al., U.S. Pat. No. 5,585,481). A nucleic acid may comprise only conventional sugars, bases and linkages, as found in RNA and DNA, or may include both conventional components and substitutions (e.g., conventional bases linked via a methoxy backbone, or a nucleic acid including conventional bases and one or more base analogs).

An “isolated nucleic acid molecule”, as is generally understood and used herein, refers to a polymer of nucleotides, and includes, but should not limited to DNA and RNA. The “isolated” nucleic acid molecule is purified from its natural in vivo state, obtained by cloning or chemically synthesized.

As used herein, the terms “gene” and “recombinant gene” refer to nucleic acid molecules which may be isolated from chromosomal DNA, and very often include an open reading frame encoding a protein, e.g., polypeptides of the present invention. A gene may include coding sequences, non-coding sequences, introns and regulatory sequences, as well known.

“Amplification” refers to any in vitro procedure for obtaining multiple copies (“amplicons”) of a target nucleic acid sequence or its complement or fragments thereof. In vitro amplification refers to production of an amplified nucleic acid that may contain less than the complete target region sequence or its complement. In vitro amplification methods include, e.g., transcription-mediated amplification, replicase-mediated amplification, polymerase chain reaction (PCR) amplification, ligase chain reaction (LCR) amplification and strand-displacement amplification (SDA including multiple strand-displacement amplification method (MSDA)). Replicase-mediated amplification uses self-replicating RNA molecules, and a replicase such as Qβ-replicase (e.g., Kramer et al., U.S. Pat. No. 4,786,600). PCR amplification is well known and uses DNA polymerase, primers and thermal cycling to synthesize multiple copies of the two complementary strands of DNA or cDNA (e.g., Mullis et al., U.S. Pat. Nos. 4,683,195, 4,683,202, and 4,800,159). LCR amplification uses at least four separate oligonucleotides to amplify a target and its complementary strand by using multiple cycles of hybridization, ligation, and denaturation (e.g., EP Pat. App. Pub. No. 0320308). SDA is a method in which a primer contains a recognition site for a restriction endonuclease that permits the endonuclease to nick one strand of a hemimodified DNA duplex that includes the target sequence, followed by amplification in a series of primer extension and strand displacement steps (e.g., Walker et al., U.S. Pat. No. 5,422,252). Two other known strand-displacement amplification methods do not require endonuclease nicking (Dattagupta et al., U.S. Pat. No. 6,087,133 and U.S. Pat. No. 6,124,120 (MSDA)). Those skilled in the art will understand that the oligonucleotide primer sequences of the present invention may be readily used in any in vitro amplification method based on primer extension by a polymerase (e.g., see Kwoh et al., 1990, Am. Biotechnol. Lab. 8:14 25 and Kwoh et al., 1989, Proc. Natl. Acad. Sci. USA 86, 1173 1177; Lizardi et al., 1988, BioTechnology 6:1197 1202; Malek et al., 1994, Methods Mol. Biol., 28:253 260; and Sambrook et al., 2000, Molecular Cloning—A Laboratory Manual, Third Edition, CSH Laboratories). As commonly known in the art, the oligos are designed to bind to a complementary sequence under selected conditions. The terminology “amplification pair” or “primer pair” refers herein to a pair of oligonucleotides (oligos) of the present invention, which are selected to be used together in amplifying a selected nucleic acid sequence by one of a number of types of amplification processes.

As used herein, the terms “hybridizing” and “hybridizes” are intended to describe conditions for hybridization and washing under which nucleotide sequences at least about 60%, at least about 70%, at least about 80%, more preferably at least about 85%, even more preferably at least about 90%, more preferably at least 95%, more preferably at least 98% or more preferably at least 99% homologous to each other typically remain hybridized to each other. A preferred, non-limiting example of such hybridization conditions are hybridization in 6× sodium chloride/sodium citrate (SSC) at about 45° C., followed by one or more washes in 1×SSC, 0.1% SDS at 50° C., preferably at 55° C., preferably at 60° C. and even more preferably at 65° C. Highly stringent conditions include, for example, hybridizing at 68° C. in 5×SSC/5×Denhardt's solution/1.0% SDS and washing in 0.2×SSC/0.1% SDS at room temperature. Alternatively, washing may be performed at 42° C. The skilled artisan will know which conditions to apply for stringent and highly stringent hybridization conditions. Additional guidance regarding such conditions is readily available in the art, for example, in Sambrook et al., supra; and Ausubel et al., supra (eds.), 1995, Current Protocols in Molecular Biology, (John Wiley & Sons, N.Y.). Of course, a polynucleotide which hybridizes only to a poly (A) sequence (such as the 3′ terminal poly(A) tract of mRNAs), or to a complementary stretch of T (or U) residues, would not be included in a polynucleotide of the invention used to specifically hybridize to a portion of a nucleic acid of the invention, since such a polynucleotide would hybridize to any nucleic acid molecule containing a poly (A) stretch or the complement thereof (e.g., practically any double-stranded cDNA clone).

The terms “identity” and “percent identity” are used interchangeably herein. For the purpose of this invention, it is defined here that in order to determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). Preferably, the two sequences are the same length. Thus, In accordance with the present invention, the term “identical” or “percent identity” in the context of two or more nucleic acid or amino acid sequences, refers to two or more sequences or subsequences that are the same, or that have a specified percentage of amino acid residues or nucleotides that are the same (e.g., 60% or 65% identity, preferably, 70-95% identity, more preferably at least 95% identity), when compared and aligned for maximum correspondence over a window of comparison, or over a designated region as measured using a sequence comparison algorithm as known in the art, or by manual alignment and visual inspection. Sequences having, for example, 60% to 95% or greater sequence identity are considered to be substantially identical. Such a definition also applies to the complement of a test sequence. Preferably, the described identity exists over a region that is at least about 15 to 25 amino acids or nucleotides in length, more preferably, over a region that is about 50 to 100 amino acids or nucleotides in length. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art. Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST and BLAST 2.0 algorithms (Altschul Nucl. Acids Res. 25 (1977), 3389-3402). The BLASTN program for nucleic acid sequences uses as defaults a word length (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff Proc. Natl. Acad. Sci., USA, 89, (1989), 10915) uses alignments (B) of 50, expectation (E) of 10, M=5, N=4, and a comparison of both strands. Moreover, the present invention also relates to nucleic acid molecules the sequence of which is degenerate in comparison with the sequence of an above-described hybridizing molecule. When used in accordance with the present invention the term “being degenerate as a result of the genetic code” means that due to the redundancy of the genetic code different nucleotide sequences code for the same amino acid. The present invention also relates to nucleic acid molecules which comprise one or more mutations or deletions, and to nucleic acid molecules which hybridize to one of the herein described nucleic acid molecules, which show (a) mutation(s) or (a) deletion(s). The skilled person will appreciate that all these different algorithms or programs will yield slightly different results but that the overall percentage identity of two sequences is not significantly altered when using different algorithms.

In a related manner, the terms “homology” or “percent homology”, refer to a similarity between two polypeptide sequences, but take into account changes between amino acids (whether conservative or not). As well known in the art, amino acids can be classified by charge, hydrophobicity, size, etc. It is also well known in the art that amino acid changes can be conservative (e.g., they do not significantly affect, or not at all, the function of the protein). A multitude of conservative changes are known in the art, Serine for threonine, isoleucine for leucine, arginine for lysine etc., Thus the term homology introduces evolutionistic notions (e.g., pressure from evolution to a retain function of essential or important regions of a sequence, while enabling a certain drift of less important regions).

The skilled person will be aware of the fact that several different computer programs are available to determine the homology between two sequences. For instance, a comparison of sequences and determination of percent identity between two sequences can be accomplished using a mathematical algorithm. In a preferred embodiment, the percent identity between two amino acid sequences is determined using the Needleman and Wunsch (J. Mol. Biol. (48): 444-453 (1970)) algorithm which has been incorporated into the GAP program in the Accelrys GCG software package (available at http://www.accelrys.com/products/gcg/), using either a Blossom 62 matrix or a PAM250 matrix, and a gap weight of 16, 14, 12, 10, 8, 6, or 4 and a length weight of 1, 2, 3, 4, 5, or 6. The skilled person will appreciate that all these different parameters will yield slightly different results but that the overall percentage identity of two sequences is not significantly altered when using different algorithms.

In yet another embodiment, the percent identity between two nucleotide sequences is determined using the GAP program in the Accelrys GCG software package (available at http://www.accelrys.com/products/gcg/), using a NWSgapdna.CMP matrix and a gap weight of 40, 50, 60, 70, or 80 and a length weight of 1, 2, 3, 4, 5, or 6. In another embodiment, the percent identity two amino acid or nucleotide sequence is determined using the algorithm of E. Meyers and W. Miller (CABIOS, 4:11-17 (1989) which has been incorporated into the ALIGN program (version 2.0) (available at the ALIGN Query using sequence data of the Genestream server IGH Montpellier France http://vega.igh.cnrs.fr/bin/align-guess.cgi) using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul et al., (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., XBLAST and NBLAST) can be used. See the homepage of the National Center for Biotechnology Information at http://www.ncbi.nlm.nih.qov/.

By “sufficiently complementary” is meant a contiguous nucleic acid base sequence that is capable of hybridizing to another sequence by hydrogen bonding between a series of complementary bases. Complementary base sequences may be complementary at each position in sequence by using standard base pairing (e.g., G:C, A:T or A:U pairing) or may contain one or more residues (including abasic residues) that are not complementary by using standard base pairing, but which allow the entire sequence to specifically hybridize with another base sequence in appropriate hybridization conditions. Contiguous bases of an oligomer are preferably at least about 80% (81, 82, 83, 84, 85, 86, 87, 88, 89, 90, 91, 92, 93, 94, 95, 96, 97, 98, 99, 100%), more preferably at least about 90% complementary to the sequence to which the oligomer specifically hybridizes. Appropriate hybridization conditions are well known to those skilled in the art, can be predicted readily based on sequence composition and conditions, or can be determined empirically by using routine testing (see Sambrook et al, Molecular Cloning, A Laboratory Manual, 2^{nd} ed. (Cold Spring Harbor Laboratory Press, Cold Spring Harbor, N.Y., 1989) at §§1.90-1.91, 7.37-7.57, 9.47-9.51 and 11.47-11.57, particularly at §§9.50-9.51, 11.12-11.13, 11.45-11.47 and 11.55-11.57).

The present invention refers to a number of units or percentages that are often listed in sequences. For example, when referring to “at least 80%, at least 85%, at least 90% . . . ”, or “at least about 80%, at least about 85%, at least about 90% . . . ”, every single unit is not listed, for the sake of brevity. For example, some units (e.g., 81, 82, 83, 84, 85, . . . 91, 92% . . . ) may not have been specifically recited but are considered encompassed by the present invention. The non-listing of such specific units should thus be considered as within the scope of the present invention.

Nucleic acid sequences may be detected by using hybridization with a complementary sequence (e.g., oligonucleotide probes) (see U.S. Pat. No. 5,503,980 (Cantor), U.S. Pat. No. 5,202,231 (Drmanac et al.), U.S. Pat. No. 5,149,625 (Church et al.), U.S. Pat. No. 5,112,736 (Caldwell et al.), U.S. Pat. No. 5,068,176 (Vijg et al.), and U.S. Pat. No. 5,002,867 (Macevicz)). Hybridization detection methods may use an array of probes (e.g., on a DNA chip) to provide sequence information about the target nucleic acid which selectively hybridizes to an exactly complementary probe sequence in a set of four related probe sequences that differ one nucleotide (see U.S. Pat. Nos. 5,837,832 and 5,861,242 (Chee et al.)).

A detection step may use any of a variety of known methods to detect the presence of nucleic acid by hybridization to an oligonucleotide probe. The types of detection methods in which probes can be used include Southern blots (DNA detection), dot or slot blots (DNA, RNA), and Northern blots (RNA detection). Labeled proteins could also be used to detect a particular nucleic acid sequence to which it binds (e.g., protein detection by far western technology: Guichet et al., 1997, Nature 385(6616): 548-552; and Schwartz et al., 2001, EMBO 20(3): 510-519). Other detection methods include kits containing reagents of the present invention on a dipstick setup and the like. Of course, it might be preferable to use a detection method which is amenable to automation. A non-limiting example thereof includes a chip or other support comprising one or more (e.g., an array) of different probes.

A “label” refers to a molecular moiety or compound that can be detected or can lead to a detectable signal. A label is joined, directly or indirectly, to a nucleic acid probe or the nucleic acid to be detected (e.g., an amplified sequence) or to a polypeptide to be detected. Direct labeling can occur through bonds or interactions that link the label to the polynucleotide or polypeptide (e.g., covalent bonds or non-covalent interactions), whereas indirect labeling can occur through the use of a “linker” or bridging moiety, such as additional nucleotides, amino acids or other chemical groups, which are either directly or indirectly labeled. Bridging moieties may amplify a detectable signal. Labels can include any detectable moiety (e.g., a radionuclide, ligand such as biotin or avidin, enzyme or enzyme substrate, reactive group, chromophore such as a dye or colored particle, luminescent compound including a bioluminescent, phosphorescent or chemiluminescent compound, and fluorescent compound).

As used herein, “expression” is meant the process by which a gene or otherwise nucleic acid sequence eventually produces a polypeptide. It involves transcription of the gene into mRNA, and the translation of such mRNA into polypeptide(s).

The terms “peptide” and “oligopeptide” are considered synonymous (as is commonly recognized) and each term can be used interchangeably as the context required to indicate a chain of at least two amino acids coupled by peptidyl linkages. The word “polypeptide” is used herein for chains containing more than seven amino acid residues. All oligopeptide and polypeptide formulas or sequences herein are written from left to right and in the direction from amino terminus to carboxyl terminus. The one-letter code of amino acids used herein is commonly known in the art and can be found in Sambrook, et al., supra. Sequence Listings programs can convert easily this one-letter code of amino acids sequence into a three-letter code.

The phrase “mature polypeptide” is defined herein as a polypeptide having biological activity a polypeptide of the present invention that is in its final form, following translation and any post-translational modifications, such as N-terminal processing, C-terminal truncation, removal of signal sequences, glycosylation, phosphorylation, etc. In one embodiment, polypeptides of the present invention comprise mature of polypeptides of any one of the polypeptides disclosed herein. Mature polypeptides of the present invention can be predicted using programs such as SignalP. The phrase “mature polypeptide coding sequence” is defined herein as a nucleotide sequence that encodes a mature polypeptide as defined above. As well known, some nucleotide sequences are non-coding.

As used herein, the term “purified” or “isolated” refers to a molecule (e.g., polynucleotide or polypeptide) having been separated from a component of the composition in which it was originally present. Thus, for example, an “isolated polynucleotide” or “isolated polypeptide” has been purified to a level not found in nature. A “substantially pure” molecule is a molecule that is lacking in most other components (e.g., 30, 40, 50, 60, 70, 75, 80, 85, 90, 95, 96, 97, 98, 99, 100% free of contaminants). By opposition, the term “crude” means molecules that have not been separated from the components of the original composition in which it was present. For the sake of brevity, the units (e.g., 66, 67 . . . 81, 82, 83, 84, 85, . . . 91, 92% . . . ) have not been specifically recited but are considered nevertheless within the scope of the present invention.

An “isolated polynucleotide” or “isolated nucleic acid molecule” is a nucleic acid molecule (DNA or RNA) that is not immediately contiguous with both of the coding sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter) sequences that are immediately contiguous to the coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment) independent of other sequences. It also includes a recombinant DNA that is part of a hybrid gene encoding an additional polypeptide that is substantially free of cellular material, viral material, or culture medium (when produced by recombinant DNA techniques), or chemical precursors or other chemicals (when chemically synthesized). Moreover, an “isolated nucleic acid fragment” is a nucleic acid fragment that is not naturally occurring as a fragment and would not be found in the natural state.

As used herein, an “isolated polypeptide” or “isolated protein” is intended to include a polypeptide or protein removed from its native environment. For example, recombinantly produced polypeptides and proteins expressed in host cells are considered isolated for the purpose of the invention, as are native or recombinant polypeptides which have been substantially purified by any suitable technique such as, for example, the single-step purification method disclosed in Smith and Johnson, Gene 67:31-40 (1988).

The term “variant” refers herein to a polypeptide, which is substantially similar in structure (e.g., amino acid sequence) to a polypeptide disclosed herein or encoded by a nucleic acid sequence disclosed herein without being identical thereto. Thus, two molecules can be considered as variants even though their primary, secondary, tertiary or quaternary structures are not identical. A variant can comprise an insertion, substitution, or deletion of one or more amino acids as compared to its corresponding native protein. A variant can comprise additional modifications (e.g., post-translational modifications such as acetylation, phosphorylation, glycosylation, sulfatation, sumoylation, prenylation, ubiquitination, etc). As used herein, the term “functional variant” is intended to include a variant which is sufficiently similar in both structure and function to a polypeptide disclosed herein or encoded by a nucleic acid sequence disclosed herein, to maintain at least one of its native biological activities.

As used herein, the term “biomass” refers to any cellulosic or lignocellulosic material and includes materials comprising cellulose, and optionally further comprising hemicellulose, lignin, starch, oligosaccharides and/or monosaccharides. Biomass may also comprise additional components, such as protein and/or lipid. Biomass may be derived from a single source, or biomass can comprise a mixture derived from more than one source; for example, biomass could comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste or a combination thereof. Examples of biomass include, but are not limited to, corn grain, corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers, and animal manure or a combination thereof. Biomass that is useful for the invention may include biomass that has a relatively high carbohydrate value, is relatively dense, and/or is relatively easy to collect, transport, store and/or handle. In one embodiment of the present invention, biomass that is useful includes corn cobs, corn stover, sawdust, and sugar cane bagasse.

As used herein, the terms “cellulosic” or “cellulose-containing material” refers to a composition comprising cellulose. As used herein, the term “lignocellulosic” refers to a composition comprising both lignin and cellulose. Lignocellulosic material may also comprise hemicellulose. The predominant polysaccharide in the primary cell wall of biomass is cellulose, the second most abundant is hemi-cellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Cellulose is generally found, for example, in the stems, leaves, hulls, husks, and cobs of plants or leaves, branches, and wood of trees. The cellulose-containing material can be, but is not limited to, herbaceous material, agricultural residues, forestry residues, municipal solid wastes, waste paper, and pulp and paper mill residues. The cellulose-containing material can be any type of biomass including, but not limited to, wood resources, municipal solid waste, wastepaper, crops, and crop residues (e.g., see Wiselogel et al., 1995, in Handbook on Bioethanol (Charles E. Wyman, editor), pp. 105-118, Taylor & Francis, Washington D.C.; Wyman. 1994. Bioresource Technology 50: 3-16; Lynd. 1990. Applied Biochemistry and Biotechnology 24/25: 695-719; Mosier et al., 1999, Recent Progress in Bioconversion of Lignocellulosics, in Advances in Biochemical Engineering/Biotechnology, T. Scheper, managing editor, Volume 65. pp. 23-40. Springer-Verlag, New York). It is understood herein that the cellulose may be in the form of lignocellulose, a plant cell wall material containing lignin, cellulose, and hemicellulose in a mixed matrix.

The phrase “cellulolytic enhancing activity” is defined herein as a biological activity which enhances the hydrolysis of a cellulose-containing material by proteins having cellulolytic activity. The term “cellulolytic activity” is defined herein as a biological activity which hydrolyzes a cellulose-containing material.

The term “thermostable”, as used herein, refers to an enzyme that retains its function or protein activity at a temperature greater than 50° C.; thus, a thermostable cellulose-degrading or cellulase-enhacing enzyme/protein retains the ability to degrade or enhance the degradation of cellulose at this elevated temperature. A protein or enzyme may have more than one enzymatic activity. For example, some polypeptide of the present invention exhibit bifunctional activities such as xylosidase/arabinosidase activity. Such bifunctional enzymes may exhibit thermostability with regard to one activity, but not another, and still be considered as “thermostable”.

### BRIEF DESCRIPTION OF DRAWINGS

In the appended drawings:

FIG. 1 is a schematic map of the pGBFIN-49 expression plasmid.

FIG. 2 shows the endoxylanase activity of various secreted proteins from Scytalidium thermophilum (panel A), Myriococcum thermophilum (panel B), and Aureobasidium pullulans (panel C).

FIG. 3 shows the xyloglucanase activity of two secreted proteins from Aureobasidium pullulans on Tamarind xyloglucan.

FIGS. 4 and 5 show enzyme activity-temperature profiles of various secreted proteins from Scytalidium thermophilum.

FIGS. 6-11 show enzyme activity-temperature profiles of various secreted proteins from Myriococcum thermophilum.

FIGS. 12-16 show enzyme activity-temperature profiles of various secreted proteins from Aureobasidium pullulans.

In the appended Sequence Listing, SEQ ID NOs: 1-855 relate to sequences from Scytalidium thermophilum; SEQ ID NOs: 856-1773 relate to sequences from Myriococcum thermophilum; and SEQ ID NOs: 1774-2934 relate to sequences from Aureobasidium pullulans.

### DESCRIPTION OF ILLUSTRATIVE EMBODIMENTS

### Polypeptides of the Invention

In one aspect, the present invention relates to isolated polypeptides secreted by Scytalidium thermophilum, Myriococcum thermophilum, or Aureobasidium pullulans, (e.g., Scytalidium thermophilum strain CBS 625.91, Myriococcum thermophilum strain CBS 389.93, or Aureobasidium pullulans strain ATCC 62921) having an activity relating to the processing or degradation of biomass (e.g., cell wall deconstruction).

In another aspect, the present invention relates to isolated polypeptides comprising the amino acid sequences shown in any one of SEQ ID NOs: 571-855, 1468-1773, or 2548-2934.

In another aspect, the present invention relates to isolated polypeptides sharing a minimum threshold of amino acid sequence identity with any one of the above-mentioned polypeptides. In specific embodiments, the present invention relates to isolated polypeptides having at least 60%, 65%, 70%, 71%, 72, 73%, 74%, 75%, 76%, 77%, 78%, 79, 80%, 81%, 82%, 83%, 84%, 85%, 86%, 87%, 88%, 89%, 90%, 91%, 92%, 93%, 94%, 95%, 96%, 97%, 98% or 99% amino acid sequence identity to any one of the above-mentioned polypeptides. Other specific percentage units that have not been specifically recited here for brevity are nevertheless considered within the scope of the present invention.

In another aspect, the present invention relates to a polypeptide encoded by a polynucleotide of the present invention, which includes genomic (e.g., SEQ ID NOs: 1-285, 856-1161, or 1774-2160), and coding (e.g., SEQ ID NOs: 286-570, 1162-1467, or 2161-2547) nucleic acid sequences disclosed herein, polynucleotides hybridizing under medium-high, high, or very high stringency conditions with a full-length complement thereof, as well as polynucleotides sharing a certain degree of nucleic acid sequence identity therewith.

In another aspect, the present invention relates to a polypeptide comprising an amino acid sequence encoded by at least one exonic nucleic acid sequence of any one of the genomic sequences corresponding to SEQ ID NOs: 1-285, 856-1161, or 1774-2160 (e.g., the intron or exon segments defined by the exon boundaries listed in Tables 2A-2C) or a functional part thereof.

In another aspect, the present invention relates to functional variants of any one of the above-mentioned polypeptides. In another embodiment, the term “functional” or “biologically active” relates to the native enzymatic (e.g., catalytic) activity of a polypeptide of the present invention. In some embodiments, the present invention relates to a polypeptide comprising a biological activity of any one of the enzymes described below, or a polynucleotide encoding same.

“Carbohydrase” refers to any protein that catalyzes the hydrolysis of carbohydrates. “Glycoside hydrolase”, “glycosyl hydrolase” or “glycosidase” refers to a protein that catalyzes the hydrolysis of the glycosidic bonds between carbohydrates or between a carbohydrate and a non-carbohydrate residue. Endoglucanases, cellobiohydrolases, beta-glucosidases, a-glucosidases, xylanases, beta-xylosidases, alpha-xylosidases, galactanases, a-galactosidases, beta-galactosidases, a-amylases, glucoamylases, endo-arabinases, arabinofuranosidases, mannanases, beta-mannosidases, pectinases, acetyl xylan esterases, acetyl mannan esterases, femlic acid esterases, coumaric acid esterases, pectin methyl esterases, and chitosanases are examples of glycosidases.

“Cellulase” refers to a protein that catalyzes the hydrolysis of 1,4-D-glycosidic linkages in cellulose (such as bacterial cellulose, cotton, filter paper, phosphoric acid swollen cellulose, Avicel®); cellulose derivatives (such as carboxymethylcellulose and hydroxyethylcellulose); plant lignocellulosic materials, beta-D-glucans or xyloglucans. Cellulose is a linear beta-(1-4) glucan consisting of anhydrocellobiose units. Endoglucanases, cellobiohydrolases, and beta-glucosidases are examples of cellulases.

“Endoglucanase” refers to a protein that catalyzes the hydrolysis of cellulose to oligosaccharide chains at random locations by means of an endoglucanase activity.

“Cellobiohydrolase” refers to a protein that catalyzes the hydrolysis of cellulose to cellobiose via an exoglucanase activity, sequentially releasing molecules of cellobiose from the reducing or non-reducing ends of cellulose or cello-oligosaccharides. “beta-glucosidase” refers to an enzyme that catalyzes the conversion of cellobiose and oligosaccharides to glucose.

“Hemicellulase” refers to a protein that catalyzes the hydrolysis of hemicellulose, such as that found in lignocellulosic materials. Hemicelluloses are complex polymers, and their composition often varies widely from organism to organism, and from one tissue type to another. Hemicelluloses include a variety of compounds, such as xylans, arabinoxylans, xyloglucans, mamians, glucomannans, and galacto(gluco)mannans. Hemicellulose can also contain glucan, which is a general term for beta-linked glucose residues. In general, a main component of hemicellulose is beta-1,4-linked xylose, a five carbon sugar. However, this xylose is often branched as beta-1,3 linkages or beta-1,2 linkages, and can be substituted with linkages to arabinose, galactose, mannose, glucuronic acid, or by esterification to acetic acid. Hemicellulolytic enzymes, i.e., hemicellulases, include both endo-acting and exo-acting enzymes, such as xylanases, beta-xylosidases. alpha-xylosidases, galactanases, a-galactosidases, beta-galactosidases, endo-arabinases, arabinofuranosidases, mannanases, and beta-mannosidases. Hemicellulases also include the accessory enzymes, such as acetylesterases, ferulic acid esterases, and coumaric acid esterases. Among these, xylanases and acetyl xylan esterases cleave the xylan and acetyl side chains of xylan and the remaining xylo-oligomers are unsubstituted and can thus be hydrolysed with beta-xylosidase only. In addition, several less known side activities have been found in enzyme preparations which hydrolyze hemicellulose. Accordingly, xylanases, acetylesterases and beta-xylosidases are examples of hemicellulases.

“Xylanase” specifically refers to an enzyme that hydrolyzes the beta-1,4 bond in the xylan backbone, producing short xylooligosaccharides.

“Beta-mannanase” or “endo-1,4-beta-mannosidase” refers to a protein that hydrolyzes mannan-based hemicelluloses (mannan, glucomannan, galacto(gluco)mannan) and produces short beta-1,4-mannooligosaccharides.

“Mannan endo-1,6-alpha-mannosidase” refers to a protein that hydrolyzes 1,6-alpha-mannosidic linkages in unbranched 1,6-mannans.

“Beta-mannosidase” (beta-1,4-mannoside mannohydrolase; EC 3.2.1.25) refers to a protein that catalyzes the removal of beta-D-mannose residues from the non-reducing ends of oligosaccharides.

“Galactanase”, “endo-beta-1,6-galactanse” or “arabinogalactan endo-1,4-beta-galactosidase” refers to a protein that catalyzes the hydrolysis of endo-1,4-beta-D-galactosidic linkages in arabinogalactans.

“Glucoamylase” refers to a protein that catalyzes the hydrolysis of terminal 1,4-linked-D-glucose residues successively from non-reducing ends of the glycosyl chains in starch with the release of beta-D-glucose.

“Beta-hexosaminidase” or “beta-N-acetylglucosaminidase” refers to a protein that catalyzes the hydrolysis of terminal N-acetyl-D-hexosamine residues in N-acetyl-beta-D-hexosamines.

“Alpha-L-arabinofuranosidase”, “alpha-N-arabmofuranosidase”, “alpha-arabinofuranosidase”, “arabinosidase” or “arabinofuranosidase” refers to a protein that hydrolyzes arabinofuranosyl-containing hemicelluloses or pectins. Some of these enzymes remove arabinofuranoside residues from 0-2 or 0-3 single substituted xylose residues, as well as from 0-2 and/or 0-3 double substituted xylose residues. Some of these enzymes remove arabinose residues from arabinan oligomers.

“Endo-arabinase” refers to a protein that catalyzes the hydrolysis of 1,5-alpha-arabinofuranosidic linkages in 1,5-arabinans.

“Exo-arabinase” refers to a protein that catalyzes the hydrolysis of 1,5-alpha-linkages in 1,5-arabinans or 1,5-alpha-L arabino-oligosaccharides, releasing mainly arabinobiose, although a small amount of arabinotriose can also be liberated.

“Beta-xylosidase” refers to a protein that hydrolyzes short 1,4-beta-D-xylooligomers into xylose.

“Cellobiose dehydrogenase” refers to a protein that oxidizes cellobiose to cellobionolactone.

“Chitosanase” refers to a protein that catalyzes the endohydrolysis of beta-1,4-linkages between D-glucosamine residues in acetylated chitosan (i.e., deacetylated chitin).

“Exo-polygalacturonase” refers to a protein that catalyzes the hydrolysis of terminal alpha 1,4-linked galacturonic acid residues from non-reducing ends thus converting polygalacturonides to galacturonic acid.

“Acetyl xylan esterase” refers to a protein that catalyzes the removal of the acetyl groups from xylose residues. “Acetyl mannan esterase” refers to a protein that catalyzes the removal of the acetyl groups from mannose residues, “ferulic esterase” or “ferulic acid esterase” refers to a protein that hydrolyzes the ester bond between the arabinose substituent group and ferulic acid. “Coumaric acid esterase” refers to a protein that hydrolyzes the ester bond between the arabinose substituent group and coumaric acid. Acetyl xylan esterases, ferulic acid esterases and pectin methyl esterases are examples of carbohydrate esterases.

“Pectate lyase” and “pectin lyases” refer to proteins that catalyze the cleavage of 1,4-alpha-D-galacturonan by beta-elimination acting on polymeric and/or oligosaccharide substrates (pectates and pectins, respectively).

“Endo-1,3-beta-glucanase” or “laminarinase” refers to a protein that catalyzes the cleavage of 1,3-linkages in beta-D-glucans such as laminarin or lichenin. Laminarin is a linear polysaccharide made up of beta-1,3-glucan with beta-1,6-linkages.

“Lichenase” refers to a protein that catalyzes the hydrolysis of lichenan, a linear, 1,3-1,4-beta-D glucan.

Rhamnogalacturonan is composed of alternating alpha-1,4-rhamnose and alpha-1,2-linked galacturonic acid, with side chains linked 1,4 to rhamnose. The side chains include Type I galactan, which is beta-1,4-linked galactose with alpha-1,3-linked arabinose substituents; Type II galactan, which is beta-1,3-1,6-linked galactoses (very branched) with arabinose substituents; and arabinan, which is alpha-1,5-linked arabinose with alpha-1,3-linked arabinose branches. The galacturonic acid substituents may be acetylated and/or methylated.

“Exo-rhamnogalacturonanase” refers to a protein that catalyzes the degradation of the rhamnogalacturonan backbone of pectin from the non-reducing end.

“Rhamnogalacturonan acetylesterase” refers to a protein that catalyzes the removal of the acetyl groups ester-linked to the highly branched rhamnogalacturonan (hairy) regions of pectin.

“Rhamnogalacturonan lyase” refers to a protein that catalyzes the degradation of the rhamnogalacturonan backbone of pectin via a beta-elimination mechanism (e.g., see Pages et al., J. Bacteria, 185:4727-4733 (2003)).

“Alpha-rhamnosidase” refers to a protein that catalyzes the hydrolysis of terminal non-reducing alpha-L-rhamnose residues in alpha-L-rhamnosides.

Certain proteins of the present invention may be classified as “Family 61 glycosidases” based on homology of the polypeptides to CAZy Family GH61. Family 61 glycosidases may exhibit cellulolytic enhancing activity or endoglucanase activity. Additional information on the properties of Family 61 glycosidases may be found in U.S. Patent Application Publication Nos. 2005/0191736, 2006/0005279, 2007/0077630, and in PCT Publication No. WO 2004/031378.

“Esterases” represent a category of various enzymes including lipases, phospholipases, cutinases, and phytases that catalyze the hydrolysis and synthesis of ester bonds in compounds.

The International Union of Biochemistry and Molecular Biology have developed a nomenclature for enzymes where each enzyme is described by a sequence of four numbers preceded by “EC”. The first number broadly classifies the enzyme based on its mechanism. According to the naming conventions, enzymes are generally classified into six main family classes and many sub-family classes: EC 1 Oxidoreductases: catalyze oxidation/reduction reactions; EC 2 Transferases: transfer a functional group (e.g. a methyl or phosphate group); EC 3 Hydrolases: catalyze the hydrolysis of various bonds; EC 4 Lyases: cleave various bonds by means other than hydrolysis and oxidation; EC 5 Isomerases: catalyze isomerization changes within a single molecule; and EC 6 Ligases: join two molecules with covalent bonds. A number of bioinformatic tools are available to the skilled person to predict which main family class and sub-family class an enzyme molecule belongs to according to its sequence information. In some instances, certain enzymes (or family of enzymes) can be re-classified, for example, to take into account newly discovered enzyme functions or properties. Accordingly, the polypeptides/enzymes of the present invention are not meant to be limited to specific enzyme classes as they currently exist. The skilled person would know how to appropriately reclassify (and assign the appropriate functions) to the enzymes of the present invention based on the amino acid sequence information provided herein. Such reclassifications are thus within the scope of the present invention.

In some embodiments, the present invention relates to a polypeptide comprising a biological activity of any one of the enzymes (or sub-classes thereof), or a polynucleotide encoding same.
Cellulose-hydrolyzing enzymes, including: endoglucanases (EC 3.2.1.4), which hydrolyze the beta-1,4-linkages between glucose units; exoglucanases (also known as cellobiohydrolases 1 and 2) (EC 3.2.1.91), which hydrolyze cellobiose, a glucose disaccharide, from the reducing and non-reducing ends of cellulose; and beta-glucosidases (EC 3.2.1.21), which hydrolyze the beta-1,4 glycoside bond of cellobiose to glucose;
Proteins that enhance or accelerate the action of cellulose-degrading enzymes, including: glycoside hydrolase family 61 (GH61) proteins (e.g., polysaccharide monooxygenases), which enhance the action of cellulose enzymes on lignocellulose substrates;
Enzymes that degrade or modify xylan and/or xylan-lignin complexes, including: xylanases, such as endo-1,4-beta-xylanase (EC 3.2.1.8), which catalyze the endohydrolysis of 1-4-beta-D-xylosidic linkages in xylans (or xyloglucans); xylosidases, such as xylan 1,4-beta-xylosidases (EC 3.2.1.37), which catalyze hydrolysis of 1,4-beta-D-xylans to remove successive D-xylose residues from the non-reducing terminals, and also cleaves xylobiose; arabinosidases, such as alpha-arabinofuranosidases (EC 3.2.1.55), which hydrolyze terminal non-reducing alpha-L-arabinofuranoside residues in alpha-L-arabinosides (including arabinoxylans and arabinogalactans); alpha-glucuronidases (EC 3.2.1.139), which hydrolyze an alpha-D-glucuronoside to the corresponding alcohol and D-glucuronate; feruloyl esterases (EC 3.1.1.73), which catalyzes hydrolysis of the 4-hydroxy-3-methoxycinnamoyl (feruloyl) group from an esterified sugar (which is usually arabinose in natural substrates); and acetylxylan esterases (EC 3.1.1.72), which catalyze deacetylation of xylans and xylo-oligosaccharides;
Enzymes that degrade or modify mannan, including: mannanases, such as mannan endo-1,4-beta-mannosidase (EC 3.2.1.78), which catalyze random hydrolysis of 1,4-beta-D-mannosidic linkages in mannans, galactomannans and glucomannans;
mannosidases (EC 3.2.1.25), which hydrolyze terminal, non-reducing beta-D-mannose residues in beta-D-mannosides; alpha-galactosidases (EC 3.2.1.22), which hydrolyzes terminal, non-reducing alpha-D-galactose residues in alpha-D-galactosides (including galactose oligosaccharides, galactomannans and galactohydrolase); and mannan acetyl esterases;
Enzymes that degrade or modify xyloglucans, including: xyloglucanases such as xyloglucan-specific endo-beta-1,4-glucanase (EC 3.2.1.151), which involves endohydrolysis of 1,4-beta-D-glucosidic linkages in xyloglucan; and xyloglucan-specific exo-beta-1,4-glucanase (EC 3.2.1.155), which catalyzes exohydrolysis of 1,4-beta-D-glucosidic linkages in xyloglucan; endoglucanases/cellulases;
Enzymes that degrade or modify glucans, including: Enzymes that degrade beta-1,4-glucan, such as endoglucanases; cellobiohydrolases; and beta-glucosidases;
Enzymes that degrade beta-1,3-1,4-glucan, such as endo-beta-1,3(4)-glucanases (EC 3.2.1.6), which catalyzes endohydrolysis of 1,3- or 1,4-linkages in beta-D-glucans when the glucose residue whose reducing group is involved in the linkage to be hydrolyzed is itself substituted at C-3; endoglucanases (beta-glucanase, cellulase), and beta-glucosidases;
Enzymes that degrade or modify galactans, including: galactanases (EC 3.2.1.23), which hydrolyze terminal non-reducing beta-D-galactose residues in beta-D-galactosides;
Enzymes that degrade or modify arabinans, including: arabinanases (EC 3.2.1.99), which catalyze endohydrolysis of 1,5-alpha-arabinofuranosidic linkages in 1,5-arabinans;
Enzymes that degrade or modify starch, including: amylases, such as alpha-amylases (EC 3.2.1.1), which catalyze endohydrolysis of 1,4-alpha-D-glucosidic linkages in polysaccharides containing three or more 1,4-alpha-linked D-glucose units; and glucosidases, such as alpha-glucosidases (EC 3.2.1.20), which hydrolyze terminal, non-reducing 1,4-linked alpha-D-glucose residues with release of alpha-D-glucose;
Enzymes that degrade or modify pectin, including: pectate lyases (EC 4.2.2.2), which carry out eliminative cleavage of pectate to give oligosaccharides with 4-deoxy-alpha-D-gluc-4-enuronosyl groups at their non-reducing ends; pectin lyases (EC 4.2.2.10), which catalyze eliminative cleavage of (1-4)-alpha-D-galacturonan methyl ester to give oligosaccharides with 4-deoxy-6-O-methyl-alpha-D-galact-4-enuronosyl groups at their non-reducing ends; polygalacturonases (EC 3.2.1.15), which carry out random hydrolysis of 1,4-alpha-D-galactosiduronic linkages in pectate and other galacturonans; pectin esterases, such as pectin acetyl esterase (EC 3.1.1.11), which hydrolyzes acetate from pectin acetyl esters; alpha-arabi nofuranosidases; beta-galactosidases; galactanases; arabinanases; rhamnogalacturonases (EC 3.2.1.-), which hydrolyze alpha-D-galacturonopyranosyl-(1,2)-alpha-L-rhamnopyranosyl linkages in the backbone of the hairy regions of pectins; rhamnogalacturonan lyases (EC 4.2.2.-), which degrade type I rhamnogalacturonan from plant cell walls and releases disaccharide products; rhamnogalacturonan acetyl esterases (EC 3.1.1.-), which hydrolyze acetate from rhamnogalacturonan; and xylogalacturonosidases and xylogalacturonases (EC 3.2.1.-), which hydrolyze xylogalacturonan (xga), a galacturonan backbone heavily substituted with xylose, and which is one important component of the hairy regions of pectin;
Enzymes that degrade or modify lignin, including: lignin peroxidases (EC 1.11.1.14), which oxidize lignin and lignin model compounds using hydrogen peroxide; manganese-dependent peroxidases (EC 1.11.1.13), which oxidizes lignin and lignin model compounds using Mn²⁺ and hydrogen peroxide; versatile peroxidases (EC 1.11.1.16), which oxidize lignin and lignin model compounds using an electron donor and hydrogen peroxide and combines the substrate-specificity characteristics of the two other ligninolytic peroxidases: manganese peroxidase (EC 1.11.1.13) and lignin peroxidase (EC 1.11.1.14); and laccases (EC 1.10.3.2), a group of multi-copper proteins of low specificity acting on both o- and p-quinols, and often acting also on lignin; and
Enzymes acting on chitin, including: chitinases (EC 3.2.1.14), which catalyze random hydrolysis of N-acetyl-beta-D-glucosaminide 1,4-beta-linkages in chitin and chitodextrins; and hexosaminidases, such as beta-N-acetylhexosaminidase (EC 3.2.1.52), which hydrolyzes terminal non-reducing N-acetyl-D-hexosamine residues in N-acetyl-beta-D-hexosaminides.

In another embodiment, the present invention includes the polypeptides and their corresponding activities as defined in Tables 1A-1C, as well as functional variants thereof.

As alluded to above, the term “functional variant” as used herein is intended to include a polypeptide which is sufficiently similar in structure and function to any one of the above-mentioned polypeptides (without being identical thereto) to maintain at least one of its native biological activities. In another embodiment, a functional variant can comprise an insertion, substitution, or deletion of one or more amino acids as compared to its corresponding native protein. In another embodiment, a functional variant can comprise additional modifications (e.g., post-translational modifications such as acetylation, phosphorylation, glycosylation, sulfatation, sumoylation, prenylation, ubiquitination, etc).

In another embodiment, functional variants of the present invention can contain one or more conservative substitutions of a polypeptide sequence disclosed herein. Such modifications can be carried out routinely using site-specific mutagenesis. The term “conservative substitution” is intended to indicate a substitution in which the amino acid residue is replaced with an amino acid residue having a similar side chain. Families of amino acids having similar side chains are known in the art and include amino acids with basic side chains (e.g., lysine, arginine and hystidine), acidic side chains (e.g., aspartic acid, glutamic acid), uncharged polar side chains (e.g., glycine, asparagines, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine tryptophan, histidine).

In another embodiment, functional variants of the present invention can contain one or more insertions, deletions or truncations of non-essential amino acids. As used herein, a “non-essential amino acid” is a residue that can be altered in a polypeptide of the present invention without substantially altering its (biological) function or protein activity. For example, amino acid residues that are conserved among the proteins of the present invention having similar biological activities (and their orthologs) are predicted to be particularly unamenable to alteration.

In another embodiment, functional variants can include functional fragments (i.e., biologically active fragments) of any one of the polypeptide sequences disclosed herein. Such fragments include fewer amino acids than the full length protein from which they are derived, but exhibit at least one biological activity of the corresponding full-length protein. Typically, biologically active fragments comprise a domain or motif with at least one activity of the full-length protein. A biologically active fragment of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the biological activities of the native form of a polypeptide of the present invention.

In another embodiment, the present invention includes other functional variants of the polypeptides disclosed herein, which can be identified by techniques known in the art. For example, functional variants can be identified by screening combinatorial libraries of mutants (e.g., truncation mutants), of polypeptides of the present invention for biological activity. In another embodiment, a variegated library of variants can be generated by combinatorial mutagenesis at the nucleic acid level. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods that can be used to produce libraries of potential variants of the polypeptides of the present invention from a degenerate oligonucleotide sequence. Methods for synthesizing degenerate oligonucleotides are known in the art (e.g., see Narang (1983) Tetrahedron 39:3; Itakura et al., (1984) Annu. Rev. Biochem. 53:323; Itakura et al., (1984) Science 198:1056; Ike et al., (1983) Nucleic Acid Res. 11:477).

In addition, libraries of fragments of the coding sequence of a polypeptide of the present invention can be used to generate a variegated population of polypeptides for screening a subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the protein of interest.

Several techniques are known in the art for screening gene products of combinatorial libraries made by point mutations of truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of polypeptides of the present invention (Arkin and Yourvan (1992) Proc. Natl. Acad. Sci. USA 89:7811-7815; Delgrave et al., (1993) Protein Engineering 6(3): 327-331).

In another embodiment, functional variants of the present invention can encompasses orthologs of the genes and polypeptides disclosed herein. Orthologs of the polypeptides disclosed herein include proteins that can be isolated from other strains or species and possess a similar or identical biological activity. Such orthologs can be identified as comprising an amino acid sequence that is substantially homologous (shares a certain degree of amino acid sequence identity) with the polypeptides disclosed herein. As used herein, the expression “substantially homologous” refers to a first amino acid or nucleotide sequence which contains a sufficient or minimum number of identical or equivalent (e.g., with similar side chain) amino acids or nucleotides to a second amino acid or nucleotide sequence such that the first and the second amino acid or nucleotide sequences have a common domain. For example, amino acid or nucleotide sequences which contain a common domain having at least 70%, 71%, 72%, 73% 74%, 75%, 76%, 77%, 78%, 79%, 80%, 81%, 82%, 83%, 84%, 85%, 86%, 87%, 88%, 89%, 90%, 91% 92%, 93%, 94%, 95%, 96%, 97%, 98% or 99% sequence identity are defined herein as sufficiently identical.

In another embodiment, the present invention includes improved proteins derived from the polypeptides of the present invention. Improved proteins are proteins wherein at least one biological activity is improved. Such proteins may be obtained by randomly introducing mutations along all or part of the coding sequences of the polypeptides of the present invention such as by saturation mutagenesis, and the resulting mutants can be expressed recombinantly and screened for biological activity. For instance, the art provides for standard assays for measuring the enzymatic activity of the resulting protein and thus improved proteins may be selected.

### Recovery and Purification

In another aspect, polypeptides of the present invention may be present alone (e.g., in an isolated or purified form), within a composition (e.g., an enzymatic composition for carrying out an industrial process), or in an appropriate host. In one embodiment, polypeptides of the present invention can be recovered and purified from cell cultures (e.g., recombinant cell cultures) by methods known in the art. In another embodiment, high performance liquid chromatography (“HPLC”) can be employed for the purification.

In another aspect, polypeptides of the present invention include naturally purified products, products of chemical synthetic procedures, and products produced by recombinant techniques from a prokaryotic or eukaryotic host, including, for example, bacterial, yeast, higher plant, insect and mammalian cells. Depending on the host employed in a recombinant production procedure, the polypeptides of the present invention may be glycosylated or may be non-glycosylated. In addition, polypeptides of the invention may also include an initial modified methionine residue, in some cases as a result of host-mediated processes.

### Fusion Proteins

In another aspect, the present invention includes fusion proteins comprising a polypeptide of the present invention or a functional variant thereof, which is operatively linked to one or more unrelated polypeptide (e.g., heterologous amino acid sequences). “Unrelated polypeptides” or “heterologous polypeptides” or “heterologous sequences” refer to polypeptides or sequences which are usually not present close to or fused to one of the polypeptides of the present invention. Such “unrelated polypeptides” or “heterologous polypeptides” having amino acid sequences corresponding to proteins which are not substantially homologous to the polypeptide sequences disclosed herein. Such “unrelated polypeptides” can be derived from the same or a different organism. In one embodiment, a fusion protein of the present invention comprises at least two biologically active portions or domains of polypeptide sequences disclosed herein. In the context of fusion proteins, the term “operatively linked” is intended to indicate that all of the different polypeptides are fused in-frame to each other. In another embodiment, an unrelated polypeptide can be fused to the N terminus or C terminus of a polypeptide of the present invention.

In another embodiment, a polypeptide of the present invention can be fused to a protein which enables or facilitates recombinant protein purification and/or detection. For example, a polypeptide of the present invention can be fused to a protein such as glutathione S-transferase (GST), and the resulting fusion protein can then be purified/detected through the high affinity of GST for glutathione.

Fusion proteins of the present invention can be produced by standard recombinant DNA techniques. For example, DNA fragments encoding different polypeptide sequences can be ligated together in frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers, which give rise to complementary overhangs between two consecutive gene fragments which can subsequently be annealed and re-amplified to generate a chimeric gene sequence (e.g., see Current Protocols in Molecular Biology, eds. Ausubel et al., John Wiley & Sons: 1992). Moreover, many expression vectors are commercially available that already encode a fusion moiety (e.g., a GST polypeptide). A nucleic acid encoding a polypeptide of the present invention can be cloned into such an expression vector so that the fusion moiety is linked in-frame to the polypeptide of interest.

### Signal Sequences

In another embodiment, a polypeptide of the present invention can be fused to a heterologous signal sequence (e.g., at its N terminus) to facilitate its isolation, expression and/or secretion from certain host cells (e.g., mammalian and yeast host cells). Signal sequences are typically characterized by a core of hydrophobic amino acids, which are generally cleaved from the mature protein during secretion in one or more cleavage events. Such signal peptides may contain processing sites that allow cleavage of the signal sequence from the mature proteins as they pass through the secretory pathway.

For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

The signal sequence can direct secretion of the protein, such as from a eukaryotic host into which the expression vector is transformed, and the signal sequence is subsequently or concurrently cleaved. The protein can then be readily purified from the extracellular medium by known methods. In another embodiment, a signal sequence can be linked to a fusion protein of the present invention to facilitate detection, purification, and/or recovery thereof. For example, the sequence encoding a fusion protein of the present invention may be fused to a marker sequence, such as a sequence encoding a peptide, which facilitates purification of the fused polypeptide. In another embodiment, the marker sequence can be a hexa-histidine peptide, such as the tag provided in a pQE vector (Qiagen, Inc.), among others, many of which are commercially available. As described in Gentz et al., Proc. Natl. Acad. Sci. USA 86:821-824 (1989), for instance, hexa-histidine provides for convenient purification of the fusion protein. In another embodiment, the HA tag is another peptide useful for purification, which corresponds to an epitope derived of influenza hemaglutinin protein, which has been described by Wilson et al., Cell 37:767 (1984), for instance.

### Polynucleotides

The nucleic acid sequences of the genes disclosed herein were determined by sequencing cDNA clones, mRNA transcripts, or genomic DNA obtained from Scytalidium thermophilum strain CBS 625.9, Myriococcum thermophilum strain CBS 389.93, or Aureobasidium pullulans strain ATCC 62921.

In another aspect, the present invention relates to polynucleotides encoding a polypeptide of the present invention, including functional variants thereof. In one embodiment, polynucleotides of the present invention comprise the coding nucleic acid sequence of any one of SEQ ID NOs: 286-570, 1162-1467, or 2161-2547, or as set forth in Tables 1A-1C.

In another aspect, the present invention relates to genomic DNA sequences corresponding to the above mentioned coding sequences. In one embodiment, polynucleotides of the present invention comprise the genomic nucleic acid sequence of any one of SEQ ID NOs: 1-285, 856-1161, or 1774-2160; or as set forth in Tables 1A-1C.

In another aspect, the present invention relates to a polynucleotide comprising at least one intronic or exonic nucleic acid sequence of any one of the genomic sequences corresponding to SEQ ID NOs: 1-285, 856-1161, or 1774-2160 (e.g., the intron or exon segments defined by the exon boundaries listed in Tables 2A-2C). Although only the positions of the exons are defined in Tables 2A-2C, a person of skill in the art would readily be able to determine the positions of the corresponding introns in view of this information. In some embodiments, polynucleotides comprising at least one these intronic segments are within the scope of the present invention.

In yet another aspect, the present invention relates to a polynucleotide comprising at least one exonic nucleic acid sequence comprised within SEQ ID NOs: 1-285, 856-1161, or 1774-2160 or as set forth in Tables 2A-2C.

In another aspect, the present invention relates to isolated polynucleotides sharing a minimum threshold of nucleic acid sequence identity with any one of the above-mentioned polynucleotides. In specific embodiments, the present invention relates to isolated polynucleotides having at least 60%, 65%, 70%, 71%, 72, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% nucleic acid sequence identity to any one of the above-mentioned polynucleotides. Other specific percentage units that have not been specifically recited here for brevity are nevertheless considered within the scope of the present invention. Polynucleotides having the aforementioned thresholds of nucleic acid sequence identity can be created by introducing one or more nucleotide substitutions, additions or deletions into the coding nucleotide sequences of the present invention such that one or more amino acid substitutions, deletions or insertions are introduced into the encoded polypeptide. Such mutations may be introduced by standard techniques, such as site-directed mutagenesis and PCR-mediated mutagenesis.

In another aspect, the present invention relates to a polynucleotide that hybridizes (or is hybridizable) under medium-high stringency conditions, high stringency conditions, or very high stringency conditions with the full-length complement of any one of the polynucleotides defined above.

As used herein, “very low stringency conditions” means for probes of at least 100 nucleotides in length, prehybridization and hybridization at 42° C. in 5×SSPE, 0.3% SDS, 200 micrograms/mL sheared and denatured salmon sperm DNA, and 25% formamide, following standard Southern blotting procedures for 12 to 24 hours. The carrier material is finally washed three times each for 15 minutes using 2×SSC, 0.2% SDS at 45° C.

As used herein, “low stringency conditions” means for probes of at least 100 nucleotides in length, prehybridization and hybridization at 42° C. in 5×SSPE, 0.3% SDS, 200 micrograms/mL sheared and denatured salmon sperm DNA, and 25% formamide, following standard Southern blotting procedures for 12 to 24 hours. The carrier material is finally washed three times each for 15 minutes using 2×SSC, 0.2% SDS at 50° C.

As used herein, “medium stringency conditions” means for probes of at least 100 nucleotides in length, prehybridization and hybridization at 42° C. in 5×SSPE, 0.3% SOS, 200 micrograms/mL sheared and denatured salmon sperm DNA, and 35% formamide, following standard Southern blotting procedures for 12 to 24 hours. The carrier material is finally washed three times each for 15 minutes using 2×SSC, 0.2% SOS at 55° C.

As used herein, “medium-high stringency conditions” means for probes of at least 100 nucleotides in length, prehybridization and hybridization at 42° C. in 5×SSPE, 0.3% SDS, 200 micrograms/mL sheared and denatured salmon sperm DNA, and 35% formamide, following standard Southern blotting procedures for 12 to 24 hours. The carrier material is finally washed three times each for 15 minutes using 2×SSC, 0.2% SDS at 60° C.

As used herein, “high stringency conditions” means for probes of at least 100 nucleotides in length, prehybridization and hybridization at 42° C. in 5×SSPE, 0.3% SDS, 200 micrograms/mL sheared and denatured salmon sperm DNA, and 50% formamide, following standard Southern blotting procedures for 12 to 24 hours. The carrier material is finally washed three times each for 15 minutes using 2×SSC, 0.2% SDS at 65° C.

As used herein, “very high stringency conditions” means for probes of at least 100 nucleotides in length, prehybridization and hybridization at 42° C. in 5×SSPE, 0.3% SDS, 200 micrograms/mL sheared and denatured salmon sperm DNA, and 50% formamide, following standard Southern blotting procedures for 12 to 24 hours. The carrier material is finally washed three times each for 15 minutes using 2×SSC, 0.2% SDS at 70° C.

In one embodiment, a polynucleotide of the present invention (or a fragment thereof) can be isolated using the sequence information provided herein in conjunction with standard molecular biology techniques (e.g., as described in Sambrook et al., supra. For example, suitable hybridization oligonucleotides (e.g., probes or primers) can be designed using all or a portion of the nucleic acid sequences disclosed herein and prepared by standard synthetic techniques (e.g., using an automated DNA synthesizer). The oligonucleotides can be employed in hybridization and/or amplification reactions, for example, to amplify a template of cDNA, mRNA or genomic DNA, according to standard PCR techniques. A polynucleotide so amplified can be cloned into an appropriate vector and characterized by DNA sequence analysis.

In another aspect, the present invention relates to polynucleotides encoding functional variants of any one of the polypeptides of the present invention, including a biologically active fragment or domain thereof.

In another aspect, the present invention can include nucleic acid molecules (e.g., oligonucleotides) sufficient for use as primers and/or hybridization probes to amplify, sequence and/or identify nucleic acid molecules encoding a polypeptide of the present invention or fragments thereof. In some embodiments, the present invention relates to polynucleotides (e.g., oligonucleotides) that comprise, span, or hybridize specifically to exon-exon or exon-intron junctions of the genomic sequences identified herein, such as those defined in Tables 2A-2C. Designing such polynucleotides/oligonucleotides would be within the grasp of a person of skill in the art in view of the target sequence information disclosed herein and are thus encompassed by the present invention.

In another aspect, the present invention relates to polynucleotides comprising silent mutations or mutations that do not significantly alter the (biological) function or protein activity of the encoded polypeptide. Guidance concerning how to make phenotypically silent amino acid substitutions is provided for example in Bowie et al., Science 247:1306-1310 (1990) and in the references cited therein. Furthermore, it will be apparent for the skilled person that DNA sequence polymorphisms of the genes disclosed herein may exist within a given population, which may differ from the sequences disclosed herein. Such genetic polymorphisms may exist in cells from different populations or within a population due to natural allelic variation. Accordingly, in one embodiment, the present invention can include natural allelic variants and homologs of polynucleotides disclosed herein.

In another aspect, polynucleotides of the present invention can comprise only a portion or a fragment of the nucleic acid sequences disclosed herein. Although such polynucleotides may not encode a functional polypeptide of the present invention, they are useful for example as probes or primers in hybridization or amplification reactions. Exemplary uses of such polynucleotides include: (1) isolating a gene (as allelic variant thereof) from cDNA library; (2) in situ hybridization (e.g., FISH) to metaphase chromosomal spreads to provide precise chromosomal location of the gene as described in Verma et al., Human Chromosomes: a Manual of Basic Techniques, Pergamon Press, New York (1988); (3) Northern blot analysis for detecting expression of mRNA corresponding to a polypeptide disclosed herein, or a homolog, ortholog or variant thereof, in specific tissues and/or cells; and (4) probes and primers that can be used as a diagnostic tool to analyze the presence of a nucleic acid hybridizable to a polynucleotide disclosed herein in a given biological (e.g., tissue) sample. It would be within the grasp of a skilled person to design specific oligonucleotides in view of the nucleic acid sequences disclosed herein. Oligonucleotides typically comprise a region of nucleotide sequence that hybridizes (preferably under highly stringent conditions) to at least 10, 11, 12, 13, 14, 15, 16, 17, 18, 19, 20, 21, 22, 23, 24, 25, 26, 27, 28, 29, 30, 31, 32, 33, 34, 35, 36, 37, 37, 39, 40, 50, 60, 70, 80, 90 or 100 contiguous nucleotides of a polynucleotide of the present invention. In one embodiment, such oligonucleotides can be used for identifying and/or cloning other family members, as well as orthologs from other species. In another embodiment, the oligonucleotide can be attached to a detectable label (e.g., a radioisotope, a fluorescent compound, an enzyme, or an enzyme cofactor). Such oligonucleotides can also be used as part of a diagnostic method or kit for identifying cells which express a polypeptide of the present invention.

As would be understood by the skilled person, full-length complements of any one of the polynucleotides of the present invention are also encompassed. In one embodiment, the full-length complements are antisense molecules with respect to the coding strands of polynucleotides of the present invention, which hybridize (preferably under highly stringent conditions) to at least 10, 11, 12, 13, 14, 15, 16, 17, 18, 19, 20, 21, 22, 23, 24, 25, 26, 27, 28, 29, 30, 31, 32, 33, 34, 35, 36, 37, 37, 39, 40, 50, 60, 70, 80, 90 or 100 contiguous nucleotides to a polynucleotide of the present invention.

### Sequencing Errors

The sequence information as provided herein should not be so narrowly construed as to require inclusion of erroneously identified bases. The specific sequences disclosed herein can be readily used to isolate the corresponding complete genes from the organism sequenced herein, which in turn can easily be subjected to further sequence analyses thereby identifying sequencing errors.

Unless otherwise indicated, all nucleotide sequences disclosed herein were determined by sequencing using an automated DNA sequencer, and all amino acid sequences of polypeptides disclosed herein were predicted by translation based on the genetic code. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

The person skilled in the art is capable of identifying such erroneously identified bases and knows how to correct such errors.

### Vectors

Another aspect of the invention pertains to vectors (e.g., expression vectors), containing a polynucleotide encoding a polypeptide of the present invention.

As used herein, the term “vector” includes a nucleic acid molecule capable of transporting another nucleic acid molecule to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors useful in recombinant DNA techniques are often in the form of plasmids. The terms “plasmid” and “vector” can be used interchangeably herein as the plasmid is the most commonly used form of vector. However, the invention is intended to include such other forms of expression vectors, such as viral vectors (e.g., replication defective retroviruses, adenoviruses and adeno-associated viruses), which serve equivalent functions.

In one embodiment, recombinant expression vectors of the invention can comprise a polynucleotide of the present invention in a form suitable for expression of the polynucleotide in a host cell, which means that the recombinant expression vector includes one or more regulatory sequences, selected on the basis of the host cells to be used for expression, which is operatively linked to the nucleic acid sequence to be expressed. Within a recombinant expression vector, “operatively linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signal). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in a certain host cell (e.g., tissue-specific regulatory sequences). It will be appreciated by those skilled in the art that the design of the expression vector can depend on such factors as the choice of the host cell to be transformed, the level of expression of protein desired, etc. The expression vectors of the present invention can be introduced into host cells to thereby produce proteins or peptides, encoded by polynucleotides as described herein (e.g., polypeptides of the present invention).

In another embodiment, recombinant expression vectors of the present invention can be designed for expression of polypeptides of the present invention in prokaryotic or eukaryotic cells. For example, these polypeptides can be expressed in bacterial cells such as E. coli, insect cells (using baculovirus expression vectors), yeast cells or mammalian cells. Suitable host cells are discussed further in Goeddel supra). In another embodiment, recombinant expression vectors of the present invention can be transcribed and translated in vitro, for example using T7 promoter regulatory sequences and T7 polymerase.

In another embodiment, expression vectors of the present invention can include chromosomal-, episomal- and virus-derived vectors, e.g., vectors derived from bacterial plasmids, bacteriophage, yeast episome, yeast chromosomal elements, viruses such as baculoviruses, papova viruses, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids.

For expression, a DNA insert should be operatively linked to an appropriate promoter, such as the phage lambda PL promoter, the E. coli lac, trp and tac promoters, the SV40 early and late promoters and promoters of retroviral LTRs, to name a few. Other suitable promoters will be known to the skilled person. In a specific embodiment, promoters are preferred that are capable of directing a high expression level of biologically active polypeptides of the present invention (e.g., lignocellulose active proteins) from fungi. Such promoters are known in the art. The expression constructs may contain sites for transcription initiation, termination, and, in the transcribed region, a ribosome binding site for translation. The coding portion of the mature transcripts expressed by the constructs will include a translation initiating AUG at the beginning and a termination codon appropriately positioned at the end of the polypeptide to be translated.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, transduction, infection, lipofection, cationic lipid-mediated transfection or electroporation. Suitable methods for transforming or transfecting host cells can be found in Sambrook, et al., (Molecular Cloning: A Laboratory Manual, 2nd ed. Cold Spring Harbor Laboratory, Cold Spring Harbor Laboratory Press, Cold Spring Harbor, N.Y., 1989), Davis et al., Basic Methods in Molecular Biology (1986) and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methatrexate. A polynucleotide encoding a selectable marker can be introduced into a host cell on the same vector as that encoding a polypeptide of the present invention, or on a separate vector. Cells stably transfected with a polynucleotide of the present invention can be identified by drug selection (e.g., cells that have incorporated the selectable marker gene will survive, while the other cells die).

Expression of proteins in prokaryotes is often carried out in E. coli with vectors containing constitutive or inducible promoters directing the expression of either fusion or non-fusion proteins. Fusion vectors add a number of amino acids to a protein encoded therein, e.g., to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein.

Vectors preferred for use in bacteria are for example disclosed in WO-A1-2004/074468. Other suitable vectors will be readily apparent to the skilled artisan. Known bacterial promoters suitable for use in the present invention include the promoters disclosed in WO-A1-2004/074468.

As indicated, the expression vectors will preferably contain selectable markers. Such markers include dihydrofolate reductase or neomycin resistance for eukaryotic cell culture and antibiotic resistance (e.g., tetracyline or ampicillin) for culturing in E. coli and other bacteria. Representative examples of appropriate host include bacterial cells, such as E. coli, Streptomyces, Salmonella typhimurium and certain Bacillus species; fungal cells such as Aspergillus species, for example A. niger, A. oryzae and A. nidulans, yeast cells such as Kluyveromyces, for example K. lactis and/or Pichia, for example P. pastoris; insect cells such as Drosophila S2 and Spodoptera Sf9; animal cells such as CHO, COS and Bowes melanoma; and plant cells. Appropriate culture mediums and conditions for the above-described host cells are known in the art.

Transcription of the DNA encoding the polypeptides of the present invention by higher eukaryotes may be increased by inserting an enhancer sequence into the vector. Enhancers are cis-acting elements of DNA, usually about from 10 to 300 by that act to increase transcriptional activity of a promoter in a given host cell-type. Examples of enhancers include the SV40 enhancer, which is located on the late side of the replication origin at by 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signal may be incorporated into the expressed polypeptide. The signals may be endogenous to the polypeptide or they may be heterologous signals. In an embodiment, a polypeptide of the present invention may be expressed in a modified form, such as a fusion protein, and may include not only secretion signals but also additional heterologous functional regions. Thus, for instance, a region of additional amino acids, particularly charged amino acids, may be added to the N terminus of the polypeptide to improve stability and persistence in the host cell, during purification or during subsequent handling and storage. Also, peptide moieties may be added to the polypeptide to facilitate purification and/or detection.

### Host Cells

In another aspect, the present invention features cells, e.g., transformed host cells or recombinant host cells that contain a polynucleotide or vector of the present invention. A “transformed cell” or “recombinant cell” is a cell into which (or into an ancestor of which) has been introduced a polynucleotide or vector of the invention by means of recombinant DNA techniques. Both prokaryotic and eukaryotic cells are included, e.g., bacteria, fungi, yeast, and the like, especially preferred are cells from filamentous fungi, in particular the strain from which the polynucleotide and polypeptide sequences disclosed herein were derived.

In one embodiment, a cell of the present invention is typically not a wild-type strain or a naturally-occurring cell. Host cells of the present invention can include, but are not limited to: fungi (e.g., Aspergillus niger, Trichoderma reesii, Myceliophthora thermophila and Talaromyces emersonii); yeasts (e.g., Saccharomyces cerevisiae, Yarrowia lipolytica and Pichia pastoris); bacteria (e.g., Escherichia coli and Bacillus sp.); and plants (e.g., Nicotiana benthamiana, Nicotiana tabacum and Medicago sativa).

In another embodiment, a polynucleotide (or a polynucleotide which is comprised within a vector) may be homologous or heterologous with respect to the cell into which it is introduced. In this context, a polynucleotide is homologous to a cell if the polynucleotide naturally occurs in that cell. A polynucleotide is heterologous to a cell if the polynucleotide does not naturally occur in that cell. Accordingly, in an embodiment, the present invention relates to a cell which comprises a heterologous or a homologous sequence corresponding to any one of the polynucleotides or polypeptides disclosed herein.

In another embodiment, a host cell can be chosen that modulates the expression of the inserted sequences, or modifies and processes the gene product in a specific, desired fashion. Such modifications (e.g., glycosylation) and processing (e.g., cleavage) of protein products may facilitate optimal functioning of the protein. Various host cells have characteristic and specific mechanisms for post-translational processing and modification of proteins and gene products. Appropriate cell lines or host systems familiar to those of skill in the art can be chosen to ensure the desired and correct modification and processing of the foreign protein expressed. To this end, eukaryotic host cells that possess the cellular machinery for proper processing of the primary transcript, glycosylation, and phosphorylation of the gene product can be used. Such host cells are well known in the art.

In another embodiment, host cells can also include, but are not limited to, mammalian cell lines such as CHO, VERO, BHK, HeLa, COS, MDCK, 293, 3T3, WI38, and choroid plexus cell lines. If desired, a stably transfected cell line can produce the polypeptides of the present invention. A number of vectors suitable for stable transfection of mammalian cells are available to the public, methods for constructing such cell lines are also publicly known, e.g., in Ausubel et al., (supra).

In another embodiment, the present invention relates to methods of inhibiting the expression of a polypeptide of the present invention in a host cell, comprising administering to the cell or expressing in the cell a double-stranded RNA (dsRNA) molecule (or a molecule comprising region of double-strandedness), wherein the dsRNA comprises a subsequence of a polynucleotide of the present invention. In a preferred aspect, the dsRNA is about 15, 16, 17, 18, 19, 20, 21, 22, 23, 24, 25 or more duplex nucleotides in length. The dsRNA is preferably a small interfering RNA (siRNA) or a micro RNA (miRNA). In a preferred aspect, the dsRNA is small interfering RNA (siRNAs) for inhibiting transcription. In another preferred aspect, the dsRNA is micro RNA (miRNAs) for inhibiting translation. The present invention also relates to such double-stranded RNA (dsRNA) molecules, comprising a portion of the mature polypeptide coding sequence of any one of the coding sequences of the polypeptides disclosed herein of inhibiting expression of that polypeptide in a cell. While the present invention is not limited by any particular mechanism of action, the dsRNA can enter a cell and cause the degradation of a single-stranded RNA (ssRNA) of similar or identical sequences, including endogenous mRNAs. When a cell is exposed to dsRNA, mRNA from the homologous gene is selectively degraded by a process called RNA interference (RNAi). The dsRNAs of the present invention can be used in gene-silencing methods. In one aspect, the invention relates to methods to selectively degrade RNA using the dsRNAi's of the present invention. The process may be practiced in vitro, ex vivo or in vivo. In one aspect, the dsRNA molecules can be used to generate a loss-of-function mutation in a cell, an organ or an organism. Methods for making and using dsRNA molecules to selectively degrade RNA are well known in the art, see, for example, U.S. Pat. No. 6,506,559; U.S. Pat. No. 6,511,824; U.S. Pat. No. 6,515,109; and U.S. Pat. No. 6,489,127. In some instances, new phylogenic analyses of fungal species have resulted in taxonomic reclassifications. For example, following their phylogenic studies reported in van den Brink et al., (“Phylogeny of the industrial relevant, thermophilic genera Myceliophthora and Corynascus”, Fungal Diversity (2012), 52:197-207), the authors proposed renaming all existing Corynascus species to Myceliophthora. Such changes in taxonomic classification are within the scope of the present invention and, regardless of future reclassifications, a person of skill in the art would be able to identify the organism used to determine the sequences disclosed herein for example based on the strain's accession number (CBS 389.93; ATCC 62921; or CBS 625.91).

It should be understood herein that the level of expression of polypeptides of the present invention could be modified by adapting the codon usage ratio of a sequence of the present invention to that of the host or hosts in which it is meant to be expressed. This adaptation and the concept of codon usage ratio are all well known in the art.

### Antibodies

In another aspect, the present invention relates to an isolated binding agent capable of selectively binding to a polypeptide of the present invention. Suitable binding agents may be selected from an antibody, an antigen binding fragment, or a binding partner. In one embodiment, the binding agent selectively binds to an amino acid sequence selected from Tables 1A-1C, including to any fragment of any of the above sequences comprising at least one antibody binding epitope.

According to the present invention, the phrase “selectively binds to” refers to the ability of an antibody, antigen binding fragment or binding partner of the present invention to preferentially bind to specified proteins. More specifically, the phrase “selectively binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA, immunoblot assays, etc.).

Antibodies are characterized in that they comprise immunoglobulin domains and as such, they are members of the immunoglobulin superfamily of proteins. An antibody of the invention includes polyclonal and monoclonal antibodies, divalent and monovalent antibodies, bi- or multi-specific antibodies, serum containing such antibodies, antibodies that have been purified to varying degrees, and any functional equivalents of whole antibodies. Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), may also be employed in the invention. Methods for the generation and production of antibodies are well known in the art.

Monoclonal antibodies may be produced according to the methodology of Kohler and Milstein (Nature 256:495-497, 1975). Non-antibody polypeptides, sometimes referred to as binding partners, may be designed to bind specifically to a protein of the invention. Examples of the design of such polypeptides, which possess a prescribed ligand specificity are given in Beste et al., (Proc. Nat'l Acad. Sci. 96:1898-1903, 1999). In one embodiment, a binding agent of the invention is immobilized on a substrate such as: artificial membranes, organic supports, biopolymer supports and inorganic supports such as for use in a screening assay.

In some embodiment, antibodies and binding agents specifically binding to polypeptides of the present invention may be produced and used even in absence of knowledge of the precise biological function and/or protein activity of the polypeptide. Such antibodies and binding agent may be useful, for example, as diagnostic, classification, and/or research tools.

### Compositions and Uses

In another aspect, the present invention relates to composition comprising one or more polypeptides or polynucleotides of the present invention. In one embodiment, the compositions are enriched in such a polypeptide. The term “enriched” indicates that the biological activity (e.g., biomass degradation or processing) of the composition has been increased, e.g., with an enrichment factor of at least 1.1. The composition may comprise a polypeptide of the present invention as the major component, e.g., a mono-component composition. Alternatively, the composition may comprise multiple enzymatic activities (e.g., those described herein).

The polypeptide compositions may be prepared in accordance with methods known in the art and may be in the form of a liquid or a dry composition. For instance, the polypeptide composition may be in the form of a granulate or a microgranulate. The polypeptide to be included in the composition may be stabilized in accordance with methods known in the art. Examples are given below of preferred uses of the polypeptide compositions of the present invention. The dosage of the polypeptide composition of the invention and other conditions under which the composition is used may be determined on the basis of methods known in the art.

In another aspect, the present invention relates to the use of the polypeptides (e.g., enzymes) of the present invention a number of industrial and other processes. Despite the long term experience obtained with these processes, there remains a need for improved polypeptides and enzymes featuring one or more significant advantages over those presently used. Depending on the specific application, these advantages can include aspects such as lower production costs, higher specificity towards the substrate, greater synergies with existing enzymes, less antigenic effect, less undesirable side activities, higher yields when produced in a suitable microorganism, more suitable pH and temperature ranges, better properties of the final product, and food grade or kosher aspects. In various embodiments, the present invention seeks to provide one or more of these advantages, or others.

### Biomass Processing or Degradation

In another aspect, the polypeptides of the present invention may be used in new or improved methods for enzymatically degrading or converting plant cell wall polysaccharides from biomass into various useful products. In addition to cellulose and hemicellulose, plant cell walls contain associated pectins and lignins, the removal of which by enzymes of the current invention can improve accessibility to cellulases and hemicellulases, or which can themselves be converted to useful products. Therefore the polypeptides of the present invention may be used to degrade biomass or pretreated biomass to sugars. These sugars may be used as such or may be, for example, fermented into ethanol.

Usually, biomass must be subjected to pre-treatment in order to make the cellulose more accessible. Accordingly, in one embodiment, polypeptides of the present invention may be used in improved methods for the processing of pretreated biomass. Pretreatment technologies may involve chemical, physical, or biological treatments. Examples of pre-treatment technologies include but are not limited to: steam explosion; ammonia; acid hydrolysis; alkaline hydrolysis; solvent extraction; crushing; milling; etc.

One example of a product produced from biomass is bioethanol. Bioethanol is usually produced by the fermentation of glucose to ethanol by yeasts such as Saccharomyces cerevisiae: in addition to ethanol, other chemicals may be synthesized starting from glucose. Ethanol, today, is produced mostly from sugars or starches, obtained from sugar cane, fruits and grains. In contrast, cellulosic ethanol is obtained from cellulose, the main component of wood, straw and much of the plants. Sources of biomass for cellulosic ethanol production comprise agricultural residues (e.g., leftover crop materials from stalks, leaves, and husks of corn plants), forestry wastes (e.g., chips and sawdust from lumber mills, dead trees, and tree branches), energy crops (e.g., dedicated fast-growing trees and grasses such as switch grass), municipal solid waste (e.g., household garbage and paper products), food processing and other industrial wastes (e.g., black liquor, paper manufacturing by-products, etc.).

Plant biomass is a mixture of plant polysaccharides, including cellulose, hemicelluloses, and pectin, together with the structural polymer, lignin. Glucose is released from cellulose by the action of mixtures of enzymes, including: endoglucanases, exoglucanases (cellobiohydrolases 1 and 2) and beta-glucosidases. Efficient large-scale conversion of cellulosic materials by such mixtures may require the full complement of enzymes, and can be enhanced by the addition of enzymes that attack the other plant cell wall components (e.g., hemicelluloses, pectins, and lignins), as well as chemical linkages between these components. Hence, polypeptides of the present invention that are highly expressed, or have high specific activity, stability, or resistance to inhibitors may improve the efficiency of the process, and lower enzyme costs. It would be an advantage to the art to improve the degradation and conversion of plant cell wall polysaccharides by composing cellulase mixtures using cellulase enzymes with such properties. Furthermore, polypeptides of the present invention that are able to function at extremes of pH and temperature are desirable, both since improved enzyme robustness decreases costs, and because enzymes that function at high temperature will allow high processing temperatures under high substrate consistency conditions that decrease viscosity and thus improve yields.

Glycoside hydrolases from the family GH61 are known to stimulate the activity of cellulose cocktails on lignocellulosic substrates and are thus considered to exhibit cellulose-enhancing activity (Harris et al., Biochemistry 49, 3305 (2010)). They have no known enzymatic activities of their own. Enhancement of cellulase cocktail efficiency by GH61 proteins of the present invention may contribute to lowering the costs of cellulase enzymes used for the production of glucose from plant cell biomass, as described above. GH61 (glycoside hydrolase family 61 or sometimes referred to as EGIV) proteins are oxygen-dependent polysaccharide monooxygenases (PMO's) according to the latest literature. Often in the literature, these proteins are mentioned as enhancing the action of cellulases on lignocellulose substrates. GH61 was originally classified as an endogluconase, based on the measurement of very weak endo-1,4-β-d-glucanase activity in one family member. The term “GH61” as used herein, is to be understood as a family of enzymes, which share common conserved sequence portions and foldings to be classified in family 61 of the well-established CAZY GH classification system (http://www.cazy.org/GH61.html). The glycoside hydrolase family 61 is a member of the family of glycoside hydrolases EC 3.2.1. GH61 is used herein as being part of the cellulases.

Enzymatic hydrolysis of plant hemicellulose yields 5-carbon sugars that either may be fermented to ethanol by some species of yeast, or converted to other types of chemical products. Enzymatic deconstruction of hemicellulose is also known to improve the accessibility of plant cell wall cellulose to cellulase enzymes for the production of glucose from lignocellulosic materials. Hemicellulase enzymes of the present invention that enhance glucose production from lignocellulose would find utility in the bioethanol industry and in other process that rely on glucose or pentose streams from lignocellulose.

Lignin is composed of methoxylated phenyl-propane units linked by ether linkages and carbon-carbon bonds. The chemical composition of lignin may, depending on species, include guaiacyl, 4-hydroxyphenyl, and syringyl groups. Enzymatic modification of lignin by the polypeptides of the present invention can be used for the production of structural materials from plant biomass, or alternatively improve the accessibility of plant cellulose and hemicelluloses to cellulase enzymes for the release of glucose from biomass as described above. Enzymes that degrade the lignin component of lignocellulose include lignin peroxidases, manganese-dependent peroxidases, versatile peroxidases, and laccases (Vicuna et al., 2000, Molecular Biotechnology 14: 173-176; Broda et al., 1996, Molecular Microbiology 19: 923-932). In some embodiments, polypeptides of the present invention may also, in certain instances, be active in the decolorization of industrial dyes, and thus useful for the treatment and detoxification of chemical wastes.

In another embodiment, pectin-degrading polypeptides of the present invention can also enhance the action of cellulases on plant biomass by improving the accessibilty of cellulase to the cellulose component of lignocellulose.

In another embodiment, polypeptides of the present invention may also be useful in other applications for hydrolyzing non-starch polysaccharide (NSP).

In another embodiment, esterases of the present invention can be useful in the bioenergy industry such as for the production of biodiesel and hydrolysis of hemicellulose.

In another embodiment, the present invention relates to methods for degrading or converting a cellulose-containing material, comprising: treating the cellulose-containing material with an effective amount of a cellulolytic enzyme composition in the presence of an effective amount of a polypeptide having cellulolytic enhancing activity of the present invention, wherein the presence of the polypeptide having cellulolytic enhancing activity increases the degradation of cellulose-containing material compared to the absence of the polypeptide having cellulolytic enhancing activity.

In another embodiment, the present invention relates to methods for producing a fermentation product, comprising: (a) saccharifying a cellulose-containing material with an effective amount of a cellulolytic enzyme composition in the presence of an effective amount of a polypeptide having cellulolytic enhancing activity of the present invention, wherein the presence of the polypeptide having cellulolytic enhancing activity increases the degradation of cellulose-containing material compared to the absence of the polypeptide having cellulolytic enhancing activity; (b) fermenting the saccharified cellulose-containing material of step (a) with one or more fermenting microorganisms to produce the fermentation product; and (c) recovering the fermentation product from the fermentation.

### Food Product Industry

In one embodiment, the present invention relates to methods for preparing a food product comprising incorporating into the food product an effective amount of a polypeptide of the present invention. This can improve one or more properties of the food product relative to a food product in which the polypeptide is not incorporated. The phrase “incorporated into the food product” is defined herein as adding a polypeptide of the present invention to the food product, to any ingredient from which the food product is to be made, and/or to any mixture of food ingredients from which the food product is to be made. In other words, a polypeptide of the present invention may be added in any step of the food product preparation and may be added in one, two or more steps. The polypeptide of the present invention is added to the ingredients of a food product which can then be treated by methods including cooking, boiling, drying, frying, steaming or baking as is known in the art.

At least in the context of food products, the term “effective amount” is defined herein as an amount of the polypeptide (e.g., enzyme) of the present invention that is sufficient for providing a measurable effect on at least one property of interest of the food product. The term “improved property” is defined herein as any property of a food product which is improved by the action of a polypeptide (e.g., enzyme) of the present invention relative to a food product in which the polypeptide is not incorporated. The improved property may be determined by comparison of a food product prepared with and without addition of a polypeptide of the present invention. Organoleptic qualities may be evaluated using procedures well established in the food industry, and may include, for example, the use of a panel of trained taste-testers.

The polypeptides of the present invention may be prepared in any form suitable for the use in question, e.g., in the form of a dry powder, agglomerated powder, or granulate, in particular a non-dusting granulate, liquid, in particular a stabilized liquid, or protected enzyme such as described in WO01/11974 and WO02/26044. Granulates and agglomerated powders may be prepared by conventional methods, e.g., by spraying the enzyme according to the invention onto a carrier in a fluid-bed granulator. The carrier may consist of particulate cores having a suitable particle size. The carrier may be soluble or insoluble, e.g., a salt (such as NaCl or sodium sulphate), sugar (such as sucrose or lactose), sugar alcohol (such as sorbitol), starch, rice, corn grits, or soy. In an embodiment, the polypeptide of the present invention (and/or additional polypeptides/enzymes) may be contained in slow-release formulations. Methods for preparing slow-release formulations are well known in the art. Adding nutritionally acceptable stabilizers such as sugar, sugar alcohol, or another polyol, and/or lactic acid or another organic acid according to established methods may for instance, stabilize liquid enzyme preparations.

In another embodiment, polypeptides of the present invention may also be incorporated in yeast-comprising compositions such as disclosed in EP-A-0619947, EP-A-0659344 and WO02/49441.

In another embodiment, one or more additional polypeptides/enzymes may be incorporated into a food product of the present invention. The additional enzyme may be of any origin, including mammalian and plant, and preferably of microbial (bacterial, yeast or fungal) origin and may be obtained by techniques conventionally used in the art. Enzymes may conveniently be produced in microorganisms. Microbial enzymes are available from a variety of sources; Bacillus species are a common source of bacterial enzymes, whereas fungal enzymes are commonly produced in Aspergillus species.

In specific embodiments, additional polypeptides/enzymes include starch degrading enzymes, xylanases, oxidizing enzymes, fatty material splitting enzymes, or protein-degrading, modifying or crosslinking enzymes. Starch degrading enzymes include endo-acting enzymes such as alpha-amylase, maltogenic amylase, pullulanase or other debranching enzymes, and exo-acting enzymes that cleave off glucose (amyloglucosidase), maltose (beta-amylase), maltotriose, maltotetraose and higher oligosaccharides. Suitable xylanases are for instance xylanases, pentosanases, hemicellulase, arabinofuranosidase, glucanase, cellulase, cellobiohydrolase, beta-glucosidase, and others. Oxidizing enzymes are for instance glucose oxidase, hexose oxidase, pyranose oxidase, sulfhydryl oxidase, lipoxygenase, laccase, polyphenol oxidases and others. Fatty material splitting enzymes are for instance triacylglycerol lipases, phospholipases (such as A1, A2, B, C and D) and galactolipases. Protein degrading, modifying or crosslinking enzymes are for instance endo-acting proteases (serine proteases, metalloproteases, aspartyl proteases, thiol proteases), exo-acting peptidases that cleave off one amino acid, or dipeptide, tripeptide etceteras from the N-terminal (aminopeptidases) or C-terminal (carboxypeptidases) ends of the polypeptide chain, asparagines or glutamine deamidating enzymes such as deamidase and peptidoglutaminase or crosslinking enzymes such as transglutaminase.

In others embodiments, additional polypeptides/enzymes can include: amylases, such as alpha-amylase (which can be useful for providing sugars that are fermentable by yeast) or beta-amylase; cyclodextrin glucanotransferase; peptidase (e.g., an exopeptidase, which can be useful in flavour enhancement); transglutaminase; lipase, which can be useful for the modification of lipids present in the food or food constituents), phospholipase, cellulase, hemicellulase, protein disulfide isomerase, peroxidase, laccase, or an oxidase (e.g., glucose oxidase, hexose oxidase, aldose oxidase, pyranose oxidase, lipoxygenase or L-amino acid oxidase).

In other embodiment, esterases of the present invention have a number of applications in the food industry including, but not limited to, degumming vegetable oils; improving the production of bread (e.g., in situ production of emulsifiers); producing crackers, noodles, and pasta; enhancing flavor development of cheese, butter, and margarine; ripening cheese; removing wax; trans-esterification of flavors and cocoa butter substitutes; synthesizing structured lipids for infant formula and nutraceuticals; improving the polyunsaturated fatty acid content in fish oil; and aiding in digestion and releasing minerals in food processing.

When one or more additional enzyme activities are to be added in accordance with the methods of the present invention, these activities may be added separately or together with the polypeptide according to the invention.

### Detergent Industry

In another aspect, polypeptides of the present invention can be useful in the detergent industry, e.g., for removal of carbohydrate-based stains from soiled laundry. Enzymes are used in detergents in order to improve its efficacy to remove most types of dirt. In some embodiments, esterases such as lipases of the present invention are particularly useful for removing fats and lipids.

### Feed Industry

In another aspect, polypeptides of the present invention can be useful in the feed enzyme industry, e.g., for increasing nutritional quality, digestibility and/or absorption of animal feed.

Feed enzymes have an important role to play in current farming systems, as they can increase the digestibility of nutrients, leading to greater efficiency in the production of animal products such as meat and eggs. At the same time, they can play a role in minimizing the environmental impact of increased animal production.

Non-starch polysaccharides (NSP) can increase the viscosity of the digesta which can, in turn, decrease nutrient availability and animal performance.

Endoxylanases and phytases are the best-known feed-enzyme products. Phytase enzymes hydrolyse phytic acid and release inorganic phosphate, thereby avoiding the need to add inorganic phosphates to the diet and reducing phosphorus excretion. Addition of xylanases to feed has also been shown to have positive effects on animal growth. Adding specific nutrients to feed improves animal digestion and thereby reduces feed costs. A lot of feed additives are being currently used and new concepts are continuously developed. Use of specific enzymes like non-starch carbohydrate degrading enzymes could breakdown fiber, releasing energy as well as increasing the protein digestibility due to better accessibility of the protein when fiber gets broken down. In this way the feed cost could come down, as well as the protein levels in the feed also could be reduced.

Non-starch polysaccharides (NSPs) are also present in virtually all feed ingredients of plant origin. NSPs are poorly utilized and can, when solubilized, exert adverse effects on digestion. Exogenous enzymes can contribute to a better utilization of these NSPs and as a consequence reduce any anti-nutritional effects. Accordingly, in a particular embodiment, hemicellulases and other polysaccharide-active polypeptides/enzymes of the present invention can be used for this purpose in cereal-based diets for poultry and, to a lesser extent, for pigs and other species.

In some embodiments, esterases of the present invention are useful in the feed industry such as for reducing the amount of phosphate in feed.

### Pulp and Paper

In another embodiment, xylanases of the present invention can be useful in the pulp and paper industry, e.g., for prebleaching of kraft pulp. Xylanases have been found to be most effective for that purpose. Xylanases attract increasing scientific and commercial attention due to applications in the pulp and paper industry for removal of hemicellulose from dissolving pulps or for enhancement of the bleachability of pulp and, thus, reduction of the use of environmentally harmful bleaching chemicals. A similar application of xylanases for pulp prebleaching is an already well-established technology and has greatly stimulated research on hemicellulases in the past decade. Although lignin-active peroxidases of the present invention may also be active in modification of lignin and hence have bleaching properties, such enzymes are generally less attractive for bleaching due to the need to use and recycle expensive redox mediators.

In a related embodiment, polypeptides such as xylanases of the present invention can be used to pre-bleach pulp to reduce the amount of bleaching chemicals to obtain a given brightness. It is suggested that xylanase depolymerises xylan blocks and increases accessibility or helps liberation of residual lignin by releasing xylan-chromophore fragments. In addition to brownstock prior to bleaching, polypeptides such as xylanases of the present invention can save on bleaching chemicals. The enzymes hydrolyze surface xylans and are able to break linkages between hemicellulose and lignin. Other polypeptides (e.g., hemicellulase active enzymes) of the present invention which can break these linkages can function effectively in bleaching or pre-bleaching of pulp, and thus such uses are also within the scope of the present invention.

In some embodiments, esterases of the present invention are useful for the removal of triglycerides, steryl esters, resin acids, free fatty acids, and sterols (e.g., lipophilic wood extractives).

### Other Uses

In another embodiment, polypeptides such as xylanases of the present invention can be used in antibacterial formulations, as well as in pharmaceutical products such as throat lozenges, toothpastes, and mouthwash.

Chitin is a beta-(1,4)-linked polymer of N-acetyl D-glucosamine (GlcNAc), found as a structural polysaccharide in fungal cell walls as well as in the exoskeleton of arthropods and the outer shell of crustaceans. Approximately 75% the total weight of shellfish, is considered waste, and a large proportion of the material making up the waste is chitin. Accordingly, in one embodiment, polypeptides such as chitin-degrading enzymes of the present invention are useful in the modification and degradation of chitin, allowing the production of chitin-derived material, such as chitooligosaccharides and N-acetyl D-glucosamine, from chitin waste. In another embodiment, polypeptides such as chitinase enzymes of the present invention can be useful as antifungal agents.

In another embodiment, polypeptides of the present invention can be used in the textile industry (e.g., for the treatment of textile substrates). More particularly, cellulases (e.g., endo-, exocellulases and cellobiohydrolases) have gained importance in the treatment of cellulose-containing fibers. During the washing of indigo-dyed denim textiles, enzymatic treatment by a polypeptide of the present invention is can be used in place of (or in addition to) a bleaching treatment to achieve a “used” look of jeans or other suitable fabrics. Polypeptides of the present invention can also improve the softness/feel of such fabrics. When used in textile detergent compositions, enzymes of the present invention can enhance cleaning ability or act as a softening agent. In another embodiment, polypeptides such as cellulases of the present invention can be used in combination with polymeric agents in processes for providing a localized variation in the color density of fibers.

In another embodiment, polypeptides of the present invention can be used in the waste treatment industry (e.g., for changing the characteristics of the waste to become more amenable to further treatment and/or for bio-conversion to value-added products). Polypeptides such as lipases, cellulases, amylases, and proteases of the present invention can be used in addition to microorganisms to break down polymeric substances like proteins, polysaccharides and lipids, thereby facilitating this process.

In another embodiment, polypeptides of the present invention can be used in industries such as biocatalysis; sewage treatment; cleaning up oil pollution; the synthesis of fragrances; and enhancing the recovery of oil (e.g., during drilling).

Other uses of the polynucleotides and polypeptides of the present invention would be apparent to a person of skill in the art in view of the sequences and biological activities disclosed herein. These other uses, even though not explicitly mentioned here, are nevertheless within the scope of the present invention.

### Diagnostic, Classification and Research Tools

In another embodiment, the polynucleotides, polypeptides and antibodies of the present invention can be useful for diagnostic and classification tools. In this regard, it would be within the capacities of a person of skill in the art to search existing sequence databases and perform a phylogenic analysis based on the nucleic acid and amino acid sequences disclosed herein. Furthermore, designing hybridization probes or primers that are specific for a particular genus, species or strain (e.g., the genus, species, or strain from which the sequences disclosed herein were derived) would be within the grasp of a skilled person, in view of the sequence information disclosed herein. Similarly, a skilled person would be able to select an epitope of a polypeptide of the present invention which is specific for a particular genus, species or strain (e.g., the genus, species, or strain from which the sequences disclosed herein were derived) and generate an antibody or binding agent that binds specifically thereto.

Such tools are useful, for example, in diagnostic methods for detecting the presence or absence of a particular organism (e.g., the organism from which the sequences disclosed herein were derived) in a sample; as research tools (e.g., for designing and producing microarrays for studying fungal gene expression); for rapidly classifying an organism of interest based the detection of a sequence or polypeptide specific for that organism. The skilled person would recognize that knowledge of the precise (biological) function or protein activity of a polypeptide of the present invention is not absolutely necessary for the aforementioned tools to be useful for diagnostic, research, or classification purposes. Sequences that are particularly useful in this regard are the genomic, coding and amino acid sequences corresponding to the polypeptides of the present invention annotated as “unknown” in Tables 1A-1C (as well as their corresponding exons and introns defined in Tables 2A-2C, where available). These sequences show little sequence identity with those in the art and thus can be useful as markers for identifying the organisms from which the sequences of the present invention were derived. The skilled person would know how to search various sequence databases to design specific hybridization oligonucleotides (e.g., probes and primers), as well as produce antibodies specifically binds to the aforementioned sequences.

In some embodiments, the present invention relates to a method for identifying and/or classifying an organism (e.g., a fungal species) based on a biological sample, the method comprising detecting the presence or absence of any one of the polynucleotides or polypeptides of the present invention (e.g., those recited in the preceding paragraph) and determining that said organism is present or classifying said organism based on the presence of the polynucleotide or polypeptide. In some embodiments, the detecting step can be carried out using one or more oligonucleotides or antibodies of the present invention. In some embodiments, the detecting step can be carried out by performing an amplification and/or hybridization reaction.

In Tables 1A-1C below, the skilled person will recognize that although the precise protein activity of a polypeptide of the present invention may not be known (e.g., in the case of “unknown” proteins), the polypeptide may be nevertheless useful for carrying out an industrial process (e.g., cellulase-enhancing, cellulose-degrading, hemicellulose-degrading, etc.).

TABLE 1A
Biomass degrading genes and polypeptides of Scytalidium thermophilum

| | | | | | | | | Provisional | | | PCT | | |
|---|---|---|---|---|---|---|---|---|---|---|---|---|---|
| Gene ID in | | Annotation in | | | | | | application | | | application | | |
| Provisional | | Provisional | | | | | CBM | SEQ ID NO: | | | SEQ ID NO: | | |
| Application No. | | Application No. | | | | CAZy | of in- | Ge- | Cod- | Amino | Ge- | Cod- | Amino |
| 61/657,082 | Target ID | 61/657,082 | Updated annotation | Function | Protein activity | family | terest | nomic | ing | acid | nomic | ing | acid |
| Scyth2p4_000006 | SCYTH_1_03938 | xylanase | xylanase GH10 | hemicellulose- | xylanase | GH10 | | 1 | 2 | 3 | 1 | 286 | 571 |
| | | | | degrading | | | | | | | | | |
| Scyth2p4_000010 | Scyth2p4_000010 | unknown | Acid phosphatase | dephosphorylation | Acid phosphatase | | | 4 | 5 | 6 | 2 | 287 | 572 |
| Scyth2p4_000016 | | Leucine | Leucine | protein | protease | | | 7 | 8 | 9 | 3 | 288 | 573 |
| | | aminopeptidase 2 | aminopeptidase 2 | hydrolysis | | | | | | | | | |
| Scyth2p4_000019 | SCYTH_2_02709 | unknown | unknown | | uncharacterized | | | 10 | 11 | 12 | 4 | 289 | 574 |
| | | | | | lignocellulose-induced | | | | | | | | |
| | | | | | protein | | | | | | | | |
| Scyth2p4_000123 | | Putative beta- | arabinoxylan | hemicellulose- | arabinofuranosidase | GH43 | | 13 | 14 | 15 | 5 | 290 | 575 |
| | | xylosidase | arabinofuranohydrolase | degrading | | | | | | | | | |
| | | | GH43 | | | | | | | | | | |
| Scyth2p4_000124 | SCYTH_2_06066 | unknown | unknown | | uncharacterized | | | 16 | 17 | 18 | 6 | 291 | 576 |
| | | | | | lignocellulose-induced | | | | | | | | |
| | | | | | protein | | | | | | | | |
| Scyth2p4_000141 | | Probable aspartic- | Candidapepsin-3 | protein | protease | | | 19 | 20 | 21 | 7 | 292 | 577 |
| | | type endopeptidase | | hydrolysis | | | | | | | | | |
| | | OPSB | | | | | | | | | | | |
| Scyth2p4_000168 | | unknown | unknown | | unknown | | | 22 | 23 | 24 | 8 | 293 | 578 |
| Scyth2p4_000230 | Scyth2p4_000230 | unknown | galactanase GH5 | hemicellulose- | galactanase | GH5 | | 25 | 26 | 27 | 9 | 294 | 579 |
| | | | | degrading | | | | | | | | | |
| Scyth2p4_000277 | | Putative lipase | Putative lipase | lipid-modifying | lipase | | | 28 | 29 | 30 | 10 | 295 | 580 |
| | | atg15 | atg15 | | | | | | | | | | |
| Scyth2p4_000610 | Scyth2p4_000610 | unknown | xylanase GH30 | hemicellulose- | xylanase | GH30 | | 31 | 32 | 33 | 11 | 296 | 581 |
| | | | | degrading | | | | | | | | | |
| Scyth2p4_000863 | SCYTH_1_00740 | hexosaminidase | hexosaminidase GH20 | chitin- | hexosaminidase | GH20 | | 34 | 35 | 36 | 12 | 297 | 582 |
| | | | | degrading | | | | | | | | | |
| Scyth2p4_000904 | Scyth2p4_000904 | Probable feruloyl | Probable feruloyl | hemicellulose- | feruloyl esterase | | | 37 | 38 | 39 | 13 | 298 | 583 |
| | | esterase A | esterase A | modifying | | | | | | | | | |
| Scyth2p4_001035 | Scyth2p4_001035 | Tyrosinase | Tyrosinase | pigment- | Tyrosinase | | | 40 | 41 | 42 | 14 | 299 | 584 |
| | | | | generating | | | | | | | | | |
| Scyth2p4_001183 | | Carboxypeptidase Y | Carboxypeptidase Y | protein | protease | | | 43 | 44 | 45 | 15 | 300 | 585 |
| | | | homolog A | hydrolysis | | | | | | | | | |
| Scyth2p4_001259 | Scyth2p4_001259 | unknown | unknown | | uncharacterized | | | 46 | 47 | 48 | 16 | 301 | 586 |
| | | | | | lignocellulose-induced | | | | | | | | |
| | | | | | protein | | | | | | | | |
| Scyth2p4_001262 | Scyth2p4_001262 | endoglucanase | endoglucanase GH5 | cellulose- | endoglucanase | GH5 | CBM | 49 | 50 | 51 | 17 | 302 | 587 |
| | | | | degrading | | | 1 | | | | | | |
| Scyth2p4_001326 | | Aspergillopepsin-2 | Aspergillopepsin-2 | protein | protease | | | 52 | 53 | 54 | 18 | 303 | 588 |
| | | | | hydrolysis | | | | | | | | | |
| Scyth2p4_001371 | Scyth2p4_001371 | Probable exo-1,4- | Beta-xylosidase GH3 | hemicellulose- | beta-xylosidase | GH3 | | 55 | 56 | 57 | 19 | 304 | 589 |
| | | beta-xylosidase | | degrading | | | | | | | | | |
| | | bxlB | | | | | | | | | | | |
| Scyth2p4_001379 | | Mannan endo-1,4- | beta-mannanase GH26 | hemicellulose- | beta-mannanase | GH26 | CBM | 58 | 59 | 60 | 20 | 305 | 590 |
| | | beta-mannosidase | | degrading | | | 35 | | | | | | |
| Scyth2p4_001450 | | Carbohydrate- | possible carbohydrate- | carbohydrate- | carbohydrate-binding | | | 61 | 62 | 63 | 21 | 306 | 591 |
| | | binding cytochrome | binding cytochrome | oxidizing | cytochrome | | | | | | | | |
| | | b562 (Fragment) | | | | | | | | | | | |
| Scyth2p4_001460 | Scyth2p4_001460 | chitinase | chitinase GH18 | chitin- | chitinase | GH18 | | 64 | 65 | 66 | 22 | 307 | 592 |
| | | | | degrading | | | | | | | | | |
| Scyth2p4_001513 | | Glucan 1,3-beta- | Glucan 1,3-beta- | cellulose- | glucan 1,3-beta- | GH55 | | 67 | 68 | 69 | 23 | 308 | 593 |
| | | glucosidase | glucosidase | degrading | glucosidase | | | | | | | | |
| Scyth2p4_001745 | Scyth2p4_001745 | Endoglucanase E1 | Endoglucanase | cellulose- | endoglucanase | GH5 | | 70 | 71 | 72 | 24 | 309 | 594 |
| | | | | degrading | | | | | | | | | |
| Scyth2p4_001867 | SCYTH_1_00384 | Probable beta- | Probable beta- | hemicellulose- | beta-mannosidase B | GH2 | | 73 | 74 | 75 | 25 | 310 | 595 |
| | | mannosidase B | mannosidase B | degrading | | | | | | | | | |
| Scyth2p4_001875 | | Metallocarboxypeptidase | Metallocarboxypeptidase | protein | protease | | | 76 | 77 | 78 | 26 | 311 | 596 |
| | | A-like protein | A-like protein | hydrolysis | | | | | | | | | |
| | | ARB_03789 | MCYG_01475 | | | | | | | | | | |
| Scyth2p4_001878 | Scyth2p4_001878 | unknown | unknown | | uncharacterized | | | 79 | 80 | 81 | 27 | 312 | 597 |
| | | | | | lignocellulose-induced | | | | | | | | |
| | | | | | protein | | | | | | | | |
| Scyth2p4_001887 | Scyth2p4_001887 | O- | O- | | oxidoreductase | | | 82 | 83 | 84 | 28 | 313 | 598 |
| | | methylsterigmatocystin | methylsterigmatocystin | | | | | | | | | | |
| | | oxidoreductase | oxidoreductase | | | | | | | | | | |
| Scyth2p4_001903 | | Probable leucine | Leucine | protein | protease | | | 85 | 86 | 87 | 29 | 314 | 599 |
| | | aminopeptidase | aminopeptidase 1 | hydrolysis | | | | | | | | | |
| | | MCYG_04170 | | | | | | | | | | | |
| Scyth2p4_001974 | | Endothiapepsin | Endothiapepsin | protein | protease | | | 88 | 89 | 90 | 30 | 315 | 600 |
| | | | | hydrolysis | | | | | | | | | |
| Scyth2p4_001995 | SCYTH_1_09959 | cellulase-enhancing | polysaccharide | cellulose- | polysaccharide | GH61 | | 91 | 92 | 93 | 31 | 316 | 601 |
| | | protein | monooxygenase | degrading | monooxygenase | | | | | | | | |
| Scyth2p4_001998 | Scyth2p4_001998 | Probable feruloyl | feruloyl esterase CE1 | hemicellulose- | feruloyl esterase | CE1 | | 94 | 95 | 96 | 32 | 317 | 602 |
| | | esterase C | | degrading | | | | | | | | | |
| Scyth2p4_002014 | Scyth2p4_002014 | unknown | unknown | | uncharacterized | | | 97 | 98 | 99 | 33 | 318 | 603 |
| | | | | | lignocellulose-induced | | | | | | | | |
| | | | | | protein | | | | | | | | |
| Scyth2p4_002032 | | unknown | unknown | | unknown | | | 100 | 101 | 102 | 34 | 319 | 604 |
| Scyth2p4_002058 | | Tripeptidyl- | Tripeptidyl- | peptide | protease | | | 103 | 104 | 105 | 35 | 320 | 605 |
| | | peptidase sed1 | peptidase sed1 | hydrolysis | | | | | | | | | |
| Scyth2p4_002089 | SCYTH_1_01777 | endo-1,5-alpha- | endo-1,5-alpha- | hemicellulose- | endo-1,5-alpha- | GH43 | | 106 | 107 | 108 | 36 | 321 | 606 |
| | | arabinanase | arabinanase GH43 | degrading | arabinanase | | | | | | | | |
| Scyth2p4_002099 | | endoglucanase | Endoglucanase GH12 | cellulose- | endoglucanase | GH12 | | 109 | 110 | 111 | 37 | 322 | 607 |
| | | | | degrading | | | | | | | | | |
| Scyth2p4_002112 | | unknown | unknown | | unknown CBM18 | | CBM | 112 | 113 | 114 | 38 | 323 | 608 |
| | | | | | | | 18 | | | | | | |
| Scyth2p4_002143 | | Glucan 1,3-beta- | exo-1,3-beta- | cellulose- | exo-1,3-beta- | GH55 | | 115 | 116 | 117 | 39 | 324 | 609 |
| | | glucosidase | glucanase GH55 | degrading | glucanase | | | | | | | | |
| Scyth2p4_002153 | Scyth2p4_002153 | Adhesin, putative | possible adhesin | | adhesin | | | 118 | 119 | 120 | 40 | 325 | 610 |
| Scyth2p4_002186 | SCYTH_2_02011 | Rhamnogalacturonan | rhamnogalacturonan | pectin- | rhamnogalacturonan | CE12 | | 121 | 122 | 123 | 41 | 326 | 611 |
| | | acetylesterase | acetylesterase CE12 | degrading | acetylesterase | | | | | | | | |
| Scyth2p4_002220 | Scyth2p4_002220 | cellulase- | polysaccharide | cellulose- | polysaccharide | GH61 | CBM | 124 | 125 | 126 | 42 | 327 | 612 |
| | | enhancing protein | monooxygenase | degrading | monooxygenase | | 1 | | | | | | |
| Scyth2p4_002225 | Scyth2p4_002225 | Cellobiose | Cellobiose | lignin- | cellobiose | | | 127 | 128 | 129 | 43 | 328 | 613 |
| | | dehydrogenase | dehydrogenase | degrading | dehydrogenase | | | | | | | | |
| Scyth2p4_002425 | | Uncharacterized | Uncharacterized | | oxidoreductase | | | 130 | 131 | 132 | 44 | 329 | 614 |
| | | oxidoreductase | oxidoreductase | | | | | | | | | | |
| | | C30D10.05c | C30D10.05c | | | | | | | | | | |
| Scyth2p4_002446 | Scyth2p4_002446 | Adhesin protein | possible adhesin | | adhesin | | | 133 | 134 | 135 | 45 | 330 | 615 |
| | | Mad1 | | | | | | | | | | | |
| Scyth2p4_002491 | | Adhesin protein | possible adhesin | | adhesin | GH18 | | 136 | 137 | 138 | 46 | 331 | 616 |
| | | Mad1 | | | | | | | | | | | |
| Scyth2p4_002582 | | Adhesin protein | possible adhesin | | adhesin | | | 139 | 140 | 141 | 47 | 332 | 617 |
| | | Mad1 | | | | | | | | | | | |
| Scyth2p4_002596 | | Subtilisin-like | Subtilisin-like | protein | protease | | | 142 | 143 | 144 | 48 | 333 | 618 |
| | | proteinase Spm1 | proteinase Spm1 | hydrolysis | | | | | | | | | |
| Scyth2p4_002639 | SCYTH_2_05416 | unknown | unknown | | uncharacterized | | | 145 | 146 | 147 | 49 | 334 | 619 |
| | | | | | lignocellulose-induced | | | | | | | | |
| | | | | | protein | | | | | | | | |
| Scyth2p4_002689 | Scyth2p4_002689 | cellulase- | polysaccharide | cellulose- | polysaccharide | GH61 | | 148 | 149 | 150 | 50 | 335 | 620 |
| | | enhancing protein | monooxygenase | degrading | monooxygenase | | | | | | | | |
| Scyth2p4_002854 | SCYTH_1_03782 | arabinogalactanase | arabinogalactanase GH53 | hemicellulose- | arabinogalactanase | GH53 | | 151 | 152 | 153 | 51 | 336 | 621 |
| | | | | degrading | | | | | | | | | |
| Scyth2p4_002859 | | Nucleotide exchange | Nucleotide exchange | | nucleotide exchange | | | 154 | 155 | 156 | 52 | 337 | 622 |
| | | factor SIL1 | factor SIL1 | | factor | | | | | | | | |
| Scyth2p4_003064 | Scyth2p4_003064 | alpha-amylase | Alpha-amylase GH13 | starch- | alpha-amylase | GH13 | | 157 | 158 | 159 | 53 | 338 | 623 |
| | | | | degrading | | | | | | | | | |
| Scyth2p4_003098 | Scyth2p4_003098 | Killer toxin subunits | Killer toxin subunits | chitin- | chitinase | GH18 | CBM | 160 | 161 | 162 | 54 | 339 | 624 |
| | | alpha/beta | alpha/beta | degrading | | | 18 | | | | | | |
| Scyth2p4_003108 | | Probable beta- | Probable beta- | cellulose- | beta-glucosidase | GH17 | | 163 | 164 | 165 | 55 | 340 | 625 |
| | | glucosidase btgE | glucosidase btgE | degrading | | | | | | | | | |
| Scyth2p4_003124 | | Probable endo-1,3(4)- | mixed-link glucanase | glucan- | mixed-link glucanase | GH16 | | 166 | 167 | 168 | 56 | 341 | 626 |
| | | beta-glucanase | GH16 | degrading | | | | | | | | | |
| | | AFUB_029980 | | | | | | | | | | | |
| Scyth2p4_003222 | Scyth2p4_003222 | Endoglucanase-5 | endoglucanase GH45 | cellulose- | endoglucanase | GH45 | | 169 | 170 | 171 | 57 | 342 | 627 |
| | | | | degrading | | | | | | | | | |
| Scyth2p4_003248 | | Lysophospholipase | Lysophospholipase | phospholipid- | lipase | | | 172 | 173 | 174 | 58 | 343 | 628 |
| | | | | modifying | | | | | | | | | |
| Scyth2p4_003738 | | Probable aspartic- | Probable aspartic- | protein | protease | | | 175 | 176 | 177 | 59 | 344 | 629 |
| | | type endopeptidase | type endopeptidase | hydrolysis | | | | | | | | | |
| | | opsB | OPSB | | | | | | | | | | |
| Scyth2p4_003766 | Scyth2p4_003766 | unknown | unknown | | unknown GH16 | GH16 | | 178 | 179 | 180 | 60 | 345 | 630 |
| Scyth2p4_003836 | Scyth2p4_003836 | Cellobiose | Cellobiose | cellulose- | cellobiose | | CBM | 181 | 182 | 183 | 61 | 346 | 631 |
| | | dehydrogenase | dehydrogenase | degrading | dehydrogenase | | 1 | | | | | | |
| Scyth2p4_003875 | SCYTH_1_01865 | cellulase-enhancing | polysaccharide | cellulose- | polysaccharide | GH61 | CBM | 184 | 185 | 186 | 62 | 347 | 632 |
| | | protein | monooxygenase | degrading | monooxygenase | | 1 | | | | | | |
| Scyth2p4_003882 | SCYTH_1_09023 | beta-glucosidase | beta-glucosidase GH3 | cellulose- | beta-glucosidase | GH3 | | 187 | 188 | 189 | 63 | 348 | 633 |
| | | | | degrading | | | | | | | | | |
| Scyth2p4_003909 | Scyth2p4_003909 | cellulase-enhancing | polysaccharide | cellulose- | polysaccharide | GH61 | | 190 | 191 | 192 | 64 | 349 | 634 |
| | | protein | monooxygenase | degrading | monooxygenase | | | | | | | | |
| Scyth2p4_003923 | Scyth2p4_003923 | unknown | xylan alpha-1,2- | hemicellulose- | xylan alpha-1,2- | GH115 | | 193 | 194 | 195 | 65 | 350 | 635 |
| | | | glucuronidase GH115 | modifying | glucuronidase | | | | | | | | |
| Scyth2p4_003925 | | cellulase-enhancing | polysaccharide | cellulose- | polysaccharide | GH61 | CBM | 196 | 197 | 198 | 66 | 351 | 636 |
| | | protein | monooxygenase | degrading | monooxygenase | | 1 | | | | | | |
| Scyth2p4_003929 | | unknown | unknown | | unknown | | | 199 | 200 | 201 | 67 | 352 | 637 |
| Scyth2p4_003943 | | exo-1,3-beta- | exo-1,3-beta- | glucan- | exo-1,3-beta- | GH55 | | 202 | 203 | 204 | 68 | 353 | 638 |
| | | glucanase | glucanase GH55 | degrading | glucanase | | | | | | | | |
| Scyth2p4_004010 | | Endo-1,4-beta- | xylanase GH10 | hemicellulose- | xylanase | GH10 | | 205 | 206 | 207 | 69 | 354 | 639 |
| | | xylanase | | degrading | | | | | | | | | |
| Scyth2p4_004018 | Scyth2p4_004018 | unknown | unknown | | uncharacterized | | | 208 | 209 | 210 | 70 | 355 | 640 |
| | | | | | lignocellulose-induced | | | | | | | | |
| | | | | | protein | | | | | | | | |
| Scyth2p4_004025 | Scyth2p4_004025 | alpha- | arabinoxylan | hemicellulose- | arabinofuranosidase | GH62 | | 211 | 212 | 213 | 71 | 356 | 641 |
| | | arabinofuranosidase | arabinofuranohydrolase | degrading | | | | | | | | | |
| | | | GH62 | | | | | | | | | | |
| Scyth2p4_004026 | SCYTH_1_04528 | Alpha-N- | Alpha-N- | hemicellulose- | arabinofuranosidase | GH43 | | 214 | 215 | 216 | 72 | 357 | 642 |
| | | arabinofuranosidase | arabinofuranosidase | degrading | | | | | | | | | |
| | | 2 | 2 | | | | | | | | | | |
| Scyth2p4_004049 | Scyth2p4_004049 | cellulase-enhancing | polysaccharide | cellulose- | polysaccharide | GH61 | | 217 | 218 | 219 | 73 | 358 | 643 |
| | | protein | monooxygenase | degrading | monooxygenase | | | | | | | | |
| Scyth2p4_004099 | Scyth2p4_004099 | cellulase-enhancing | polysaccharide | cellulose- | polysaccharide | GH61 | | 220 | 221 | 222 | 74 | 359 | 644 |
| | | protein | monooxygenase | degrading | monooxygenase | | | | | | | | |
| Scyth2p4_004162 | Scyth2p4_004162 | Probable | Acetylxylan esterase 1 | hemicellulose- | acetylxylan esterase | CE1 | | 223 | 224 | 225 | 75 | 360 | 645 |
| | | acetylxylan esterase | CE1 | modifying | | | | | | | | | |
| | | A | | | | | | | | | | | |
| Scyth2p4_004197 | Scyth2p4_004197 | unknown | exo-1,3-beta- | galactan- | exo-1,3-beta- | GH43 | CBM | 226 | 227 | 228 | 76 | 361 | 646 |
| | | | galactanase GH43 | degrading | galactanase | | 35 | | | | | | |
| Scyth2p4_004205 | SCYTH_1_00248 | endoglucanase | endoglucanase GH6 | cellulose- | endoglucanase | GH6 | CBM | 229 | 230 | 231 | 77 | 362 | 647 |
| | | | | degrading | | | 1 | | | | | | |
| Scyth2p4_004235 | | Aspergillopepsin A | Aspartic protease PEP3 | protein | protease | | | 232 | 233 | 234 | 78 | 363 | 648 |
| | | | | hydrolysis | | | | | | | | | |
| Scyth2p4_004237 | SCYTH_1_01221 | beta-glucosidase | beta-glucosidase GH3 | cellulose- | beta-glucosidase | GH3 | | 235 | 236 | 237 | 79 | 364 | 649 |
| | | | | degrading | | | | | | | | | |
| Scyth2p4_004263 | | Non-Catalytic | xylanase GH10 | Hemicellulose- | xylanase | GH10 | CBM | 238 | 239 | 240 | 80 | 365 | 650 |
| | | module family | | degrading | | | 1 | | | | | | |
| | | expansin | | | | | | | | | | | |
| Scyth2p4_004293 | SCYTH_1_08979 | cellobiohydrolase | cellobiohydrolase GH7 | cellulose- | cellobiohydrolase | GH7 | CBM | 241 | 242 | 243 | 81 | 366 | 651 |
| | | | | degrading | | | 1 | | | | | | |
| Scyth2p4_004317 | Scyth2p4_004317 | unknown | unknown | | uncharacterized | | | 244 | 245 | 246 | 82 | 367 | 652 |
| | | | | | lignocellulose-induced | | | | | | | | |
| | | | | | protein | | | | | | | | |
| Scyth2p4_004650 | Scyth2p4_004650 | unknown | Uncharacterized protein | | uncharacterized | | | 247 | 248 | 249 | 83 | 368 | 653 |
| | | | SAOUHSC_02143 | | lignocellulose-induced | | | | | | | | |
| | | | | | protein | | | | | | | | |
| Scyth2p4_004945 | Scyth2p4_004945 | cellulase-enhancing | polysaccharide | cellulose- | polysaccharide | GH61 | | 250 | 251 | 252 | 84 | 369 | 654 |
| | | protein | monooxygenase | degrading | monooxygenase | | | | | | | | |
| Scyth2p4_004976 | | N-acyl- | phospholipase | phospholipid- | lipase | | | 253 | 254 | 255 | 85 | 370 | 655 |
| | | phosphatidylethanol | | modifying | | | | | | | | | |
| | | amine-hydrolyzing | | | | | | | | | | | |
| | | phospholipase D | | | | | | | | | | | |
| Scyth2p4_005037 | Scyth2p4_005037 | Pectate lyase plyB | Pectate lyase plyB | pectin- | pectate lyase | PL1 | | 256 | 257 | 258 | 86 | 371 | 656 |
| | | | | degrading | | | | | | | | | |
| Scyth2p4_005092 | | unknown | unknown | | unknown CE3 | CE3 | | 259 | 260 | 261 | 87 | 372 | 657 |
| Scyth2p4_005093 | Scyth2p4_005093 | Cutinase | Cutinase CE5 | cutin-degrading | cutinase | CE5 | | 262 | 263 | 264 | 88 | 373 | 658 |
| Scyth2p4_005094 | Scyth2p4_005094 | Cutinase | Cutinase CE5 | cutin-degrading | cutinase | CE5 | | 265 | 266 | 267 | 89 | 374 | 659 |
| Scyth2p4_005146 | | Leucine | Leucine | protein | protease | | | 268 | 269 | 270 | 90 | 375 | 660 |
| | | aminopeptidase 1 | aminopeptidase 1 | hydrolysis | | | | | | | | | |
| Scyth2p4_005307 | | unknown | unknown | | unknown CBM18 | | CBM | 271 | 272 | 273 | 91 | 376 | 661 |
| | | | | | | | 18 | | | | | | |
| Scyth2p4_005334 | Scyth2p4_005334 | Pectinesterase | pectin methylesterase | pectin- | pectinesterase | CE8 | | 274 | 275 | 276 | 92 | 377 | 662 |
| | | | CE8 | modifying | | | | | | | | | |
| Scyth2p4_005335 | Scyth2p4_005335 | unknown | Beta-glucanase | glucan- | beta-glucanase | GH16 | | 277 | 278 | 279 | 93 | 378 | 663 |
| | | | | degrading | | | | | | | | | |
| Scyth2p4_005384 | | unknown | unknown | | unknown | | | 280 | 281 | 282 | 94 | 379 | 664 |
| Scyth2p4_005465 | Scyth2p4_005465 | unknown | unknown | | unknown CE16 | CE16 | | 283 | 284 | 285 | 95 | 380 | 665 |
| Scyth2p4_005588 | Scyth2p4_005588 | unknown | unknown | | uncharacterized | | | 286 | 287 | 288 | 96 | 381 | 666 |
| | | | | | lignocellulose-induced | | | | | | | | |
| | | | | | protein | | | | | | | | |
| Scyth2p4_005596 | | unknown | unknown | | unknown CE4 | CE4 | | 289 | 290 | 291 | 97 | 382 | 667 |
| Scyth2p4_005646 | SCYTH_2_00017 | unknown | unknown | | uncharacterized | | | 292 | 293 | 294 | 98 | 383 | 668 |
| | | | | | lignocellulose-induced | | | | | | | | |
| | | | | | protein | | | | | | | | |
| Scyth2p4_005692 | | Bifunctional | acetylxylan esterase CE4 | hemicellulose- | acetylxylan esterase | CE4 | | 295 | 296 | 297 | 99 | 384 | 669 |
| | | xylanase/deacetylase | | degrading | | | | | | | | | |
| Scyth2p4_005696 | Scyth2p4_005696 | unknown | unknown | | uncharacterized | | | 298 | 299 | 300 | 100 | 385 | 670 |
| | | | | | lignocellulose-induced | | | | | | | | |
| | | | | | protein | | | | | | | | |
| Scyth2p4_005712 | SCYTH_2_07654 | Probable 1,4-beta- | carbohydrate esterase | hemicellulose- | unknown CE16 | CE16 | CBM | 301 | 302 | 303 | 101 | 386 | 671 |
| | | D-glucan | | modifying | | | 1 | | | | | | |
| | | cellobiohydrolase C | | | | | | | | | | | |
| Scyth2p4_005714 | SCYTH_2_02004 | Acetylxylan | acetylxylan esterase CE1 | hemicellulose- | acetylxylan esterase | CE1 | CBM | 304 | 305 | 306 | 102 | 387 | 672 |
| | | esterase A | | modifying | | | 1 | | | | | | |
| Scyth2p4_005722 | Scyth2p4_005722 | Aldose 1-epimerase | Aldose 1-epimerase | | aldose epimerase | | | 307 | 308 | 309 | 103 | 388 | 673 |
| Scyth2p4_005760 | SCYTH_1_00672 | cellulase- | polysaccharide | cellulose- | polysaccharide | GH61 | | 310 | 311 | 312 | 104 | 389 | 674 |
| | | enhancing protein | monooxygenase | degrading | monooxygenase | | | | | | | | |
| Scyth2p4_005775 | Scyth2p4_005775 | Expansin-like | possible expansin | cellulase- | expansin | | | 313 | 314 | 315 | 105 | 390 | 675 |
| | | protein | | enhancing | | | | | | | | | |
| Scyth2p4_005777 | Scyth2p4_005777 | non-catalytic | possible expansin | cellulase- | expansin | | | 316 | 317 | 318 | 106 | 391 | 676 |
| | | module family | | enhancing | | | | | | | | | |
| | | expansin | | | | | | | | | | | |
| Scyth2p4_005792 | SCYTH_1_00771 | alpha-glucuronidase | alpha-glucuronidase | hemicellulose- | alpha-glucuronidase | GH67 | | 319 | 320 | 321 | 107 | 392 | 677 |
| | | | GH67 | modifying | GH67 | | | | | | | | |
| Scyth2p4_005865 | SCYTH_1_09242 | cellulase-enhancing | polysaccharide | cellulose- | polysaccharide | GH61 | CBM | 322 | 323 | 324 | 108 | 393 | 678 |
| | | protein | monooxygenase | degrading | monooxygenase | | 1 | | | | | | |
| Scyth2p4_005894 | | Uncharacterized | Uncharacterized | | unknown CE4 | CE4 | | 325 | 326 | 327 | 109 | 394 | 679 |
| | | protein yjeA | protein yjeA | | | | | | | | | | |
| Scyth2p4_006005 | Scyth2p4_006005 | unknown | exo-arabinanase GH93 | hemicellulose- | exo-arabinanase | GH93 | | 328 | 329 | 330 | 110 | 395 | 680 |
| | | | | degrading | GH93 | | | | | | | | |
| Scyth2p4_006013 | Scyth2p4_006013 | Probable 1,4-beta- | Exoglucanase 1 | cellulose- | Exoglucanase | GH7 | | 331 | 332 | 333 | 111 | 396 | 681 |
| | | D-glucan | | degrading | | | | | | | | | |
| | | cellobiohydrolase B | | | | | | | | | | | |
| Scyth2p4_006014 | Scyth2p4_006014 | Beta-galactosidase | Beta-glucuronidase | hemicellulose- | Beta-glucuronidase | GH2 | | 334 | 335 | 336 | 112 | 397 | 682 |
| | | | | degrading | | | | | | | | | |
| Scyth2p4_006016 | | cellulase-enhancing | polysaccharide | cellulose- | polysaccharide | GH61 | CBM | 337 | 338 | 339 | 113 | 398 | 683 |
| | | protein | monooxygenase | degrading | monooxygenase | | 1 | | | | | | |
| Scyth2p4_006040 | Scyth2p4_006040 | unknown | unknown | | uncharacterized | | | 340 | 341 | 342 | 114 | 399 | 684 |
| | | | | | lignocellulose-induced | | | | | | | | |
| | | | | | protein | | | | | | | | |
| Scyth2p4_006061 | | Putative | Putative | protein | protease | | | 343 | 344 | 345 | 115 | 400 | 685 |
| | | metallocarboxypeptidase | metallocarboxypeptidase | hydrolysis | | | | | | | | | |
| | | MCYG_04493 | ecm14 | | | | | | | | | | |
| Scyth2p4_006263 | | Lipase | Lipase | lipid-degrading | lipase | CE5 | | 346 | 347 | 348 | 116 | 401 | 686 |
| Scyth2p4_006265 | Scyth2p4_006265 | beta-glucosidase | beta-glucosidase GH3 | cellulose- | beta-glucosidase GH3 | GH3 | | 349 | 350 | 351 | 117 | 402 | 687 |
| | | | | degrading | | | | | | | | | |
| Scyth2p4_006499 | | Neutral alpha- | Glucosidase 2 subunit | glucoside- | glucosidase | GH31 | | 352 | 353 | 354 | 118 | 403 | 688 |
| | | glucosidase AB | alpha | degrading | | | | | | | | | |
| Scyth2p4_006556 | | Probable | Probable glycosidase crf1 | glucoside- | glycosidase | GH16 | | 355 | 356 | 357 | 119 | 404 | 689 |
| | | glycosidase crf1 | | degrading | | | | | | | | | |
| Scyth2p4_006566 | SCYTH_2_05810 | unknown | unknown | | uncharacterized | GH43 | | 358 | 359 | 360 | 120 | 405 | 690 |
| | | | | | lignocellulose-induced | | | | | | | | |
| | | | | | protein | | | | | | | | |
| Scyth2p4_006586 | | cellulase-enhancing | polysaccharide | cellulose- | polysaccharide | GH61 | | 361 | 362 | 363 | 121 | 406 | 691 |
| | | protein | monooxygenase | degrading | monooxygenase | | | | | | | | |
| Scyth2p4_006591 | Scyth2p4_006591 | endoglucanase | xyloglucanase GH74 | xyloglucan- | xyloglucanase | GH74 | CBM | 364 | 365 | 366 | 122 | 407 | 692 |
| | | | | degrading | | | 1 | | | | | | |
| Scyth2p4_006628 | Scyth2p4_006628 | Carbohydrate- | possible carbohydrate- | carbohydrate- | carbohydrate-binding | | | 367 | 368 | 369 | 123 | 408 | 693 |
| | | binding cytochrome | binding cytochrome | oxidizing | cytochrome | | | | | | | | |
| | | b562 | | | | | | | | | | | |
| Scyth2p4_006768 | | exo-1,3-beta- | exo-1,3-beta- | cellulose- | exo-1,3-beta- | GH55 | | 370 | 371 | 372 | 124 | 409 | 694 |
| | | glucanase | glucanase GH55 | degrading | glucanase | | | | | | | | |
| Scyth2p4_006914 | SCYTH_2_07965 | Putative | rhamnogalacturonan lyase | pectin- | rhamnogalacturonase | PL4 | | 373 | 374 | 375 | 125 | 410 | 695 |
| | | rhamnogalacturonase | PL4 | degrading | | | | | | | | | |
| Scyth2p4_006916 | Scyth2p4_006916 | Carbohydrate- | Cellobiose dehydrogenase | cellulose- | carbohydrate-binding | | | 376 | 377 | 378 | 126 | 411 | 696 |
| | | binding cytochrome | | degrading | cytochrome | | | | | | | | |
| | | b562 (Fragment) | | | | | | | | | | | |
| Scyth2p4_006920 | SCYTH_2_04020 | Exoglucanase-6A | possible expansin | cellulase- | expansin | CE3 | CBM | 379 | 380 | 381 | 127 | 412 | 697 |
| | | | | enhancing | | | 1 | | | | | | |
| Scyth2p4_006931 | | Aspergillopepsin-2 | Aspergillopepsin-2 | protein | protease | | | 382 | 383 | 384 | 128 | 413 | 698 |
| | | | | hydrolysis | | | | | | | | | |
| Scyth2p4_006993 | SCYTH_1_03721 | cellobiohydrolase | cellobiohydrolase GH6 | cellulose- | cellobiohydrolase¹ | GH6 | CBM | 385 | 386 | 387 | 129 | 414 | 699 |
| | | | | degrading | | | 1 | | | | | | |
| Scyth2p4_007002 | Scyth2p4_007002 | Swollenin | possible swollenin | cellulase- | swollenin | CE15 | CBM | 388 | 389 | 390 | 130 | 415 | 700 |
| | | | | enhancing | | | 1 | | | | | | |
| Scyth2p4_007064 | | Vacuolar protease A | Vacuolar protease A | protein | protease | | | 391 | 392 | 393 | 131 | 416 | 701 |
| | | | | hydrolysis | | | | | | | | | |
| Scyth2p4_007097 | SCYTH_1_03940 | cellulase-enhancing | polysaccharide | cellulose- | polysaccharide | GH61 | CBM | 394 | 395 | 396 | 132 | 417 | 702 |
| | | protein | monooxygenase | degrading | monooxygenase | | 1 | | | | | | |
| Scyth2p4_007200 | Scyth2p4_007200 | Carbohydrate- | possible carbohydrate- | carbohydrate- | carbohydrate-binding | | | 397 | 398 | 399 | 133 | 418 | 703 |
| | | binding cytochrome | binding cytochrome | oxidizing | cytochrome | | | | | | | | |
| | | b562 | | | | | | | | | | | |
| Scyth2p4_007231 | Scyth2p4_007231 | cellulase-enhancing | polysaccharide | cellulose- | polysaccharide | GH61 | | 400 | 401 | 402 | 134 | 419 | 704 |
| | | protein | monooxygenase | degrading | monooxygenase | | | | | | | | |
| Scyth2p4_007246 | | unknown | unknown | | unknown CBM18 | | CBM | 403 | 404 | 405 | 135 | 420 | 705 |
| | | | | | | | 18 | | | | | | |
| Scyth2p4_007249 | Scyth2p4_007249 | unknown | unknown | | unknown CE15 | CE15 | | 406 | 407 | 408 | 136 | 421 | 706 |
| Scyth2p4_007259 | Scyth2p4_007259 | arabinoxylan | arabinoxylan | hemicellulose- | arabinofuranosidase | GH62 | | 409 | 410 | 411 | 137 | 422 | 707 |
| | | arabinofuranohydrolase | arabinofuranosidase | modifying | | | | | | | | | |
| | | | GH62 | | | | | | | | | | |
| Scyth2p4_007263 | | Adhesin protein, | possible adhesin | | adhesin | | | 412 | 413 | 414 | 138 | 423 | 708 |
| | | putative | | | | | | | | | | | |
| Scyth2p4_007266 | Scyth2p4_007266 | unknown | xylan alpha-1,2- | hemicellulose- | xylan alpha-1,2- | GH115 | | 415 | 416 | 417 | 139 | 424 | 709 |
| | | | glucuronidase GH115 | modifying | glucuronidase | | | | | | | | |
| Scyth2p4_007287 | Scyth2p4_007287 | unknown | unknown | | uncharacterized | | | 418 | 419 | 420 | 140 | 425 | 710 |
| | | | | | lignocellulose-induced | | | | | | | | |
| | | | | | protein | | | | | | | | |
| Scyth2p4_007304 | Scyth2p4_007304 | unknown | unknown | | uncharacterized | | | 421 | 422 | 423 | 141 | 426 | 711 |
| | | | | | lignocellulose-induced | | | | | | | | |
| | | | | | protein | | | | | | | | |
| Scyth2p4_007313 | | Probable exo-1,4- | Beta-xylosidase GH3 | hemicellulose- | beta-xylosidase | GH3 | | 424 | 425 | 426 | 142 | 427 | 712 |
| | | beta-xylosidase | | degrading | | | | | | | | | |
| | | bxlB | | | | | | | | | | | |
| Scyth2p4_007314 | Scyth2p4_007314 | Probable feruloyl | feruloyl esterase CE1 | hemicellulose- | feruloyl esterase | CE1 | | 427 | 428 | 429 | 143 | 428 | 713 |
| | | esterase C | | degrading | | | | | | | | | |
| Scyth2p4_007531 | | unknown | unknown | | unknown CE2 | CE2 | | 430 | 431 | 432 | 144 | 429 | 714 |
| Scyth2p4_007556 | SCYTH_1_05275 | cellulase-enhancing | polysaccharide | cellulose- | polysaccharide | GH61 | CBM | 433 | 434 | 435 | 145 | 430 | 715 |
| | | protein | monooxygenase | degrading | monooxygenase | | 1 | | | | | | |
| Scyth2p4_007557 | Scyth2p4_007557 | cellulase-enhancing | polysaccharide | cellulose- | polysaccharide | GH61 | | 436 | 437 | 438 | 146 | 431 | 716 |
| | | protein | monooxygenase | degrading | monooxygenase | | | | | | | | |
| Scyth2p4_007647 | Scyth2p4_007647 | Probable beta- | Beta-galactosidase GH35 | hemicellulose- | beta-galactosidase | GH35 | | 439 | 440 | 441 | 147 | 432 | 717 |
| | | galactosidase B | | degrading | | | | | | | | | |
| Scyth2p4_007651 | SCYTH_1_05320 | cellulase-enhancing | polysaccharide | cellulose- | polysaccharide | GH61 | | 442 | 443 | 444 | 148 | 433 | 718 |
| | | protein | monooxygenase | degrading | monooxygenase | | | | | | | | |
| Scyth2p4_007699 | Scyth2p4_007699 | Endo-1,4-beta- | xylanase GH10 | hemicellulose- | xylanase | GH10 | | 445 | 446 | 447 | 149 | 434 | 719 |
| | | xylanase | | degrading | | | | | | | | | |
| Scyth2p4_007856 | Scyth2p4_007856 | Endo-1,4-beta- | xylanase GH10 | hemicellulose- | xylanase | GH10 | | 448 | 449 | 450 | 150 | 435 | 720 |
| | | xylanase | | degrading | | | | | | | | | |
| Scyth2p4_007921 | | Serine-type | Serine-type | protein | protease | | | 451 | 452 | 453 | 151 | 436 | 721 |
| | | carboxypeptidase F | carboxypeptidase F | hydrolysis | | | | | | | | | |
| Scyth2p4_008285 | Scyth2p4_008285 | unknown | unknown | | uncharacterized | | | 454 | 455 | 456 | 152 | 437 | 722 |
| | | | | | lignocellulose-induced | | | | | | | | |
| | | | | | protein | | | | | | | | |
| Scyth2p4_008294 | Scyth2p4_008294 | unknown | unknown | cellulase- | uncharacterized | | | 457 | 458 | 459 | 153 | 438 | 723 |
| | | | | enhancing | lignocellulose- | | | | | | | | |
| | | | | | induced protein² | | | | | | | | |
| Scyth2p4_008312 | Scyth2p4_008312 | unknown | unknown | | uncharacterized | | | 460 | 461 | 462 | 154 | 439 | 724 |
| | | | | | lignocellulose-induced | | | | | | | | |
| | | | | | protein | | | | | | | | |
| Scyth2p4_008328 | SCYTH_1_09441 | xylanase | xylanase GH11 | hemicellulose- | xylanase³ | GH11 | CBM | 463 | 464 | 465 | 155 | 440 | 725 |
| | | | | degrading | | | 1 | | | | | | |
| Scyth2p4_008336 | | Cuticle-degrading | Proteinase R | protein | protease | | | 466 | 467 | 468 | 156 | 441 | 726 |
| | | protease | | hydrolysis | | | | | | | | | |
| Scyth2p4_008341 | SCYTH_1_05851 | cellulase- | polysaccharide | cellulose- | polysaccharide | GH61 | | 469 | 470 | 471 | 157 | 442 | 727 |
| | | enhancing protein | monooxygenase | degrading | monooxygenase | | | | | | | | |
| Scyth2p4_008344 | | unknown | unknown | cellulose- | unknown CBM1 | | CBM | 472 | 473 | 474 | 158 | 443 | 728 |
| | | | | binding | | | 1 | | | | | | |
| Scyth2p4_008363 | SCYTH_1_00589 | endoglucanase | endoglucanase GH6 | cellulose- | endoglucanase | GH6 | | 475 | 476 | 477 | 159 | 444 | 729 |
| | | | | degrading | | | | | | | | | |
| Scyth2p4_008372 | SCYTH_1_01623 | cellobiohydrolase | cellobiohydrolase GH7 | cellulose- | cellobiohydrolase | GH7 | | 478 | 479 | 480 | 160 | 445 | 730 |
| | | | | degrading | | | | | | | | | |
| Scyth2p4_008392 | Scyth2p4_008392 | unknown | unknown | | uncharacterized | | | 481 | 482 | 483 | 161 | 446 | 731 |
| | | | | | lignocellulose-induced | | | | | | | | |
| | | | | | protein | | | | | | | | |
| Scyth2p4_008399 | Scyth2p4_008399 | Cellobiose | Cellobiose | Icellulose- | cellobiose | | | 484 | 485 | 486 | 162 | 447 | 732 |
| | | dehydrogenase | dehydrogenase | degrading | dehydrogenase | | | | | | | | |
| Scyth2p4_008411 | | Podosporapepsin | Podosporapepsin | protein | protease | | | 487 | 488 | 489 | 163 | 448 | 733 |
| | | | | hydrolysis | | | | | | | | | |
| Scyth2p4_008417 | | cellulase-enhancing | polysaccharide | cellulose- | polysaccharide | GH61 | | 490 | 491 | 492 | 164 | 449 | 734 |
| | | protein | monooxygenase | degrading | monooxygenase | | | | | | | | |
| Scyth2p4_008418 | Scyth2p4_008418 | cellulase-enhancing | polysaccharide | cellulose- | polysaccharide | GH61 | | 493 | 494 | 495 | 165 | 450 | 735 |
| | | protein | monooxygenase | degrading | monooxygenase | | | | | | | | |
| Scyth2p4_008663 | Scyth2p4_008663 | Putative | Putative | | oxidoreductase | | | 496 | 497 | 498 | 166 | 451 | 736 |
| | | uncharacterized | uncharacterized | | | | | | | | | | |
| | | oxidoreductase | oxidoreductase | | | | | | | | | | |
| | | YDR541C | YDR541C | | | | | | | | | | |
| Scyth2p4_008755 | Scyth2p4_008755 | glucoamylase | glucoamylase GH15 | starch- | glucoamylase | GH15 | | 499 | 500 | 501 | 167 | 452 | 737 |
| | | | | degrading | | | | | | | | | |
| Scyth2p4_008830 | | Phospholipase D | Alkaline phosphatase D | phospholipid- | lipase | | | 502 | 503 | 504 | 168 | 453 | 738 |
| | | | | modifying | | | | | | | | | |
| Scyth2p4_008896 | SCYTH_1_01504 | cellulase-enhancing | polysaccharide | cellulose- | polysaccharide | GH61 | | 505 | 506 | 507 | 169 | 454 | 739 |
| | | protein | monooxygenase | degrading | monooxygenase | | | | | | | | |
| Scyth2p4_009014 | | Gamma- | Gamma- | peptide- | protease | | | 508 | 509 | 510 | 170 | 455 | 740 |
| | | glutamyltranspeptidase 2 | glutamyltranspeptidase 1 | modifying | | | | | | | | | |
| Scyth2p4_009047 | Scyth2p4_009047 | Aldose 1-epimerase | Aldose 1-epimerase | | aldose epimerase | | | 511 | 512 | 513 | 171 | 456 | 741 |
| Scyth2p4_009244 | | Aspartic-type | Aspartic-type | protein | protease | | | 514 | 515 | 516 | 172 | 457 | 742 |
| | | endopeptidase ctsD | endopeptidase ctsD | hydrolysis | | | | | | | | | |
| Scyth2p4_009303 | Scyth2p4_009303 | Probable alpha-N- | alpha-arabinofuranosidase | hemicellulose- | arabinofuranosidase | GH51 | | 517 | 518 | 519 | 173 | 458 | 743 |
| | | arabinofuranosidase | GH51 | degrading | | | | | | | | | |
| | | A | | | | | | | | | | | |
| Scyth2p4_009308 | SCYTH_2_07268 | Feruloyl esterase B | feruloyl esterase CE1 | hemicellulose- | feruloyl esterase | CE1 | | 520 | 521 | 522 | 174 | 459 | 744 |
| | | | | degrading | | | | | | | | | |
| Scyth2p4_009393 | | Probable acetylxylan | Probable acetylxylan | hemicellulose- | acetylxylan esterase | CE1 | | 523 | 524 | 525 | 175 | 460 | 745 |
| | | esterase | esterase A | degrading | | | | | | | | | |
| Scyth2p4_009418 | | Endothiapepsin | Aspartic protease pep1 | protein | protease | | | 526 | 527 | 528 | 176 | 461 | 746 |
| | | | | hydrolysis | | | | | | | | | |
| Scyth2p4_009426 | Scyth2p4_009426 | Probable acetylxylan | Probable acetylxylan | hemicellulose- | acetylxylan esterase | CE1 | | 529 | 530 | 531 | 177 | 462 | 747 |
| | | esterase A | esterase A | modifying | | | | | | | | | |
| Scyth2p4_009442 | Scyth2p4_009442 | unknown | unknown | | uncharacterized | | CBM | 532 | 533 | 534 | 178 | 463 | 748 |
| | | | | | lignocellulose- | | 1 | | | | | | |
| | | | | | induced protein | | | | | | | | |
| Scyth2p4_009463 | | Probable leucine | Leucine aminopeptidase 2 | protein | protease | | | 535 | 536 | 537 | 179 | 464 | 749 |
| | | aminopeptidase 2 | | hydrolysis | | | | | | | | | |
| Scyth2p4_009475 | | Tripeptidyl- | Tripeptidyl-peptidase sed2 | peptide | protease | | | 538 | 539 | 540 | 180 | 465 | 750 |
| | | peptidase sed3 | | hydrolysis | | | | | | | | | |
| Scyth2p4_009509 | Scyth2p4_009509 | beta-mannanase | Beta-mannanase GH5 | hemicellulose- | beta-mannanase | GH5 | | 541 | 542 | 543 | 181 | 466 | 751 |
| | | | | degrading | | | | | | | | | |
| Scyth2p4_009510 | Scyth2p4_009510 | Glucoamylase | Glucoamylase | starch- | glucoamylase | GH119 | | 544 | 545 | 546 | 182 | 467 | 752 |
| | | | | degrading | | | | | | | | | |
| Scyth2p4_009516 | | unknown | unknown | | unknown PL20 | PL20 | | 547 | 548 | 549 | 183 | 468 | 753 |
| Scyth2p4_009525 | Scyth2p4_009525 | unknown | unknown | | uncharacterized | | | 550 | 551 | 552 | 184 | 469 | 754 |
| | | | | | lignocellulose-induced | | | | | | | | |
| | | | | | protein | | | | | | | | |
| Scyth2p4_009550 | | Cuticle-degrading | Cuticle-degrading | protein | protease | | | 553 | 554 | 555 | 185 | 470 | 755 |
| | | protease | protease | hydrolysis | | | | | | | | | |
| Scyth2p4_009554 | | unknown | unknown | | unknown CE3 | CE3 | | 556 | 557 | 558 | 186 | 471 | 756 |
| Scyth2p4_009565 | SCYTH_1_00574 | endoglucanase | endoglucanase GH7 | cellulose- | endoglucanase | GH7 | | 559 | 560 | 561 | 187 | 472 | 757 |
| | | | | degrading | | | | | | | | | |
| Scyth2p4_009569 | | Putative serine | Putative serine | protein | protease | | | 562 | 563 | 564 | 188 | 473 | 758 |
| | | protease K12H4.7 | protease K12H4.7 | hydrolysis | | | | | | | | | |
| Scyth2p4_009610 | | unknown | Beta-glucanase | glucan- | beta-glucanase | GH16 | | 565 | 566 | 567 | 189 | 474 | 759 |
| | | | | degrading | | | | | | | | | |
| Scyth2p4_009620 | SCYTH_1_08974 | endoglucanase | endoglucanase GH45 | cellulose- | endoglucanase | GH45 | CBM | 568 | 569 | 570 | 190 | 475 | 760 |
| | | | | degrading | | | 1 | | | | | | |
| Scyth2p4_009626 | SCYTH_1_01831 | arabinoxylan | arabinoxylan | hemicellulose- | arabinofuranosidase⁴ | GH62 | CBM | 571 | 572 | 573 | 191 | 476 | 761 |
| | | arabinofuranosidase | arabinofuranosidase | degrading | | | 1 | | | | | | |
| | | | GH62 | | | | | | | | | | |
| Scyth2p4_009629 | Scyth2p4_009629 | unknown | unknown | | uncharacterized | | | 574 | 575 | 576 | 192 | 477 | 762 |
| | | | | | lignocellulose-induced | | | | | | | | |
| | | | | | protein | | | | | | | | |
| Scyth2p4_009651 | Scyth2p4_009651 | unknown | Endo-1,4-beta- | hemicellulose- | endo-1,4-beta- | CE1 | | 577 | 578 | 579 | 193 | 478 | 763 |
| | | | xylanase Z | degrading | xylanase | | | | | | | | |
| Scyth2p4_009653 | Scyth2p4_009653 | xylanase | xylanase GH10 | hemicellulose- | xylanase | GH10 | | 580 | 581 | 582 | 194 | 479 | 764 |
| | | | | degrading | | | | | | | | | |
| Scyth2p4_009700 | Scyth2p4_009700 | cellobiohydrolase | cellobiohydrolase GH6 | cellulose- | cellobiohydrolase | GH6 | | 583 | 584 | 585 | 195 | 480 | 765 |
| | | | | degrading | | | | | | | | | |
| Scyth2p4_009707 | Scyth2p4_009707 | Cellobiose | Cellobiose | cellulose- | cellobiose | | | 586 | 587 | 588 | 196 | 481 | 766 |
| | | dehydrogenase | dehydrogenase | degrading | dehydrogenase | | | | | | | | |
| Scyth2p4_009711 | | unknown | unknown | | unknown | | | 589 | 590 | 591 | 197 | 482 | 767 |
| Scyth2p4_009720 | Scyth2p4_009720 | unknown | unknown | | uncharacterized | | | 592 | 593 | 594 | 198 | 483 | 768 |
| | | | | | lignocellulose-induced | | | | | | | | |
| | | | | | protein | | | | | | | | |
| Scyth2p4_009765 | | Adhesin protein | possible adhesin | | adhesin | | | 595 | 596 | 597 | 199 | 484 | 769 |
| | | Mad1 | | | | | | | | | | | |
| Scyth2p4_009796 | SCYTH_1_00755 | alpha-glucosidase | Alpha-glucosidase GH31 | starch- | alpha-glucosidase | GH31 | | 598 | 599 | 600 | 200 | 485 | 770 |
| | | | | degrading | | | | | | | | | |
| Scyth2p4_009823 | Scyth2p4_009823 | Expansin family | possible expansin | cellulase- | expansin | | | 601 | 602 | 603 | 201 | 486 | 771 |
| | | protein | | enhancing | | | | | | | | | |
| Scyth2p4_009929 | Scyth2p4_009929 | Chitinase 3 | Chitinase-like protein | chitin- | chitinase | GH18 | | 604 | 605 | 606 | 202 | 487 | 772 |
| | | | PB1E7.04c | degrading | | | | | | | | | |
| Scyth2p4_010021 | SCYTH_1_01020 | cellulase-enhancing | polysaccharide | cellulose- | polysaccharide | GH61 | | 607 | 608 | 609 | 203 | 488 | 773 |
| | | protein | monooxygenase | degrading | monooxygenase | | | | | | | | |
| Scyth2p4_010034 | | Adhesin protein | possible adhesin | | adhesion | | | 610 | 611 | 612 | 204 | 489 | 774 |
| | | Mad1 | | | | | | | | | | | |
| Scyth2p4_010146 | | unknown | unknown | | unknown CE1 | CE1 | | 613 | 614 | 615 | 205 | 490 | 775 |
| Scyth2p4_010149 | Scyth2p4_010149 | Exoglucanase 1 | Cellobiohydrolase GH7 | cellulose- | cellobiohydrolase | GH7 | | 616 | 617 | 618 | 206 | 491 | 776 |
| | | | | degrading | | | | | | | | | |
| Scyth2p4_010269 | Scyth2p4_010269 | Probable feruloyl | feruloyl esterase CE1 | hemicellulose- | feruloyl esterase | CE1 | CBM | 619 | 620 | 621 | 207 | 492 | 777 |
| | | esterase C | | modifying | | | 1 | | | | | | |
| Scyth2p4_010278 | Scyth2p4_010278 | Endochitinase | Killer toxin subunits | chitin- | chitinase | GH18 | CBM | 622 | 623 | 624 | 208 | 493 | 778 |
| | | | alpha/beta | degrading | | | 18 | | | | | | |
| Scyth2p4_010280 | | unknown | unknown | | unknown | | | 625 | 626 | 627 | 209 | 494 | 779 |
| Scyth2p4_010281 | | unknown | unknown | | unknown | | | 628 | 629 | 630 | 210 | 495 | 780 |
| Scyth2p4_010291 | | probable aspartic- | probable aspartic- | protein | protease | | | 631 | 632 | 633 | 211 | 496 | 781 |
| | | type endopeptidase | type endopeptidase | hydrolysis | | | | | | | | | |
| | | opsB | opsB | | | | | | | | | | |
| Scyth2p4_010295 | Scyth2p4_010295 | cutinase | cutinase CE5 | cutin-degrading | cutinase | CE5 | | 634 | 635 | 636 | 212 | 497 | 782 |
| Scyth2p4_010361 | | choline | possible pyranose | sugar | pyranose | | | 637 | 638 | 639 | 213 | 498 | 783 |
| | | dehydrogenase | dehydrogenase | modifying | dehydrogenase | | | | | | | | |
| Scyth2p4_010373 | Scyth2p4_010373 | carbohydrate- | possible carbohydrate- | carbohydrate- | carbohydrate-binding | | | 640 | 641 | 642 | 214 | 499 | 784 |
| | | binding cytochrome | binding cytochrome | oxidizing | cytochrome | | | | | | | | |
| | | b562 | | | | | | | | | | | |
| Scyth2p4_010387 | | exo-1,3-beta- | exo-1,3-beta- | cellulose- | exo-1,3-beta- | GH55 | | 643 | 644 | 645 | 215 | 500 | 785 |
| | | glucanase | glucanase GH55 | degrading | glucanase | | | | | | | | |
| Scyth2p4_010416 | | Choline | Choline | | Choline | | | 646 | 647 | 648 | 216 | 501 | 786 |
| | | dehydrogenase | dehydrogenase | | dehydrogenase | | | | | | | | |
| Scyth2p4_010423 | Scyth2p4_010423 | unknown | Uncharacterized | | uncharacterized | | | 649 | 650 | 651 | 217 | 502 | 787 |
| | | | protein YkgB | | lignocellulose-induced | | | | | | | | |
| | | | | | protein | | | | | | | | |
| Scyth2p4_010457 | SCYTH_1_01114 | xylanase | xylanase GH11 | hemicellulose- | xylanase⁵ | GH11 | | 652 | 653 | 654 | 218 | 503 | 788 |
| | | | | degrading | | | | | | | | | |
| Scyth2p4_010462 | Scyth2p4_010462 | Probable endo-1,4- | Probable endo-1,4- | hemicellulose- | endo-1,4-beta- | GH11 | | 655 | 656 | 657 | 219 | 504 | 789 |
| | | beta-xylanase B | beta-xylanase B | degrading | xylanase B | | | | | | | | |
| Scyth2p4_010469 | | choline | possible pyranose | sugar- | pyranose | | | 658 | 659 | 660 | 220 | 505 | 790 |
| | | dehydrogenase | dehydrogenase | modifying | dehydrogenase | | | | | | | | |
| Scyth2p4_010519 | | Peptidase M20 | Peptidase M20 domain- | protein | protease | | | 661 | 662 | 663 | 221 | 506 | 791 |
| | | domain-containing | containing protein | hydrolysis | | | | | | | | | |
| | | protein C757.05c | SMAC_03666.2 | | | | | | | | | | |
| Scyth2p4_010552 | | choline | possible pyranose | sugar- | pyranose | | | 664 | 665 | 666 | 222 | 507 | 792 |
| | | dehydrogenase | dehydrogenase | modifying | dehydrogenase | | | | | | | | |
| Scyth2p4_010553 | | chitinase A1 | chitinase GH18 | chitin- | chitinase | GH18 | | 667 | 668 | 669 | 223 | 508 | 793 |
| | | | | degrading | | | | | | | | | |
| Scyth2p4_010743 | | cellulase-enhancing | polysaccharide | cellulose- | polysaccharide | GH61 | | 670 | 671 | 672 | 224 | 509 | 794 |
| | | protein | monooxygenase | degrading | monooxygenase | | | | | | | | |
| Scyth2p4_010756 | Scyth2p4_010756 | unknown | unknown | | uncharacterized | | | 673 | 674 | 675 | 225 | 510 | 795 |
| | | | | | lignocellulose-induced | | | | | | | | |
| | | | | | protein | | | | | | | | |
| Scyth2p4_010779 | SCYTH_1_01186 | probable chitinase 3 | acidic mammalian | chitin- | chitinase | GH18 | CBM | 676 | 677 | 678 | 226 | 511 | 796 |
| | | | chitinase | degrading | | | 18 | | | | | | |
| Scyth2p4_010780 | | unknown | unknown | | unknown | | | 679 | 680 | 681 | 227 | 512 | 797 |
| Scyth2p4_010784 | | exo-beta-D- | exo-glucosaminidase GH2 | chitin- | exo-glucosaminidase | GH2 | | 682 | 683 | 684 | 228 | 513 | 798 |
| | | glucosaminidase | | degrading | | | | | | | | | |
| Scyth2p4_010822 | Scyth2p4_010822 | Probable pectate | pectate lyase PL3 | pectin- | pectate lyase | PL3 | | 685 | 686 | 687 | 229 | 514 | 799 |
| | | lyase D | | degrading | | | | | | | | | |
| Scyth2p4_010823 | | laminarinase | laminarinase GH55 | glucan- | laminarinase | GH55 | | 688 | 689 | 690 | 230 | 515 | 800 |
| | | | | degrading | | | | | | | | | |
| Scyth2p4_010825 | Scyth2p4_010825 | cellulase-GH5 | galactanase GH5 | hemicellulose- | galactanase | GH5 | | 691 | 692 | 693 | 231 | 516 | 801 |
| | | | | degrading | | | | | | | | | |
| Scyth2p4_010857 | Scyth2p4_010857 | endo-1,4-beta- | xylanase GH11 | hemicellulose- | xylanase | GH11 | | 694 | 695 | 696 | 232 | 517 | 802 |
| | | xylanase A | | degrading | | | | | | | | | |
| Scyth2p4_010865 | SCYTH_1_04962 | cellulase-enhancing | polysaccharide | cellulose- | polysaccharide | GH61 | | 697 | 698 | 699 | 233 | 518 | 803 |
| | | protein | monooxygenase | degrading | monooxygenase | | | | | | | | |
| Scyth2p4_010870 | | unknown | unknown | | unknown | | | 700 | 701 | 702 | 234 | 519 | 804 |
| Scyth2p4_010884 | Scyth2p4_010884 | unknown | unknown | | uncharacterized | | | 703 | 704 | 705 | 235 | 520 | 805 |
| | | | | | lignocellulose-induced | | | | | | | | |
| | | | | | protein | | | | | | | | |
| Scyth2p4_010894 | Scyth2p4_010894 | Acetylxylan esterase | acetylxylan esterase CE5 | hemicellulose- | acetylxylan esterase | CE5 | CBM | 706 | 707 | 708 | 236 | 521 | 806 |
| | | | | modifying | | | 1 | | | | | | |
| Scyth2p4_010898 | SCYTH_1_00286 | endo-1,4-beta- | xylanase GH10 | hemicellulose- | xylanase | GH10 | CBM | 709 | 710 | 711 | 237 | 522 | 807 |
| | | xylanase | | degrading | | | 1 | | | | | | |
| Scyth2p4_010899 | | aminopeptidase Y | aminopeptidase Y | protein | protease | | | 712 | 713 | 714 | 238 | 523 | 808 |
| | | | | hydrolysis | | | | | | | | | |
| | Scyth2p4_001141 | | Probable glycosidase crf1 | carbohydrate- | glycosidase | GH16 | | | | | 239 | 524 | 809 |
| | | | | modifying | | | | | | | | | |
| | Scyth2p4_001257 | | unknown | | uncharacterized | | | | | | 240 | 525 | 810 |
| | | | | | lignocellulose-induced | | | | | | | | |
| | | | | | protein | | | | | | | | |
| | Scyth2p4_001442 | | unknown | | uncharacterized | | | | | | 241 | 526 | 811 |
| | | | | | lignocellulose-induced | | | | | | | | |
| | | | | | protein | | | | | | | | |
| | Scyth2p4_001768 | | trehalase | starch- | trehalase | GH37 | | | | | 242 | 527 | 812 |
| | | | | degrading | | | | | | | | | |
| | Scyth2p4_002054 | | alpha-amylase GH13 | starch- | alpha-amylase | GH13 | | | | | 243 | 528 | 813 |
| | | | | degrading | | | | | | | | | |
| | Scyth2p4_003709 | | unknown | | uncharacterized | | | | | | 244 | 529 | 814 |
| | | | | | lignocellulose-induced | | | | | | | | |
| | | | | | protein | | | | | | | | |
| | Scyth2p4_003954 | | unknown | | uncharacterized | | CBM | | | | 245 | 530 | 815 |
| | | | | | lignocellulose-induced | | 1 | | | | | | |
| | | | | | protein | | | | | | | | |
| | Scyth2p4_004342 | | unknown | | uncharacterized | | | | | | 246 | 531 | 816 |
| | | | | | lignocellulose-induced | | | | | | | | |
| | | | | | protein | | | | | | | | |
| | Scyth2p4_004817 | | unknown | | uncharacterized | | | | | | 247 | 532 | 817 |
| | | | | | lignocellulose-induced | | | | | | | | |
| | | | | | protein | | | | | | | | |
| | Scyth2p4_005217 | | unknown | | uncharacterized | | | | | | 248 | 533 | 818 |
| | | | | | lignocellulose-induced | | | | | | | | |
| | | | | | protein | | | | | | | | |
| | Scyth2p4_007345 | | tyrosinase | pigment- | tyrosinase | | | | | | 249 | 534 | 819 |
| | | | | producing | | | | | | | | | |
| | Scyth2p4_007869 | | unknown | | uncharacterized | | | | | | 250 | 535 | 820 |
| | | | | | lignocellulose-induced | | | | | | | | |
| | | | | | protein | | | | | | | | |
| | Scyth2p4_009477 | | unknown | | uncharacterized | | | | | | 251 | 536 | 821 |
| | | | | | lignocellulose-induced | | | | | | | | |
| | | | | | protein | | | | | | | | |
| | Scyth2p4_009552 | | unknown | | uncharacterized | | | | | | 252 | 537 | 822 |
| | | | | | lignocellulose-induced | | | | | | | | |
| | | | | | protein | | | | | | | | |
| | Scyth2p4_009704 | | Uncharacterized | | uncharacterized | | | | | | 253 | 538 | 823 |
| | | | protein L662 | | lignocellulose-induced | | | | | | | | |
| | | | | | protein | | | | | | | | |
| | Scyth2p4_010302 | | unknown | | uncharacterized | | | | | | 254 | 539 | 824 |
| | | | | | lignocellulose-induced | | | | | | | | |
| | | | | | protein | | | | | | | | |
| | Scyth2p4_010820 | | unknown | | uncharacterized | | | | | | 255 | 540 | 825 |
| | | | | | lignocellulose-induced | | | | | | | | |
| | | | | | protein | | | | | | | | |
| | SCYTH_1_00385 | | Probable beta- | hemicellulose- | beta-mannosidase B | GH2 | | | | | 256 | 541 | 826 |
| | | | mannosidase B | degrading | | | | | | | | | |
| | SCYTH_1_00739 | | hexosaminidase GH20 | chitin- | hexosaminidase | GH20 | | | | | 257 | 542 | 827 |
| | | | | degrading | | | | | | | | | |
| [Scyth2p4_006265]⁶ | SCYTH_1_02579 | | beta-glucosidase GH3 | cellulose- | beta-glucosidase | GH3 | | | | | 258 | 543 | 828 |
| | | | | degrading | | | | | | | | | |
| | SCYTH_1_03688 | | arabinoxylan | hemicellulose- | arabinoxylan | GH62 | | | | | 259 | 544 | 829 |
| | | | arabinofuranohydrolase | degrading | arabinofurano- | | | | | | | | |
| | | | GH62 | | hydrolase | | | | | | | | |
| | SCYTH_1_09019 | | xylanase GH10 | hemicellulose- | xylanase⁷ | GH10 | | | | | 260 | 545 | 830 |
| | | | | degrading | | | | | | | | | |
| | SCYTH_2_05417 | | unknown | | uncharacterized | | | | | | 261 | 546 | 831 |
| | | | | | lignocellulose-induced | | | | | | | | |
| | | | | | protein | | | | | | | | |
| | SCYTH_2_07393 | | Acetylxylan esterase 1 | hemicellulose- | acetylxylan esterase 1 | CE1 | CBM | | | | 262 | 547 | 832 |
| | | | CE1 | modifying | | | 1 | | | | | | |
| | Scyth2p4_000071 | | Dipeptidyl peptidase 1 | protein | protease | | | | | | 263 | 548 | 833 |
| | | | (Fragment) | hydrolysis | | | | | | | | | |
| | Scyth2p4_000786 | | unknown | | unknown CE1 | CE1 | | | | | 264 | 549 | 834 |
| | Scyth2p4_000879 | | unknown | | unknown CE3 | CE3 | | | | | 265 | 550 | 835 |
| | Scyth2p4_001265 | | Putative sterigmatocystin | lignin- | peroxidase | | | | | | 266 | 551 | 836 |
| | | | biosynthesis peroxidase | degrading | | | | | | | | | |
| | | | stcC | | | | | | | | | | |
| | Scyth2p4_001349 | | Probable serine protease | protein | protease | | | | | | 267 | 552 | 837 |
| | | | EDA2 | hydrolysis | | | | | | | | | |
| | Scyth2p4_002059 | | Aspartic proteinase | protein | protease | | | | | | 268 | 553 | 838 |
| | | | yapsin-3 | hydrolysis | | | | | | | | | |
| | Scyth2p4_002062 | | Lipase 4 | lipid-modifying | lipase | CE10 | | | | | 269 | 554 | 839 |
| | Scyth2p4_002618 | | possible pyranose | sugar- | pyranose | | | | | | 270 | 555 | 840 |
| | | | dehydrogenase | modifying | dehydrogenase | | | | | | | | |
| | Scyth2p4_002885 | | possible adhesin | | adhesin | | | | | | 271 | 556 | 841 |
| | Scyth2p4_003845 | | unknown | | unknown CBM18 | | CBM | | | | 272 | 557 | 842 |
| | | | | | | | 18 | | | | | | |
| | Scyth2p4_003921 | | Dipeptidyl peptidase 4 | protein | protease | CE1 | | | | | 273 | 558 | 843 |
| | | | | hydrolysis | | | | | | | | | |
| | Scyth2p4_003974 | | Lipase 2 | lipid-modifying | lipase | CE10 | | | | | 274 | 559 | 844 |
| | Scyth2p4_003996 | | Minor extracellular | protein | protease | | | | | | 275 | 560 | 845 |
| | | | protease vpr | hydrolysis | | | | | | | | | |
| | Scyth2p4_004891 | | possible adhesin | | adhesin | | | | | | 276 | 561 | 846 |
| | Scyth2p4_005785 | | Probable isoaspartyl | protein | protease | | | | | | 277 | 562 | 847 |
| | | | peptidase/L-asparaginase | hydrolysis | | | | | | | | | |
| | | | 3 | | | | | | | | | | |
| | Scyth2p4_006840 | | Aspergillopepsin-2 | protein | protease | | | | | | 278 | 563 | 848 |
| | | | | hydrolysis | | | | | | | | | |
| | Scyth2p4_007340 | | Alcohol dehydrogenase | sugar- | pyranose | | | | | | 279 | 564 | 849 |
| | | | [acceptor] | modifiying | dehydrogenase | | | | | | | | |
| | Scyth2p4_007698 | | Uncharacterized FAD- | | oxidoreductase | | | | | | 280 | 565 | 850 |
| | | | linked oxidoreductase | | | | | | | | | | |
| | | | yvdP | | | | | | | | | | |
| | Scyth2p4_008300 | | Extracellular | protein | protease | | | | | | 281 | 566 | 851 |
| | | | metalloprotease | hydrolysis | | | | | | | | | |
| | | | Pa_2_14170 | | | | | | | | | | |
| | Scyth2p4_009549 | | Uncharacterized FAD- | | oxidoreductase | | | | | | 282 | 567 | 852 |
| | | | linked oxidoreductase | | | | | | | | | | |
| | | | yvdP | | | | | | | | | | |
| | Scyth2p4_010449 | | Probable aspartic-type | protein | protease | GH109 | | | | | 283 | 568 | 853 |
| | | | endopeptidase | hydrolysis | | | | | | | | | |
| | | | AFUA_3G01220 | | | | | | | | | | |
| | Scyth2p4_010575 | | Subtilisin-like protease | protein | protease | | | | | | 284 | 569 | 854 |
| | | | CPC735_066880 | hydrolysis | | | | | | | | | |
| | Scyth2p4_010881 | | Uncharacterized FAD- | | oxidoreductase | | | | | | 285 | 570 | 855 |
| | | | linked oxidoreductase | | | | | | | | | | |
| | | | yvdP | | | | | | | | | | |

¹For example, exoglucanase-6A
²Simiar to aromatic ring-cleavage diooxygenases, upregulated by organism upon growth on biomass
³For example, endo-1,4-beta-xylanase B
⁴For example, alpha-L-arabinofuranosidase axhA-2.
⁵For example, endo-1,4-beta-xylanase 1
⁶Square brackets (“[” and “]”) used in this column in Tables 1A-1C are meant to indicate the possibility that the Gene IDs may have been modified from the provisional application.
⁷For example, Endo-1,4-beta-xylanase

TABLE 1B
Biomass degrading genes and polypeptides of Myriococcum thermophilum

| | | | | | | | | Provisional | | | PCT | | |
|---|---|---|---|---|---|---|---|---|---|---|---|---|---|
| Gene ID in | | Annotation in | | | | | | application | | | application | | |
| Provisional | | Provisional | | | | | CBM | SEQ ID NO: | | | SEQ ID NO: | | |
| Application No. | | Application No. | | | | CAZy | of in- | Ge- | Cod- | Amino | Ge- | Cod- | Amino |
| 61/657,075 | Target ID | 61/657,075 | Updated annotation | Function | Protein activity | family | terest | nomic | ing | acid | nomic | ing | acid |
| Myrth2p4_000015 | Myrth2p4_000015 | Putative beta- | arabinoxylan | hemicellulose- | arabinofuranosidase | GH43 | | 1 | 2 | 3 | 856 | 1162 | 1468 |
| | | xylosidase | arabinofuranohydrolase | degrading | | | | | | | | | |
| | | | GH43 | | | | | | | | | | |
| Myrth2p4_000358 | MYRTH_2_03236 | cellulase- | polysaccharide | cellulose- | polysaccharide | GH61 | | 4 | 5 | 6 | 857 | 1163 | 1469 |
| | | enhancing protein | monooxygenase | degrading | monooxygenase | | | | | | | | |
| Myrth2p4_000359 | Myrth2p4_000359 | Cellobiose | Cellobiose | lignin- | cellobiose | | | 7 | 8 | 9 | 858 | 1164 | 1470 |
| | | dehydrogenase | dehydrogenase | degrading | dehydrogenase | | | | | | | | |
| Myrth2p4_000363 | | Podosporapepsin | Podosporapepsin | protein | protease | | | 10 | 11 | 12 | 859 | 1165 | 1471 |
| | | | | hydrolysis | | | | | | | | | |
| Myrth2p4_000376 | Myrth2p4_000376 | unknown | unknown | | uncharacterized | | | 13 | 14 | 15 | 860 | 1166 | 1472 |
| | | | | | lignocellulose- | | | | | | | | |
| | | | | | induced protein | | | | | | | | |
| Myrth2p4_000388 | MYRTH_2_00256 | cellobiohydrolase | cellobiohydrolase GH7 | cellulose- | cellobiohydrolase | GH7 | | 16 | 17 | 18 | 861 | 1167 | 1473 |
| | | | | degrading | | | | | | | | | |
| Myrth2p4_000417 | Myrth2p4_000417 | Acid phosphatase | Acid phosphatase | dephosphorylating | Acid phosphatase | | | 19 | 20 | 21 | 862 | 1168 | 1474 |
| Myrth2p4_000486 | | Aspergillopepsin-2 | Aspergillopepsin-2 | protein | protease | | | 22 | 23 | 24 | 863 | 1169 | 1475 |
| | | | | hydrolysis | | | | | | | | | |
| Myrth2p4_000495 | Myrth2p4_000495 | unknown | arabinoxylan | hemicellulose- | arabinofuranosidase | GH43 | | 25 | 26 | 27 | 864 | 1170 | 1476 |
| | | | arabinofuranohydrolase | degrading | | | | | | | | | |
| | | | GH43 | | | | | | | | | | |
| Myrth2p4_000510 | MYRTH_2_02564 | unknown | unknown | | uncharacterized | | | 28 | 29 | 30 | 865 | 1171 | 1477 |
| | | | | | lignocellulose- | | | | | | | | |
| | | | | | induced protein | | | | | | | | |
| Myrth2p4_000524 | | unknown | unknown | | unknown | | | 31 | 32 | 33 | 866 | 1172 | 1478 |
| Myrth2p4_000531 | Myrth2p4_000531 | Uncharacterized | chitin deacetylase CE4 | chitin- | chitin deacetylase | CE4 | | 34 | 35 | 36 | 867 | 1173 | 1479 |
| | | protein yjeA | | degrading | | | | | | | | | |
| Myrth2p4_000543 | | Probable aspartic- | Candidapepsin-8 | protein | protease | | | 37 | 38 | 39 | 868 | 1174 | 1480 |
| | | type endopeptidase | | hydrolysis | | | | | | | | | |
| | | opsB | | | | | | | | | | | |
| Myrth2p4_000545 | | unknown | unknown | | unknown | | | 40 | 41 | 42 | 869 | 1175 | 1481 |
| Myrth2p4_000589 | | unknown | carbohydrate esterase | hemicellulose- | unknown CE15 | CE15 | | 43 | 44 | 45 | 870 | 1176 | 1482 |
| | | | | modifying | | | | | | | | | |
| Myrth2p4_000694 | | Putative lipase | Putative lipase | lipid- | lipase | | | 46 | 47 | 48 | 871 | 1177 | 1483 |
| | | atg15 | atg15 | degrading | | | | | | | | | |
| Myrth2p4_000867 | | unknown | xylanase GH30 | hemicellulose- | xylanase | GH30 | | 49 | 50 | 51 | 872 | 1178 | 1484 |
| | | | | degrading | | | | | | | | | |
| Myrth2p4_000999 | Myrth2p4_000999 | unknown | unknown | | uncharacterized | | | 52 | 53 | 54 | 873 | 1179 | 1485 |
| | | | | | lignocellulose- | | | | | | | | |
| | | | | | induced protein | | | | | | | | |
| Myrth2p4_001083 | | Carboxypeptidase Y | Carboxypeptidase Y | protein | protease | | | 55 | 56 | 57 | 874 | 1180 | 1486 |
| | | | homolog A | hydrolysis | | | | | | | | | |
| Myrth2p4_001208 | | Probable aspartic- | Probable aspartic- | protein | protease | | | 58 | 59 | 60 | 875 | 1181 | 1487 |
| | | type endopeptidase | type endopeptidase | hydrolysis | | | | | | | | | |
| | | OPSB | OPSB | | | | | | | | | | |
| Myrth2p4_001304 | Myrth2p4_001304 | Cellobiose | Cellobiose | lignin- | cellobiose | | CBM | 61 | 62 | 63 | 876 | 1182 | 1488 |
| | | dehydrogenase | dehydrogenase | degrading | dehydrogenase | | 1 | | | | | | |
| Myrth2p4_001319 | | unknown | endo-beta-1,3- | hemicellulose- | endo-beta-1,3- | GH16 | | 64 | 65 | 66 | 877 | 1183 | 1489 |
| | | | galactanase GH16 | degrading | galactanase | | | | | | | | |
| Myrth2p4_001328 | Myrth2p4_001328 | unknown | unknown | | uncharacterized | | | 67 | 68 | 69 | 878 | 1184 | 1490 |
| | | | | | lignocellulose- | | | | | | | | |
| | | | | | induced protein | | | | | | | | |
| Myrth2p4_001333 | MYRTH_3_00119 | cellulase-enhancing | polysaccharide | cellulose- | polysaccharide | GH61 | | 70 | 71 | 72 | 879 | 1185 | 1491 |
| | | protein | monooxygenase | degrading | monooxygenase | | | | | | | | |
| Myrth2p4_001339 | Myrth2p4_001339 | beta-glucosidase | beta-glucosidase GH3 | cellulose- | beta-glucosidase | GH3 | | 73 | 74 | 75 | 880 | 1186 | 1492 |
| | | | | degrading | | | | | | | | | |
| Myrth2p4_001354 | Myrth2p4_001354 | endoglucanase | Probable xyloglucan- | hemicellulose- | xyloglucan-specific | GH12 | | 76 | 77 | 78 | 881 | 1187 | 1493 |
| | | | specificendo-beta-1,4- | degrading | endo-beta-1,4- | | | | | | | | |
| | | | glucanase A | | glucanase A | | | | | | | | |
| Myrth2p4_001362 | MYRTH_3_00118 | cellulase-enhancing | polysaccharide | cellulose- | polysaccharide | GH61 | | 79 | 80 | 81 | 882 | 1188 | 1494 |
| | | protein | monooxygenase | degrading | monooxygenase | | | | | | | | |
| Myrth2p4_001366 | Myrth2p4_001366 | unknown | unknown | | uncharacterized | | | 82 | 83 | 84 | 883 | 1189 | 1495 |
| | | | | | lignocellulose- | | | | | | | | |
| | | | | | induced protein | | | | | | | | |
| Myrth2p4_001368 | | exo-1,3-beta- | exo-1,3-beta- | cellulose- | exo-1,3-beta- | GH55 | | 85 | 86 | 87 | 884 | 1190 | 1496 |
| | | glucanase | glucanase GH55 | degrading | glucanase | | | | | | | | |
| Myrth2p4_001374 | MYRTH_3_00100 | cellulase-enhancing | polysaccharide | cellulose- | polysaccharide | GH61 | | 88 | 89 | 90 | 885 | 1191 | 1497 |
| | | protein | monooxygenase | degrading | monooxygenase | | | | | | | | |
| Myrth2p4_001375 | MYRTH_2_03396 | xylanase | xylanase GH10 | hemicellulose- | xylanase | GH10 | | 91 | 92 | 93 | 886 | 1192 | 1498 |
| | | | | degrading | | | | | | | | | |
| Myrth2p4_001378 | Myrth2p4_001378 | unknown | unknown | | uncharacterized | | | 94 | 95 | 96 | 887 | 1193 | 1499 |
| | | | | | lignocellulose- | | | | | | | | |
| | | | | | induced protein | | | | | | | | |
| Myrth2p4_001403 | MYRTH_2_02621 | Probable | Acetylxylan esterase 1 | Hemicellulose- | acetylxylan esterase | CE1 | | 97 | 98 | 99 | 888 | 1194 | 1500 |
| | | acetylxylan esterase | CE1 | modifying | | | | | | | | | |
| | | A | | | | | | | | | | | |
| Myrth2p4_001451 | Myrth2p4_001451 | xylanase | xylanase GH11 | hemicellulose- | xylanase | GH11 | | 100 | 101 | 102 | 889 | 1195 | 1501 |
| | | | | degrading | | | | | | | | | |
| Myrth2p4_001463 | MYRTH_1_00071 | chitinase | Chitinase GH18 | chitin- | chitinase | GH18 | | 103 | 104 | 105 | 890 | 1196 | 1502 |
| | | | | degrading | | | | | | | | | |
| Myrth2p4_001467 | | unknown | exo-1,3-beta- | hemicellulose- | exo-1,3-beta- | GH43 | CBM | 106 | 107 | 108 | 891 | 1197 | 1503 |
| | | | galactanase GH43 | degrading | galactanase | | 35 | | | | | | |
| Myrth2p4_001469 | | Tripeptidyl- | Tripeptidyl- | peptide | protease | | | 109 | 110 | 111 | 892 | 1198 | 1504 |
| | | peptidase sed2 | peptidase sed3 | hydrolysis | | | | | | | | | |
| Myrth2p4_001494 | Myrth2p4_001494 | Probable beta- | beta-glucosidase GH3 | cellulose- | beta-glucosidase | GH3 | | 112 | 113 | 114 | 893 | 1199 | 1505 |
| | | glucosidase L | | degrading | | | | | | | | | |
| Myrth2p4_001496 | Myrth2p4_001496 | Probable exo-1,4- | beta-xylosidase GH3 | hemicellulose- | beta-xylosidase⁸ | GH3 | | 115 | 116 | 117 | 894 | 1200 | 1506 |
| | | beta-xylosidase | | degrading | | | | | | | | | |
| | | bxlB | | | | | | | | | | | |
| Myrth2p4_001537 | Myrth2p4_001537 | Acetylxylan | Acetylxylan | hemicellulose- | acetylxylan | CE5 | | 118 | 119 | 120 | 895 | 1201 | 1507 |
| | | esterase 2 | esterase 2 CE5 | modifying | esterase | | | | | | | | |
| Myrth2p4_001550 | Myrth2p4_001550 | unknown | unknown | | uncharacterized | | | 121 | 122 | 123 | 896 | 1202 | 1508 |
| | | | | | lignocellulose- | | | | | | | | |
| | | | | | induced protein | | | | | | | | |
| Myrth2p4_001581 | MYRTH_2_03760 | cellulase- | polysaccharide | cellulose- | polysaccharide | GH61 | | 124 | 125 | 126 | 897 | 1203 | 1509 |
| | | enhancing protein | monooxygenase | degrading | monooxygenase | | | | | | | | |
| Myrth2p4_001582 | MYRTH_1_00083 | Beta-galactosidase | Beta-galactosidase | hemicellulose- | Beta-galactosidase | GH2 | | 127 | 128 | 129 | 898 | 1204 | 1510 |
| | | | | degrading | | | | | | | | | |
| Myrth2p4_001589 | | Putative serine | Probable serine | protein | protease | | | 130 | 131 | 132 | 899 | 1205 | 1511 |
| | | protease F56F10.1 | protease EDA2 | hydrolysis | | | | | | | | | |
| Myrth2p4_001667 | | unknown | unknown | | unknown CE4 | CE4 | | 133 | 134 | 135 | 900 | 1206 | 1512 |
| Myrth2p4_001718 | Myrth2p4_001718 | unknown | unknown | | unknown CE15 | CE15 | | 136 | 137 | 138 | 901 | 1207 | 1513 |
| Myrth2p4_001719 | MYRTH_2_02768 | arabinogalactanase | arabinogalactanase | hemicellulose- | arabinogalactanase | GH53 | | 139 | 140 | 141 | 902 | 1208 | 1514 |
| | | | GH53 | degrading | | | | | | | | | |
| Myrth2p4_001916 | Myrth2p4_001916 | Probable beta- | Probable beta- | cellulose- | beta-glucosidase | GH17 | | 142 | 143 | 144 | 903 | 1209 | 1515 |
| | | glucosidase btgE | glucosidase btgE | degrading | | | | | | | | | |
| Myrth2p4_001926 | | Probable endo-1,3(4)- | mixed-link glucanase | glucan- | mixed-link | GH16 | | 145 | 146 | 147 | 904 | 1210 | 1516 |
| | | beta-glucanase | GH16 | degrading | glucanase | | | | | | | | |
| | | NFIA_089530 | | | | | | | | | | | |
| Myrth2p4_001996 | Myrth2p4_001996 | endoglucanase | endoglucanase GH45 | cellulose- | endoglucanase | GH45 | | 148 | 149 | 150 | 905 | 1211 | 1517 |
| | | | | degrading | | | | | | | | | |
| Myrth2p4_002010 | | Lysophospholipase | Lysophospholipase | lipid- | lipase | | | 151 | 152 | 153 | 906 | 1212 | 1518 |
| | | | | modifying | | | | | | | | | |
| Myrth2p4_002134 | | Putative | Aspartic protease pep1 | protein | protease | | | 154 | 155 | 156 | 907 | 1213 | 1519 |
| | | aspergillopepsin A- | | hydrolysis | | | | | | | | | |
| | | like aspartic | | | | | | | | | | | |
| | | endopeptidase | | | | | | | | | | | |
| | | AFUA_2G15950 | | | | | | | | | | | |
| Myrth2p4_002293 | Myrth2p4_002293 | endoglucanase | Endoglucanase GH5 | cellulose- | Endoglucanase | GH5 | CBM | 157 | 158 | 159 | 908 | 1214 | 1520 |
| | | | | degrading | | | 1 | | | | | | |
| Myrth2p4_002328 | | Aspergillopepsin-2 | Aspergillopepsin-2 | protein | protease | | | 160 | 161 | 162 | 909 | 1215 | 1521 |
| | | | | hydrolysis | | | | | | | | | |
| Myrth2p4_002394 | MYRTH_2_04289 | Mannanendo-1,4- | Beta-mannanase GH26 | hemicellulose- | Beta-mannanase | GH26 | CBM | 163 | 164 | 165 | 910 | 1216 | 1522 |
| | | beta-mannosidase | | degrading | | | 35 | | | | | | |
| Myrth2p4_002434 | MYRTH_1_00022 | alpha-glucosidase | alpha-glucosidase GH31 | starch- | alpha-glucosidase | GH31 | | 166 | 167 | 168 | 911 | 1217 | 1523 |
| | | | | degrading | | | | | | | | | |
| Myrth2p4_002456 | | Carbohydrate- | unknown | | Carbohydrate- | | | 169 | 170 | 171 | 912 | 1218 | 1524 |
| | | binding cytochrome | | | binding cytochrome | | | | | | | | |
| | | b562 | | | | | | | | | | | |
| Myrth2p4_002548 | | unknown | unknown | | unknown | | | 172 | 173 | 174 | 913 | 1219 | 1525 |
| Myrth2p4_002549 | Myrth2p4_002549 | cellobiohydrolase | cellobiohydrolase GH7 | cellulose- | cellobiohydrolase | GH7 | | 175 | 176 | 177 | 914 | 1220 | 1526 |
| | | | | degrading | | | | | | | | | |
| Myrth2p4_002563 | | Hybrid signal | Hybrid signal | | kinase | | | 178 | 179 | 180 | 915 | 1221 | 1527 |
| | | transduction | transduction | | | | | | | | | | |
| | | histidine kinase J | histidine kinase J | | | | | | | | | | |
| Myrth2p4_002601 | Myrth2p4_002601 | Probable feruloyl | Probable feruloyl | hemicellulose- | feruloyl esterase | | | 181 | 182 | 183 | 916 | 1222 | 1528 |
| | | esterase A | esterase A | degrading | | | | | | | | | |
| Myrth2p4_002632 | | unknown | Protoporphyrinogen | | oxidoreductase | | | 184 | 185 | 186 | 917 | 1223 | 1529 |
| | | | oxidase | | | | | | | | | | |
| Myrth2p4_002634 | MYRTH_2_04579 | hexosaminidase | Hexosaminidase GH20 | chitin- | Hexosaminidase | GH20 | | 187 | 188 | 189 | 918 | 1224 | 1530 |
| | | | | degrading | | | | | | | | | |
| Myrth2p4_002638 | Myrth2p4_002638 | Probable | Probable glycosidase | carbohydrate- | glycosidase | GH16 | | 190 | 191 | 192 | 919 | 1225 | 1531 |
| | | glycosidase CRH1 | crf1 | modifying | | | | | | | | | |
| Myrth2p4_002915 | | Uncharacterized | Uncharacterized | | oxidoreductase | | | 193 | 194 | 195 | 920 | 1226 | 1532 |
| | | oxidoreductase | oxidoreductase yusZ | | | | | | | | | | |
| | | C977.08/C1348.09 | | | | | | | | | | | |
| Myrth2p4_002916 | | Uncharacterized | Uncharacterized | | oxidoreductase | | | 196 | 197 | 198 | 921 | 1227 | 1533 |
| | | oxidoreductase dltE | oxidoreductase dltE | | | | | | | | | | |
| Myrth2p4_002917 | | Glutaminyl-peptide | Glutaminyl-peptide | peptide- | Glutaminyl-peptide | | | 199 | 200 | 201 | 922 | 1228 | 1534 |
| | | cyclotransferase | cyclotransferase-like | modifying | cyclotransferase- | | | | | | | | |
| | | | protein | | like protein | | | | | | | | |
| Myrth2p4_002930 | Myrth2p4_002930 | beta-glucuronidase | beta-glucuronidase | hemicellulose- | beta-glucuronidase | GH79 | | 202 | 203 | 204 | 923 | 1229 | 1535 |
| | | | GH79 | degrading | | | | | | | | | |
| Myrth2p4_003005 | Myrth2p4_003005 | Non-Catalytic | unknown | | expansin | | | 205 | 206 | 207 | 924 | 1230 | 1536 |
| | | module family | | | | | | | | | | | |
| | | expansin | | | | | | | | | | | |
| Myrth2p4_003034 | Myrth2p4_003034 | unknown | Uncharacterized protein | | Uncharacterized | | | 208 | 209 | 210 | 925 | 1231 | 1537 |
| | | | YkgB | | lignocellulose- | | | | | | | | |
| | | | | | induced protein | | | | | | | | |
| Myrth2p4_003051 | Myrth2p4_003051 | unknown | unknown | | uncharacterized | | | 211 | 212 | 213 | 926 | 1232 | 1538 |
| | | | | | lignocellulose- | | | | | | | | |
| | | | | | induced protein | | | | | | | | |
| Myrth2p4_003065 | Myrth2p4_003065 | unknown | unknown | | uncharacterized | | | 214 | 215 | 216 | 927 | 1233 | 1539 |
| | | | | | lignocellulose- | | | | | | | | |
| | | | | | induced protein | | | | | | | | |
| Myrth2p4_003070 | Myrth2p4_003070 | exo-1,3-beta- | exo-1,3-beta- | cellulose- | exo-1,3-beta- | GH55 | | 217 | 218 | 219 | 928 | 1234 | 1540 |
| | | glucanase | glucanase GH55 | degrading | glucanase | | | | | | | | |
| Myrth2p4_003103 | MYRTH_3_00121 | cellulase-enhancing | polysaccharide | cellulose- | polysaccharide | GH61 | | 220 | 221 | 222 | 929 | 1235 | 1541 |
| | | protein | monooxygenase | degrading | monooxygenase | | | | | | | | |
| Myrth2p4_003203 | Myrth2p4_003203 | endoglucanase | cellobiohydrolase GH7 | cellulose- | cellobiohydrolase | GH7 | CBM | 223 | 224 | 225 | 930 | 1236 | 1542 |
| | | | | degrading | | | 1 | | | | | | |
| Myrth2p4_003274 | Myrth2p4_003274 | Probable rhamno- | Probable rhamno- | pectin- | rhamno- | | | 226 | 227 | 228 | 931 | 1237 | 1543 |
| | | galacturonate | galacturonate | degrading | galacturonase | | | | | | | | |
| | | lyase C | lyase C | | | | | | | | | | |
| Myrth2p4_003333 | | unknown | exo-1,3-beta- | hemicellulose- | exo-1,3-beta- | GH43 | | 229 | 230 | 231 | 932 | 1238 | 1544 |
| | | | galactanase GH43 | degrading | galactanase | | | | | | | | |
| Myrth2p4_003368 | MYRTH_1_00068 | xylanase | Xylanase GH11 | hemicellulose- | Xylanase | GH11 | | 232 | 233 | 234 | 933 | 1239 | 1545 |
| | | | | degrading | | | | | | | | | |
| Myrth2p4_003495 | Myrth2p4_003495 | unknown | Uncharacterized protein | | uncharacterized | | | 235 | 236 | 237 | 934 | 1240 | 1546 |
| | | | SAOUHSC_02143 | | lignocellulose- | | | | | | | | |
| | | | | | induced protein | | | | | | | | |
| Myrth2p4_003633 | MYRTH_2_01655 | cellulase-enhancing | polysaccharide | cellulose- | polysaccharide | GH61 | | 238 | 239 | 240 | 935 | 1241 | 1547 |
| | | protein | monooxygenase | degrading | monooxygenase | | | | | | | | |
| Myrth2p4_003679 | | Lipase 1 | Lipase 1 | lipid- | lipase | | | 241 | 242 | 243 | 936 | 1242 | 1548 |
| | | | | degrading | | | | | | | | | |
| Myrth2p4_003685 | Myrth2p4_003685 | unknown | xylanase GH30 | hemicellulose- | xylanase | GH30 | | 244 | 245 | 246 | 937 | 1243 | 1549 |
| | | | | degrading | | | | | | | | | |
| Myrth2p4_003686 | | N-acyl- | N-acyl- | phospholipid- | lipase | | | 247 | 248 | 249 | 938 | 1244 | 1550 |
| | | phosphatidylethanol- | phosphatidylethanol- | modifying | | | | | | | | | |
| | | amine-hydrolyzing | amine-hydrolyzing | | | | | | | | | | |
| | | phospholipase D | phospholipase D | | | | | | | | | | |
| Myrth2p4_003747 | | unknown | unknown | | unknown | | | 250 | 251 | 252 | 939 | 1245 | 1551 |
| Myrth2p4_003793 | | Probable leucine | Leucine aminopeptidase | protein | protease | | | 253 | 254 | 255 | 940 | 1246 | 1552 |
| | | aminopeptidase 1 | 1 | hydrolysis | | | | | | | | | |
| Myrth2p4_003921 | | unknown | unknown | | unknown CBM18 | | CBM | 256 | 257 | 258 | 941 | 1247 | 1553 |
| | | | | | | | 18 | | | | | | |
| Myrth2p4_003927 | MYRTH_3_00104 | cellulase-enhancing | polysaccharide | cellulose- | polysaccharide | GH61 | | 259 | 260 | 261 | 942 | 1248 | 1554 |
| | | protein | monooxygenase | degrading | monooxygenase | | | | | | | | |
| Myrth2p4_003941 | MYRTH_1_00024 | unknown | Beta-glucanase | glucan- | Beta-glucanase | GH16 | | 262 | 263 | 264 | 943 | 1249 | 1555 |
| | | | | degrading | | | | | | | | | |
| Myrth2p4_003942 | Myrth2p4_003942 | Pectinesterase A | pectin methylesterase | pectin- | pectinesterase | CE8 | | 265 | 266 | 267 | 944 | 1250 | 1556 |
| | | | CE8 | degrading | | | | | | | | | |
| Myrth2p4_003966 | | unknown | unknown | | unknown CBM18 | | CBM | 268 | 269 | 270 | 945 | 1251 | 1557 |
| | | | | | | | 18 | | | | | | |
| Myrth2p4_004088 | | Lipase | Lipase | lipid- | lipase | | | 271 | 272 | 273 | 946 | 1252 | 1558 |
| | | | | degrading | | | | | | | | | |
| Myrth2p4_004089 | Myrth2p4_004089 | beta-glucosidase | beta-glucosidase GH3 | cellulose- | beta-glucosidase | GH3 | | 274 | 275 | 276 | 947 | 1253 | 1559 |
| | | | | degrading | | | | | | | | | |
| Myrth2p4_004201 | | Putative | Putative | protein | protease | | | 277 | 278 | 279 | 948 | 1254 | 1560 |
| | | metallocarboxypeptidase | metallocarboxypeptidase | hydrolysis | | | | | | | | | |
| | | MCYG_04493 | ecm14 | | | | | | | | | | |
| Myrth2p4_004260 | MYRTH_2_04381 | cellulase-enhancing | polysaccharide | cellulose- | polysaccharide | GH61 | CBM | 280 | 281 | 282 | 949 | 1255 | 1561 |
| | | protein | monooxygenase | degrading | monooxygenase | | 1 | | | | | | |
| Myrth2p4_004335 | MYRTH_2_03391 | cellulase- | polysaccharide | cellulose- | polysaccharide | GH61 | | 283 | 284 | 285 | 950 | 1256 | 1562 |
| | | enhancing protein | monooxygenase | degrading | monooxygenase | | | | | | | | |
| Myrth2p4_004336 | Myrth2p4_004336 | endoglucanase | endoglucanase GH5 | cellulose- | endoglucanase | GH5 | | 286 | 287 | 288 | 951 | 1257 | 1563 |
| | | | | degrading | | | | | | | | | |
| Myrth2p4_004345 | | tripeptidyl-peptidase | tripeptidyl-peptidase | peptide | protease | | | 289 | 290 | 291 | 952 | 1258 | 1564 |
| | | sed2 | sed2 | hydrolysis | | | | | | | | | |
| Myrth2p4_004391 | MYRTH_201413 | cellulase- | polysaccharide | cellulose- | polysaccharide | GH61 | CBM | 292 | 293 | 294 | 953 | 1259 | 1565 |
| | | enhancing protein | monooxygenase | degrading | monooxygenase | | 1 | | | | | | |
| Myrth2p4_004393 | Myrth2p4_004393 | unknown | uncharacterized protein | | uncharacterized | | | 295 | 296 | 297 | 954 | 1260 | 1566 |
| | | | R656 | | lignocellulose- | | | | | | | | |
| | | | | | induced protein | | | | | | | | |
| Myrth2p4_004397 | MYRTH_300116 | cellulase-enhancing | polysaccharide | cellulose- | polysaccharide | GH61 | | 298 | 299 | 300 | 955 | 1261 | 1567 |
| | | protein | monooxygenase | degrading | monooxygenase | | | | | | | | |
| Myrth2p4_004415 | MYRTH_100074 | chitinase | Killer toxin subunits | chitin- | chitinase | GH18 | CBM | 301 | 302 | 303 | 956 | 1262 | 1568 |
| | | | alpha/beta | degrading | | | 18 | | | | | | |
| Myrth2p4_004442 | | Metallocarboxy- | Metallocarboxy- | protein | protease | | | 304 | 305 | 306 | 957 | 1263 | 1569 |
| | | peptidase A-like | peptidase A-like | hydrolysis | | | | | | | | | |
| | | protein | protein | | | | | | | | | | |
| | | MCYG_01475 | MCYG_01475 | | | | | | | | | | |
| Myrth2p4_004455 | | galactanase | galactanase GH5 | hemicellulose- | galactanase | GH30 | | 307 | 308 | 309 | 958 | 1264 | 1570 |
| | | | | degrading | | | | | | | | | |
| Myrth2p4_004476 | Myrth2p4_004476 | unknown | unknown | | uncharacterized | | | 310 | 311 | 312 | 959 | 1265 | 1571 |
| | | | | | lignocellulose- | | | | | | | | |
| | | | | | induced protein | | | | | | | | |
| Myrth2p4_004487 | MYRTH_406966 | cellulase-enhancing | polysaccharide | cellulose- | polysaccharide | GH61 | | 313 | 314 | 315 | 960 | 1266 | 1572 |
| | | protein | monooxygenase | degrading | monooxygenase | | | | | | | | |
| Myrth2p4_004497 | Myrth2p4_004497 | Probable beta- | Beta-galactosidase | hemicellulose- | Beta-galactosidase | GH35 | | 316 | 317 | 318 | 961 | 1267 | 1573 |
| | | galactosidase B | GH35 | degrading | | | | | | | | | |
| Myrth2p4_004508 | MYRTH_200518 | cellulase-enhancing | polysaccharide | cellulose- | polysaccharide | GH61 | | 319 | 320 | 321 | 962 | 1268 | 1574 |
| | | protein | monooxygenase | degrading | monooxygenase | | | | | | | | |
| Myrth2p4_004535 | Myrth2p4_004535 | Xylosidase/arabinosidase | Xylosidase/arabinosidase | Hemicellulose- | Xylosidase/arabinosidase | GH43 | | 322 | 323 | 324 | 963 | 1269 | 1575 |
| | | | | modifying | | | | | | | | | |
| Myrth2p4_004704 | Myrth2p4_004704 | Putative galacturan | exo- | pectin- | rhamno- | GH28 | | 325 | 326 | 327 | 964 | 1270 | 1576 |
| | | 1,4-alpha- | rhamnogalacturonase | degrading | galacturonase | | | | | | | | |
| | | galacturonidase B | GH28 | | | | | | | | | | |
| Myrth2p4_004725 | | Carboxypeptidase | Carboxypeptidase | protein | protease | | | 328 | 329 | 330 | 965 | 1271 | 1577 |
| | | cpdS | cpdS | hydrolysis | | | | | | | | | |
| Myrth2p4_004787 | | beta-glucosidase | beta-glucosidase GH3 | cellulose- | beta-glucosidase | GH3 | | 331 | 332 | 333 | 966 | 1272 | 1578 |
| | | | | degrading | | | | | | | | | |
| Myrth2p4_004788 | MYRTH_1_00011 | Endo-1,4-beta- | xylanase GH10 | hemicellulose- | xylanase | GH10 | | 334 | 335 | 336 | 967 | 1273 | 1579 |
| | | xylanase | | degrading | | | | | | | | | |
| Myrth2p4_004953 | Myrth2p4_004953 | unknown | unknown | | uncharacterized | | | 337 | 338 | 339 | 968 | 1274 | 1580 |
| | | | | | lignocellulose- | | | | | | | | |
| | | | | | induced protein | | | | | | | | |
| Myrth2p4_004960 | Myrth2p4_004960 | unknown | unknown | | uncharacterized | | | 340 | 341 | 342 | 969 | 1275 | 1581 |
| | | | | | lignocellulose- | | | | | | | | |
| | | | | | induced protein | | | | | | | | |
| Myrth2p4_004965 | Myrth2p4_004965 | Probable feruloyl | feruloyl esterase CE1 | hemicellulose- | feruloyl esterase | CE1 | | 343 | 344 | 345 | 970 | 1276 | 1582 |
| | | esterase C | | modifying | | | | | | | | | |
| Myrth2p4_004966 | Myrth2p4_004966 | Feruloyl esterase B | feruloyl esterase CE1 | Hemicellulose- | feruloyl esterase | CE1 | | 346 | 347 | 348 | 971 | 1277 | 1583 |
| | | | | modifyin | | | | | | | | | |
| Myrth2p4_004986 | MYRTH_2_01976 | xylanase | xylanase GH11 | hemicellulose- | xylanase | GH11 | CBM | 349 | 350 | 351 | 972 | 1278 | 1584 |
| | | | | degrading | | | 1 | | | | | | |
| Myrth2p4_004993 | | Cuticle-degrading | Cuticle-degrading | protein | protease | | | 352 | 353 | 354 | 973 | 1279 | 1585 |
| | | protease | protease | hydrolysis | | | | | | | | | |
| Myrth2p4_005017 | MYRTH_3_00097 | endoglucanase | endoglucanase GH6 | cellulose- | endoglucanase | GH6 | | 355 | 356 | 357 | 974 | 1280 | 1586 |
| | | | | degrading | | | | | | | | | |
| Myrth2p4_005025 | | Glucan 1,3-beta- | Glucan 1,3-beta- | cellulose- | Glucan 1,3-beta- | GH55 | | 358 | 359 | 360 | 975 | 1281 | 1587 |
| | | glucosidase | glucosidase | degrading | glucosidase | | | | | | | | |
| Myrth2p4_005037 | Myrth2p4_005037 | Carbohydrate- | Cellobiose | cellulose- | Carbohydrate- | | | 361 | 362 | 363 | 976 | 1282 | 1588 |
| | | binding cytochrome | dehydrogenase | degrading | binding cytochrome | | | | | | | | |
| | | b562 (Fragment) | | | | | | | | | | | |
| Myrth2p4_005039 | | unknown | unknown | | unknown CE15 | CE15 | | 364 | 365 | 366 | 977 | 1283 | 1589 |
| Myrth2p4_005084 | MYRTH_1_00077 | xylanase | xylanase GH11 | hemicellulose- | xylanase | GH11 | | 367 | 368 | 369 | 978 | 1284 | 1590 |
| | | | | degrading | | | | | | | | | |
| Myrth2p4_005133 | Myrth2p4_005133 | unknown | unknown | | uncharacterized | | | 370 | 371 | 372 | 979 | 1285 | 1591 |
| | | | | | lignocellulose- | | | | | | | | |
| | | | | | induced protein | | | | | | | | |
| Myrth2p4_005148 | MYRTH_2_01934 | unknown | carbohydrate esterase | hemicellulose- | unknown CE16 | CE16 | | 373 | 374 | 375 | 980 | 1286 | 1592 |
| | | | | modifying | | | | | | | | | |
| Myrth2p4_005149 | Myrth2p4_005149 | Acetylxylan | acetylxylan esterase CE1 | hemicellulose- | acetylxylan esterase | CE1 | | 376 | 377 | 378 | 981 | 1287 | 1593 |
| | | esterase A | | modifying | | | | | | | | | |
| Myrth2p4_005155 | Myrth2p4_005155 | Aldose 1-epimerase | Aldose 1-epimerase | | Aldose epimerase | | | 379 | 380 | 381 | 982 | 1288 | 1594 |
| Myrth2p4_005177 | Myrth2p4_005177 | unknown | unknown | | uncharacterized | | | 382 | 383 | 384 | 983 | 1289 | 1595 |
| | | | | | lignocellulose- | | | | | | | | |
| | | | | | induced protein | | | | | | | | |
| Myrth2p4_005191 | Myrth2p4_005191 | Pectate lyase A | pectate lyase PL1 | pectin- | pectate lyase | PL1 | | 385 | 386 | 387 | 984 | 1290 | 1596 |
| | | | | degrading | | | | | | | | | |
| Myrth2p4_005222 | MYRTH_2_03793 | alpha-glucuronidase | alpha-glucuronidase | hemicellulose- | alpha-glucuronidase | GH67 | | 388 | 389 | 390 | 985 | 1291 | 1597 |
| | | | GH67 | modifyinging | | | | | | | | | |
| Myrth2p4_005269 | Myrth2p4_005269 | unknown | unknown | | uncharacterized | | | 391 | 392 | 393 | 986 | 1292 | 1598 |
| | | | | | lignocellulose- | | | | | | | | |
| | | | | | induced protein | | | | | | | | |
| Myrth2p4_005317 | Myrth2p4_005317 | unknown | xylan alpha-1,2- | hemicellulose- | xylan alpha-1,2- | GH11 | | 394 | 395 | 396 | 987 | 1293 | 1599 |
| | | | glucuronidase GH115 | modifying | glucuronidase | 5 | | | | | | | |
| | | | | | GH115 | | | | | | | | |
| Myrth2p4_005320 | MYRTH_2_00848 | arabinoxylan | arabinoxylan | hemicellulose- | arabinofuranosidase | GH62 | | 397 | 398 | 399 | 988 | 1294 | 1600 |
| | | arabinofuranohydrolase | arabinofuranosidase | degrading | | | | | | | | | |
| | | | GH62 | | | | | | | | | | |
| Myrth2p4_005321 | | Adhesin protein, | Major allergen Asp f 2 | | adhesin | | | 400 | 401 | 402 | 989 | 1295 | 1601 |
| | | putative | | | | | | | | | | | |
| Myrth2p4_005328 | Myrth2p4_005328 | unknown | carbohydrate esterase | hemicellulose- | unknown CE15 | CE15 | | 403 | 404 | 405 | 990 | 1296 | 1602 |
| | | | | modfiying | | | | | | | | | |
| Myrth2p4_005329 | Myrth2p4_005329 | cellulase-enhancing | polysaccharide | cellulose- | polysaccharide | GH61 | | 406 | 407 | 408 | 991 | 1297 | 1603 |
| | | protein | monooxygenase | degrading | monooxygenase | | | | | | | | |
| Myrth2p4_005340 | Myrth2p4_005340 | exo- | exo- | pectin- | exo- | GH28 | | 409 | 410 | 411 | 992 | 1298 | 1604 |
| | | polygalacturonase | polygalacturonase GH28 | degrading | polygalacturonase | | | | | | | | |
| Myrth2p4_005343 | MYRTH_2_04093 | cellulase-enhancing | polysaccharide | cellulose- | polysaccharide | GH61 | | 412 | 413 | 414 | 993 | 1299 | 1605 |
| | | protein | monooxygenase | degrading | monooxygenase | | | | | | | | |
| Myrth2p4_005368 | Myrth2p4_005368 | Carbohydrate- | unknown | | Carbohydrate- | | | 415 | 416 | 417 | 994 | 1300 | 1606 |
| | | binding cytochrome | | | binding cytochrome | | | | | | | | |
| | | b562 | | | | | | | | | | | |
| Myrth2p4_005452 | Myrth2p4_005452 | beta-glucosidase | Beta-glucosidase GH3 | cellulose- | Beta-glucosidase | GH3 | | 418 | 419 | 420 | 995 | 1301 | 1607 |
| | | | | degrading | | | | | | | | | |
| Myrth2p4_005454 | MYRTH_3_00103 | cellulase-enhancing | polysaccharide | cellulose- | polysaccharide | GH61 | | 421 | 422 | 423 | 996 | 1302 | 1608 |
| | | protein | monooxygenase | degrading | monooxygenase | | | | | | | | |
| Myrth2p4_005463 | | Carboxypeptidase | Carboxypeptidase | protein | protease | | | 424 | 425 | 426 | 997 | 1303 | 1609 |
| | | S1 homolog A | S1 homolog B | hydrolysis | | | | | | | | | |
| Myrth2p4_005484 | | Vacuolar protease A | Vacuolar protease A | protein | protease | | | 427 | 428 | 429 | 998 | 1304 | 1610 |
| | | | | hydrolysis | | | | | | | | | |
| Myrth2p4_005539 | Myrth2p4_005539 | Laccase-2 | Laccase-2 | lignin- | laccase | | | 430 | 431 | 432 | 999 | 1305 | 1611 |
| | | | | degrading | | | | | | | | | |
| Myrth2p4_005561 | Myrth2p4_005561 | Probable feruloyl | Probable feruloyl | hemicellulose- | feruloyl esterase | | | 433 | 434 | 435 | 1000 | 1306 | 1612 |
| | | esterase B-1 | esterase B-2 | modifying | | | | | | | | | |
| Myrth2p4_005590 | | Peptidase M20 | Peptidase M20 domain- | protein | protease | | | 436 | 437 | 438 | 1001 | 1307 | 1613 |
| | | domain-containing | containing protein | hydrolysis | | | | | | | | | |
| | | protein C757.05c | SMAC_03666.2 | | | | | | | | | | |
| Myrth2p4_005626 | | Oryzin (protease) | Subtilisin-like protease 6 | protein | protease | | | 439 | 440 | 441 | 1002 | 1308 | 1614 |
| | | | | hydrolysis | | | | | | | | | |
| Myrth2p4_005639 | | chitinase | chitinase GH18 | chitin- | chitinase | GH18 | | 442 | 443 | 444 | 1003 | 1309 | 1615 |
| | | | | degrading | | | | | | | | | |
| Myrth2p4_005750 | MYRTH_2_03494 | cellulase-enhancing | polysaccharide | cellulose- | polysaccharide | GH61 | | 445 | 446 | 447 | 1004 | 1310 | 1616 |
| | | protein | monooxygenase | degrading | monooxygenase | | | | | | | | |
| Myrth2p4_005752 | MYRTH_2_01610 | unknown | unknown | | uncharacterized | | CBM | 448 | 449 | 450 | 1005 | 1311 | 1617 |
| | | | | | lignocellulose- | | 1 | | | | | | |
| | | | | | induced protein | | | | | | | | |
| Myrth2p4_005753 | Myrth2p4_005753 | unknown | unknown | | uncharacterized | | | 451 | 452 | 453 | 1006 | 1312 | 1618 |
| | | | | | lignocellulose- | | | | | | | | |
| | | | | | induced protein | | | | | | | | |
| Myrth2p4_005819 | | laminarinase | Laminarinase GH55 | Glucan- | Laminarinase | GH55 | | 454 | 455 | 456 | 1007 | 1313 | 1619 |
| | | | | degrading | | | | | | | | | |
| Myrth2p4_005822 | Myrth2p4_005822 | unknown | unknown | | uncharacterized | | | 457 | 458 | 459 | 1008 | 1314 | 1620 |
| | | | | | lignocellulose- | | | | | | | | |
| | | | | | induced protein | | | | | | | | |
| Myrth2p4_005854 | MYRTH_1_00070 | Probable endo-1,4- | Xylanase GH11 | hemicellulose- | Xylanase | GH11 | | 460 | 461 | 462 | 1009 | 1315 | 1621 |
| | | beta-xylanase A | | degrading | | | | | | | | | |
| Myrth2p4_005856 | MYRTH_1_00073 | unknown | Beta-glucanase | glucan- | Beta-glucanase | GH16 | | 463 | 464 | 465 | 1010 | 1316 | 1622 |
| | | | | degrading | | | | | | | | | |
| Myrth2p4_005886 | Myrth2p4_005886 | unknown | unknown | | uncharacterized | | | 466 | 467 | 468 | 1011 | 1317 | 1623 |
| | | | | | lignocellulose- | | | | | | | | |
| | | | | | induced protein | | | | | | | | |
| Myrth2p4_005920 | | Leucine | Aminopeptidase Y | protein | protease | | | 469 | 470 | 471 | 1012 | 1318 | 1624 |
| | | aminopeptidase 2 | | hydrolysis | | | | | | | | | |
| Myrth2p4_005923 | Myrth2p4_005923 | Acetylxylan esterase | acetylxylan esterase CE5 | hemicellulose- | acetylxylan esterase | CE5 | CBM | 472 | 473 | 474 | 1013 | 1319 | 1625 |
| | | | | degrading | | | 1 | | | | | | |
| Myrth2p4_005937 | | Aminopeptidase Y | Aminopeptidase Y | protein | protease | | | 475 | 476 | 477 | 1014 | 1320 | 1626 |
| | | | | hydrolysis | | | | | | | | | |
| Myrth2p4_005945 | MYRTH_1_00007 | endo-1,5-alpha- | endo-1,5-alpha- | hemicellulose- | endo-1,5-alpha- | GH43 | | 478 | 479 | 480 | 1015 | 1321 | 1627 |
| | | arabinanase | arabinanase GH43 | degrading | arabinanase | | | | | | | | |
| Myrth2p4_005946 | Myrth2p4_005946 | Alpha-N- | Alpha-N- | hemicellulose- | arabinofuranosidase | GH43 | | 481 | 482 | 483 | 1016 | 1322 | 1628 |
| | | arabinofuranosidase | arabinofuranosidase | degrading | | | | | | | | | |
| | | 2 | 2 | | | | | | | | | | |
| Myrth2p4_005976 | Myrth2p4_005976 | endoglucanase | endoglucanase GH5 | cellulose- | endoglucanase | GH5 | | 484 | 485 | 486 | 1017 | 1323 | 1629 |
| | | | | degrading | | | | | | | | | |
| Myrth2p4_006001 | Myrth2p4_006001 | Laccase-1 | Laccase-1 | lignin- | laccase | | | 487 | 488 | 489 | 1018 | 1324 | 1630 |
| | | | | degrading | | | | | | | | | |
| Myrth2p4_006022 | Myrth2p4_006022 | Probable pectin | pectin lyase PL1 | pectin- | pectin lyase | PL1 | | 490 | 491 | 492 | 1019 | 1325 | 1631 |
| | | lyase A | | degrading | | | | | | | | | |
| Myrth2p4_006028 | Myrth2p4_006028 | unknown | galactanase GH5 | hemicellulose- | galactanase | GH5 | | 493 | 494 | 495 | 1020 | 1326 | 1632 |
| | | | | degrading | | | | | | | | | |
| Myrth2p4_006058 | | Bifunctional | chitin deacetylase CE4 | chitin- | chitin deacetylase | CE4 | | 496 | 497 | 498 | 1021 | 1327 | 1633 |
| | | xylanase/deacetylase | | modifying | | | | | | | | | |
| Myrth2p4_006119 | MYRTH_203560 | endo-1,4-beta- | xylanase GH10 | hemicellulose- | xylanase | GH10 | | 499 | 500 | 501 | 1022 | 1328 | 1634 |
| | | xylanase | | degrading | | | | | | | | | |
| Myrth2p4_006140 | MYRTH_2_01176 | alpha-arabino- | arabinoxylan arabino- | hemicellulose- | arabino- | GH62 | | 502 | 503 | 504 | 1023 | 1329 | 1635 |
| | | furanosidase | furanohydrolase GH62 | degrading | furanosidase | | | | | | | | |
| Myrth2p4_006141 | Myrth2p4_006141 | Alpha-N- | Alpha-N- | hemicellulose- | arabinofuranosidase | GH43 | | 505 | 506 | 507 | 1024 | 1330 | 1636 |
| | | arabinofuranosidase | arabinofuranosidase | degrading | | | | | | | | | |
| | | 2 | 2 | | | | | | | | | | |
| Myrth2p4_006201 | Myrth2p4_006201 | Cutinase | Cutinase CE5 | cutin- | cutinase | CE5 | | 508 | 509 | 510 | 1025 | 1331 | 1637 |
| | | | | degrading | | | | | | | | | |
| Myrth2p4_006226 | Myrth2p4_006226 | Probable pectate | pectate lyase PL1 | pectin- | pectate lyase | PL1 | | 511 | 512 | 513 | 1026 | 1332 | 1638 |
| | | lyase B | | degrading | | | | | | | | | |
| Myrth2p4_006305 | Myrth2p4_006305 | cellobiohydrolase | Cellobiohydrolase GH7 | cellulose- | Cellobiohydrolase | GH7 | | 514 | 515 | 516 | 1027 | 1333 | 1639 |
| | | | | degrading | | | | | | | | | |
| Myrth2p4_006387 | | Probable | Probable | carbohydrate- | glycosidase | GH16 | | 517 | 518 | 519 | 1028 | 1334 | 1640 |
| | | glycosidase crf1 | glycosidase crf1 | modifying | | | | | | | | | |
| Myrth2p4_006397 | Myrth2p4_006397 | beta-xylosidase | xylosidase/ | hemicellulose- | xylosidase/ | GH43 | | 520 | 521 | 522 | 1029 | 1335 | 1641 |
| | | | arabinosidase | degrading | arabinosidase | | | | | | | | |
| Myrth2p4_006400 | Myrth2p4_006400 | unknown | unknown | | uncharacterized | GH43 | | 523 | 524 | 525 | 1030 | 1336 | 1642 |
| | | | | | lignocellulose- | | | | | | | | |
| | | | | | induced protein | | | | | | | | |
| Myrth2p4_006403 | MYRTH_2_04242 | cellulase-enhancing | polysaccharide | cellulose- | polysaccharide | GH61 | | 526 | 527 | 528 | 1031 | 1337 | 1643 |
| | | protein | monooxygenase | degrading | monooxygenase | | | | | | | | |
| Myrth2p4_006408 | Myrth2p4_006408 | endoglucanase | xyloglucanase GH74 | hemicellulose- | xyloglucanase | GH74 | | 529 | 530 | 531 | 1032 | 1338 | 1644 |
| | | | | degrading | GH74 | | | | | | | | |
| Myrth2p4_006434 | | Carbohydrate- | unknown | carbohydrate- | Carbohydrate- | | | 532 | 533 | 534 | 1033 | 1339 | 1645 |
| | | binding cytochrome | | oxidizing | binding cytochrome | | | | | | | | |
| | | b562 | | | | | | | | | | | |
| Myrth2p4_006514 | | Subtilisin-like | Subtilisin-like | protein | protease | | | 535 | 536 | 537 | 1034 | 1340 | 1646 |
| | | proteinase Spm1 | proteinase Spm1 | hydrolysis | | | | | | | | | |
| Myrth2p4_006524 | Myrth2p4_006524 | Adhesin protein | possible adhesin | | adhesin | | | 538 | 539 | 540 | 1035 | 1341 | 1647 |
| | | Mad1 | | | | | | | | | | | |
| Myrth2p4_006587 | MYRTH_1_00040 | Chitinase 3 | Endochitinase 2 | chitin- | chitinase | | | 541 | 542 | 543 | 1036 | 1342 | 1648 |
| | | | | degrading | | | | | | | | | |
| Myrth2p4_006646 | | Uncharacterized | Uncharacterized | | oxidoreductase | | | 544 | 545 | 546 | 1037 | 1343 | 1649 |
| | | oxidoreductase | oxidoreductase | | | | | | | | | | |
| | | C30D10.05c | C30D10.05c | | | | | | | | | | |
| Myrth2p4_006765 | Myrth2p4_006765 | Probable feruloyl | feruloyl esterase CE1 | hemicellulose- | feruloyl esterase | CE1 | | 547 | 548 | 549 | 1038 | 1344 | 1650 |
| | | esterase C | | modifying | | | | | | | | | |
| Myrth2p4_006772 | Myrth2p4_006772 | Cellobiose | Cellobiose | cellulose- | cellobiose | | | 550 | 551 | 552 | 1039 | 1345 | 1651 |
| | | dehydrogenase | dehydrogenase | degrading | dehydrogenase | | | | | | | | |
| Myrth2p4_006795 | MYRTH_2_04272 | cellulase- | polysaccharide | cellulose- | polysaccharide | GH61 | CBM | 553 | 554 | 555 | 1040 | 1346 | 1652 |
| | | enhancing protein | monooxygenase | degrading | monooxygenase | | 1 | | | | | | |
| Myrth2p4_006807 | MYRTH_2_02340 | Rhamnogalacturonan | rhamnogalacturonan | pectin- | rhamnogalacturonan | CE12 | | 556 | 557 | 558 | 1041 | 1347 | 1653 |
| | | acetylesterase | acetylesterase CE12 | degrading | acetylesterase | | | | | | | | |
| Myrth2p4_006821 | Myrth2p4_006821 | Rhamnogalacturonan | Rhamnogalacturonan | pectin- | rhamnogalacturonan | CE12 | | 559 | 560 | 561 | 1042 | 1348 | 1654 |
| | | acetylesterase rhgT | acetylesterase rhgT | degrading | acetylesterase rhgT | | | | | | | | |
| Myrth2p4_006837 | Myrth2p4_006837 | Laccase-1 | Laccase-1 | lignin- | laccase | | | 562 | 563 | 564 | 1043 | 1349 | 1655 |
| | | | | degrading | | | | | | | | | |
| Myrth2p4_007013 | | unknown | unknown | | unknown | | | 565 | 566 | 567 | 1044 | 1350 | 1656 |
| Myrth2p4_007061 | Myrth2p4_007061 | Aldose 1-epimerase | Aldose 1-epimerase | | Aldose epimerase | | | 568 | 569 | 570 | 1045 | 1351 | 1657 |
| Myrth2p4_007109 | | unknown | unknown | | unknown | | | 571 | 572 | 573 | 1046 | 1352 | 1658 |
| Myrth2p4_007127 | | Beta- | Beta- | chitin- | Beta- | GH3 | | 574 | 575 | 576 | 1047 | 1353 | 1659 |
| | | hexosaminidase | hexosaminidase | degrading | hexosaminidase | | | | | | | | |
| Myrth2p4_007150 | Myrth2p4_007150 | Probable | Probable | hemicellulose- | acetylxylan esterase | CE1 | | 577 | 578 | 579 | 1048 | 1354 | 1660 |
| | | acetylxylan | acetylxylan | modifying | | | | | | | | | |
| | | esterase A | esterase A | | | | | | | | | | |
| Myrth2p4_007367 | MYRTH_2_02197 | Feruloyl esterase B | feruloyl esterase CE1 | hemicellulose- | feruloyl esterase | CE1 | | 580 | 581 | 582 | 1049 | 1355 | 1661 |
| | | | | modifying | | | | | | | | | |
| Myrth2p4_007409 | Myrth2p4_007409 | xylanase | xylanase GH11 | hemicellulose- | xylanase | GH11 | | 583 | 584 | 585 | 1050 | 1356 | 1662 |
| | | | | degrading | | | | | | | | | |
| Myrth2p4_007425 | Myrth2p4_007425 | unknown | unknown | | uncharacterized | | CBM | 586 | 587 | 588 | 1051 | 1357 | 1663 |
| | | | | | lignocellulose- | | 1 | | | | | | |
| | | | | | induced protein | | | | | | | | |
| Myrth2p4_007444 | Myrth2p4_007444 | Cellobiose | Cellobiose | cellulose- | cellobiose | | | 589 | 590 | 591 | 1052 | 1358 | 1664 |
| | | dehydrogenase | dehydrogenase | degrading | dehydrogenase | | | | | | | | |
| Myrth2p4_007447 | | Carbohydrate- | Cellobiose | cellulose- | Carbohydrate- | | | 592 | 593 | 594 | 1053 | 1359 | 1665 |
| | | binding cytochrome | dehydrogenase | degrading | binding cytochrome | | | | | | | | |
| | | b562 (Fragment) | | | | | | | | | | | |
| Myrth2p4_007461 | MYRTH_3_00099 | cellobiohydrolase | cellobiohydrolase GH6 | cellulose- | cellobiohydrolase | GH6 | | 595 | 596 | 597 | 1054 | 1360 | 1666 |
| | | | | degrading | | | | | | | | | |
| Myrth2p4_007538 | MYRTH_2_00570 | Putative rhamno- | rhamnogalacturonan | pectin- | rhamno- | PL4 | | 598 | 599 | 600 | 1055 | 1361 | 1667 |
| | | galacturonase | lyase PL4 | degrading | galacturonate lyase | | | | | | | | |
| Myrth2p4_007539 | | Probable leucine | Leucine aminopeptidase | protein | protease | | | 601 | 602 | 603 | 1056 | 1362 | 1668 |
| | | aminopeptidase | 1 | hydrolysis | | | | | | | | | |
| | | MCYG_04170 | | | | | | | | | | | |
| Myrth2p4_007540 | | Carbohydrate- | unknown | carbohydrate- | Carbohydrate- | | | 604 | 605 | 606 | 1057 | 1363 | 1669 |
| | | binding cytochrome | | oxidizing | binding cytochrome | | | | | | | | |
| | | b562 (Fragment) | | | | | | | | | | | |
| Myrth2p4_007556 | | Aspergillopepsin-2 | Aspergillopepsin-2 | protein | protease | | | 607 | 608 | 609 | 1058 | 1364 | 1670 |
| | | | | hydrolysis | | | | | | | | | |
| Myrth2p4_007648 | | exo-1,3-beta- | exo-1,3-beta- | cellulose- | exo-1,3-beta- | GH55 | | 610 | 611 | 612 | 1059 | 1365 | 1671 |
| | | glucanase | glucanase GH55 | degrading | glucanase | | | | | | | | |
| Myrth2p4_007688 | Myrth2p4_007688 | Manganese | Manganese | lignin- | manganese | | | 613 | 614 | 615 | 1060 | 1366 | 1672 |
| | | peroxidase 3 | peroxidase 3 | degrading | peroxidase | | | | | | | | |
| Myrth2p4_007726 | Myrth2p4_007726 | GLEYA adhesin | possible adhesin | | adhesin | | | 616 | 617 | 618 | 1061 | 1367 | 1673 |
| | | domain | | | | | | | | | | | |
| Myrth2p4_007729 | MYRTH_1_00035 | beta-mannanase | Beta-mannanase GH5 | hemicellulose- | Beta-mannanase | GH5 | | 619 | 620 | 621 | 1062 | 1368 | 1674 |
| | | | | degrading | | | | | | | | | |
| Myrth2p4_007771 | | Alpha-galactosidase | alpha-galactosidase | hemicellulose- | alpha-galactosidase | GH27 | | 622 | 623 | 624 | 1063 | 1369 | 1675 |
| | | A | GH27 | degrading | | | | | | | | | |
| Myrth2p4_007781 | | Probable leucine | Probable leucine | protein | protease | | | 625 | 626 | 627 | 1064 | 1370 | 1676 |
| | | aminopeptidase 2 | aminopeptidase 2 | hydrolysis | | | | | | | | | |
| Myrth2p4_007801 | Myrth2p4_007801 | unknown | unknown | | unknown | | | 628 | 629 | 630 | 1065 | 1371 | 1677 |
| Myrth2p4_007815 | Myrth2p4_007815 | xylanase | Xylanase GH10 | hemicellulose- | Xylanase | GH10 | | 631 | 632 | 633 | 1066 | 1372 | 1678 |
| | | | | degrading | | | | | | | | | |
| Myrth2p4_007838 | Myrth2p4_007838 | Pectate lyase H | pectate lyase PL3 | pectin- | pectate lyase | PL3 | | 634 | 635 | 636 | 1067 | 1373 | 1679 |
| | | | | degrading | | | | | | | | | |
| Myrth2p4_007849 | Myrth2p4_007849 | unknown | unknown | | uncharacterized | | | 637 | 638 | 639 | 1068 | 1374 | 1680 |
| | | | | | lignocellulose- | | | | | | | | |
| | | | | | induced protein | | | | | | | | |
| Myrth2p4_007850 | Myrth2p4_007850 | unknown | unknown | | uncharacterized | | | 640 | 641 | 642 | 1069 | 1375 | 1681 |
| | | | | | lignocellulose- | | | | | | | | |
| | | | | | induced protein | | | | | | | | |
| Myrth2p4_007861 | | Putative serine | Putative serine | protein | protease | | | 643 | 644 | 645 | 1070 | 1376 | 1682 |
| | | protease K12H4.7 | protease K12H4.7 | hydrolysis | | | | | | | | | |
| Myrth2p4_007867 | MYRTH_4_06111 | endoglucanase | endoglucanase GH7 | cellulose- | endoglucanase | GH7 | | 646 | 647 | 648 | 1071 | 1377 | 1683 |
| | | | | degrading | | | | | | | | | |
| Myrth2p4_007877 | MYRTH_2_00938 | unknown | unknown | | uncharacterized | CE3 | | 649 | 650 | 651 | 1072 | 1378 | 1684 |
| | | | | | lignocellulose- | | | | | | | | |
| | | | | | induced protein | | | | | | | | |
| Myrth2p4_007915 | MYRTH_2_03335 | unknown | Glucan endo-1,3-beta- | glucan- | Glucan endo-1,3- | GH16 | | 652 | 653 | 654 | 1073 | 1379 | 1685 |
| | | | glucosidase A1 | degrading | beta-glucosidase | | | | | | | | |
| Myrth2p4_007920 | | Subtilisin-like | Proteinase R | protein | protease | | | 655 | 656 | 657 | 1074 | 1380 | 1686 |
| | | protease 7 | | hydrolysis | | | | | | | | | |
| Myrth2p4_007924 | MYRTH_1_00018 | Beta-glucuronidase | Beta-galactosidase | hemicellulose- | Beta-galactosidase | GH2 | | 658 | 659 | 660 | 1075 | 1381 | 1687 |
| | | | | degrading | | | | | | | | | |
| Myrth2p4_007956 | | unknown | unknown | | unknown PL20 | PL20 | | 661 | 662 | 663 | 1076 | 1382 | 1688 |
| Myrth2p4_007996 | | Probable endo- | Probable endo- | glucan- | endo-1,3(4)-beta- | GH16 | | 664 | 665 | 666 | 1077 | 1383 | 1689 |
| | | 1,3(4)-beta- | 1,3(4)- beta- | degrading | glucanase | | | | | | | | |
| | | glucanase | glucanase | | | | | | | | | | |
| | | AFUB_029980 | AFUA_2G14360 | | | | | | | | | | |
| Myrth2p4_008028 | MYRTH_2_00811 | cellulase-enhancing | polysaccharide | cellulose- | polysaccharide | GH61 | | 667 | 668 | 669 | 1078 | 1384 | 1690 |
| | | protein | monooxygenase | degrading | monooxygenase | | | | | | | | |
| Myrth2p4_008123 | MYRTH_3_00077 | unknown | unknown | | unknown GH43 | GH43 | | 670 | 671 | 672 | 1079 | 1385 | 1691 |
| Myrth2p4_008179 | Myrth2p4_008179 | unknown | unknown | | unknown CE16 | CE16 | | 673 | 674 | 675 | 1080 | 1386 | 1692 |
| Myrth2p4_008220 | Myrth2p4_008220 | unknown | unknown | | uncharacterized | | | 676 | 677 | 678 | 1081 | 1387 | 1693 |
| | | | | | lignocellulose- | | | | | | | | |
| | | | | | induced protein | | | | | | | | |
| Myrth2p4_008285 | Myrth2p4_008285 | unknown | unknown | | unknown CE4 | CE4 | | 679 | 680 | 681 | 1082 | 1388 | 1694 |
| Myrth2p4_008298 | | | endoglucanase GH12 | cellulose- | endoglucanase | GH12 | | 682 | 683 | 684 | 1083 | 1389 | 1695 |
| | | | | degrading | | | | | | | | | |
| Myrth2p4_008299 | Myrth2p4_008299 | Probable exo-1,4- | Beta-xylosidase GH3 | hemicellulose- | Beta-xylosidase | GH3 | | 685 | 686 | 687 | 1084 | 1390 | 1696 |
| | | beta-xylosidase | | degrading | | | | | | | | | |
| | | xlnD | | | | | | | | | | | |
| Myrth2p4_008353 | Myrth2p4_008353 | Periplasmic beta- | Periplasmic beta- | cellulose- | beta-glucosidase | GH3 | | 688 | 689 | 690 | 1085 | 1391 | 1697 |
| | | glucosidase | glucosidase | degrading | | | | | | | | | |
| Myrth2p4_008360 | | Alpha-L-fucosidase | Alpha-L-fucosidase | carbohydrate- | Alpha-L-fucosidase | GH95 | | 691 | 692 | 693 | 1086 | 1392 | 1698 |
| | | 2 | 2 | modifying | | | | | | | | | |
| Myrth2p4_008429 | | Putative | Putative | | hydrolase | | | 694 | 695 | 696 | 1087 | 1393 | 1699 |
| | | uncharacterized | uncharacterized | | | | | | | | | | |
| | | hydrolase | hydrolase | | | | | | | | | | |
| | | YOR131C | YOR131C | | | | | | | | | | |
| Myrth2p4_008437 | Myrth2p4_008437 | Non-Catalytic | Allergen Asp f 7 | cellulase- | expansin | | | 697 | 698 | 699 | 1088 | 1394 | 1700 |
| | | module family | | enhancing | | | | | | | | | |
| | | expansin | | | | | | | | | | | |
| Myrth2p4_008501 | Myrth2p4_008501 | Carbohydrate- | unknown | carbohydrate- | Carbohydrate- | | | 700 | 701 | 702 | 1089 | 1395 | 1701 |
| | | binding cytochrome | | oxidizing | binding cytochrome | | | | | | | | |
| | | b562 | | | | | | | | | | | |
| Myrth2p4_008515 | MYRTH_4_03993 | cellobiohydrolase | cellobiohydrolase GH6 | cellulose- | cellobiohydrolase | GH6 | CBM | 703 | 704 | 705 | 1090 | 1396 | 1702 |
| | | | | degrading | | | 1 | | | | | | |
| Myrth2p4_008522 | Myrth2p4_008522 | Probable 1,4-beta- | possible swollenin | cellulase- | swollenin | CE15 | CBM | 706 | 707 | 708 | 1091 | 1397 | 1703 |
| | | D-glucan | | enhancing | | | 1 | | | | | | |
| | | cellobiohydrolase C | | | | | | | | | | | |
| Myrth2p4_008530 | Myrth2p4_008530 | cellulase-enhancing | polysaccharide | cellulose- | polysaccharide | GH61 | | 709 | 710 | 711 | 1092 | 1398 | 1704 |
| | | protein | monooxygenase | degrading | monooxygenase | | | | | | | | |
| Myrth2p4_008541 | | unknown | unknown | | unknown CBM18 | | CBM | 712 | 713 | 714 | 1093 | 1399 | 1705 |
| | | | | | | | 18 | | | | | | |
| Myrth2p4_008564 | MYRTH_2_04212 | unknown | unknown | | uncharacterized | | | 715 | 716 | 717 | 1094 | 1400 | 1706 |
| | | | | | lignocellulose- | | | | | | | | |
| | | | | | induced protein | | | | | | | | |
| Myrth2p4_008615 | | exo- | exo-glucosaminidase | chitin- | exo- | GH2 | | 718 | 719 | 720 | 1095 | 1401 | 1707 |
| | | glucosaminidase | GH2 | degrading | glucosaminidase | | | | | | | | |
| Myrth2p4_008650 | | unknown | unknown | | unknown | | | 721 | 722 | 723 | 1096 | 1402 | 1708 |
| Myrth2p4_008756 | Myrth2p4_008756 | unknown | Endoglucanase | cellulose- | Endoglucanase | GH5 | | 724 | 725 | 726 | 1097 | 1403 | 1709 |
| | | | | degrading | | | | | | | | | |
| | Myrth2p4_000413 | | Cytochrome P450-DIT2 | | Cytochrome P450 | | | | | | 1098 | 1404 | 1710 |
| | Myrth2p4_000624 | | unknown | | uncharacterized | | | | | | 1099 | 1405 | 1711 |
| | | | | | lignocellulose- | | | | | | | | |
| | | | | | induced protein | | | | | | | | |
| | Myrth2p4_001189 | | Carboxylesterase 5A | carbohydrate- | carboxylesterase | CE10 | | | | | 1100 | 1406 | 1712 |
| | | | | modifiying | | | | | | | | | |
| | Myrth2p4_001457 | | Cytochrome P450 52A12 | | Cytochrome P450 | | | | | | 1101 | 1407 | 1713 |
| | Myrth2p4_001536 | | O- | | oxidoreductase | | | | | | 1102 | 1408 | 1714 |
| | | | methylsterigmatocystin | | | | | | | | | | |
| | | | oxidoreductase | | | | | | | | | | |
| | Myrth2p4_001740 | | possible adhesin | | adhesin | | | | | | 1103 | 1409 | 1715 |
| | Myrth2p4_003589 | | possible adhesin | | adhesin | | | | | | 1104 | 1410 | 1716 |
| | Myrth2p4_003938 | | Tyrosinase | pigment- | Tyrosinase | | | | | | 1105 | 1411 | 1717 |
| | | | | producing | | | | | | | | | |
| | Myrth2p4_006092 | | unknown | | uncharacterized | | | | | | 1106 | 1412 | 1718 |
| | | | | | lignocellulose- | | | | | | | | |
| | | | | | induced protein | | | | | | | | |
| | Myrth2p4_006213 | | Tyrosinase | pigment- | Tyrosinase | | | | | | 1107 | 1413 | 1719 |
| | | | | producing | | | | | | | | | |
| | Myrth2p4_008350 | | O- | | oxidoreductase | | | | | | 1108 | 1414 | 1720 |
| | | | methylsterigmatocystin | | | | | | | | | | |
| | | | oxidoreductase | | | | | | | | | | |
| | MYRTH_1_00002 | | Alpha-L-arabino- | hemicellulose- | Alpha-L-arabino- | GH62 | | | | | 1109 | 1415 | 1721 |
| | | | furanosidase | degrading | furanosidase | | | | | | | | |
| | | | (arabinoxylan | | (arabinoxylan | | | | | | | | |
| | | | arabinofuranosidase) | | arabino- | | | | | | | | |
| | | | GH62 | | furanosidase) | | | | | | | | |
| [Myrth2p4_008299] | MYRTH_1_00003 | | Probable exo-1,4-beta- | hemicellulose- | exo-1,4-beta- | GH3 | | | | | 1110 | 1416 | 1722 |
| | | | xylosidase xlnD | degrading | xylosidase | | | | | | | | |
| | MYRTH_1_00009 | | exo-polygalacturonase | pectin- | exo- | GH28 | | | | | 1111 | 1417 | 1723 |
| | | | GH28 | degrading | polygalacturonase | | | | | | | | |
| | MYRTH_1_00020 | | exo-1,3-beta- | hemicellulose- | exo-1,3-beta- | GH43 | | | | | 1112 | 1418 | 1724 |
| | | | galactanase GH43 | degrading | galactanase | | | | | | | | |
| [Myrth2p4_001494] | MYRTH_1_00021 | | Probable beta- | cellulose- | beta-glucosidase | GH3 | | | | | 1113 | 1419 | 1725 |
| | | | glucosidase L | degrading | | | | | | | | | |
| | MYRTH_1_00025 | | Endo-1,4-beta-xylanase | hemicellulose- | Endo-1,4-beta- | GH10 | | | | | 1114 | 1420 | 1726 |
| | | | A | degrading | xylanase | | | | | | | | |
| | MYRTH_1_00031 | | beta-galactosidase GH35 | hemicellulose- | beta-galactosidase | GH35 | | | | | 1115 | 1421 | 1727 |
| | | | | degrading | | | | | | | | | |
| | MYRTH_1_00032 | | beta-galactosidase GH35 | hemicellulose- | beta-galactosidase | GH35 | | | | | 1116 | 1422 | 1728 |
| | | | | degrading | | | | | | | | | |
| | MYRTH_1_00037 | | Alpha-N- | hemicellulose- | Alpha-N- | GH43 | | | | | 1117 | 1423 | 1729 |
| | | | arabinofuranosidase 2 | degrading | arabinofuranosidase | | | | | | | | |
| | MYRTH_1_00069 | | hexosaminidase GH20 | chitin- | hexosaminidase | GH20 | | | | | 1118 | 1424 | 1730 |
| | | | | degrading | | | | | | | | | |
| | MYRTH_1_00080 | | unknown | | unknown CE3 | CE3 | | | | | 1119 | 1425 | 1731 |
| | MYRTH_1_00084 | | xylanase GH30 | hemicellulose- | xylanase | GH30 | | | | | 1120 | 1426 | 1732 |
| | | | | degrading | | | | | | | | | |
| | MYRTH_1_00087 | | beta-glucosidase GH3 | cellulose- | beta-glucosidase | GH3 | | | | | 1121 | 1427 | 1733 |
| | | | | degrading | | | | | | | | | |
| | MYRTH_1_00098 | | Probable glycosidase | carbohydrate- | glycosidase | GH16 | | | | | 1122 | 1428 | 1734 |
| | | | crf1 | modifying | | | | | | | | | |
| | MYRTH_2_00218 | | endoglucanase GH12 | cellulose- | endoglucanase | GH12 | | | | | 1123 | 1429 | 1735 |
| | | | | degrading | | | | | | | | | |
| | MYRTH_2_00583 | | Chitinase 3 | chitin- | Chitinase 3 | | | | | | 1124 | 1430 | 1736 |
| | | | | degrading | | | | | | | | | |
| | MYRTH_2_00740 | | unknown | | unknown GH16 | GH16 | | | | | 1125 | 1431 | 1737 |
| [Myrth2p4_000495] | MYRTH_2_00959 | | arabinoxylan arabino- | hemicellulose- | arabinoxylan | GH43 | | | | | 1126 | 1432 | 1738 |
| | | | furanohydrolase GH43 | degrading | arabinofurano- | | | | | | | | |
| | | | | | hydrolase⁹ | | | | | | | | |
| | MYRTH_2_01076 | | Chitinase GH18 | chitin- | Chitinase | GH18 | | | | | 1127 | 1433 | 1739 |
| | | | | degrading | | | | | | | | | |
| | MYRTH_2_01077 | | Chitinase GH18 | chitin- | Chitinase | GH18 | | | | | 1128 | 1434 | 1740 |
| | | | | degrading | | | | | | | | | |
| | MYRTH_2_01097 | | cellobiohydrolase GH7 | cellulose- | cellobiohydrolase | GH7 | | | | | 1129 | 1435 | 1741 |
| | | | | degrading | | | | | | | | | |
| | MYRTH_2_01279 | | Beta-xylosidase GH3 | hemicellulose- | Beta-xylosidase | GH3 | | | | | 1130 | 1436 | 1742 |
| | | | | degrading | | | | | | | | | |
| | MYRTH_2_01280 | | Beta-xylosidase GH3 | hemicellulose- | Beta-xylosidase | GH3 | | | | | 1131 | 1437 | 1743 |
| | | | | degrading | | | | | | | | | |
| | MYRTH_2_02633 | | Periplasmic beta- | cellulose- | beta-glucosidase | GH3 | | | | | 1132 | 1438 | 1744 |
| | | | glucosidase | degrading | | | | | | | | | |
| | MYRTH_2_04091 | | xylanase GH10 | hemicellulose- | xylanase | GH10 | CBM | | | | 1133 | 1439 | 1745 |
| | | | | degrading | | | 1 | | | | | | |
| | MYRTH_2_04186 | | endoglucanase GH7 | cellulose- | endoglucanase | GH7 | | | | | 1134 | 1440 | 1746 |
| | | | | degrading | | | | | | | | | |
| | MYRTH_2_04244 | | endoglucanase GH6 | cellulose- | endoglucanase | GH6 | | | | | 1135 | 1441 | 1747 |
| | | | | degrading | | | | | | | | | |
| | MYRTH_2_04271 | | hexosaminidase GH20 | chitin- | hexosaminidase | GH20 | | | | | 1136 | 1442 | 1748 |
| | | | | degrading | | | | | | | | | |
| | MYRTH_2_04288 | | Mannan endo-1,4-beta- | hemicellulose- | Mannan endo-1,4- | GH26 | CBM | | | | 1137 | 1443 | 1749 |
| | | | mannosidase | degrading | beta-mannosidase | | 35 | | | | | | |
| | MYRTH_3_00003 | | Beta-mannanase GH5 | hemicellulose- | Beta-mannanase | GH5 | | | | | 1138 | 1444 | 1750 |
| | | | | degrading | | | | | | | | | |
| | MYRTH_3_00016 | | unknown | | unknown GH16 | GH16 | | | | | 1139 | 1445 | 1751 |
| | MYRTH_3_00086 | | exo-1,3-beta- | hemicellulose- | exo-1,3-beta- | GH43 | | | | | 1140 | 1446 | 1752 |
| | | | galactanase GH43 | degrading | galactanase | | | | | | | | |
| | MYRTH_3_00105 | | Polysaccharide | cellulose- | Polysaccharide | GH61 | | | | | 1141 | 1447 | 1753 |
| | | | monooxygenase | degrading | monooxygenase | | | | | | | | |
| | MYRTH_3_00120 | | Polysaccharide | cellulose- | Polysaccharide | GH61 | CBM | | | | 1142 | 1448 | 1754 |
| | | | monooxygenase GH61 | degrading | monooxygenase | | 1 | | | | | | |
| | MYRTH_3_00124 | | Polysaccharide | cellulose- | Polysaccharide | GH61 | | | | | 1143 | 1449 | 1755 |
| | | | monooxygenase GH61 | degrading | monooxygenase | | | | | | | | |
| | MYRTH_3_00127 | | Alpha-L- | hemicellulose- | Alpha-L-arabino- | GH62 | | | | | 1144 | 1450 | 1756 |
| | | | arabinofuranosidase C | degrading | furanosidase C | | | | | | | | |
| | | | (arabinoxylan arabino- | | (arabinoxylan | | | | | | | | |
| | | | furanohydrolase) GH62 | | arabino- | | | | | | | | |
| | | | | | furanohydrolase) | | | | | | | | |
| | MYRTH_4_05758 | | arabinoxylan | hemicellulose- | arabinoxylan | GH62 | | | | | 1145 | 1451 | 1757 |
| | | | arabinofuranohydrolase | degrading | arabinofurano- | | | | | | | | |
| | | | GH62 | | hydrolase | | | | | | | | |
| | MYRTH_4_09372 | | xylanase GH10 | hemicellulose- | xylanase | GH10 | CBM | | | | 1146 | 1452 | 1758 |
| | | | | degrading | | | 1 | | | | | | |
| | MYRTH_4_09820 | | endoglucanase GH12 | cellulose- | endoglucanase | GH12 | | | | | 1147 | 1453 | 1759 |
| | | | | degrading | | | | | | | | | |
| | Myrth2p4_000387 | | possible pyranose | sugar- | pyranose | | | | | | 1148 | 1454 | 1760 |
| | | | dehydrogenase | modifying | dehydrogenase | | | | | | | | |
| | Myrth2p4_000489 | | Lipase 1 | lipid- | lipase | CE10 | | | | | 1149 | 1455 | 1761 |
| | | | | degrading | | | | | | | | | |
| | Myrth2p4_001363 | | Probable dipeptidyl | protein | protease | CE10 | | | | | 1150 | 1456 | 1762 |
| | | | peptidase 4 | hydrolysis | | | | | | | | | |
| | Myrth2p4_001546 | | possible pyranose | sugar- | pyranose | | | | | | 1151 | 1457 | 1763 |
| | | | dehydrogenase | modifying | dehydrogenase | | | | | | | | |
| | Myrth2p4_002267 | | possible pyranose | sugar- | pyranose | | | | | | 1152 | 1458 | 1764 |
| | | | dehydrogenase | modifying | dehydrogenase | | | | | | | | |
| | Myrth2p4_002365 | | Probable serine protease | protein | protease | | | | | | 1153 | 1459 | 1765 |
| | | | EDA2 | hydrolysis | | | | | | | | | |
| | Myrth2p4_003086 | | possible pyranose | sugar- | pyranose | | | | | | 1154 | 1460 | 1766 |
| | | | dehydrogenase | modifying | dehydrogenase | | | | | | | | |
| | Myrth2p4_004152 | | unknown | | unknown GH61 | GH61 | | | | | 1155 | 1461 | 1767 |
| | Myrth2p4_004330 | | unknown | | unknown CE3 | CE3 | | | | | 1156 | 1462 | 1768 |
| | Myrth2p4_004961 | | Extracellular | protein | protease | | | | | | 1157 | 1463 | 1769 |
| | | | metalloprotease | hydrolysis | | | | | | | | | |
| | | | Pa_2_14170 | | | | | | | | | | |
| | Myrth2p4_005807 | | Uncharacterized | | oxidoreductase | | | | | | 1158 | 1464 | 1770 |
| | | | oxidoreductase dltE | | | | | | | | | | |
| | Myrth2p4_005966 | | Lipase 4 | lipid- | lipase | CE10 | | | | | 1159 | 1465 | 1771 |
| | | | | degrading | | | | | | | | | |
| | Myrth2p4_006645 | | Putative oxidoreductase | | oxidoreductase | | | | | | 1160 | 1466 | 1772 |
| | | | C1F5.03c | | | | | | | | | | |
| | Myrth2p4_008594 | | Uncharacterized FAD- | | oxidoreductase | | | | | | 1161 | 1467 | 1773 |
| | | | linked oxidoreductase | | | | | | | | | | |
| | | | yvdP | | | | | | | | | | |

⁸For example, xylan 1,4-beta-xylosidase
⁹A minor activity of xylan 1,4-beta-xylosidase was detected for this protein.

TABLE 1C
Biomass degrading genes and polypeptides of Aureobasidium pullulans

| | | | | | | | | Provisional | | | | | |
|---|---|---|---|---|---|---|---|---|---|---|---|---|---|
| Gene ID in | | Annotation in | | | | | | application | | | PCT application | | |
| prov. | | Provisional | | | | | | SEQ ID NO: | | | SEQ ID NO: | | |
| appn. | | application No. | | | | CAZy | CBM of | Ge- | | Amino | Ge- | | Amino |
| 61/657,078 | Target ID | 61/657,078 | Updated annotation | Function | Protein activity | family | interest | nomic | Coding | acid | nomic | Coding | acid |
| Aurpu2p4_000013 | Aurpu2p4_000013 | Beta-glucosidase | beta-glucosidase GH1 | cellulose- | beta-glucosidase | GH1 | | 1 | 2 | 3 | 1774 | 2161 | 2548 |
| | | 14 | | degrading | | | | | | | | | |
| Aurpu2p4_000017 | Aurpu2p4_000017 | Probable rhamno- | Endo-rhamno- | pectin- | rhamno- | GH28 | | 4 | 5 | 6 | 1775 | 2162 | 2549 |
| | | galacturonase A | galacturonase GH28 | degrading | galacturonase | | | | | | | | |
| Aurpu2p4_000070 | Aurpu2p4_000070 | endoglucanase | Endoglucanase GH5 | cellulose- | endoglucanase | GH5 | CBM1 | 7 | 8 | 9 | 1776 | 2163 | 2550 |
| | | | | degrading | | | | | | | | | |
| Aurpu2p4_000074 | AURPU_3_00185 | beta-glucosidase | avenacinase GH3 | | avenacinase | GH3 | | 10 | 11 | 12 | 1777 | 2164 | 2551 |
| Aurpu2p4_000163 | AURPU_3_00030 | xyloglucanase | xyloglucanase GH12 | hemicellulose- | xyloglucanase | GH12 | | 13 | 14 | 15 | 1778 | 2165 | 2552 |
| | | | | degrading | | | | | | | | | |
| Aurpu2p4_000184 | | N-acyl- | phospholipase | phospholipid- | lipase | | | 16 | 17 | 18 | 1779 | 2166 | 2553 |
| | | phosphatidylethanolamine- | | modifying | | | | | | | | | |
| | | hydrolyzing | | | | | | | | | | | |
| | | phospholipase D | | | | | | | | | | | |
| Aurpu2p4_000224 | Aurpu2p4_000224 | Acetylxylan | Acetylxylan esterase 1 | hemicellulose- | acetylxylan | CE1 | | 19 | 20 | 21 | 1780 | 2167 | 2554 |
| | | esterase A | CE1 | degrading | esterase | | | | | | | | |
| Aurpu2p4_000225 | Aurpu2p4_000225 | Putative Expansin- | Expansin-B5 | cellulase- | expansin | | | 22 | 23 | 24 | 1781 | 2168 | 2555 |
| | | like protein 1 | | enhancing | | | | | | | | | |
| Aurpu2p4_000232 | Aurpu2p4_000232 | unknown | unknown | | unknown CE1 | CE1 | | 25 | 26 | 27 | 1782 | 2169 | 2556 |
| Aurpu2p4_000354 | | unknown | unknown | | unknown GH79 | GH79 | | 28 | 29 | 30 | 1783 | 2170 | 2557 |
| Aurpu2p4_000408 | Aurpu2p4_000408 | Putative cell wall | possible adhesin | | adhesin | | | 31 | 32 | 33 | 1784 | 2171 | 2558 |
| | | adhesin | | | | | | | | | | | |
| Aurpu2p4_000459 | Aurpu2p4_000459 | exo-1,3-beta- | exo-1,3-beta- | cellulose- | exo-1,3-beta- | GH5 | | 34 | 35 | 36 | 1785 | 2172 | 2559 |
| | | glucanase | glucanase GH5 | degrading | glucanase | | | | | | | | |
| Aurpu2p4_000533 | | exo-1,3-beta- | Exo-1,3-beta- | cellulose- | Exo-1,3-beta- | GH55 | | 37 | 38 | 39 | 1786 | 2173 | 2560 |
| | | glucanase | glucanase GH55 | degrading | glucanase | | | | | | | | |
| Aurpu2p4_000568 | AURPU_3_00012 | xylanase | xylanase GH10 | hemicellulose- | xylanase | GH10 | CBM1 | 40 | 41 | 42 | 1787 | 2174 | 2561 |
| | | | | degrading | | | | | | | | | |
| Aurpu2p4_000586 | | unknown | unknown | | unknown | | | 43 | 44 | 45 | 1788 | 2175 | 2562 |
| Aurpu2p4_000590 | AURPU_3_00002 | Beta-glucosidase | beta-glucosidase GH1 | cellulose- | beta-glucosidase | GH1 | | 46 | 47 | 48 | 1789 | 2176 | 2563 |
| | | 40 | | degrading | | | | | | | | | |
| Aurpu2p4_000594 | | alpha- | alpha-galactosidase | hemicellulose- | alpha- | GH27 | | 49 | 50 | 51 | 1790 | 2177 | 2564 |
| | | galactosidase | GH27 | degrading | galactosidase | | | | | | | | |
| Aurpu2p4_000617 | Aurpu2p4_000617 | Carboxylesterase 8 | Para-nitrobenzyl | | carboxylesterase | CE10 | | 52 | 53 | 54 | 1791 | 2178 | 2565 |
| | | | esterase | | | | | | | | | | |
| Aurpu2p4_000662 | | Aspergillopepsin-F | Aspartic protease | protein | protease | | | 55 | 56 | 57 | 1792 | 2179 | 2566 |
| | | | pep1 | hydrolysis | | | | | | | | | |
| Aurpu2p4_000692 | | beta-glucosidase | beta-glucosidase GH1 | cellulose- | beta-glucosidase | GH1 | | 58 | 59 | 60 | 1793 | 2180 | 2567 |
| | | | | degrading | | | | | | | | | |
| Aurpu2p4_000730 | | Carboxypeptidase Y | Carboxypeptidase Y | protein | protease | | | 61 | 62 | 63 | 1794 | 2181 | 2568 |
| | | | homolog A | hydrolysis | | | | | | | | | |
| Aurpu2p4_000792 | Aurpu2p4_000792 | Probable pectin | pectin lyase PL1 | pectin-degrading | pectin lyase | PL1 | | 64 | 65 | 66 | 1795 | 2182 | 2569 |
| | | lyase D | | | | | | | | | | | |
| Aurpu2p4_000799 | | unknown | unknown | | unknown CE1 | CE1 | | 67 | 68 | 69 | 1796 | 2183 | 2570 |
| Aurpu2p4_000819 | | Putative serine | Putative serine | protein | protease | | | 70 | 71 | 72 | 1797 | 2184 | 2571 |
| | | protease K12H4.7 | protease K12H4.7 | hydrolysis | | | | | | | | | |
| Aurpu2p4_000860 | Aurpu2p4_000860 | Probable alpha-N- | alpha | hemicellulose- | arabinofuranosidase | GH51 | | 73 | 74 | 75 | 1798 | 2185 | 2572 |
| | | arabinofuranosidase A | arabinofuranosidase | degrading | | | | | | | | | |
| | | | GH51 | | | | | | | | | | |
| Aurpu2p4_000919 | AURPU_3_00164 | Putative | exo- | pectin-degrading | rhamno- | GH28 | | 76 | 77 | 78 | 1799 | 2186 | 2573 |
| | | galacturan 1,4- | rhamnogalacturonase | | galacturonase | | | | | | | | |
| | | alpha- | GH28 | | | | | | | | | | |
| | | galacturonidase B | | | | | | | | | | | |
| Aurpu2p4_000934 | AURPU_3_00165 | Putative | exo- | pectin-degrading | rhamno- | GH28 | | 79 | 80 | 81 | 1800 | 2187 | 2574 |
| | | galacturan 1,4- | rhamnogalacturonase | | galacturonase | | | | | | | | |
| | | alpha- | GH28 | | | | | | | | | | |
| | | galacturonidase B | | | | | | | | | | | |
| Aurpu2p4_000947 | AURPU_3_00284 | Inulinase | invertase GH32 | | invertase | GH32 | | 82 | 83 | 84 | 1801 | 2188 | 2575 |
| Aurpu2p4_000948 | AURPU_3_00288 | Invertase | exo-inulinase GH32 | | exo-inulinase | GH32 | | 85 | 86 | 87 | 1802 | 2189 | 2576 |
| Aurpu2p4_000984 | AURPU_3_00187 | beta-glucosidase | beta-glucosidase GH3 | cellulose- | beta-glucosidase | GH3 | | 88 | 89 | 90 | 1803 | 2190 | 2577 |
| | | | | degrading | | | | | | | | | |
| Aurpu2p4_000995 | AURPU_3_00068 | Probable endo- | mixed-link glucanase | glucan- | mixed-link | GH16 | | 91 | 92 | 93 | 1804 | 2191 | 2578 |
| | | 1,3(4)-beta- | GH16 | degrading | glucanase | | | | | | | | |
| | | glucanase | | | | | | | | | | | |
| | | ACLA_073210 | | | | | | | | | | | |
| Aurpu2p4_001037 | Aurpu2p4_001037 | Cellobiose | Cellobiose | lignin-degrading | cellobiose | | | 94 | 95 | 96 | 1805 | 2192 | 2579 |
| | | dehydrogenase | dehydrogenase | | dehydrogenase | | | | | | | | |
| Aurpu2p4_001097 | Aurpu2p4_001097 | Adhesin protein | possible adhesin | | adhesin | | | 97 | 98 | 99 | 1806 | 2193 | 2580 |
| | | Mad1 | | | | | | | | | | | |
| Aurpu2p4_001104 | | unknown | galactanase GH5 | hemicellulose- | galactanase | GH5 | | 100 | 101 | 102 | 1807 | 2194 | 2581 |
| | | | | degrading | | | | | | | | | |
| Aurpu2p4_001152 | | Glucan 1,3-beta- | Probable glucan 1,3- | cellulose- | glucan 1,3-beta- | GH5 | | 103 | 104 | 105 | 1808 | 2195 | 2582 |
| | | glucosidase 1 | beta-glucosidase A | degrading | glucosidase | | | | | | | | |
| Aurpu2p4_001194 | Aurpu2p4_001194 | Pectinesterase | Pectinesterase | pectin-degrading | pectinesterase | CE8 | | 106 | 107 | 108 | 1809 | 2196 | 2583 |
| Aurpu2p4_001195 | | Probable leucine | Leucine | protein | protease | | | 109 | 110 | 111 | 1810 | 2197 | 2584 |
| | | aminopeptidase 1 | aminopeptidase 1 | hydrolysis | | | | | | | | | |
| Aurpu2p4_001256 | AURPU_3_00017 | xylanase | xylanase GH11 | hemicellulose- | xylanase¹⁰ | GH11 | | 112 | 113 | 114 | 1811 | 2198 | 2585 |
| | | | | degrading | | | | | | | | | |
| Aurpu2p4_001441 | Aurpu2p4_001441 | Probable alpha-N- | alpha- | hemicellulose- | arabinofuranosidase | GH51 | | 115 | 116 | 117 | 1812 | 2199 | 2586 |
| | | arabinofuranosidase A | arabinofuranosidase | degrading | | | | | | | | | |
| | | | GH51 | | | | | | | | | | |
| Aurpu2p4_001503 | AURPU_3_00393 | cellobiohydrolase | cellobiohydrolase GH6 | cellulose- | cellobiohydrolase | GH6 | CBM1 | 118 | 119 | 120 | 1813 | 2200 | 2587 |
| | | | | degrading | | | | | | | | | |
| Aurpu2p4_001504 | AURPU_3_00429 | cellobiohydrolase | cellobiohydrolase | cellulose- | cellobio- | GH7 | | 121 | 122 | 123 | 1814 | 2201 | 2588 |
| | | | GH7 | degrading | hydrolase¹¹ | | | | | | | | |
| Aurpu2p4_001512 | Aurpu2p4_001512 | Rhamnogalacturonase B | rhamnogalacturonan | pectin-degrading | rhamno- | PL4 | | 124 | 125 | 126 | 1815 | 2202 | 2589 |
| | | | lyase PL4 | | galacturonase | | | | | | | | |
| Aurpu2p4_001553 | Aurpu2p4_001553 | Liver | Acetylcholinesterase 4 | | carboxylesterase | CE10 | | 127 | 128 | 129 | 1816 | 2203 | 2590 |
| | | carboxylesterase | | | | | | | | | | | |
| Aurpu2p4_001599 | Aurpu2p4_001599 | Tannase | Tannase | tannin- | tannase | | | 130 | 131 | 132 | 1817 | 2204 | 2591 |
| | | | | degrading | | | | | | | | | |
| Aurpu2p4_001600 | | Carboxypeptidase | Carboxypeptidase | protein | protease | | | 133 | 134 | 135 | 1818 | 2205 | 2592 |
| | | cpdS | cpdS | hydrolysis | | | | | | | | | |
| Aurpu2p4_001633 | Aurpu2p4_001633 | endoglucanase | Endoglucanase GH5 | cellulose- | Endoglucanase | GH5 | | 136 | 137 | 138 | 1819 | 2206 | 2593 |
| | | | | degrading | | | | | | | | | |
| Aurpu2p4_001665 | | Gamma- | Gamma- | protein | protease | | | 139 | 140 | 141 | 1820 | 2207 | 2594 |
| | | glutamyltranspeptidase 2 | glutamyltranspeptidase 1 | hydrolysis | | | | | | | | | |
| Aurpu2p4_001680 | | Peptidase M20 | Probable | protein | protease | | | 142 | 143 | 144 | 1821 | 2208 | 2595 |
| | | domain-containing | carboxypeptidase | hydrolysis | | | | | | | | | |
| | | protein C757.05c | AFLA_037450 | | | | | | | | | | |
| Aurpu2p4_001713 | Aurpu2p4_001713 | Versatile | Manganese | lignin-degrading | versatile | | | 145 | 146 | 147 | 1822 | 2209 | 2596 |
| | | peroxidase VPL1 | peroxidase 1 | | peroxidase | | | | | | | | |
| Aurpu2p4_001718 | Aurpu2p4_001718 | Endochitinase | chitinase GH18 | chitin-degrading | chitinase | GH18 | | 148 | 149 | 150 | 1823 | 2210 | 2597 |
| Aurpu2p4_001807 | AURPU_3_00342 | unknown | Alpha-N- | hemicellulose- | arabino- | GH43 | | 151 | 152 | 153 | 1824 | 2211 | 2598 |
| | | | arabinofuranosidase 2 | degrading | furanosidase¹² | | | | | | | | |
| Aurpu2p4_001825 | AURPU_3_00390 | Probable | arabinogalactanase | hemicellulose- | arabino- | GH53 | | 154 | 155 | 156 | 1825 | 2212 | 2599 |
| | | arabinogalactan | GH53 | degrading | galactanase | | | | | | | | |
| | | endo-1,4-beta- | | | | | | | | | | | |
| | | galactosidase A | | | | | | | | | | | |
| Aurpu2p4_001892 | AURPU_3_00112 | Probable | Probable glycosidase | carbohydrate- | glycosidase | GH16 | | 157 | 158 | 159 | 1826 | 2213 | 2600 |
| | | glycosidase CRH1 | crf1 | modifying | | | | | | | | | |
| Aurpu2p4_001986 | Aurpu2p4_001986 | unknown | alpha-rhamnosidase | hemicellulose- | alpha- | GH78 | | 160 | 161 | 162 | 1827 | 2214 | 2601 |
| | | | GH78 | degrading | rhamnosidase | | | | | | | | |
| Aurpu2p4_002000 | | Aspergillopepsin-2 | Aspergillopepsin-2 | protein | protease | | | 163 | 164 | 165 | 1828 | 2215 | 2602 |
| | | | | hydrolysis | | | | | | | | | |
| Aurpu2p4_002005 | | exo-arabinanase | exo-arabinanase | hemicellulose- | exo-arabinanase | GH93 | | 166 | 167 | 168 | 1829 | 2216 | 2603 |
| | | | GH93 | degrading | | | | | | | | | |
| Aurpu2p4_002047 | AURPU_3_00027 | xylanase | xylanase GH11 | hemicellulose- | xylanase | GH11 | | 169 | 170 | 171 | 1830 | 2217 | 2604 |
| | | | | degrading | | | | | | | | | |
| Aurpu2p4_002086 | | Carboxypeptidase S | Carboxypeptidase S | protein | protease | | | 172 | 173 | 174 | 1831 | 2218 | 2605 |
| | | | | hydrolysis | | | | | | | | | |
| Aurpu2p4_002155 | | unknown | unknown | | unknown | | | 175 | 176 | 177 | 1832 | 2219 | 2606 |
| Aurpu2p4_002166 | AURPU_3_00351 | unknown | unknown | | unknown | GH43 | | 178 | 179 | 180 | 1833 | 2220 | 2607 |
| Aurpu2p4_002167 | Aurpu2p4_002167 | Probable exo-1,4- | beta-xylosidase GH3 | hemicellulose | beta-xylosidase | GH3 | | 181 | 182 | 183 | 1834 | 2221 | 2608 |
| | | beta-xylosidase | | degrading | | | | | | | | | |
| | | bxlB | | | | | | | | | | | |
| Aurpu2p4_002190 | | Vacuolar protease A | Vacuolar protease A | protein | protease | | | 184 | 185 | 186 | 1835 | 2222 | 2609 |
| | | | | hydrolysis | | | | | | | | | |
| Aurpu2p4_002220 | Aurpu2p4_002220 | Aldose 1- | Aldose 1-epimerase | | aldose epimerase | | | 187 | 188 | 189 | 1836 | 2223 | 2610 |
| | | epimerase | | | | | | | | | | | |
| Aurpu2p4_002256 | AURPU_3_00237 | Periplasmic beta- | beta-glucosidase GH3 | cellulose- | beta-glucosidase | GH3 | | 190 | 191 | 192 | 1837 | 2224 | 2611 |
| | | glucosidase | | degrading | | | | | | | | | |
| Aurpu2p4_002267 | Aurpu2p4_002267 | Acetylxylan | Acetylxylan esterase 2 | hemicellulose- | acetylxylan | CE5 | | 193 | 194 | 195 | 1838 | 2225 | 2612 |
| | | esterase | | degrading | esterase | | | | | | | | |
| Aurpu2p4_002284 | Aurpu2p4_002284 | alpha-arabino- | alpha- | hemicellulose- | arabino- | GH54 | | 196 | 197 | 198 | 1839 | 2226 | 2613 |
| | | furanosidase | arabinofuranosidase | degrading | furanosidase | | | | | | | | |
| | | | GH54 | | | | | | | | | | |
| Aurpu2p4_002399 | | unknown | unknown | | unknown CBM18 | | CBM18 | 199 | 200 | 201 | 1840 | 2227 | 2614 |
| Aurpu2p4_002518 | | Probable aspartic- | Probable aspartic-type | protein | protease | | | 202 | 203 | 204 | 1841 | 2228 | 2615 |
| | | type | endopeptidase OPSB | hydrolysis | | | | | | | | | |
| | | endopeptidase | | | | | | | | | | | |
| | | opsB | | | | | | | | | | | |
| Aurpu2p4_002522 | Aurpu2p4_002522 | unknown | unknown | | unknown GH43 | GH43 | | 205 | 206 | 207 | 1842 | 2229 | 2616 |
| Aurpu2p4_002533 | Aurpu2p4_002533 | Laccase | Laccase-3 (Fragment) | lignin-degrading | laccase | | | 208 | 209 | 210 | 1843 | 2230 | 2617 |
| Aurpu2p4_002671 | AURPU_3_00176 | endo- | Endo- | pectin-degrading | Endo- | GH28 | | 211 | 212 | 213 | 1844 | 2231 | 2618 |
| | | polygalacturonase | polygalacturonase | | polygalacturonase | | | | | | | | |
| | | | GH28 | | | | | | | | | | |
| Aurpu2p4_002672 | AURPU_3_00239 | beta-glucosidase | avenacinase GH3 | | avenacinase | GH3 | | 214 | 215 | 216 | 1845 | 2232 | 2619 |
| Aurpu2p4_002750 | | unknown | unknown | | unknown CE16 | CE16 | | 217 | 218 | 219 | 1846 | 2233 | 2620 |
| Aurpu2p4_002860 | AURPU_3_00296 | unknown | invertase GH32 | | invertase GH32 | GH32 | | 220 | 221 | 222 | 1847 | 2234 | 2621 |
| Aurpu2p4_002907 | AURPU_3_00353 | unknown | unknown | cellulase - | unknown GH43¹³ | GH43 | | 223 | 224 | 225 | 1848 | 2235 | 2622 |
| | | | | enhacing | | | | | | | | | |
| Aurpu2p4_002940 | Aurpu2p4_002940 | Laccase-2 | Laccase-1 | lignin-degrading | laccase | | | 226 | 227 | 228 | 1849 | 2236 | 2623 |
| Aurpu2p4_002942 | | Aspergillopepsin-F | Aspartic protease | protein | protease | | | 229 | 230 | 231 | 1850 | 2237 | 2624 |
| | | | pepB | hydrolysis | | | | | | | | | |
| Aurpu2p4_002955 | | Putative | Putative | protein | protease | | | 232 | 233 | 234 | 1851 | 2238 | 2625 |
| | | metallocarboxypeptidase | metallocarboxypeptidase | hydrolysis | | | | | | | | | |
| | | MCYG_04493 | ECM14 | | | | | | | | | | |
| Aurpu2p4_002987 | | unknown | unknown | | unknown GH79 | GH79 | | 235 | 236 | 237 | 1852 | 2239 | 2626 |
| Aurpu2p4_003029 | Aurpu2p4_003029 | Pectinesterase | pectin methylesterase | pectin-degrading | pectinesterase | CE8 | | 238 | 239 | 240 | 1853 | 2240 | 2627 |
| | | | CE8 | | | | | | | | | | |
| Aurpu2p4_003104 | | Probable glucan | Probable glucan 1,3- | cellulose- | glucan 1,3-beta- | GH5 | | 241 | 242 | 243 | 1854 | 2241 | 2628 |
| | | 1,3-beta- | beta-glucosidase A | degrading | glucosidase | | | | | | | | |
| | | glucosidase D | | | | | | | | | | | |
| Aurpu2p4_003184 | AURPU_3_00468 | Cellulase 1 | Cellulase 1 | cellulose- | cellulase | GH9 | | 244 | 245 | 246 | 1855 | 2242 | 2629 |
| | | | | degrading | | | | | | | | | |
| Aurpu2p4_003313 | Aurpu2p4_003313 | Tannase | Tannase | Tannin- | tannase | | | 247 | 248 | 249 | 1856 | 2243 | 2630 |
| | | | | degrading | | | | | | | | | |
| Aurpu2p4_003364 | Aurpu2p4_003364 | unknown | unknown | | unknown CE1 | CE1 | | 250 | 251 | 252 | 1857 | 2244 | 2631 |
| Aurpu2p4_003555 | | Lipase B | Lipase B | Lipid-degrading | lipase | | | 253 | 254 | 255 | 1858 | 2245 | 2632 |
| Aurpu2p4_003594 | AURPU_3_00167 | endo- | endo- | pectin-degrading | endo- | GH28 | | 256 | 257 | 258 | 1859 | 2246 | 2633 |
| | | polygalacturonase | polygalacturonase | | polygalacturonase | | | | | | | | |
| | | | GH28 | | | | | | | | | | |
| Aurpu2p4_003606 | Aurpu2p4_003606 | Manganese | Manganese | lignin-degrading | manganese | | | 259 | 260 | 261 | 1860 | 2247 | 2634 |
| | | peroxidase 3 | peroxidase 1 | | peroxidase | | | | | | | | |
| Aurpu2p4_003607 | Aurpu2p4_003607 | Versatile | Ligninase LG5 | lignin-degrading | versatile | | | 262 | 263 | 264 | 1861 | 2248 | 2635 |
| | | peroxidase VPL1 | | | peroxidase | | | | | | | | |
| Aurpu2p4_003685 | | unknown | unknown | | unknown | | | 265 | 266 | 267 | 1862 | 2249 | 2636 |
| Aurpu2p4_003727 | | Putative | Putative | protein | protease | | | 268 | 269 | 270 | 1863 | 2250 | 2637 |
| | | aspergillopepsin A- | aspergillopepsin A-like | hydrolysis | | | | | | | | | |
| | | like aspartic | aspartic | | | | | | | | | | |
| | | endopeptidase | endopeptidase | | | | | | | | | | |
| | | AFUA_2G15950 | AFUA_2G15950 | | | | | | | | | | |
| Aurpu2p4_003747 | AURPU_3_00306 | beta-galactosidase | Beta-galactosidase | hemicellulose- | beta-galactosidase | GH35 | | 271 | 272 | 273 | 1864 | 2251 | 2638 |
| | | | GH35 | degrading | | | | | | | | | |
| Aurpu2p4_003884 | AURPU_3_00389 | arabinogalactanase | arabinogalactanase | hemicellulose- | arabino- | GH53 | | 274 | 275 | 276 | 1865 | 2252 | 2639 |
| | | | GH53 | degrading | galactanase | | | | | | | | |
| Aurpu2p4_003888 | Aurpu2p4_003888 | Cutinase 3 | Cutinase 2 | cutin-degrading | cutinase | CE5 | | 277 | 278 | 279 | 1866 | 2253 | 2640 |
| Aurpu2p4_003893 | Aurpu2p4_003893 | Expansin family | unknown | | expansin | | | 280 | 281 | 282 | 1867 | 2254 | 2641 |
| | | protein | | | | | | | | | | | |
| Aurpu2p4_003941 | Aurpu2p4_003941 | unknown | unknown | | unknown CE2 | CE2 | | 283 | 284 | 285 | 1868 | 2255 | 2642 |
| Aurpu2p4_004107 | | Carboxypeptidase | Carboxypeptidase S1 | protein | protease | | | 286 | 287 | 288 | 1869 | 2256 | 2643 |
| | | S1 homolog A | homolog A | hydrolysis | | | | | | | | | |
| Aurpu2p4_004115 | AURPU_3_00326 | endo-1,5-alpha- | endo-1,5-alpha- | hemicellulose- | endo-1,5-alpha- | GH43 | | 289 | 290 | 291 | 1870 | 2257 | 2644 |
| | | arabinanase | arabinanase GH43 | degrading | arabinanase | | | | | | | | |
| Aurpu2p4_004128 | AURPU_3_00242 | unknown | xylanase GH30 | hemicellulose | xylanase | GH30 | | 292 | 293 | 294 | 1871 | 2258 | 2645 |
| | | | | degrading | | | | | | | | | |
| Aurpu2p4_004186 | Aurpu2p4_004186 | unknown | unknown | | unknown CE5 | CE5 | | 295 | 296 | 297 | 1872 | 2259 | 2646 |
| Aurpu2p4_004265 | AURPU_3_00191 | beta-glucosidase | beta-glucosidase GH3 | cellulose- | beta-glucosidase | GH3 | | 298 | 299 | 300 | 1873 | 2260 | 2647 |
| | | | | degrading | | | | | | | | | |
| Aurpu2p4_004286 | | Glucan 1,3-beta- | Glucan 1,3-beta- | glucan- | glucan 1,3-beta- | | | 301 | 302 | 303 | 1874 | 2261 | 2648 |
| | | glucosidase | glucosidase I/II | degrading | glucosidase | | | | | | | | |
| Aurpu2p4_004297 | | GLEYA adhesin | possible adhesin | | adhesin | GH16 | | 304 | 305 | 306 | 1875 | 2262 | 2649 |
| | | domain | | | | | | | | | | | |
| Aurpu2p4_004347 | | Probable aspartic- | Probable aspartic-type | protein | protease | | | 307 | 308 | 309 | 1876 | 2263 | 2650 |
| | | type | endopeptidase opsB | hydrolysis | | | | | | | | | |
| | | endopeptidase | | | | | | | | | | | |
| | | opsB | | | | | | | | | | | |
| Aurpu2p4_004477 | | Glucan 1,3-beta- | exo-1,3-beta- | cellulose- | exo-1,3-beta- | GH55 | | 310 | 311 | 312 | 1877 | 2264 | 2651 |
| | | glucosidase | glucanase GH55 | degrading | glucanase | | | | | | | | |
| Aurpu2p4_004489 | | Carboxypeptidase | Carboxypeptidase | protein | protease | | | 313 | 314 | 315 | 1878 | 2265 | 2652 |
| | | cpdS | cpdS | hydrolysis | | | | | | | | | |
| Aurpu2p4_004524 | Aurpu2p4_004524 | Acetylxylan | unknown | hemicellulose- | acetylxylan | CE5 | | 316 | 317 | 318 | 1879 | 2266 | 2653 |
| | | esterase 2 | | degrading | esterase | | | | | | | | |
| Aurpu2p4_004527 | | Probable endo- | Probable endo-1,3(4)- | cellulose- | endo-1,3(4)-beta- | GH16 | | 319 | 320 | 321 | 1880 | 2267 | 2654 |
| | | 1,3(4)-beta- | beta-glucanase | degrading | glucanase | | | | | | | | |
| | | glucanase | NFIA_089530 | | | | | | | | | | |
| | | AFUB_029980 | | | | | | | | | | | |
| Aurpu2p4_004550 | AURPU_3_00396 | cellulase- | polysaccharide | cellulose- | polysaccharide | GH61 | | 322 | 323 | 324 | 1881 | 2268 | 2655 |
| | | enhancing protein | monooxygenase | degrading | monooxygenase | | | | | | | | |
| Aurpu2p4_004694 | Aurpu2p4_004694 | unknown | unknown | | unknown CE16 | CE16 | | 325 | 326 | 327 | 1882 | 2269 | 2656 |
| Aurpu2p4_004762 | | unknown | unknown | | unknown | | | 328 | 329 | 330 | 1883 | 2270 | 2657 |
| Aurpu2p4_004776 | Aurpu2p4_004776 | Expansin-like | Expansin-yoaJ | cellulose- | expansin | | | 331 | 332 | 333 | 1884 | 2271 | 2658 |
| | | protein 5 | | enhancing | | | | | | | | | |
| Aurpu2p4_004801 | | Carboxypeptidase Y | Carboxypeptidase S1 | protein | protease | | | 334 | 335 | 336 | 1885 | 2272 | 2659 |
| | | | homolog B | hydrolysis | | | | | | | | | |
| Aurpu2p4_004899 | AURPU_3_00009 | beta-glucosidase | beta-glucosidase GH1 | cellulose- | beta-glucosidase | GH1 | | 337 | 338 | 339 | 1886 | 2273 | 2660 |
| | | | | degrading | | | | | | | | | |
| Aurpu2p4_004916 | Aurpu2p4_004916 | GLEYA adhesin | possible adhesin | | adhesin | | | 340 | 341 | 342 | 1887 | 2274 | 2661 |
| | | domain | | | | | | | | | | | |
| Aurpu2p4_004926 | AURPU_3_00324 | Probable arabinan | endo-1,5-alpha- | hemicellulose- | endo-1,5-alpha- | GH43 | | 343 | 344 | 345 | 1888 | 2275 | 2662 |
| | | endo-1,5-alpha-L- | arabinanase GH43 | degrading | arabinanase | | | | | | | | |
| | | arabinosidase B | | | | | | | | | | | |
| Aurpu2p4_004937 | Aurpu2p4_004937 | Probable | rhamnogalacturonan | pectin-degrading | rhamno- | PL4 | | 346 | 347 | 348 | 1889 | 2276 | 2663 |
| | | rhamnogalacturonate | lyase PL4 | | galacturonase | | | | | | | | |
| | | lyase B | | | | | | | | | | | |
| Aurpu2p4_004986 | Aurpu2p4_004986 | Probable glucan | Probable glucan 1,3- | cellulose- | glucan 1,3-beta- | GH5 | | 349 | 350 | 351 | 1890 | 2277 | 2664 |
| | | 1,3-beta- | beta-glucosidase A | degrading | glucosidase A | | | | | | | | |
| | | glucosidase D | | | | | | | | | | | |
| Aurpu2p4_005056 | AURPU_3_00397 | cellulase- | polysaccharide | cellulose- | polysaccharide | GH61 | | 352 | 353 | 354 | 1891 | 2278 | 2665 |
| | | enhancing protein | monooxygenase | degrading | monooxygenase | | | | | | | | |
| Aurpu2p4_005097 | AURPU_3_00100 | Probable | Probable glycosidase | Carbohydrate- | glycosidase | GH16 | | 355 | 356 | 357 | 1892 | 2279 | 2666 |
| | | glycosidase crf1 | crf1 | modifying | | | | | | | | | |
| Aurpu2p4_005194 | AURPU_3_00166 | endo- | endo- | pectin-degrading | endo- | GH28 | | 358 | 359 | 360 | 1893 | 2280 | 2667 |
| | | polygalacturonase | polygalacturonase | | polygalacturonase | | | | | | | | |
| | | | GH28 | | | | | | | | | | |
| Aurpu2p4_005236 | AURPU_3_00290 | exo-inulinase | exo-inulinase | | exo-inulinase | GH32 | | 361 | 362 | 363 | 1894 | 2281 | 2668 |
| | | | GH32/GH43 | | GH32/GH43 | | | | | | | | |
| Aurpu2p4_005278 | Aurpu2p4_005278 | Bifunctional | chitin deacetylase CE4 | chitin-degrading | chitin deacetylase | CE4 | | 364 | 365 | 366 | 1895 | 2282 | 2669 |
| | | xylanase/deacetylase | | | CE4 | | | | | | | | |
| Aurpu2p4_005399 | AURPU_3_00295 | unknown | unknown | | unknown GH43 | GH43 | | 367 | 368 | 369 | 1896 | 2283 | 2670 |
| Aurpu2p4_005401 | Aurpu2p4_005401 | alpha- | alpha- | hemicellulose- | arabinofuranosidase | GH51 | | 370 | 371 | 372 | 1897 | 2284 | 2671 |
| | | arabinofuranosidase | arabinofuranosidase | degrading | | | | | | | | | |
| | | | GH51 | | | | | | | | | | |
| Aurpu2p4_005519 | AURPU_3_00354 | unknown | unknown | Hemicellulose- | unknown GH43 | GH43 | | 373 | 374 | 375 | 1898 | 2285 | 2672 |
| | | | | modifying | | | | | | | | | |
| Aurpu2p4_005580 | Aurpu2p4_005580 | Probable glucan | Probable glucan 1,3- | glucan- | glucan 1,3-beta- | GH5 | | 376 | 377 | 378 | 1899 | 2286 | 2673 |
| | | 1,3-beta- | beta-glucosidase A | degrading | glucosidase | | | | | | | | |
| | | glucosidase A | | | | | | | | | | | |
| Aurpu2p4_005825 | Aurpu2p4_005825 | Probable endo- | mixed-link glucanase | Glucan- | mixed-link | GH16 | | 379 | 380 | 381 | 1900 | 2287 | 2674 |
| | | 1,3(4)-beta- | GH16 | degrading | glucanase | | | | | | | | |
| | | glucanase | | | | | | | | | | | |
| | | An02g00850 | | | | | | | | | | | |
| Aurpu2p4_005865 | | Probable beta- | Probable glycosidase | Carbohydrate- | glycosidase | GH16 | | 382 | 383 | 384 | 1901 | 2288 | 2675 |
| | | fructosidase | crf1 | modfying | | | | | | | | | |
| Aurpu2p4_005914 | AURPU_3_00184 | Probable exo-1,4- | beta-xylosidase GH3 | hemicellulose- | beta-xylosidase¹⁴ | GH3 | | 385 | 386 | 387 | 1902 | 2289 | 2676 |
| | | beta-xylosidase | | degrading | | | | | | | | | |
| | | bxlB | | | | | | | | | | | |
| Aurpu2p4_005929 | Aurpu2p4_005929 | Uncharacterized | Uncharacterized | | oxidoreductase | | | 388 | 389 | 390 | 1903 | 2290 | 2677 |
| | | oxidoreductase | oxidoreductase | | | | | | | | | | |
| | | C521.03 | C521.03 | | | | | | | | | | |
| Aurpu2p4_006113 | AURPU_3_00395 | cellulase- | polysaccharide | Cellulose- | polysaccharide | GH61 | CBM1 | 391 | 392 | 393 | 1904 | 2291 | 2678 |
| | | enhancing | monooxygenase | degrading | monooxygenase | | | | | | | | |
| | | protein | | | | | | | | | | | |
| Aurpu2p4_006128 | AURPU_3_00058 | unknown | unknown | | unknown GH16 | GH16 | | 394 | 395 | 396 | 1905 | 2292 | 2679 |
| Aurpu2p4_006160 | AURPU_3_00320 | Xylosidase/arabinosidase | Xylosidase/arabinosidase | Hemicellulose- | Xylosidase/ | GH43 | | 397 | 398 | 399 | 1906 | 2293 | 2680 |
| | | | | modifying | arabinosidase | | | | | | | | |
| Aurpu2p4_006162 | | unknown | unknown | | unknown GH79 | GH79 | | 400 | 401 | 402 | 1907 | 2294 | 2681 |
| Aurpu2p4_006176 | | Probable alpha- | alpha-galactosidase | hemicellulose- | alpha- | GH27 | | 403 | 404 | 405 | 1908 | 2295 | 2682 |
| | | galactosidase A | GH27 | degrading | galactosidase | | | | | | | | |
| Aurpu2p4_006179 | | Lipase 2 | Lipase 1 | Lipid-degrading | lipase | | | 406 | 407 | 408 | 1909 | 2296 | 2683 |
| Aurpu2p4_006195 | | Carboxypeptidase | Carboxypeptidase S1 | protein | protease | | | 409 | 410 | 411 | 1910 | 2297 | 2684 |
| | | S1 homolog A | homolog A | hydrolysis | | | | | | | | | |
| Aurpu2p4_006206 | | unknown | unknown | | unknown CE1 | CE1 | | 412 | 413 | 414 | 1911 | 2298 | 2685 |
| Aurpu2p4_006207 | Aurpu2p4_006207 | GLEYA adhesin | possible adhesin | | adhesin | | | 415 | 416 | 417 | 1912 | 2299 | 2686 |
| | | domain | | | | | | | | | | | |
| Aurpu2p4_006222 | Aurpu2p4_006222 | Tannase | Tannase | Tannin- | tannase | | | 418 | 419 | 420 | 1913 | 2300 | 2687 |
| | | | | degrading | | | | | | | | | |
| Aurpu2p4_006237 | Aurpu2p4_006237 | Probable pectate | pectate lyase PL3 | pectin-degrading | pectate lyase | PL3 | | 421 | 422 | 423 | 1914 | 2301 | 2688 |
| | | lyase F | | | | | | | | | | | |
| Aurpu2p4_006246 | AURPU_3_00192 | beta-glucosidase | beta-glucosidase GH3 | cellulose- | beta-glucosidase | GH3 | | 424 | 425 | 426 | 1915 | 2302 | 2689 |
| | | | | degrading | | | | | | | | | |
| Aurpu2p4_006312 | AURPU_3_00032 | unknown | xyloglucanase GH12 | hemicellulose- | xyloglucanase | GH12 | | 427 | 428 | 429 | 1916 | 2303 | 2690 |
| | | | | degrading | | | | | | | | | |
| Aurpu2p4_006313 | Aurpu2p4_006313 | Probable | pectin methylesterase | pectin-degrading | pectinesterase | CE8 | | 430 | 431 | 432 | 1917 | 2304 | 2691 |
| | | pectinesterase A | CE8 | | | | | | | | | | |
| Aurpu2p4_006392 | AURPU_3_00331 | unknown | unknown | | unknown GH43 | GH43 | | 433 | 434 | 435 | 1918 | 2305 | 2692 |
| Aurpu2p4_006557 | | Glucan 1,3-beta- | Probable glucan 1,3- | cellulose- | glucan 1,3-beta- | GH5 | | 436 | 437 | 438 | 1919 | 2306 | 2693 |
| | | glucosidase 1 | beta-glucosidase A | degrading | glucosidase A | | | | | | | | |
| Aurpu2p4_006782 | Aurpu2p4_006782 | beta-glucosidase | beta-glucosidase | cellulose- | beta-glucosidase | GH3 | | 439 | 440 | 441 | 1920 | 2307 | 2694 |
| | | | GH3 | degrading | | | | | | | | | |
| Aurpu2p4_006900 | Aurpu2p4_006900 | GLEYA adhesin | possible adhesin | | adhesin | | | 442 | 443 | 444 | 1921 | 2308 | 2695 |
| | | domain | | | | | | | | | | | |
| Aurpu2p4_006933 | | Tripeptidyl- | Tripeptidyl-peptidase | protein | protease | | | 445 | 446 | 447 | 1922 | 2309 | 2696 |
| | | peptidase sed1 | SED1 | hydrolysis | | | | | | | | | |
| Aurpu2p4_007070 | Aurpu2p4_007070 | GLEYA adhesin | possible adhesin | | adhesin | | | 448 | 449 | 450 | 1923 | 2310 | 2697 |
| | | domain | | | | | | | | | | | |
| Aurpu2p4_007082 | AURPU_3_00013 | xylanase | xylanase GH10 | hemicellulose- | xylanase¹⁵ | GH10 | | 451 | 452 | 453 | 1924 | 2311 | 2698 |
| | | | | degrading | | | | | | | | | |
| Aurpu2p4_007093 | AURPU_3_00019 | xylanase | xylanase GH11 | hemicellulose- | xylanase¹⁶ | GH11 | | 454 | 455 | 456 | 1925 | 2312 | 2699 |
| | | | | degrading | | | | | | | | | |
| Aurpu2p4_007113 | | unknown | unknown PL22 | Pectin- | unknown PL22 | PL22 | | 457 | 458 | 459 | 1926 | 2313 | 2700 |
| | | | | degrading | | | | | | | | | |
| Aurpu2p4_007124 | | unknown | unknown | | unknown CBM1 | | CBM1 | 460 | 461 | 462 | 1927 | 2314 | 2701 |
| Aurpu2p4_007126 | AURPU_3_00356 | unknown | unknown | | unknown GH43 | GH43 | | 463 | 464 | 465 | 1928 | 2315 | 2702 |
| Aurpu2p4_007149 | Aurpu2p4_007149 | unknown | unknown | | unknown CBM1 | | CBM1 | 466 | 467 | 468 | 1929 | 2316 | 2703 |
| Aurpu2p4_007160 | | Putative lipase | Putative lipase | Lipid-degrading | lipase | | | 469 | 470 | 471 | 1930 | 2317 | 2704 |
| | | ATG15-1 | ATG15-1 | | | | | | | | | | |
| Aurpu2p4_007177 | AURPU_3_00035 | Beta-galactosidase | Beta-galactosidase | hemicellulose- | Beta-galactosidase | GH42 | | 472 | 473 | 474 | 1931 | 2318 | 2705 |
| | | | | degrading | | | | | | | | | |
| Aurpu2p4_007190 | Aurpu2p4_007190 | Endoglucanase B | Endoglucanase B | cellulose- | Endoglucanase B | GH5 | | 475 | 476 | 477 | 1932 | 2319 | 2706 |
| | | | | degrading | | | | | | | | | |
| Aurpu2p4_007196 | Aurpu2p4_007196 | Probable | Probable | pectin-degrading | pectinesterase | CE8 | | 478 | 479 | 480 | 1933 | 2320 | 2707 |
| | | pectinesterase/pectinesterase | pectinesterase A | | | | | | | | | | |
| | | inhibitor 41 | | | | | | | | | | | |
| Aurpu2p4_007206 | AURPU_3_00177 | exo- | exo-polygalacturonase | pectin-degrading | exo- | GH28 | | 481 | 482 | 483 | 1934 | 2321 | 2708 |
| | | polygalacturonase | GH28 | | polygalacturonase | | | | | | | | |
| Aurpu2p4_007220 | | Chitinase 1 | Chitinase 3 | chitin-degrading | chitinase | GH18 | | 484 | 485 | 486 | 1935 | 2322 | 2709 |
| Aurpu2p4_007270 | AURPU_3_00241 | beta-glucosidase | avenacinase GH3 | | avenacinase | GH3 | | 487 | 488 | 489 | 1936 | 2323 | 2710 |
| Aurpu2p4_007272 | | Carboxypeptidase | Carboxypeptidase | protein | protease | | | 490 | 491 | 492 | 1937 | 2324 | 2711 |
| | | cpdS | cpdS | hydrolysis | | | | | | | | | |
| Aurpu2p4_007292 | | Putative | Putative NADPH- | | oxidoreductase | | | 493 | 494 | 495 | 1938 | 2325 | 2712 |
| | | uncharacterized | dependent | | | | | | | | | | |
| | | oxidoreductase | methylglyoxal | | | | | | | | | | |
| | | YGL157W | reductase GRP2 | | | | | | | | | | |
| Aurpu2p4_007342 | | unknown | unknown | | unknown CE16 | CE16 | | 496 | 497 | 498 | 1939 | 2326 | 2713 |
| Aurpu2p4_007356 | AURPU_3_00178 | exo- | exo-polygalacturonase | pectin-degrading | exo- | GH28 | | 499 | 500 | 501 | 1940 | 2327 | 2714 |
| | | polygalacturonase | GH28 | | polygalacturonase | | | | | | | | |
| Aurpu2p4_007383 | Aurpu2p4_007383 | unknown | unknown CE1 | hemicellulose- | xylan alpha-1,2- | GH115 | | 502 | 503 | 504 | 1941 | 2328 | 2715 |
| | | | | degrading | glucuronidase | | | | | | | | |
| Aurpu2p4_007404 | | Probable glucan | Probable glucan 1,3- | cellulose- | glucan 1,3-beta- | GH5 | | 505 | 506 | 507 | 1942 | 2329 | 2716 |
| | | 1,3-beta- | beta-glucosidase A | degrading | glucosidase | | | | | | | | |
| | | glucosidase A | | | | | | | | | | | |
| Aurpu2p4_007424 | Aurpu2p4_007424 | unknown | galactanase GH5 | hemicellulose- | galactanase | GH5 | | 508 | 509 | 510 | 1943 | 2330 | 2717 |
| | | | | degrading | | | | | | | | | |
| Aurpu2p4_007428 | | unknown | exo-arabinanase | hemicellulose- | exo-arabinanase | GH93 | | 511 | 512 | 513 | 1944 | 2331 | 2718 |
| | | | GH93 | degrading | | | | | | | | | |
| Aurpu2p4_007429 | AURPU_3_00314 | Alpha-N- | Alpha-N- | hemicellulose- | arabino- | GH43 | | 514 | 515 | 516 | 1945 | 2332 | 2719 |
| | | arabinofuranosidase 2 | arabinofuranosidase 2 | degrading | furanosidase | | | | | | | | |
| Aurpu2p4_007455 | Aurpu2p4_007455 | Probable feruloyl | feruloyl esterase CE1 | hemicellulose- | feruloyl esterase | | | 517 | 518 | 519 | 1946 | 2333 | 2720 |
| | | esterase B | | degrading | | | | | | | | | |
| Aurpu2p4_007488 | AURPU_3_00028 | unknown | Probable xyloglucan- | Hemicellulose- | xyloglucan- | GH12 | | 520 | 521 | 522 | 1947 | 2334 | 2721 |
| | | | specific endo-beta- | degrading | specific endo- | | | | | | | | |
| | | | 1,4-glucanase A | | beta-1,4- | | | | | | | | |
| | | | | | glucanase | | | | | | | | |
| Aurpu2p4_007493 | | Subtilisin-like | Alkaline protease 2 | protein | protease | | | 523 | 524 | 525 | 1948 | 2335 | 2722 |
| | | proteinase Spm1 | | hydrolysis | | | | | | | | | |
| Aurpu2p4_007511 | AURPU_3_00315 | Alpha-N- | Alpha-N- | hemicellulose- | arabino- | GH43 | | 526 | 527 | 528 | 1949 | 2336 | 2723 |
| | | arabinofuranosidase 2 | arabinofuranosidase 2 | degrading | furanosidase | | | | | | | | |
| Aurpu2p4_007612 | Aurpu2p4_007612 | Cutinase | cutinase CE5 | cutin-degrading | cutinase | CE5 | | 529 | 530 | 531 | 1950 | 2337 | 2724 |
| Aurpu2p4_007614 | Aurpu2p4_007614 | Probable pectin | pectin lyase PL1 | pectin-degrading | pectin lyase | PL1 | | 532 | 533 | 534 | 1951 | 2338 | 2725 |
| | | lyase A | | | | | | | | | | | |
| Aurpu2p4_007621 | AURPU_3_00155 | endo- | Endo- | pectin-degrading | Endo- | GH28 | | 535 | 536 | 537 | 1952 | 2339 | 2726 |
| | | polygalacturonase | polygalacturonase | | polygalacturonase | | | | | | | | |
| | | | GH28 | | | | | | | | | | |
| Aurpu2p4_007662 | | Aspergillopepsin-2 | Aspergillopepsin-2 | protein | protease | | | 538 | 539 | 540 | 1953 | 2340 | 2727 |
| | | | | hydrolysis | | | | | | | | | |
| Aurpu2p4_007707 | AURPU_3_00394 | cellulase- | polysaccharide | cellulose- | polysaccharide | GH61 | | 541 | 542 | 543 | 1954 | 2341 | 2728 |
| | | enhancing protein | monooxygenase | degrading | monooxygenase | | | | | | | | |
| Aurpu2p4_007805 | Aurpu2p4_007805 | Laccase-3 | Laccase | lignin-degrading | laccase | | | 544 | 545 | 546 | 1955 | 2342 | 2729 |
| | | (Fragment) | | | | | | | | | | | |
| Aurpu2p4_007919 | Aurpu2p4_007919 | unknown | unknown | | unknown CE5 | CE5 | | 547 | 548 | 549 | 1956 | 2343 | 2730 |
| Aurpu2p4_008001 | AURPU_3_00054 | unknown | unknown | | unknown GH16 | GH16 | | 550 | 551 | 552 | 1957 | 2344 | 2731 |
| Aurpu2p4_008021 | Aurpu2p4_008021 | Mannan endo-1,4- | Mannan endo-1,4- | hemicellulose- | Mannan endo-1,4- | GH5 | | 553 | 554 | 555 | 1958 | 2345 | 2732 |
| | | beta-mannosidase 3 | beta-mannosidase 4 | degrading | beta-mannosidase | | | | | | | | |
| Aurpu2p4_008140 | Aurpu2p4_008140 | Probable feruloyl | feruloyl esterase CE1 | hemicellulose- | feruloyl esterase | | | 556 | 557 | 558 | 1959 | 2346 | 2733 |
| | | esterase B-2 | | modifying | | | | | | | | | |
| Aurpu2p4_008212 | AURPU_3_00357 | Putative | Putative | cellulose- | endoglucanase | GH45 | | 559 | 560 | 561 | 1960 | 2347 | 2734 |
| | | endoglucanase | endoglucanase type K | degrading | | | | | | | | | |
| | | type K | | | | | | | | | | | |
| Aurpu2p4_008231 | AURPU_3_00157 | Endo- | Probable endo- | pectin-degrading | endo- | CE8 | | 562 | 563 | 564 | 1961 | 2348 | 2735 |
| | | xylogalacturonan | xylogalacturonan | | xylogalacturonan | | | | | | | | |
| | | hydrolase A | hydrolase A | | hydrolase | | | | | | | | |
| Aurpu2p4_008239 | AURPU_3_00323 | unknown | exo-1,3-beta- | hemicellulose- | exo-1,3-beta- | GH43 | CBM35 | 565 | 566 | 567 | 1962 | 2349 | 2736 |
| | | | galactanase GH43 | degrading | galactanase | | | | | | | | |
| Aurpu2p4_008212 | AURPU_3_00357 | Putative | Putative | cellulose- | endoglucanase | GH45 | | 559 | 560 | 561 | 1960 | 2347 | 2734 |
| | | endoglucanase | endoglucanase type K | degrading | | | | | | | | | |
| | | type K | | | | | | | | | | | |
| Aurpu2p4_008231 | AURPU_3_00157 | Endo- | Probable endo- | pectin-degrading | endo- | CE8 | | 562 | 563 | 564 | 1961 | 2348 | 2735 |
| | | xylogalacturonan | xylogalacturonan | | xylogalacturonan | | | | | | | | |
| | | hydrolase A | hydrolase A | | hydrolase | | | | | | | | |
| Aurpu2p4_008239 | AURPU_3_00323 | unknown | exo-1,3-beta- | hemicellulose- | exo-1,3-beta- | GH43 | CBM35 | 565 | 566 | 567 | 1962 | 2349 | 2736 |
| | | | galactanase GH43 | degrading | galactanase | | | | | | | | |
| Aurpu2p4_008255 | AURPU_3_00064 | Beta-glucanase | unknown | | unknown GH16 | GH16 | | 568 | 569 | 570 | 1963 | 2350 | 2737 |
| Aurpu2p4_008271 | | Putative lipase | Putative lipase | lipid-degrading | lipase | | | 571 | 572 | 573 | 1964 | 2351 | 2738 |
| | | ATG15-1 | ATG15-1 | | | | | | | | | | |
| Aurpu2p4_008282 | | Probable | Probable tripeptidyl- | protein | protease | | | 574 | 575 | 576 | 1965 | 2352 | 2739 |
| | | tripeptidyl- | peptidase SED2 | hydrolysis | | | | | | | | | |
| | | peptidase SED2 | | | | | | | | | | | |
| Aurpu2p4_008385 | Aurpu2p4_008385 | Liver | Carboxylesterase 5A | | carboxylesterase | CE10 | | 577 | 578 | 579 | 1966 | 2353 | 2740 |
| | | carboxylesterase | | | | | | | | | | | |
| Aurpu2p4_008412 | AURPU_3_00305 | Probable beta- | Beta-galactosidase | hemicellulose- | Beta-galactosidase | GH35 | | 580 | 581 | 582 | 1967 | 2354 | 2741 |
| | | galactosidase C | GH35 | degrading | | | | | | | | | |
| Aurpu2p4_008485 | AURPU_3_00101 | Probable endo- | mixed-link glucanase | Glucan- | mixed-link | GH16 | | 583 | 584 | 585 | 1968 | 2355 | 2742 |
| | | 1,3(4)-beta- | GH16 | degrading | glucanase | | | | | | | | |
| | | glucanase | | | | | | | | | | | |
| | | AFUB_029980 | | | | | | | | | | | |
| Aurpu2p4_008495 | Aurpu2p4_008495 | Unknown-Esterase | unknown | | unknown CE12 | CE12 | | 586 | 587 | 588 | 1969 | 2356 | 2743 |
| Aurpu2p4_008503 | Aurpu2p4_008503 | unknown | probable beta- | cellulase- | beta- | GH79 | | 589 | 590 | 591 | 1970 | 2357 | 2744 |
| | | | glucuronidase GH79 | enhacing | glucuronidase | | | | | | | | |
| Aurpu2p4_008585 | Aurpu2p4_008585 | Cellobiose | Cellobiose | lignin-degrading | cellobiose | | CBM1 | 592 | 593 | 594 | 1971 | 2358 | 2745 |
| | | dehydrogenase | dehydrogenase | | dehydrogenase | | | | | | | | |
| Aurpu2p4_008692 | | Carboxypeptidase | Carboxypeptidase S1 | protein | protease | | | 595 | 596 | 597 | 1972 | 2359 | 2746 |
| | | S1 homolog B | homolog A | hydrolysis | | | | | | | | | |
| Aurpu2p4_008705 | Aurpu2p4_008705 | GLEYA adhesin | possible adhesin | | adhesin | | | 598 | 599 | 600 | 1973 | 2360 | 2747 |
| | | domain | | | | | | | | | | | |
| Aurpu2p4_008725 | AURPU_3_00334 | Arabinan endo- | endo-1,5-alpha- | hemicellulose- | endo-1,5-alpha- | GH43 | | 601 | 602 | 603 | 1974 | 2361 | 2748 |
| | | 1,5-alpha-L- | arabinanase GH43 | degrading | arabinanase | | | | | | | | |
| | | arabinosidase | | | | | | | | | | | |
| Aurpu2p4_008775 | Aurpu2p4_008775 | Putative | Putative galacturan | pectin-degrading | galacturan 1,4- | GH28 | | 604 | 605 | 606 | 1975 | 2362 | 2749 |
| | | galacturan 1,4- | 1,4-alpha- | | alpha- | | | | | | | | |
| | | alpha- | galacturonidase A | | galacturonidase | | | | | | | | |
| | | galacturonidase A | | | | | | | | | | | |
| Aurpu2p4_008807 | AURPU_3_00341 | unknown | unknown | Hemicellulose- | unknown GH43¹⁷ | GH43 | | 607 | 608 | 609 | 1976 | 2363 | 2750 |
| | | | | modfiying | | | | | | | | | |
| Aurpu2p4_008838 | | unknown | unknown | | unknown | | | 610 | 611 | 612 | 1977 | 2364 | 2751 |
| Aurpu2p4_008906 | AURPU_3_00175 | endo- | endo- | pectin-degrading | endo- | GH28 | | 613 | 614 | 615 | 1978 | 2365 | 2752 |
| | | polygalacturonase | polygalacturonase | | polygalacturonase | | | | | | | | |
| | | | GH28 | | | | | | | | | | |
| Aurpu2p4_008972 | Aurpu2p4_008972 | Probable pectin | pectin lyase PL1 | pectin-degrading | pectin lyase | PL1 | | 616 | 617 | 618 | 1979 | 2366 | 2753 |
| | | lyase A | | | | | | | | | | | |
| Aurpu2p4_008980 | AURPU_3_00147 | Probable beta- | beta-mannosidase | hemicellulose- | beta-mannosidase | GH2 | | 619 | 620 | 621 | 1980 | 2367 | 2754 |
| | | mannosidase A | GH2 | degrading | | | | | | | | | |
| Aurpu2p4_009032 | | Carboxypeptidase | Carboxypeptidase S1 | protein | protease | | | 622 | 623 | 624 | 1981 | 2368 | 2755 |
| | | S1 homolog B | homolog B | hydrolysis | | | | | | | | | |
| Aurpu2p4_009051 | Aurpu2p4_009051 | Cellobiose | Cellobiose | lignin-degrading | cellobiose | | | 625 | 626 | 627 | 1982 | 2369 | 2756 |
| | | dehydrogenase | dehydrogenase | | dehydrogenase | | | | | | | | |
| Aurpu2p4_009071 | AURPU_3_00110 | unknown | unknown | | unknown GH16 | GH16 | | 628 | 629 | 630 | 1983 | 2370 | 2757 |
| Aurpu2p4_009125 | | Cytosolic | Cytosolic | Phospholipid- | lipase | | | 631 | 632 | 633 | 1984 | 2371 | 2758 |
| | | phospholipase A2 | phospholipase A2 | modifying | | | | | | | | | |
| Aurpu2p4_009223 | Aurpu2p4_009223 | Alpha-fucosidase A | Alpha-fucosidase A | Carbohydrate- | Alpha-fucosidase | GH95 | | 634 | 635 | 636 | 1985 | 2372 | 2759 |
| | | | | modifying | | | | | | | | | |
| Aurpu2p4_009233 | AURPU_3_00016 | Endo-1,4-beta- | tomatinase GH10 | Tomatin | tomatinase | GH10 | | 637 | 638 | 639 | 1986 | 2373 | 2760 |
| | | xylanase C | | degrading | | | | | | | | | |
| Aurpu2p4_009300 | AURPU_3_00153 | Beta- | Beta-galactosidase | hemicellulose- | Beta-galactosidase | GH2 | | 640 | 641 | 642 | 1987 | 2374 | 2761 |
| | | glucuronidase | | degrading | | | | | | | | | |
| Aurpu2p4_009394 | Aurpu2p4_009394 | Probable feruloyl | feruloyl esterase CE1 | hemicellulose- | feruloyl esterase | CE1 | | 643 | 644 | 645 | 1988 | 2375 | 2762 |
| | | esterase B-1 | | modfiying | | | | | | | | | |
| Aurpu2p4_009401 | Aurpu2p4_009401 | unknown | unknown | | unknown CE5 | CE5 | | 646 | 647 | 648 | 1989 | 2376 | 2763 |
| Aurpu2p4_009472 | | unknown | Unsaturated | pectin-degrading | rhamno- | GH105 | | 649 | 650 | 651 | 1990 | 2377 | 2764 |
| | | | rhamnogalacturonyl | | galacturonyl | | | | | | | | |
| | | | hydrolase YteR | | hydrolase | | | | | | | | |
| Aurpu2p4_009494 | | Lipase B | Lipase B | Lipid-degrading | lipase | | | 652 | 653 | 654 | 1991 | 2378 | 2765 |
| Aurpu2p4_009495 | AURPU_3_00410 | arabinoxylan | arabinoxylan | hemicellulose- | arabino- | GH62 | | 655 | 656 | 657 | 1992 | 2379 | 2766 |
| | | arabino- | arabino-furanosidase | degrading | furanosidase¹⁸ | | | | | | | | |
| | | furanosidase | GH62 | | | | | | | | | | |
| Aurpu2p4_009496 | AURPU_3_00333 | Beta-xylosidase | Xylosidase/arabinosidase | Hemicellulose- | Xylosidase/ | GH43 | | 658 | 659 | 660 | 1993 | 2380 | 2767 |
| | | | | degrading | arabinosidase | | | | | | | | |
| Aurpu2p4_009563 | | Adhesin protein, | possible adhesin | | adhesin | | | 661 | 662 | 663 | 1994 | 2381 | 2768 |
| | | putative | | | | | | | | | | | |
| Aurpu2p4_009597 | Aurpu2p4_009597 | unknown | GDSL esterase/lipase | Lipid-degrading | lipase | CE16 | | 664 | 665 | 666 | 1995 | 2382 | 2769 |
| | | | EXL5 | | | | | | | | | | |
| Aurpu2p4_009603 | | Expansin family | unknown | Cellulase- | expansin | | | 667 | 668 | 669 | 1996 | 2383 | 2770 |
| | | protein | | enhancing | | | | | | | | | |
| Aurpu2p4_009751 | Aurpu2p4_009751 | xylanase | Xylanase GH10 | hemicellulose- | xylanase | GH10 | | 670 | 671 | 672 | 1997 | 2384 | 2771 |
| | | | | degrading | | | | | | | | | |
| Aurpu2p4_009762 | Aurpu2p4_009762 | endo- | endo- | pectin-degrading | rhamno- | GH28 | | 673 | 674 | 675 | 1998 | 2385 | 2772 |
| | | rhamnogalacturonase | rhamnogalacturonase | | galacturonase | | | | | | | | |
| | | | GH28 | | | | | | | | | | |
| Aurpu2p4_009775 | | Glucoamylase | Glucoamylase | starch- | Glucoamylase | | | 676 | 677 | 678 | 1999 | 2386 | 2773 |
| | | | | degrading | | | | | | | | | |
| Aurpu2p4_009782 | AURPU_3_00011 | Beta-glucosidase | beta-glucosidase GH1 | cellulose- | beta-glucosidase | GH1 | | 679 | 680 | 681 | 2000 | 2387 | 2774 |
| | | 26 | | degrading | | | | | | | | | |
| Aurpu2p4_009845 | AURPU_3_00402 | cellulase- | polysaccharide | cellulose- | polysaccharide | GH61 | CBM1 | 682 | 683 | 684 | 2001 | 2388 | 2775 |
| | | enhancing | monooxygenase | degrading | monooxygenase | | | | | | | | |
| | | protein | | | | | | | | | | | |
| Aurpu2p4_009863 | AURPU_3_00105 | unknown | unknown | | unknown GH16 | GH16 | | 685 | 686 | 687 | 2002 | 2389 | 2776 |
| Aurpu2p4_009889 | Aurpu2p4_009889 | Probable feruloyl | feruloyl esterase CE1 | hemicellulose- | feruloyl esterase | | | 688 | 689 | 690 | 2003 | 2390 | 2777 |
| | | esterase B-2 | | modifying | | | | | | | | | |
| Aurpu2p4_009890 | Aurpu2p4_009890 | Probable beta- | beta-glucosidase GH3 | cellulose- | beta-glucosidase | GH3 | | 691 | 692 | 693 | 2004 | 2391 | 2778 |
| | | glucosidase D | | degrading | | | | | | | | | |
| Aurpu2p4_009910 | AURPU_3_00219 | beta-glucosidase | beta-glucosidase GH3 | cellulose- | beta-glucosidase | GH3 | | 694 | 695 | 696 | 2005 | 2392 | 2779 |
| | | | | degrading | | | | | | | | | |
| Aurpu2p4_010058 | Aurpu2p4_010058 | Probable alpha- | alpha-galactosidase | hemicellulose- | alpha- | GH27 | CBM35 | 697 | 698 | 699 | 2006 | 2393 | 2780 |
| | | galactosidase D | GH27 | degrading | galactosidase | | | | | | | | |
| Aurpu2p4_010070 | Aurpu2p4_010070 | beta-glucosidase | beta-glucosidase GH3 | cellulose- | beta-glucosidase | GH3 | | 700 | 701 | 702 | 2007 | 2394 | 2781 |
| | | | | degrading | | | | | | | | | |
| Aurpu2p4_010087 | AURPU_3_00339 | Xylosidase/arabinosidase | Xylosidase/arabinosidase | hemicellulose- | Xylosidase/ | GH43 | | 703 | 704 | 705 | 2008 | 2395 | 2782 |
| | | | | modifying | arabinosidase | | | | | | | | |
| Aurpu2p4_010088 | Aurpu2p4_010088 | alpha- | alpha-glucuronidase | hemicellulose- | alpha- | GH67 | | 706 | 707 | 708 | 2009 | 2396 | 2783 |
| | | glucuronidase | GH67 | degrading | glucuronidase | | | | | | | | |
| Aurpu2p4_010125 | AURPU_3_00407 | cellulase- | polysaccharide | cellulose- | polysaccharide | GH61 | | 709 | 710 | 711 | 2010 | 2397 | 2784 |
| | | enhancing | monooxygenase | degrading | monooxygenase | | | | | | | | |
| | | protein | | | | | | | | | | | |
| Aurpu2p4_010146 | | Tripeptidyl- | Tripeptidyl-peptidase | Protein | protease | | | 712 | 713 | 714 | 2011 | 2398 | 2785 |
| | | peptidase sed4 | sed4 | hydrolysis | | | | | | | | | |
| Aurpu2p4_010192 | Aurpu2p4_010192 | Probable beta- | Beta-galactosidase | hemicellulose- | beta-galactosidase | GH35 | | 715 | 716 | 717 | 2012 | 2399 | 2786 |
| | | galactosidase B | GH35 | degrading | | | | | | | | | |
| Aurpu2p4_010196 | AURPU_3_00340 | Putative beta- | arabinoxylan | hemicellulose- | arabino- | GH43 | | 718 | 719 | 720 | 2013 | 2400 | 2787 |
| | | xylosidase | arabinofuranohydrolase | degrading | furanosidase | | | | | | | | |
| | | | GH43 | | | | | | | | | | |
| Aurpu2p4_010203 | Aurpu2p4_010203 | Rhamnogalacturonan | rhamnogalacturonan | pectin-degrading | rhamno- | CE12 | | 721 | 722 | 723 | 2014 | 2401 | 2788 |
| | | acetylesterase | acetylesterase CE12 | | galacturonan | | | | | | | | |
| | | | | | acetylesterase | | | | | | | | |
| Aurpu2p4_010291 | Aurpu2p4_010291 | Probable pectate | pectate lyase PL3 | pectin-degrading | pectate lyase | PL3 | | 724 | 725 | 726 | 2015 | 2402 | 2789 |
| | | lyase E | | | | | | | | | | | |
| Aurpu2p4_010300 | AURPU_3_00015 | beta-mannanase | beta-mannanase GH5 | Hemicellulose- | beta-mannanase | GH5 | CBM1 | 727 | 728 | 729 | 2016 | 2403 | 2790 |
| | | | | degrading | | | | | | | | | |
| Aurpu2p4_010313 | Aurpu2p4_010313 | Chitin deacetylase | Bifunctional | hemicellulose- | bifunctional | CE4 | CBM18 | 730 | 731 | 732 | 2017 | 2404 | 2791 |
| | | | xylanase/deacetylase | degrading | xylanase/ | | | | | | | | |
| | | | | | deacetylase | | | | | | | | |
| Aurpu2p4_010319 | Aurpu2p4_010319 | Laccase-2 | Laccase-2 | lignin-degrading | laccase | | | 733 | 734 | 735 | 2018 | 2405 | 2792 |
| Aurpu2p4_010388 | Aurpu2p4_010388 | Putative | exo-polygalacturonase | pectin-degrading | exo- | GH28 | | 736 | 737 | 738 | 2019 | 2406 | 2793 |
| | | galacturan 1,4- | GH28 | | polygalacturonase | | | | | | | | |
| | | alpha- | | | | | | | | | | | |
| | | galacturonidase C | | | | | | | | | | | |
| Aurpu2p4_010455 | AURPU_3_00312 | Probable glucan | exo-1,3-beta- | glucan- | exo-1,3-beta- | GH5 | | 739 | 740 | 741 | 2020 | 2407 | 2794 |
| | | 1,3-beta- | glucanase GH5 | degrading | glucanase | | | | | | | | |
| | | glucosidase A | | | | | | | | | | | |
| Aurpu2p4_010457 | AURPU_3_00408 | unknown | unknown | cellulose- | polysaccharide | GH61 | | 742 | 743 | 744 | 2021 | 2408 | 2795 |
| | | | | degrading | monooxygenase | | | | | | | | |
| Aurpu2p4_010464 | Aurpu2p4_010464 | Rhamnogalacturonate | Rhamnogalacturonate | pectin-degrading | rhamno- | PL4 | | 745 | 746 | 747 | 2022 | 2409 | 2796 |
| | | lyase | lyase | | galacturonase | | | | | | | | |
| Aurpu2p4_010466 | Aurpu2p4_010466 | Acetylxylan | Acetylxylan esterase 2 | hemicellulose- | acetylxylan | CE5 | | 748 | 749 | 750 | 2023 | 2410 | 2797 |
| | | esterase 2 | CE5 | degrading | esterase | | | | | | | | |
| Aurpu2p4_010484 | | Leucine | Aminopeptidase Y | Protein | protease | | | 751 | 752 | 753 | 2024 | 2411 | 2798 |
| | | aminopeptidase 2 | | hydrolysis | | | | | | | | | |
| Aurpu2p4_010534 | Aurpu2p4_010534 | Probable pectate | pectate lyase PL1 | pectin-degrading | pectate lyase | PL1 | | 754 | 755 | 756 | 2025 | 2412 | 2799 |
| | | lyase A | | | | | | | | | | | |
| Aurpu2p4_010571 | AURPU_3_00294 | unknown | unknown | | unknown GH43 | GH43 | | 757 | 758 | 759 | 2026 | 2413 | 2800 |
| Aurpu2p4_010592 | | Alkaline proteinase | Alkaline protease 1 | Protein | protease | | | 760 | 761 | 762 | 2027 | 2414 | 2801 |
| | | | | hydrolysis | | | | | | | | | |
| Aurpu2p4_010596 | Aurpu2p4_010596 | Probable pectate | pectate lyase PL1 | pectin-degrading | pectate lyase | PL1 | | 763 | 764 | 765 | 2028 | 2415 | 2802 |
| | lyase A | | | | | | | | | | | | |
| Aurpu2p4_010603 | Aurpu2p4_010603 | Probable glucan | Probable glucan 1,3- | cellulose- | glucan 1,3-beta- | GH5 | | 766 | 767 | 768 | 2029 | 2416 | 2803 |
| | | 1,3-beta- | beta-glucosidase A | degrading | glucosidase A | | | | | | | | |
| | | glucosidase A | | | | | | | | | | | |
| Aurpu2p4_010618 | | Probable | Tripeptidyl-peptidase | Protein | protease | | | 769 | 770 | 771 | 2030 | 2417 | 2804 |
| | | tripeptidyl- | SED2 | hydrolysis | | | | | | | | | |
| | | peptidase SED3 | | | | | | | | | | | |
| Aurpu2p4_010680 | | Carbohydrate- | unknown | | unknown | | | 772 | 773 | 774 | 2031 | 2418 | 2805 |
| | | binding | | | | | | | | | | | |
| | | cytochrome b562 | | | | | | | | | | | |
| | | (Fragment) | | | | | | | | | | | |
| Aurpu2p4_010683 | Aurpu2p4_010683 | Probable feruloyl | feruloyl esterase CE1 | hemicellulose- | feruloyl esterase | | | 775 | 776 | 777 | 2032 | 2419 | 2806 |
| | | esterase B-1 | | modifying | | | | | | | | | |
| Aurpu2p4_010701 | AURPU_3_00115 | unknown | unknown | | unknown GH16 | GH16 | | 778 | 779 | 780 | 2033 | 2420 | 2807 |
| Aurpu2p4_010884 | Aurpu2p4_010884 | unknown | unknown | | uncharacterized | | | 781 | 782 | 783 | 2034 | 2421 | 2808 |
| | | | | | lignocellulose- | | | | | | | | |
| | | | | | induced protein | | | | | | | | |
| Aurpu2p4_010891 | | Carboxypeptidase | Carboxypeptidase S1 | Protein | protease | | | 784 | 785 | 786 | 2035 | 2422 | 2809 |
| | | S1 homolog B | homolog A | hydrolysis | | | | | | | | | |
| Aurpu2p4_010898 | Aurpu2p4_010898 | hexosaminidase | hexosaminidase GH20 | chitin-degrading | hexosaminidase | GH20 | | 787 | 788 | 789 | 2036 | 2423 | 2810 |
| | | | | | GH20 | | | | | | | | |
| Aurpu2p4_010982 | AURPU_3_00409 | unknown | unknown | cellulose- | polysaccharide | GH61 | | 790 | 791 | 792 | 2037 | 2424 | 2811 |
| | | | | degrading | monooxygenase | | | | | | | | |
| Aurpu2p4_010999 | | Lysophospholipase 1 | Lysophospholipase 2 | Phospholipid- | lipase | | | 793 | 794 | 795 | 2038 | 2425 | 2812 |
| | | | | modifying | | | | | | | | | |
| Aurpu2p4_011049 | AURPU_3_00183 | beta-mannanase | beta-mannanase GH5 | hemicellulose- | beta-mannanase | GH5 | | 796 | 797 | 798 | 2039 | 2426 | 2813 |
| | | | | degrading | | | | | | | | | |
| Aurpu2p4_011071 | Aurpu2p4_011071 | Rhamnogalacturonate | Rhamnogalacturonate | pectin-degrading | rhamno- | PL4 | | 799 | 800 | 801 | 2040 | 2427 | 2814 |
| | | lyase | lyase | | galacturonase | | | | | | | | |
| Aurpu2p4_011080 | AURPU_3_00240 | Probable beta- | beta-glucosidase GH3 | cellulose- | beta-glucosidase | GH3 | | 802 | 803 | 804 | 2041 | 2428 | 2815 |
| | | glucosidase M | | degrading | | | | | | | | | |
| Aurpu2p4_011097 | | Aspergillopepsin-F | Aspartic protease | Protein | protease | | | 805 | 806 | 807 | 2042 | 2429 | 2816 |
| | | | PEP1 | hydrolysis | | | | | | | | | |
| Aurpu2p4_011162 | | Tripeptidyl- | Tripeptidyl-peptidase | Peptide | protease | | | 808 | 809 | 810 | 2043 | 2430 | 2817 |
| | | peptidase sed2 | sed2 | hydrolysis | | | | | | | | | |
| | Aurpu2p4_000066 | | unknown | | uncharacterized | | | | | | 2044 | 2431 | 2818 |
| | | | | | lignocellulose- | | | | | | | | |
| | | | | | induced protein | | | | | | | | |
| | Aurpu2p4_000166 | | Trans-1,2- | | Dehydrogenase | GH109 | | | | | 2045 | 2432 | 2819 |
| | | | dihydrobenzene-1,2- | | | | | | | | | | |
| | | | diol dehydrogenase | | | | | | | | | | |
| | Aurpu2p4_000811 | | O- | | oxidoreductase | | | | | | 2046 | 2433 | 2820 |
| | | | methylsterigmatocystin | | | | | | | | | | |
| | | | oxidoreductase | | | | | | | | | | |
| | Aurpu2p4_001233 | | Sterol-4-alpha- | | Dehydrogenase | | | | | | 2047 | 2434 | 2821 |
| | | | carboxylate 3- | | | | | | | | | | |
| | | | dehydrogenase, | | | | | | | | | | |
| | | | decarboxylating | | | | | | | | | | |
| | Aurpu2p4_002002 | | Retinol | | Dehydrogenase | | | | | | 2048 | 2435 | 2822 |
| | | | dehydrogenase 10-B | | | | | | | | | | |
| | Aurpu2p4_002244 | | Cytochrome P450 | | Cytochrome P450 | | | | | | 2049 | 2436 | 2823 |
| | | | 3A11 | | | | | | | | | | |
| | Aurpu2p4_002270 | | Tyrosinase | Pigment- | Tyrosinase | | | | | | 2050 | 2437 | 2824 |
| | | | | producing | | | | | | | | | |
| | Aurpu2p4_002403 | | unknown | | uncharacterized | | | | | | 2051 | 2438 | 2825 |
| | | | | | lignocellulose- | | | | | | | | |
| | | | | | induced protein | | | | | | | | |
| | Aurpu2p4_002547 | | Uncharacterized | | oxidoreductase | | | | | | 2052 | 2439 | 2826 |
| | | | oxidoreductase | | | | | | | | | | |
| | | | C26F1.07 | | | | | | | | | | |
| | Aurpu2p4_003458 | | NADPH--cytochrome | | NADPH-- | | | | | | 2053 | 2440 | 2827 |
| | | | P450 reductase | | cytochrome P450 | | | | | | | | |
| | | | | | reductase | | | | | | | | |
| | Aurpu2p4_003964 | | unknown | | uncharacterized | | | | | | 2054 | 2441 | 2828 |
| | | | | | lignocellulose- | | | | | | | | |
| | | | | | induced protein | | | | | | | | |
| | Aurpu2p4_004483 | | Uncharacterized | | oxidoreductase | | | | | | 2055 | 2442 | 2829 |
| | | | oxidoreductase dltE | | | | | | | | | | |
| | Aurpu2p4_004802 | | O- | | oxidoreductase | | | | | | 2056 | 2443 | 2830 |
| | | | methylsterigmatocystin | | | | | | | | | | |
| | | | oxidoreductase | | | | | | | | | | |
| | Aurpu2p4_005858 | | unknown | | uncharacterized | GH128 | | | | | 2057 | 2444 | 2831 |
| | | | | | lignocellulose- | | | | | | | | |
| | | | | | induced protein | | | | | | | | |
| | Aurpu2p4_006413 | | Saccharopine | | Dehydrogenase | | | | | | 2058 | 2445 | 2832 |
| | | | dehydrogenase | | | | | | | | | | |
| | | | [NADP(+), L- | | | | | | | | | | |
| | | | glutamate-forming] | | | | | | | | | | |
| | Aurpu2p4_007081 | | Tannase | Tannin- | tannase | | | | | | 2059 | 2446 | 2833 |
| | | | | degrading | | | | | | | | | |
| | Aurpu2p4_007695 | | unknown | | unknown GH16 | GH16 | | | | | 2060 | 2447 | 2834 |
| | Aurpu2p4_008408 | | unknown | | uncharacterized | | | | | | 2061 | 2448 | 2835 |
| | | | | | lignocellulose- | | | | | | | | |
| | | | | | induced protein | | | | | | | | |
| | Aurpu2p4_008733 | | Carboxylesterase 4A | | carboxylesterase | CE10 | | | | | 2062 | 2449 | 2836 |
| | Aurpu2p4_009064 | | unknown | | uncharacterized | | | | | | 2063 | 2450 | 2837 |
| | | | | | lignocellulose- | | | | | | | | |
| | | | | | induced protein | | | | | | | | |
| | Aurpu2p4_009608 | | unknown | | uncharacterized | | | | | | 2064 | 2451 | 2838 |
| | | | | | lignocellulose- | | | | | | | | |
| | | | | | induced protein | | | | | | | | |
| | Aurpu2p4_009911 | | Liver carboxylesterase 1 | | carboxylesterase | CE10 | | | | | 2065 | 2452 | 2839 |
| | Aurpu2p4_009938 | | unknown | | uncharacterized | | | | | | 2066 | 2453 | 2840 |
| | | | | | lignocellulose- | | | | | | | | |
| | | | | | induced protein | | | | | | | | |
| | Aurpu2p4_010261 | | unknown | | uncharacterized | | | | | | 2067 | 2454 | 2841 |
| | | | | | lignocellulose- | | | | | | | | |
| | | | | | induced protein | | | | | | | | |
| | Aurpu2p4_010853 | | Cytochrome P450 1A1 | | Cytochrome P450 | | | | | | 2068 | 2455 | 2842 |
| | Aurpu2p4_011048 | | unknown | | uncharacterized | | | | | | 2069 | 2456 | 2843 |
| | | | | | lignocellulose- | | | | | | | | |
| | | | | | induced protein | | | | | | | | |
| | AURPU_00050 | | unknown | | unknown CE5 | CE5 | | | | | 2070 | 2457 | 2844 |
| | AURPU_00052 | | Xylanase GH10 | hemicellulose- | xylanase | GH10 | | | | | 2071 | 2458 | 2845 |
| | | | | degrading | | | | | | | | | |
| | AURPU_00075 | | alpha- | hemicellulose- | alpha- | GH51 | | | | | 2072 | 2459 | 2846 |
| | | | arabinofuranosidase | degrading | arabinofuranosidase | | | | | | | | |
| | | | GH51 | | | | | | | | | | |
| | AURPU_00077 | | Pectinesterase | pectin-degrading | Pectinesterase | CE8 | | | | | 2073 | 2460 | 2847 |
| | AURPU_00104 | | Cutinase | cutin-degrading | Cutinase | CE5 | | | | | 2074 | 2461 | 2848 |
| | AURPU_00109 | | Cellobiose | lignin-degrading | Cellobiose | | | | | | 2075 | 2462 | 2849 |
| | | | dehydrogenase | | dehydrogenase | | | | | | | | |
| | AURPU_00159 | | Probable glycosidase | Carbohydrate- | glycosidase | GH16 | | | | | 2076 | 2463 | 2850 |
| | | | crf1 | modifying | | | | | | | | | |
| | AURPU_00163 | | Polysaccharide | cellulose- | polysaccharide | GH61 | | | | | 2077 | 2464 | 2851 |
| | | | monooxygenase | degrading | monooxygenase | | | | | | | | |
| | AURPU_00174 | | Tyrosinase | Pigment- | Tyrosinase | | | | | | 2078 | 2465 | 2852 |
| | | | | producing | | | | | | | | | |
| | AURPU_00225 | | Endo- | pectin-degrading | endo- | GH28 | | | | | 2079 | 2466 | 2853 |
| | | | polygalacturonase | | polygalacturonase | | | | | | | | |
| | | | GH28 | | | | | | | | | | |
| | AURPU_00252 | | unknown | | unknown | | | | | | 2080 | 2467 | 2854 |
| | AURPU_00265 | | xylanase GH10 | hemicellulose- | xylanase | GH10 | | | | | 2081 | 2468 | 2855 |
| | | | | degrading | | | | | | | | | |
| | AURPU_00300 | | xyloglucanase GH12 | hemicellulose- | xyloglucanase | GH12 | | | | | 2082 | 2469 | 2856 |
| | | | | degrading | | | | | | | | | |
| | AURPU_00305 | | Putative | cellulose- | endoglucanase | GH45 | | | | | 2083 | 2470 | 2857 |
| | | | endoglucanase type K | degrading | | | | | | | | | |
| | AURPU_00319 | | arabinogalactanase | hemicellulose- | arabino- | GH53 | | | | | 2084 | 2471 | 2858 |
| | | | GH53 | degrading | galactanase | | | | | | | | |
| | AURPU_00344 | | Endo-beta-1,4- | Glucan- | endo-beta-1,4- | GH5 | | | | | 2085 | 2472 | 2859 |
| | | | glucanase A | degrading | glucanase | | | | | | | | |
| | AURPU_00374 | | xylanase GH11 | hemicellulose- | xylanase | GH11 | | | | | 2086 | 2473 | 2860 |
| | | | | degrading | | | | | | | | | |
| | AURPU_00399 | | unknown | | unknown | | | | | | 2087 | 2474 | 2861 |
| | AURPU_00402 | | exo-1,3-beta- | cellulose- | exo-1,3-beta- | GH5 | | | | | 2088 | 2475 | 2862 |
| | | | glucanase GH5 | degrading | glucanase | | | | | | | | |
| | AURPU_00430 | | unknown | | unknown CE5 | CE5 | | | | | 2089 | 2476 | 2863 |
| | AURPU_00431 | | Acetylxylan esterase 1 | hemicellulose- | acetylxylan | CE1 | | | | | 2090 | 2477 | 2864 |
| | | | CE1 | degrading | esterase | | | | | | | | |
| | AURPU_00439 | | Polysaccharide | cellulose- | Polysaccharide | GH61 | CBM1 | | | | 2091 | 2478 | 2865 |
| | | | monooxygenase | degrading | monooxygenase | | | | | | | | |
| | | | GH61 | | | | | | | | | | |
| | AURPU_00457 | | Probable beta- | cellulose- | Beta-glucosidase | GH3 | | | | | 2092 | 2479 | 2866 |
| | | | glucosidase A | degrading | | | | | | | | | |
| | AURPU_00470 | | xylanase GH11 | hemicellulose- | xylanase | GH11 | | | | | 2093 | 2480 | 2867 |
| | | | | degrading | | | | | | | | | |
| | AURPU_00475 | | Probable endo-1,3(4)- | cellulose- | endo-1,3(4)-beta- | GH16 | | | | | 2094 | 2481 | 2868 |
| | | | beta-glucanase | degrading | glucanase | | | | | | | | |
| | | | AFUA_2G14360 | | | | | | | | | | |
| | AURPU_00476 | | Probable endo-1,3(4)- | cellulose- | endo-1,3(4)-beta- | GH16 | | | | | 2095 | 2482 | 2869 |
| | | | beta-glucanase | degrading | glucanase | | | | | | | | |
| | | | NFIA_089530 | | | | | | | | | | |
| | AURPU_2_00209 | | Alpha-N- | hemicellulose- | Alpha-N-arabino- | GH43 | | | | | 2096 | 2483 | 2870 |
| | | | arabinofuranosidase 2 | degrading | furanosidase | | | | | | | | |
| | AURPU_2_00581 | | Probable | pectin-degrading | exopoly- | GH28 | | | | | 2097 | 2484 | 2871 |
| | | | exopolygalacturonase B | | galacturonase | | | | | | | | |
| | AURPU_2_01541 | | xylanase GH11 | hemicellulose- | xylanase | GH11 | | | | | 2098 | 2485 | 2872 |
| | | | | degrading | | | | | | | | | |
| | AURPU_2_01594 | | beta-glucosidase GH3 | cellulose- | beta-glucosidase | GH3 | | | | | 2099 | 2486 | 2873 |
| | | | | degrading | | | | | | | | | |
| | AURPU_2_02646 | | unknown | | uncharacterized | | | | | | 2100 | 2487 | 2874 |
| | | | | | lignocellulose- | | | | | | | | |
| | | | | | induced protein | | | | | | | | |
| | AURPU_2_03623 | | Endo- | pectin-degrading | Endo- | GH28 | | | | | 2101 | 2488 | 2875 |
| | | | polygalacturonase | | polygalacturonase | | | | | | | | |
| | | | GH28 | | | | | | | | | | |
| | AURPU_2_04552 | | Xylanase GH10 | hemicellulose- | Xylanase | GH10 | | | | | 2102 | 2489 | 2876 |
| | | | | degrading | | | | | | | | | |
| | AURPU_2_04949 | | Probable glycosidase | Carbohydrate- | glycosidase | GH16 | | | | | 2103 | 2490 | 2877 |
| | | | crf1 | modifying | | | | | | | | | |
| | AURPU_2_05877 | | Polysaccharide | cellulose- | Polysaccharide | GH61 | | | | | 2104 | 2491 | 2878 |
| | | | monooxygenase | degrading | monooxygenase | | | | | | | | |
| | AURPU_2_06264 | | arabinoxylan | hemicellulose- | arabinoxylan | GH43 | | | | | 2105 | 2492 | 2879 |
| | | | arabinofuranohydrolase | degrading | arabinofurano- | | | | | | | | |
| | | | GH43 | | hydrolase | | | | | | | | |
| | AURPU_3_00001 | | beta-glucosidase GH1 | cellulose- | beta-glucosidase | GH1 | | | | | 2106 | 2493 | 2880 |
| | | | | degrading | | | | | | | | | |
| | AURPU_3_00014 | | Xylanase GH10 | hemicellulose- | Xylanase | GH10 | | | | | 2107 | 2494 | 2881 |
| | | | | degrading | | | | | | | | | |
| | AURPU_3_00018 | | xylanase GH11 | hemicellulose- | xylanase¹⁹ | GH11 | | | | | 2108 | 2495 | 2882 |
| | | | | degrading | | | | | | | | | |
| | AURPU_3_00023 | | Endo-1,4-beta- | hemicellulose- | Endo-1,4-beta- | GH11 | | | | | 2109 | 2496 | 2883 |
| | | | xylanase B | degrading | xylanase | | | | | | | | |
| | AURPU_3_00024 | | Endo-1,4-beta- | hemicellulose- | Endo-1,4-beta- | GH11 | | | | | 2110 | 2497 | 2884 |
| | | | xylanase B | degrading | xylanase | | | | | | | | |
| | AURPU_3_00051 | | unknown | | unknown GH16 | GH16 | | | | | 2111 | 2498 | 2885 |
| | AURPU_3_00113 | | Probable glycosidase | Carbohydrate | glycosidase | GH16 | | | | | 2112 | 2499 | 2886 |
| | | | crf1 | modifying | | | | | | | | | |
| | AURPU_3_00118 | | Probable glycosidase | Carbohydrate- | glycosidase | GH16 | CBM18 | | | | 2113 | 2500 | 2887 |
| | | | crf2 | modifying | | | | | | | | | |
| | AURPU_3_00139 | | Chitinase GH18 | chitin-degrading | Chitinase | GH18 | | | | | 2114 | 2501 | 2888 |
| | AURPU_3_00156 | | Endo- | pectin-degrading | Endo-rhamno- | GH28 | | | | | 2115 | 2502 | 2889 |
| | | | rhamnogalacturonase | | galacturonase | | | | | | | | |
| | | | GH28 | | | | | | | | | | |
| | AURPU_3_00173 | | exo-polygalacturonase | pectin-degrading | exo- | GH28 | | | | | 2116 | 2503 | 2890 |
| | | | GH28 | | polygalacturonase | | | | | | | | |
| | AURPU_3_00174 | | exo- | pectin-degrading | exo-rhamno- | GH28 | | | | | 2117 | 2504 | 2891 |
| | | | rhamnogalacturonase | | galacturonase | | | | | | | | |
| | | | GH28 | | | | | | | | | | |
| | AURPU_3_00208 | | beta-glucosidase | cellulose- | beta-glucosidase | GH3 | | | | | 2118 | 2505 | 2892 |
| | | | GH3 | degrading | | | | | | | | | |
| | AURPU_3_00209 | | Probable beta- | cellulose- | Beta-glucosidase | GH3 | | | | | 2119 | 2506 | 2893 |
| | | | glucosidase D | degrading | | | | | | | | | |
| | AURPU_3_00307 | | Beta-galactosidase | hemicellulose- | Beta-galactosidase | GH35 | | | | | 2120 | 2507 | 2894 |
| | | | GH35 | degrading | | | | | | | | | |
| | AURPU_3_00428 | | Probable alpha- | hemicellulose- | Alpha- | GH67 | | | | | 2121 | 2508 | 2895 |
| | | | glucuronidase A | degrading | glucuronidase | | | | | | | | |
| | Aurpu2p4_000157 | | Probable serine | Protein | protease | | | | | | 2122 | 2509 | 2896 |
| | | | protease EDA2 | hydrolysis | | | | | | | | | |
| | Aurpu2p4_000356 | | Putative | lignin-degrading | peroxidase | | | | | | 2123 | 2510 | 2897 |
| | | | sterigmatocystin | | | | | | | | | | |
| | | | biosynthesis | | | | | | | | | | |
| | | | peroxidase stcC | | | | | | | | | | |
| | Aurpu2p4_000818 | | unknown | | unknown GH121 | GH121 | | | | | 2124 | 2511 | 2898 |
| | Aurpu2p4_000960 | | Lipase 1 | Lipid-degrading | lipase | CE10 | | | | | 2125 | 2512 | 2899 |
| | Aurpu2p4_001076 | | Lipase 4 | Lipid-degrading | lipase | CE10 | | | | | 2126 | 2513 | 2900 |
| | Aurpu2p4_001476 | | feruloyl esterase CE1 | hemicellulose- | feruloyl esterase | | | | | | 2127 | 2514 | 2901 |
| | | | | degrading | | | | | | | | | |
| | Aurpu2p4_001745 | | possible hydrophobin | | hydrophobin | | | | | | 2128 | 2515 | 2902 |
| | Aurpu2p4_001987 | | Probable glucan 1,3- | cellulose- | glucan 1,3-beta- | GH5 | | | | | 2129 | 2516 | 2903 |
| | | | beta-glucosidase A | degrading | glucosidase | | | | | | | | |
| | Aurpu2p4_002339 | | Lipase 2 | Lipid-degrading | lipase | CE10 | | | | | 2130 | 2517 | 2904 |
| | Aurpu2p4_002490 | | Minor extracellular | Protein | protease | | | | | | 2131 | 2518 | 2905 |
| | | | protease vpr | hydrolysis | | | | | | | | | |
| | Aurpu2p4_002528 | | Probable aspartic-type | Protein | protease | | | | | | 2132 | 2519 | 2906 |
| | | | endopeptidase OPSB | hydrolysis | | | | | | | | | |
| | Aurpu2p4_003052 | | Gluconolactonase | | gluconolactonase | | | | | | 2133 | 2520 | 2907 |
| | Aurpu2p4_003108 | | unknown | | unknown CE1 | CE1 | | | | | 2134 | 2521 | 2908 |
| | Aurpu2p4_003243 | | possible pyranose | Sugar-modifying | pyranose | | | | | | 2135 | 2522 | 2909 |
| | | | dehydrogenase | | dehydrogenase | | | | | | | | |
| | Aurpu2p4_003247 | | possible pyranose | Sugar-modifying | pyranose | | | | | | 2136 | 2523 | 2910 |
| | | | dehydrogenase | | dehydrogenase | | | | | | | | |
| | Aurpu2p4_003704 | | Neutral protease 2 | Protein | protease | | | | | | 2137 | 2524 | 2911 |
| | | | homolog | hydrolysis | | | | | | | | | |
| | | | SNOG_10522 | | | | | | | | | | |
| | Aurpu2p4_004187 | | Probable glucan 1,3- | cellulose- | glucan 1,3-beta- | GH5 | | | | | 2138 | 2525 | 2912 |
| | | | beta-glucosidase A | degrading | glucosidase | | | | | | | | |
| | Aurpu2p4_004476 | | Probable endo-1,3(4)- | cellulose- | endo-1,3(4)-beta- | GH16 | | | | | 2139 | 2526 | 2913 |
| | | | beta-glucanase | degrading | glucanase | | | | | | | | |
| | | | ACLA_073210 | | | | | | | | | | |
| | Aurpu2p4_004865 | | Extracellular | Protein | protease | | | | | | 2140 | 2527 | 2914 |
| | | | metalloprotease | hydrolysis | | | | | | | | | |
| | | | AO090012001025 | | | | | | | | | | |
| | Aurpu2p4_005304 | | Uncharacterized | | unknown CE7 | CE7 | | | | | 2141 | 2528 | 2915 |
| | | | protein PA2218 | | | | | | | | | | |
| | Aurpu2p4_005861 | | WSC domain- | | unknown CE1 | CE1 | | | | | 2142 | 2529 | 2916 |
| | | | containing protein 1 | | | | | | | | | | |
| | Aurpu2p4_005992 | | possible adhesin | | adhesin | | | | | | 2143 | 2530 | 2917 |
| | Aurpu2p4_006091 | | Probable aspartic-type | Protein | protease | | | | | | 2144 | 2531 | 2918 |
| | | | endopeptidase | hydrolysis | | | | | | | | | |
| | | | AFUA_3G01220 | | | | | | | | | | |
| | Aurpu2p4_006277 | | Lipase 1 | Lipid-degrading | lipase | CE10 | | | | | 2145 | 2532 | 2919 |
| | Aurpu2p4_007520 | | Gluconolactonase | | gluconolactonase | | | | | | 2146 | 2533 | 2920 |
| | Aurpu2p4_007546 | | possible adhesin | | adhesin | | | | | | 2147 | 2534 | 2921 |
| | Aurpu2p4_007951 | | Lipase 1 | Lipid-degrading | lipase | CE10 | | | | | 2148 | 2535 | 2922 |
| | Aurpu2p4_008628 | | Lipase 4 | Lipid-degrading | lipase | CE10 | | | | | 2149 | 2536 | 2923 |
| | Aurpu2p4_008719 | | Putative | lignin-degrading | peroxidase | | | | | | 2150 | 2537 | 2924 |
| | | | sterigmatocystin | | | | | | | | | | |
| | | | biosynthesis | | | | | | | | | | |
| | | | peroxidase stcC | | | | | | | | | | |
| | Aurpu2p4_009254 | | Lipase 1 | Lipid-degrading | lipase | CE10 | | | | | 2151 | 2538 | 2925 |
| | Aurpu2p4_009278 | | Lipase 1 | Lipid-degrading | lipase | CE10 | | | | | 2152 | 2539 | 2926 |
| | Aurpu2p4_009437 | | possible pyranose | Sugar-modifying | pyranose | | | | | | 2153 | 2540 | 2927 |
| | | | dehydrogenase | | dehydrogenase | | | | | | | | |
| | Aurpu2p4_009445 | | Lipase 2 | Lipid-degrading | lipase | CE10 | | | | | 2154 | 2541 | 2928 |
| | Aurpu2p4_010136 | | unknown | | unknown GH2 | GH2 | | | | | 2155 | 2542 | 2929 |
| | Aurpu2p4_010244 | | Lipase 1 | Lipid-degrading | lipase | CE10 | | | | | 2156 | 2543 | 2930 |
| | Aurpu2p4_010617 | | Uncharacterized | | unknown CE7 | CE7 | | | | | 2157 | 2544 | 2931 |
| | | | protein PA2218 | | | | | | | | | | |
| | Aurpu2p4_010719 | | Bacilysin biosynthesis | | oxidoreductase | | | | | | 2158 | 2545 | 2932 |
| | | | oxidoreductase BacC | | | | | | | | | | |
| | Aurpu2p4_010798 | | Lipase 1 | | Lipid-degrading | lipase | CE10 | | | | 2159 | 2546 | 2933 |
| | Aurpu2p4_010869 | | possible hydrophobin | | hydrophobin | | | | | | 2160 | 2547 | 2934 |

¹⁰For example, endo-1,4-beta-xylanase.
¹¹For example, cellulose 1,4-beta-cellobiosidase
¹²For example, alpha-N-arabinofuranosidase
¹³Probable arabinosidase or beta-galactanase.
¹⁴For example, xylan 1,4-beta-xylanase
¹⁵For example, endo-1,4-beta-xylanase.
¹⁶For example, endo-1,4-beta-xylanase.
¹⁷Demonstrates arabinosidase or arabino(furano)sidases activity (see Example 22).
¹⁸For example, alpha-L-arabinofuranosidase axhA-1
¹⁹For example, endo-1,4-beta-xylanase

TABLE 2A
List of genes of Scytalidium thermophilum with reference to exon boundaries

| | Genomic | Genomic | |
|---|---|---|---|
| | sequence | sequence | |
| Gene ID | (SEQ ID NO:) | length | Exon boundaries (nucleotide positions) and exons |
| Scyth2p4_000006 | 1 | 1405 | 1 . . . 83, 138 . . . 541, 633 . . . 679, 799 . . . 1212, 1271 . . . 1405 |
| Scyth2p4_000010 | 2 | 964 | 1 . . . 178, 246 . . . 964 |
| Scyth2p4_000016 | 3 | 1809 | 1 . . . 161, 234 . . . 894, 968 . . . 1019, 1077 . . . 1555, 1627 . . . 1809 |
| Scyth2p4_000019 | 4 | 656 | 1 . . . 310, 385 . . . 656 |
| Scyth2p4_000123 | 5 | 1677 | 1 . . . 1677 |
| Scyth2p4_000124 | 6 | 873 | 1 . . . 78, 179 . . . 312, 373 . . . 529, 622 . . . 873 |
| Scyth2p4_000141 | 7 | 1560 | 1 . . . 1120, 1184 . . . 1560 |
| Scyth2p4_000168 | 8 | 971 | 1 . . . 261, 327 . . . 971 |
| Scyth2p4_000230 | 9 | 1325 | 1 . . . 1073, 1145 . . . 1325 |
| Scyth2p4_000277 | 10 | 2072 | 1 . . . 204, 280 . . . 1652, 1712 . . . 2072 |
| Scyth2p4_000610 | 11 | 1515 | 1 . . . 421, 503 . . . 1515 |
| Scyth2p4_000863 | 12 | 1946 | 1 . . . 165, 280 . . . 1446, 1518 . . . 1946 |
| Scyth2p4_000904 | 13 | 1332 | 1 . . . 1332 |
| Scyth2p4_001035 | 14 | 2348 | 1 . . . 301, 417 . . . 547, 640 . . . 906, 972 . . . 2348 |
| Scyth2p4_001183 | 15 | 1728 | 1 . . . 493, 563 . . . 1728 |
| Scyth2p4_001259 | 16 | 652 | 1 . . . 360, 428 . . . 652 |
| Scyth2p4_001262 | 17 | 1331 | 1 . . . 360, 440 . . . 1184, 1270 . . . 1331 |
| Scyth2p4_001326 | 18 | 988 | 1 . . . 423, 491 . . .633, 691 . . . 988 |
| Scyth2p4_001371 | 19 | 2659 | 1 . . . 191, 257 . . . 560, 623 . . . 997, 1061 . . . 2659 |
| Scyth2p4_001379 | 20 | 1751 | 1 . . . 208, 287 . . . 545, 612 . . . 1356, 1418 . . . 1609, 1689 . . . 1751 |
| Scyth2p4_001450 | 21 | 1451 | 1 . . . 43, 112 . . . 974, 1065 . . . 1451 |
| Scyth2p4_001460 | 22 | 1721 | 1 . . . 323, 388 . . . 486, 538, 776, 847 . . . 1721 |
| Scyth2p4_001513 | 23 | 5221 | 1 . . . 615, 680 . . . 782, 836 . . . 894, 987 . . . 1272, 1334 . . . 1399, 1467 . . . 1552, |
| | | | 1622 . . . 2274, 2344 . . . 2498, 2557 . . . 2719, 2776 . . . 2818, 2881 . . .3232, |
| | | | 3297 . . . 3686, 3791 . . . 4397, 4492 . . . 5221 |
| Scyth2p4_001745 | 24 | 1251 | 1 . . . 1251 |
| Scyth2p4_001867 | 25 | 2739 | 1 . . . 639, 691 . . .2739 |
| Scyth2p4_001875 | 26 | 1504 | 1 . . . 331, 397 . . . 629, 689, 797, 854 . . . 1135, 1194 . . . 1504 |
| Scyth2p4_001878 | 27 | 1185 | 1 . . . 342, 396 . . . 683, 745 . . . 1093, 1148 . . . 1185 |
| Scyth2p4_001887 | 28 | 6439 | 1 . . . 135, 191 . . . 1674, 2408 . . . 2497, 2576 . . . 2647, 2710 . . . 3205, 3965 . . . 4543, |
| | | | 5548 . . . 5672, 5743 . . . 6439 |
| Scyth2p4_001903 | 29 | 1287 | 1 . . . 210, 262 . . . 425, 483 . . . 1143, 1204 . . . 1287 |
| Scyth2p4_001974 | 30 | 1340 | 1 . . . 745, 817 . . . 1340 |
| Scyth2p4_001995 | 31 | 859 | 1 . . . 104, 161 . . . 731, 788 . . . 859 |
| Scyth2p4_001998 | 32 | 867 | 1 . . . 867 |
| Scyth2p4_002014 | 33 | 1285 | 1 . . . 271, 348 . . . 438, 497 . . . 542, 607 . . . 1195, 1248 . . . 1285 |
| Scyth2p4_002032 | 34 | 908 | 1 . . . 489, 558 . . . 908 |
| Scyth2p4_002058 | 35 | 2823 | 1 . . . 260, 375 . . . 531, 614 . . . 1151, 1216 . . . 1336, 1397 . . . 1614, 1674 . . . 2512, |
| | | | 2588 . . . 2694, 2775 . . . 2823 |
| Scyth2p4_002089 | 36 | 1082 | 1 . . . 270, 329 . . . 919, 978 . . . 1082 |
| Scyth2p4_002099 | 37 | 823 | 1 . . . 685, 744 . . . 823 |
| Scyth2p4_002112 | 38 | 945 | 1 . . . 945 |
| Scyth2p4_002143 | 39 | 2612 | 1 . . . 157, 209 . . . 469, 533 . . . 1140, 1188 . . . 2612 |
| Scyth2p4_002153 | 40 | 3458 | 1 . . . 83, 177 . . . 200, 261 . . . 415, 3088 . . . 3458 |
| Scyth2p4_002186 | 41 | 858 | 1 . . . 145, 209 . . . 858 |
| Scyth2p4_002220 | 42 | 1104 | 1 . . . 105, 155 . . . 973, 1042 . . . 1104 |
| Scyth2p4_002225 | 43 | 822 | 1 . . . 46, 126 . . . 414, 501 . . . 822 |
| Scyth2p4_002425 | 44 | 927 | 1 . . . 186, 258 . . . 419, 517 . . . 927 |
| Scyth2p4_002446 | 45 | 666 | 1 . . . 666 |
| Scyth2p4_002491 | 46 | 3064 | 1 . . . 556, 618 . . . 3064 |
| Scyth2p4_002582 | 47 | 1938 | 1 . . . 51, 112 . . . 1938 |
| Scyth2p4_002596 | 48 | 1669 | 1 . . . 363, 425 . . . 1669 |
| Scyth2p4_002639 | 49 | 1631 | 1 . . . 419, 472 . . . 557, 613 . . . 1631 |
| Scyth2p4_002689 | 50 | 705 | 1 . . . 705 |
| Scyth2p4_002854 | 51 | 1114 | 1 . . . 599, 664 . . . 1114 |
| Scyth2p4_002859 | 52 | 1380 | 1 . . . 1380 |
| Scyth2p4_003064 | 53 | 2677 | 1 . . . 290, 344 . . . 452, 519 . . . 608, 670 . . . 728, 809 . . . 838, 925 . . . 948, 1015 . . . 1074, |
| | | | 1140 . . . 1301, 1356 . . . 1533, 1611 . . . 1638, 1695 . . . 2237, 2297 . . . 2378, |
| | | | 2437 . . . 2677 |
| Scyth2p4_003098 | 54 | 5182 | 1 . . . 2154, 2215 . . . 2458, 2532 . . . 2567, 2626 . . . 2592, 2947 . . . 3583, 3632 . . . 3703, |
| | | | 3849 . . . 3943, 4035 . . . 5182 |
| Scyth2p4_003108 | 55 | 1621 | 1 . . . 166, 228 . . . 1621 |
| Scyth2p4_003124 | 56 | 1020 | 1 . . . 1020 |
| Scyth2p4_003222 | 57 | 875 | 1 . . . 426, 516 . . . 673, 788 . . . 875 |
| Scyth2p4_003248 | 58 | 1992 | 1 . . . 1992 |
| Scyth2p4_003738 | 59 | 3794 | 1 . . . 201, 308 . . . 1518, 2392 . . . 2692, 2905 . . . 3359, 3428 . . . 3794 |
| Scyth2p4_003766 | 60 | 1338 | 1 . . . 552, 851 . . . 1249, 1312 . . . 1338 |
| Scyth2p4_003836 | 61 | 2740 | 1 . . . 49, 116 . . . 300, 364 . . . 1791, 1856 . . . 2090, 2151 . . . 2740 |
| Scyth2p4_003875 | 62 | 1057 | 1 . . . 243, 308 . . . 1057 |
| Scyth2p4_003882 | 63 | 3067 | 1 . . . 81, 384 . . . 571, 624 . . . 1012, 1068 . . . 3067 |
| Scyth2p4_003909 | 64 | 1035 | 1 . . . 1035 |
| Scyth2p4_003923 | 65 | 3163 | 1 . . . 774, 833 . . . 3163 |
| Scyth2p4_003925 | 66 | 1023 | 1 . . . 303, 365 . . . 861, 927 . . . 1023 |
| Scyth2p4_003929 | 67 | 459 | 1 . . . 459 |
| Scyth2p4_003943 | 68 | 2358 | 1 . . . 2358 |
| Scyth2p4_004010 | 69 | 1479 | 1 . . . 127, 183 . . . 544, 613 . . . 1479 |
| Scyth2p4_004018 | 70 | 798 | 1 . . . 798 |
| Scyth2p4_004025 | 71 | 1059 | 1 . . . 1059 |
| Scyth2p4_004026 | 72 | 1446 | 1 . . . 1446 |
| Scyth2p4_004049 | 73 | 928 | 1 . . . 126, 192 . . . 285, 354 . . . 700, 764 . . . 928 |
| Scyth2p4_004099 | 74 | 1536 | 1 . . . 178, 242 . . . 1536 |
| Scyth2p4_004162 | 75 | 998 | 1 . . . 645, 705 . . . 998 |
| Scyth2p4_004197 | 76 | 1607 | 1 . . . 90, 159 . . . 511, 576 . . . 648, 707 . . . 955, 1017 . . . 1607 |
| Scyth2p4_004205 | 77 | 1404 | 1 . . . 664, 731 . . . 1404 |
| Scyth2p4_004235 | 78 | 1324 | 1 . . . 1138, 1200 . . . 1324 |
| Scyth2p4_004237 | 79 | 2264 | 1 . . . 1795, 1858 . . . 2264 |
| Scyth2p4_004263 | 80 | 1586 | 1 . . . 628, 693 . . . 1117, 1260 . . . 1586 |
| Scyth2p4_004293 | 81 | 1662 | 1 . . . 412, 473 . . . 1662 |
| Scyth2p4_004317 | 82 | 1358 | 1 . . . 698, 756 . . . 1162, 1222 . . . 1358 |
| Scyth2p4_004650 | 83 | 1474 | 1 . . . 419, 512 . . . 743, 823 . . . 1059, 1142 . . . 1474 |
| Scyth2p4_004945 | 84 | 936 | 1 . . . 400, 510 . . . 721, 808 . . . 855, 925 . . . 936 |
| Scyth2p4_004976 | 85 | 1476 | 1 . . . 317, 384 . . . 1476 |
| Scyth2p4_005037 | 86 | 2101 | 1 . . . 740, 806 . . . 981, 1054 . . . 1279, 1356 . . . 1532, 1594 . . . 2101 |
| Scyth2p4_005092 | 87 | 1032 | 1 . . . 34, 91 . . . 930, 998 . . . 1032 |
| Scyth2p4_005093 | 88 | 849 | 1 . . . 216, 358 . . . 849 |
| Scyth2p4_005094 | 89 | 749 | 1 . . . 204, 267, 749 |
| Scyth2p4_005146 | 90 | 1437 | 1 . . . 195, 271 . . . 471, 531 . . . 1076, 1147 . . . 1437 |
| Scyth2p4_005307 | 91 | 1156 | 1 . . . 724, 830 . . . 908, 1001 . . . 1019, 1106 . . . 1156 |
| Scyth2p4_005334 | 92 | 1005 | 1 . . . 1005 |
| Scyth2p4_005335 | 93 | 989 | 1 . . . 520, 586 . . . 842, 906 . . . 989 |
| Scyth2p4_005384 | 94 | 874 | 1 . . . 441, 498 . . . 618, 690 . . . 874 |
| Scyth2p4_005465 | 95 | 1101 | 1 . . . 958, 1031 . . . 1101 |
| Scyth2p4_005588 | 96 | 1253 | 1 . . . 373, 436 . . . 1253 |
| Scyth2p4_005596 | 97 | 2460 | 1 . . . 145, 220 . . . 627, 686 . . . 2460 |
| Scyth2p4_005646 | 98 | 781 | 1 . . . 275, 340, 781 |
| Scyth2p4_005692 | 99 | 893 | 1 . . . 320, 394, 761, 820 . . . 893 |
| Scyth2p4_005696 | 100 | 791 | 1 . . . 417, 484 . . . 657, 723, 791 |
| Scyth2p4_005712 | 101 | 1317 | 1 . . . 172, 229 . . . 924, 989 . . . 1317 |
| Scyth2p4_005714 | 102 | 1394 | 1 . . . 1172, 1230 . . . 1305, 1371 . . . 1394 |
| Scyth2p4_005722 | 103 | 1412 | 1 . . . 120, 179 . . . 278, 340 . . . 666, 752 . . . 1265, 1325 . . . 1412 |
| Scyth2p4_005760 | 104 | 1031 | 1 . . . 92, 156 . . . 576, 648 . . . 1031 |
| Scyth2p4_005775 | 105 | 478 | 1 . . . 183, 254 . . . 478 |
| Scyth2p4_005777 | 106 | 477 | 1 . . . 198, 262 . . . 477 |
| Scyth2p4_005792 | 107 | 2586 | 1 . . . 2586 |
| Scyth2p4_005865 | 108 | 1032 | 1 . . . 301, 377 . . . 1032 |
| Scyth2p4_005894 | 109 | 885 | 1 . . . 885 |
| Scyth2p4_006005 | 110 | 1158 | 1 . . . 1158 |
| Scyth2p4_006013 | 111 | 3539 | 1 . . . 334, 415 . . . 932, 1003 . . . 1182, 1254 . . . 1307, 2019 . . . 2842, 2919 . . . 3168 |
| | | | 3280 . . . 3413, 3482 . . . 3539 |
| Scyth2p4_006014 | 112 | 2165 | 1 . . . 512, 574, 752, 812 . . . 1088, 1207 . . . 1634, 1685 . . . 1814, 1883 . . . 2165 |
| Scyth2p4_006016 | 113 | 1090 | 1 . . . 284, 348 . . . 681, 751 . . . 926, 994 . . . 1090 |
| Scyth2p4_006040 | 114 | 1267 | 1 . . . 194, 252 . . . 1047, 1106 . . . 1267 |
| Scyth2p4_006061 | 115 | 1755 | 1 . . . 621, 680 . . . 1484, 1541 . . . 1755 |
| Scyth2p4_006263 | 116 | 1173 | 1 . . . 85, 141 . . . 303, 366 . . . 470, 528 . . . 1173 |
| Scyth2p4_006265 | 117 | 2874 | 1 . . . 250, 406 . . . 822, 878 . . . 2874 |
| Scyth2p4_006499 | 118 | 3030 | 1 . . . 82, 143 . . . 3030 |
| Scyth2p4_006556 | 119 | 1203 | 1 . . . 88, 146 . . . 1203 |
| Scyth2p4_006566 | 120 | 1244 | 1 . . . 348, 405 . . . 561, 622 . . . 653, 723 . . . 1244 |
| Scyth2p4_006586 | 121 | 1092 | 1 . . . 1092 |
| Scyth2p4_006591 | 122 | 2668 | 1 . . . 230, 290 . . . 1163, 1220 . . . 2668 |
| Scyth2p4_006628 | 123 | 1560 | 1 . . . 333, 388 . . . 1560 |
| Scyth2p4_006768 | 124 | 2631 | 1 . . . 79, 135 . . . 206, 273 . . . 346, 399 . . . 891, 955 . . . 2043, 2117 . . . 2631 |
| Scyth2p4_006914 | 125 | 1687 | 1 . . . 1587, 1655 . . . 1687 |
| Scyth2p4_006916 | 126 | 776 | 1 . . . 52, 109 . . . 385, 455 . . . 776 |
| Scyth2p4_006920 | 127 | 1053 | 1 . . . 70, 150 . . . 511, 589 . . . 1053 |
| Scyth2p4_006931 | 128 | 1105 | 1 . . . 423, 480 . . . 505, 562 . . . 620, 696 . . . 756, 826 . . . 1105 |
| Scyth2p4_006993 | 129 | 1746 | 1 . . . 89, 148 . . . 641, 776 . . . 1395, 1450 . . . 1623, 1693 . . . 1746 |
| Scyth2p4_007002 | 130 | 1437 | 1 . . . 1437 |
| Scyth2p4_007064 | 131 | 1321 | 1 . . . 99, 165 . . . 318, 384 . . . 1321 |
| Scyth2p4_007097 | 132 | 1027 | 1 . . . 537, 635 . . . 1027 |
| Scyth2p4_007200 | 133 | 1465 | 1 . . . 297, 356, 745, 805 . . . 1160, 1231 . . . 1465 |
| Scyth2p4_007231 | 134 | 818 | 1 . . . 55, 114 . . . 455, 532 . . . 818 |
| Scyth2p4_007246 | 135 | 1179 | 1 . . . 140, 213 . . . 1179 |
| Scyth2p4_007249 | 136 | 1197 | 1 . . . 1197 |
| Scyth2p4_007259 | 137 | 1053 | 1 . . . 358, 434 . . . 1053 |
| Scyth2p4_007263 | 138 | 888 | 1 . . . 448, 524 . . . 888 |
| Scyth2p4_007266 | 139 | 3446 | 1 . . . 303, 368 . . . 801, 867 . . . 2167, 2224 . . . 3446 |
| Scyth2p4_007287 | 140 | 1321 | 1 . . . 260, 335 . . . 497, 562 . . . 801, 856 . . . 1207, 1278 . . . 1321 |
| Scyth2p4_007304 | 141 | 1655 | 1 . . . 91, 160 . . . 351, 413 . . . 479, 578 . . . 674, 731 . . . 815, 871 . . . 988, 1044 . . . 1089, |
| | | | 1160 . . . 1517, 1615 . . . 1655 |
| Scyth2p4_007313 | 142 | 2402 | 1 . . . 191, 260 . . . 2402 |
| Scyth2p4_007314 | 143 | 928 | 1 . . . 753, 812 . . . 928 |
| Scyth2p4_007531 | 144 | 1713 | 1 . . . 209, 275 . . . 649, 771 . . . 928, 994 . . . 1172, 1249 . . . 1583, 1668 . . . 1713 |
| Scyth2p4_007556 | 145 | 1062 | 1 . . . 1062 |
| Scyth2p4_007557 | 146 | 1053 | 1 . . . 830, 1020 . . . 1053 |
| Scyth2p4_007647 | 147 | 3340 | 1 . . . 819, 873 . . . 1866, 1950 . . . 3340 |
| Scyth2p4_007651 | 148 | 844 | 1 . . . 46, 111 . . . 691, 758 . . . 844 |
| Scyth2p4_007699 | 149 | 1322 | 1 . . . 373, 466 . . . 1322 |
| Scyth2p4_007856 | 150 | 1104 | 1 . . . 1104 |
| Scyth2p4_007921 | 151 | 1860 | 1 . . . 211, 265 . . . 440, 505 . . . 1860 |
| Scyth2p4_008285 | 152 | 534 | 1 . . . 534 |
| Scyth2p4_008294 | 153 | 1406 | 1 . . . 218, 287 . . . 435, 507 . . . 649, 706 . . . 874, 931 . . . 1406 |
| Scyth2p4_008312 | 154 | 1508 | 1 . . . 201, 264 . . . 1508 |
| Scyth2p4_008328 | 155 | 1003 | 1 . . . 269, 323 . . . 827, 893 . . . 1003 |
| Scyth2p4_008336 | 156 | 1363 | 1 . . . 297, 358, 715, 770 . . . 1116, 1169 . . . 1363 |
| Scyth2p4_008341 | 157 | 1066 | 1 . . . 58, 126 . . . 183, 236 . . . 623, 691 . . . 883, 960 . . . 1066 |
| Scyth2p4_008344 | 158 | 595 | 1 . . . 181, 258 . . . 324, 391 . . . 595 |
| Scyth2p4_008363 | 159 | 1210 | 1 . . . 932, 994 . . . 1210 |
| Scyth2p4_008372 | 160 | 1583 | 1 . . . 570, 628 . . . 980, 1036 . . . 1279, 1336 . . . 1466, 1526 . . . 1583 |
| Scyth2p4_008392 | 161 | 1458 | 1 . . . 269, 389 . . . 536, 601 . . . 797, 874 . . . 1089, 1163 . . . 1341, 1415 . . . 1458 |
| Scyth2p4_008399 | 162 | 717 | 1 . . . 335, 411 . . . 717 |
| Scyth2p4_008411 | 163 | 1377 | 1 . . . 784, 869 . . . 1377 |
| Scyth2p4_008417 | 164 | 753 | 1 . . . 397, 470, 753 |
| Scyth2p4_008418 | 165 | 854 | 1 . . . 52, 134 . . . 487, 565 . . . 854 |
| Scyth2p4_008663 | 166 | 1293 | 1 . . . 22, 81 . . . 101, 222 . . . 455, 530 . . . 1293 |
| Scyth2p4_008755 | 167 | 2176 | 1 . . . 316, 386 . . . 829, 895 . . . 1026, 1080 . . . 2176 |
| Scyth2p4_008830 | 168 | 1910 | 1 . . . 441, 499 . . . 661, 727 . . . 1910 |
| Scyth2p4_008896 | 169 | 1088 | 1 . . . 389, 496 . . . 571, 658 . . . 922, 1033 . . . 1088 |
| Scyth2p4_009014 | 170 | 2281 | 1 . . . 4, 66 . . . 238, 302 . . . 565, 642 . . . 702, 769 . . . 867, 1013 . . . 1061, 1112 . . . 1167, |
| | | | 1218 . . . 1755, 1813 . . . 2281 |
| Scyth2p4_009047 | 171 | 1284 | 1 . . . 856, 926 . . . 1284 |
| Scyth2p4_009244 | 172 | 1742 | 1 . . . 219, 375 . . . 1742 |
| Scyth2p4_009303 | 173 | 2191 | 1 . . . 250, 304 . . . 499, 568 . . . 2191 |
| Scyth2p4_009308 | 174 | 1070 | 1 . . . 159, 246 . . . 591, 700 . . . 1070 |
| Scyth2p4_009393 | 175 | 2615 | 1 . . . 83, 149 . . . 659, 733 . . . 854, 2501 . . . 2615 |
| Scyth2p4_009418 | 176 | 1497 | 1 . . . 398, 462 . . . 580, 648 . . . 1192, 1264 . . . 1497 |
| Scyth2p4_009426 | 177 | 2237 | 1 . . . 447, 521 . . . 642, 1874 . . . 1891, 2123 . . . 2237 |
| Scyth2p4_009442 | 178 | 1157 | 1 . . . 145, 201 . . . 839, 898 . . . 1157 |
| Scyth2p4_009463 | 179 | 1667 | 1 . . . 794, 853 . . . 1048, 1113 . . . 1667 |
| Scyth2p4_009475 | 180 | 1842 | 1 . . . 853, 911 . . . 978, 1099 . . . 1842 |
| Scyth2p4_009509 | 181 | 1362 | 1 . . . 177, 259 . . . 588, 637 . . . 1362 |
| Scyth2p4_009510 | 182 | 1197 | 1 . . . 102, 151 . . . 1197 |
| Scyth2p4_009516 | 183 | 792 | 1 . . . 179, 318 . . . 792 |
| Scyth2p4_009525 | 184 | 1502 | 1 . . . 209, 269 . . . 426, 511 . . . 585, 669 . . . 1088, 1150 . . . 1286, 1341 . . . 1502 |
| Scyth2p4_009550 | 185 | 1479 | 1 . . . 255, 312 . . . 497, 587 . . . 827, 884 . . . 1173, 1288 . . . 1479 |
| Scyth2p4_009554 | 186 | 875 | 1 . . . 100, 162 . . . 523, 618 . . . 875 |
| Scyth2p4_009565 | 187 | 1370 | 1 . . . 615, 678 . . . 1370 |
| Scyth2p4_009569 | 188 | 1788 | 1 . . . 1042, 1105 . . . 1252, 1317 . . . 1788 |
| Scyth2p4_009610 | 189 | 1020 | 1 . . . 155, 250 . . . 876, 939 . . . 1020 |
| Scyth2p4_009620 | 190 | 974 | 1 . . . 335, 392 . . . 974 |
| Scyth2p4_009626 | 191 | 1236 | 1 . . . 1156, 1217 . . . 1236 |
| Scyth2p4_009629 | 192 | 1559 | 1 . . . 113, 186 . . . 240, 308 . . . 410, 483 . . . 616, 677 . . . 879, 935 . . . 1076, 1140 . . . 1225, |
| | | | 1286 . . . 1458, 1522 . . . 1559 |
| Scyth2p4_009651 | 193 | 1407 | 1 . . . 1407 |
| Scyth2p4_009653 | 194 | 1131 | 1 . . . 444, 521 . . . 964, 1036 . . . 1131 |
| Scyth2p4_009700 | 195 | 1320 | 1 . . . 430, 513 . . . 837, 900 . . . 1320 |
| Scyth2p4_009707 | 196 | 2834 | 1 . . . 49, 106 . . . 266, 325 . . . 806, 880 . . . 1219, 1275 . . . 1764, 1824 . . . 2089, |
| | | | 2147 . . . 2240, 2293 . . . 2525, 2592 . . . 2834 |
| Scyth2p4_009711 | 197 | 4308 | 1 . . . 204, 269 . . . 1250, 2172 . . . 2881, 4132 . . . 4308 |
| Scyth2p4_009720 | 198 | 1074 | 1 . . . 1074 |
| Scyth2p4_009765 | 199 | 3723 | 1 . . . 23, 311 . . . 446, 1486 . . . 3723 |
| Scyth2p4_009796 | 200 | 3125 | 1 . . . 235, 309 . . . 961, 1077 . . . 1160, 1240 . . . 2017, 2083 . . . 3125 |
| Scyth2p4_009823 | 201 | 789 | 1 . . . 789 |
| Scyth2p4_009929 | 202 | 4377 | 1 . . . 457, 506 . . . 3441, 3490 . . . 3769, 3818 . . . 4052, 4107 . . . 4377 |
| Scyth2p4_010021 | 203 | 878 | 1 . . . 66, 132 . . . 436, 509 . . . 878 |
| Scyth2p4_010034 | 204 | 1404 | 1 . . . 137, 204 . . . 1404 |
| Scyth2p4_010146 | 205 | 898 | 1 . . . 108, 173 . . . 898 |
| Scyth2p4_010149 | 206 | 1449 | 1 . . . 364, 413 . . . 521, 576, 791, 859 . . . 1349, 1403 . . . 1449 |
| Scyth2p4_010269 | 207 | 1108 | 1 . . . 466, 528 . . . 1108 |
| Scyth2p4_010278 | 208 | 5924 | 1 . . . 248, 315 . . . 401, 463 . . . 779, 838 . . . 924, 1001 . . . 4447, 4522 . . . 4602, |
| | | | 4736 . . . 5029, 5116 . . . 5214, 5353 . . . 5802, 5878 . . . 5924 |
| Scyth2p4_010280 | 209 | 1887 | 1 . . . 398, 458 . . . 943, 1001 . . . 1060, 1118 . . . 1183, 1251 . . . 1755, 1810 . . . 1887 |
| Scyth2p4_010281 | 210 | 1919 | 1 . . . 361, 422 . . . 503, 571 . . . 599, 673 . . . 807, 884 . . . 965, 1017 . . . 1159, 1234 . . . 1315, |
| | | | 1371 . . . 1585, 1667 . . . 1776, 1863 . . . 1919 |
| Scyth2p4_010291 | 211 | 1518 | 1 . . . 433, 494 . . . 1518 |
| Scyth2p4_010295 | 212 | 657 | 1 . . . 222, 295 . . . 657 |
| Scyth2p4_010361 | 213 | 2163 | 1 . . . 181, 246 . . . 428, 529 . . . 2069, 2131 . . . 2163 |
| Scyth2p4_010373 | 214 | 1526 | 1 . . . 288, 341 . . . 1085, 1144 . . . 1526 |
| Scyth2p4_010387 | 215 | 3027 | 1 . . . 3027 |
| Scyth2p4_010416 | 216 | 2030 | 1 . . . 1078, 1139 . . . 1914, 1986 . . . 2030 |
| Scyth2p4_010423 | 217 | 1254 | 1 . . . 377, 450 . . . 1254 |
| Scyth2p4_010457 | 218 | 815 | 1 . . . 278, 353 . . . 683, 741 . . . 815 |
| Scyth2p4_010462 | 219 | 1779 | 1 . . . 298, 364 . . . 713, 1244 . . . 1314, 1387 . . . 1458, 1557 . . . 1779 |
| Scyth2p4_010469 | 220 | 1917 | 1 . . . 592, 650 . . . 1917 |
| Scyth2p4_010519 | 221 | 1418 | 1 . . . 1162, 1231 . . . 1418 |
| Scyth2p4_010552 | 222 | 2203 | 1 . . . 208, 292 . . . 578, 676 . . . 802, 860 . . . 1321, 1389 . . . 2203 |
| Scyth2p4_010553 | 223 | 2644 | 1 . . . 88, 1200 . . . 1752, 1831 . . . 2644 |
| Scyth2p4_010743 | 224 | 886 | 1 . . . 55, 121 . . . 480, 600 . . . 886 |
| Scyth2p4_010756 | 225 | 846 | 1 . . . 846 |
| Scyth2p4_010779 | 226 | 6427 | 1 . . . 137, 192 . . . 449, 512 . . . 637, 710 . . . 733, 790 . . . 940, 1009 . . . 1204, 1257 . . . 1375, |
| | | | 1449 . . . 1654, 1742 . . . 2087, 2253 . . . 4325, 4700 . . . 6427 |
| Scyth2p4_010780 | 227 | 2746 | 1 . . . 427, 481 . . . 1299, 1380 . . . 2746 |
| Scyth2p4_010784 | 228 | 2733 | 1 . . . 2733 |
| Scyth2p4_010822 | 229 | 1217 | 1 . . . 317, 411 . . . 803, 1124 . . . 1217 |
| Scyth2p4_010823 | 230 | 2353 | 1 . . . 384, 437 . . . 2353 |
| Scyth2p4_010825 | 231 | 1500 | 1 . . . 429, 484 . . . 657, 715 . . . 1500 |
| Scyth2p4_010857 | 232 | 752 | 1 . . . 260, 329 . . . 752 |
| Scyth2p4_010865 | 233 | 906 | 1 . . . 906 |
| Scyth2p4_010870 | 234 | 1979 | 1 . . . 934, 1024 . . . 1979 |
| Scyth2p4_010884 | 235 | 1514 | 1 . . . 399, 486 . . . 658, 874 . . . 1277, 1474 . . . 1514 |
| Scyth2p4_010894 | 236 | 1143 | 1 . . . 369, 433 . . . 612, 712 . . . 1143 |
| Scyth2p4_010898 | 237 | 1426 | 1 . . . 336, 403 . . . 629, 703 . . . 1426 |
| Scyth2p4_010899 | 238 | 1682 | 1 . . . 116, 182 . . . 612, 687 . . . 1275, 1337 . . . 1682 |
| Scyth2p4_001141 | 239 | 1447 | 1 . . . 112, 172 . . . 1085, 1196 . . . 1447 |
| Scyth2p4_001257 | 240 | 1005 | 1 . . . 1005 |
| Scyth2p4_001442 | 241 | 759 | 1 . . . 104, 201 . . . 604, 692 . . . 759 |
| Scyth2p4_001768 | 242 | 2206 | 1 . . . 179, 253 . . . 912, 970 . . . 2206 |
| Scyth2p4_002054 | 243 | 1751 | 1 . . . 217, 303 . . . 1130, 1195 . . . 1751 |
| Scyth2p4_003709 | 244 | 336 | 1 . . . 336 |
| Scyth2p4_003954 | 245 | 2366 | 1 . . . 116, 181 . . . 239, 297 . . . 335, 485 . . . 540, 595 . . . 699, 765 . . . 780, 834 . . . 1279, |
| | | | 1335 . . . 1657, 1720 . . . 1782, 2075 . . . 2366 |
| Scyth2p4_004342 | 246 | 1363 | 1 . . . 230, 306 . . . 555, 617 . . . 1363 |
| Scyth2p4_004817 | 247 | 1785 | 1 . . . 1785 |
| Scyth2p4_005217 | 248 | 1210 | 1 . . . 291, 357 . . . 576, 645 . . . 1210 |
| Scyth2p4_007345 | 249 | 1540 | 1 . . . 167, 233 . . . 395, 535 . . . 621, 688 . . . 851, 955 . . . 1540 |
| Scyth2p4_007869 | 250 | 579 | 1 . . . 579 |
| Scyth2p4_009477 | 251 | 2054 | 1 . . . 386, 445 . . . 929, 994 . . . 1811, 1887 . . . 2054 |
| Scyth2p4_009552 | 252 | 940 | 1 . . . 88, 256 . . . 617, 701 . . . 940 |
| Scyth2p4_009704 | 253 | 1311 | 1 . . . 1311 |
| Scyth2p4_010302 | 254 | 551 | 1 . . . 141, 198 . . . 551 |
| Scyth2p4_010820 | 255 | 565 | 1 . . . 208, 288 . . . 565 |
| SCYTH_1_00385 | 256 | 2721 | 1 . . . 621, 673 . . . 2721 |
| SCYTH_1_00739 | 257 | 1958 | 1 . . . 177, 292 . . . 1458, 1530 . . . 1958 |
| SCYTH_1_03688 | 259 | 1059 | 1 . . . 1059 |
| SCYTH_1_09019 | 260 | 1199 | 1 . . . 264, 333 . . . 1199 |
| SCYTH_2_05417 | 261 | 1625 | 1 . . . 413, 466 . . . 551, 607 . . . 1625 |
| Scyth2p4_000071 | 263 | 2145 | 1 . . . 899, 959 . . . 1427, 1519 . . . 2145 |
| Scyth2p4_000786 | 264 | 1472 | 1 . . . 42, 141 . . . 298, 361 . . . 1295, 1360 . . . 1472 |
| Scyth2p4_000879 | 265 | 1375 | 1 . . . 153, 218 . . . 1375 |
| Scyth2p4_001265 | 266 | 990 | 1 . . . 88, 170 . . . 410, 483 . . . 757, 827 . . . 990 |
| Scyth2p4_001349 | 267 | 2050 | 1 . . . 255, 331 . . . 466, 537 . . . 691, 750 . . . 1066, 1122 . . . 1297, 1360 . . . 1726, |
| | | | 1801 . . . 2050 |
| Scyth2p4_002059 | 268 | 1768 | 1 . . . 400, 481 . . . 714, 780 . . . 971, 1045 . . . 1147, 1202 . . . 1587, 1650 . . . 1768 |
| Scyth2p4_002062 | 269 | 1792 | 1 . . . 471, 530 . . . 1792 |
| Scyth2p4_002618 | 270 | 2078 | 1 . . . 548, 605 . . . 2078 |
| Scyth2p4_002885 | 271 | 1352 | 1 . . . 1242, 1299 . . . 1352 |
| Scyth2p4_003845 | 272 | 2564 | 1 . . . 370, 442 . . . 559, 629 . . . 966, 1536 . . . 1596, 1718 . . . 1769, 2022 . . . 2564 |
| Scyth2p4_003921 | 273 | 2399 | 1 . . . 2090, 2153 . . . 2399 |
| Scyth2p4_003974 | 274 | 2051 | 1 . . . 305, 383 . . . 1135, 1199 . . . 2051 |
| Scyth2p4_003996 | 275 | 2901 | 1 . . . 129, 196 . . . 552, 613 . . . 2901 |
| Scyth2p4_004891 | 276 | 1507 | 1 . . . 333, 393 . . . 622, 676 . . . 1507 |
| Scyth2p4_005785 | 277 | 1098 | 1 . . . 1098 |
| Scyth2p4_006840 | 278 | 914 | 1 . . . 419, 500 . . . 914 |
| Scyth2p4_007340 | 279 | 1926 | 1 . . . 111, 178 . . . 1926 |
| Scyth2p4_007698 | 280 | 1624 | 1 . . . 470, 560 . . . 1094, 1154 . . . 1624 |
| Scyth2p4_008300 | 281 | 990 | 1 . . . 261, 324 . . . 850, 912 . . . 990 |
| Scyth2p4_009549 | 282 | 1648 | 1 . . . 295, 346 . . . 689, 752 . . . 1648 |
| Scyth2p4_010449 | 283 | 3157 | 1 . . . 607, 730 . . . 1125, 1872 . . . 2083, 2138 . . . 2789, 2844 . . . 3157 |
| Scyth2p4_010575 | 284 | 1398 | 1 . . . 606, 669 . . . 822, 881 . . . 1398 |
| Scyth2p4_010881 | 285 | 1640 | 1 . . . 500, 602 . . . 1640 |

TABLE 2B
List of genes of Myriococcum thermophilum with reference to exon boundaries

| | Genomic | | |
|---|---|---|---|
| | sequence | Genomic | |
| | (SEQ | sequence | |
| Gene ID | ID NO:) | length | Exon boundaries (nucleotide positions) and exons |
| Myrth2p4_000015 | 856 | 1707 | 1 . . . 1707 |
| Myrth2p4_000358 | 857 | 745 | 1 . . . 394, 462 . . . 745 |
| Myrth2p4_000359 | 858 | 2483 | 1 . . . 52, 189 . . . 1034, 1144 . . . 1265, 1394 . . . 1487, 1569 . . . 1618, |
| | | | 1683 . . . 1761, 1823 . . . 2055, 2129 . . . 2171, 2266 . . . 2483 |
| Myrth2p4_000363 | 859 | 1856 | 1 . . . 781, 858 . . . 943, 1497 . . . 1856 |
| Myrth2p4_000376 | 860 | 1528 | 1 . . . 96, 159 . . . 416, 639 . . . 835, 930 . . . 1142, 1232 . . . 1410, |
| | | | 1485 . . . 1528 |
| Myrth2p4_000388 | 861 | 2404 | 1 . . . 561, 1278 . . . 1414, 1510 . . . 1722, 1806 . . . 2052, 2156 . . . 2286, |
| | | | 2347 . . . 2404 |
| Myrth2p4_000417 | 862 | 1017 | 1 . . . 178, 302 . . . 1017 |
| Myrth2p4_000486 | 863 | 732 | 1 . . . 732 |
| Myrth2p4_000495 | 864 | 2067 | 1 . . . 261, 718 . . . 2067 |
| Myrth2p4_000510 | 865 | 898 | 1 . . . 224, 352 . . . 898 |
| Myrth2p4_000524 | 866 | 832 | 1 . . . 99, 188 . . . 832 |
| Myrth2p4_000531 | 867 | 780 | 1 . . . 780 |
| Myrth2p4_000543 | 868 | 1606 | 1 . . . 208, 276 . . . 1184, 1254 . . . 1606 |
| Myrth2p4_000545 | 869 | 1589 | 1 . . . 159, 216 . . . 456, 527 . . . 1221, 1284 . . . 1368, 1423 . . . 1589 |
| Myrth2p4_000589 | 870 | 1212 | 1 . . . 1212 |
| Myrth2p4_000694 | 871 | 2139 | 1 . . . 204, 289 . . . 1559, 1616 . . . 1711, 1767 . . . 2139 |
| Myrth2p4_000867 | 872 | 1534 | 1 . . . 442, 522 . . . 1534 |
| Myrth2p4_000999 | 873 | 966 | 1 . . . 552, 733 . . . 966 |
| Myrth2p4_001083 | 874 | 1722 | 1 . . . 502, 560 . . . 1722 |
| Myrth2p4_001208 | 875 | 1570 | 1 . . . 192, 287 . . . 1570 |
| Myrth2p4_001304 | 876 | 2903 | 1 . . . 43, 102 . . . 286, 339 . . . 1772, 1842 . . . 1984, 2042 . . . 2133, |
| | | | 2202 . . . 2434, 2547 . . . 2903 |
| Myrth2p4_001319 | 877 | 908 | 1 . . . 644, 729 . . . 781, 859 . . . 908 |
| Myrth2p4_001328 | 878 | 1329 | 1 . . . 417, 528 . . . 685, 803 . . . 1191, 1304 . . . 1329 |
| Myrth2p4_001333 | 879 | 862 | 1 . . . 243, 323 . . . 862 |
| Myrth2p4_001339 | 880 | 2984 | 1 . . . 72, 319 . . . 473, 534 . . . 922, 985 . . . 2984 |
| Myrth2p4_001354 | 881 | 1147 | 1 . . . 79, 181 . . . 230, 290 . . . 690, 763 . . . 1147 |
| Myrth2p4_001362 | 882 | 975 | 1 . . . 975 |
| Myrth2p4_001366 | 883 | 1144 | 1 . . . 116, 208 . . . 1144 |
| Myrth2p4_001368 | 884 | 2334 | 1 . . . 2334 |
| Myrth2p4_001374 | 885 | 832 | 1 . . . 63, 156 . . . 545, 623 . . . 832 |
| Myrth2p4_001375 | 886 | 1418 | 1 . . . 80, 148 . . . 548, 696 . . . 1156, 1269 . . . 1418 |
| Myrth2p4_001378 | 887 | 1336 | 1 . . . 362, 445 . . . 490, 626 . . . 1211, 1305 . . . 1336 |
| Myrth2p4_001403 | 888 | 1045 | 1 . . . 489, 593 . . . 1045 |
| Myrth2p4_001451 | 889 | 1399 | 1 . . . 275, 982 . . . 1399 |
| Myrth2p4_001463 | 890 | 1501 | 1 . . . 152, 263 . . . 411, 522 . . . 1501 |
| Myrth2p4_001467 | 891 | 1758 | 1 . . . 90, 189 . . . 541, 631 . . . 703, 774 . . . 1022, 1168 . . . 1758 |
| Myrth2p4_001469 | 892 | 1980 | 1 . . . 1980 |
| Myrth2p4_001494 | 893 | 2445 | 1 . . . 2445 |
| Myrth2p4_001496 | 894 | 2357 | 1 . . . 212, 276 . . . 1086, 1137 . . . 2357 |
| Myrth2p4_001537 | 895 | 856 | 1 . . . 121, 177 . . . 301, 362 . . . 619, 674 . . . 856 |
| Myrth2p4_001550 | 896 | 1373 | 1 . . . 515, 619 . . . 892, 969 . . . 1042, 1109 . . . 1281, 1336 . . . 1373 |
| Myrth2p4_001581 | 897 | 992 | 1 . . . 284, 358 . . . 431, 503 . . . 544, 639 . . . 856, 936 . . . 992 |
| Myrth2p4_001582 | 898 | 2779 | 1 . . . 524, 653 . . . 1227, 1324 . . . 1411, 1573 . . . 1810, 1913 . . . 1924, |
| | | | 2091 . . . 2221, 2500 . . . 2779 |
| Myrth2p4_001589 | 899 | 1968 | 1 . . . 111, 219 . . . 1339, 1443 . . . 1870, 1943 . . . 1968 |
| Myrth2p4_001667 | 900 | 813 | 1 . . . 813 |
| Myrth2p4_001718 | 901 | 1199 | 1 . . . 549, 642 . . . 1199 |
| Myrth2p4_001719 | 902 | 1137 | 1 . . . 602, 687 . . . 1137 |
| Myrth2p4_001916 | 903 | 2201 | 1 . . . 175, 262 . . . 1542, 2161 . . . 2201 |
| Myrth2p4_001926 | 904 | 1185 | 1 . . . 1185 |
| Myrth2p4_001996 | 905 | 1648 | 1 . . . 432, 1188 . . . 1218, 1290 . . . 1416, 1561 . . . 1648 |
| Myrth2p4_002010 | 906 | 2007 | 1 . . . 2007 |
| Myrth2p4_002134 | 907 | 1607 | 1 . . . 337, 447 . . . 932, 1063 . . . 1607 |
| Myrth2p4_002293 | 908 | 1709 | 1 . . . 363, 467 . . . 1211, 1648 . . . 1709 |
| Myrth2p4_002328 | 909 | 1092 | 1 . . . 426, 561 . . . 703, 795 . . . 1092 |
| Myrth2p4_002394 | 910 | 1739 | 1 . . . 190, 315 . . . 1318, 1376 . . . 1567, 1680 . . . 1739 |
| Myrth2p4_002434 | 911 | 3417 | 1 . . . 667, 723 . . . 1169, 1240 . . . 1503, 1572 . . . 1663, 1744 . . . 1773, |
| | | | 1836 . . . 3071, 3172 . . . 3417 |
| Myrth2p4_002456 | 912 | 1392 | 1 . . . 43, 109 . . . 965, 1048 . . . 1392 |
| Myrth2p4_002548 | 913 | 6869 | 1 . . . 6781, 6853 . . . 6869 |
| Myrth2p4_002549 | 914 | 1714 | 1 . . . 156, 277 . . . 735, 792 . . . 999, 1098 . . . 1452, 1522 . . . 1714 |
| Myrth2p4_002563 | 915 | 4539 | 1 . . . 787, 911 . . . 943, 1060 . . . 1886, 2009 . . . 2275, 2372 . . . 3871, |
| | | | 3958 . . . 4539 |
| Myrth2p4_002601 | 916 | 1392 | 1 . . . 1392 |
| Myrth2p4_002632 | 917 | 2387 | 1 . . . 61, 165 . . . 586, 708 . . . 1379, 1500 . . . 2387 |
| Myrth2p4_002634 | 918 | 1987 | 1 . . . 153, 262 . . . 1428, 1556 . . . 1987 |
| Myrth2p4_002638 | 919 | 1431 | 1 . . . 109, 219 . . . 1145, 1355 . . . 1431 |
| Myrth2p4_002915 | 920 | 1074 | 1 . . . 287, 381 . . . 550, 647 . . . 1074 |
| Myrth2p4_002916 | 921 | 983 | 1 . . . 218, 280 . . . 632, 766 . . . 983 |
| Myrth2p4_002917 | 922 | 1449 | 1 . . . 791, 936 . . . 1449 |
| Myrth2p4_002930 | 923 | 2236 | 1 . . . 148, 208 . . . 838, 901 . . . 1192, 1259 . . . 1445, 1679 . . . 2052, |
| | | | 2213 . . . 2236 |
| Myrth2p4_003005 | 924 | 420 | 1 . . . 420 |
| Myrth2p4_003034 | 925 | 1297 | 1 . . . 380, 493 . . . 1297 |
| Myrth2p4_003051 | 926 | 1461 | 1 . . . 388, 581 . . . 806, 868 . . . 1083, 1165 . . . 1337, 1421 . . . 1461 |
| Myrth2p4_003065 | 927 | 1444 | 1 . . . 370, 512 . . . 876, 980 . . . 1053, 1136 . . . 1308, 1392 . . . 1444 |
| Myrth2p4_003070 | 928 | 2553 | 1 . . . 2553 |
| Myrth2p4_003103 | 929 | 1017 | 1 . . . 126, 211 . . . 304, 396 . . . 742, 853 . . . 1017 |
| Myrth2p4_003203 | 930 | 1679 | 1 . . . 409, 502 . . . 1679 |
| Myrth2p4_003274 | 931 | 1313 | 1 . . . 611, 722 . . . 803, 945 . . . 1313 |
| Myrth2p4_003333 | 932 | 1614 | 1 . . . 259, 326 . . . 414, 490 . . . 1614 |
| Myrth2p4_003368 | 933 | 836 | 1 . . . 281, 431 . . . 836 |
| Myrth2p4_003495 | 934 | 1463 | 1 . . . 404, 533 . . . 764, 834 . . . 1067, 1131 . . . 1463 |
| Myrth2p4_003633 | 935 | 953 | 1 . . . 406, 464 . . . 561, 631 . . . 744, 838 . . . 885, 942 . . . 953 |
| Myrth2p4_003679 | 936 | 1377 | 1 . . . 1377 |
| Myrth2p4_003685 | 937 | 1817 | 1 . . . 620, 721 . . . 792, 912 . . . 1183, 1264 . . . 1817 |
| Myrth2p4_003686 | 938 | 1412 | 1 . . . 323, 404 . . . 1412 |
| Myrth2p4_003747 | 939 | 880 | 1 . . . 152, 255 . . . 358, 448 . . . 558, 741 . . . 880 |
| Myrth2p4_003793 | 940 | 1421 | 1 . . . 204, 279 . . . 479, 537 . . . 1082, 1152 . . . 1421 |
| Myrth2p4_003921 | 941 | 1423 | 1 . . . 793, 997 . . . 1075, 1291 . . . 1423 |
| Myrth2p4_003927 | 942 | 1386 | 1 . . . 93, 168 . . . 176, 249 . . . 281, 481 . . . 713, 814 . . . 874, 983 . . . 1235, |
| | | | 1343 . . . 1386 |
| Myrth2p4_003941 | 943 | 1073 | 1 . . . 523, 630 . . . 871, 993 . . . 1073 |
| Myrth2p4_003942 | 944 | 1023 | 1 . . . 1023 |
| Myrth2p4_003966 | 945 | 630 | 1 . . . 377, 498 . . . 630 |
| Myrth2p4_004088 | 946 | 1334 | 1 . . . 73, 206 . . . 368, 497 . . . 601, 683 . . . 1334 |
| Myrth2p4_004089 | 947 | 3049 | 1 . . . 257, 445 . . . 872, 1021 . . . 2572, 2644 . . . 3049 |
| Myrth2p4_004201 | 948 | 1715 | 1 . . . 585, 639 . . . 1443, 1498 . . . 1715 |
| Myrth2p4_004260 | 949 | 1063 | 1 . . . 298, 375 . . . 641, 714 . . . 1063 |
| Myrth2p4_004335 | 950 | 1101 | 1 . . . 212, 294 . . . 356, 445 . . . 495, 586 . . . 931, 994 . . . 1101 |
| Myrth2p4_004336 | 951 | 1271 | 1 . . . 352, 451 . . . 1271 |
| Myrth2p4_004345 | 952 | 2164 | 1 . . . 962, 1017 . . . 1035, 1114 . . . 1304, 1387 . . . 1458, 1543 . . . 2164 |
| Myrth2p4_004391 | 953 | 1023 | 1 . . . 1023 |
| Myrth2p4_004393 | 954 | 1516 | 1 . . . 198, 257 . . . 1516 |
| Myrth2p4_004397 | 955 | 747 | 1 . . . 747 |
| Myrth2p4_004415 | 956 | 4416 | 1 . . . 4416 |
| Myrth2p4_004442 | 957 | 1619 | 1 . . . 328, 419 . . . 651, 723 . . . 831, 902 . . . 1183, 1315 . . . 1619 |
| Myrth2p4_004455 | 958 | 1449 | 1 . . . 1449 |
| Myrth2p4_004476 | 959 | 1406 | 1 . . . 153, 294 . . . 946, 1073 . . . 1242, 1360 . . . 1406 |
| Myrth2p4_004487 | 960 | 890 | 1 . . . 568, 648 . . . 706, 804 . . . 890 |
| Myrth2p4_004497 | 961 | 3374 | 1 . . . 837, 940 . . . 1927, 2026 . . . 3374 |
| Myrth2p4_004508 | 962 | 922 | 1 . . . 98, 175 . . . 742, 821 . . . 922 |
| Myrth2p4_004535 | 963 | 1827 | 1 . . . 1827 |
| Myrth2p4_004704 | 964 | 1510 | 1 . . . 545, 618 . . . 792, 852 . . . 1125, 1233 . . . 1510 |
| Myrth2p4_004725 | 965 | 2181 | 1 . . . 199, 267 . . . 442, 521 . . . 795, 878 . . . 1262, 1331 . . . 1937, |
| | | | 2129 . . . 2181 |
| Myrth2p4_004787 | 966 | 3029 | 1 . . . 84, 127 . . . 337, 433 . . . 518, 582 . . . 879, 934 . . . 1022, 1141 . . . 1244, |
| | | | 1301 . . . 1597, 1649 . . . 3029 |
| Myrth2p4_004788 | 967 | 1107 | 1 . . . 1107 |
| Myrth2p4_004953 | 968 | 540 | 1 . . . 540 |
| Myrth2p4_004960 | 969 | 1494 | 1 . . . 230, 336 . . . 484, 578 . . . 720, 813 . . . 981, 1043 . . . 1494 |
| Myrth2p4_004965 | 970 | 894 | 1 . . . 894 |
| Myrth2p4_004966 | 971 | 1094 | 1 . . . 162, 276 . . . 389, 486 . . . 1094 |
| Myrth2p4_004986 | 972 | 1542 | 1 . . . 263, 942 . . . 1542 |
| Myrth2p4_004993 | 973 | 1507 | 1 . . . 282, 363 . . . 548, 615 . . . 787, 861 . . . 1206, 1316 . . . 1507 |
| Myrth2p4_005017 | 974 | 1218 | 1 . . . 929, 1002 . . . 1218 |
| Myrth2p4_005025 | 975 | 4739 | 1 . . . 123, 186 . . . 437, 516 . . . 4739 |
| Myrth2p4_005037 | 976 | 777 | 1 . . . 353, 474 . . . 777 |
| Myrth2p4_005039 | 977 | 1352 | 1 . . . 1121, 1220 . . . 1352 |
| Myrth2p4_005084 | 978 | 744 | 1 . . . 242, 321 . . . 744 |
| Myrth2p4_005133 | 979 | 1232 | 1 . . . 156, 237 . . . 428, 503 . . . 1133, 1195 . . . 1232 |
| Myrth2p4_005148 | 980 | 1017 | 1 . . . 574, 686 . . . 1017 |
| Myrth2p4_005149 | 981 | 972 | 1 . . . 972 |
| Myrth2p4_005155 | 982 | 1668 | 1 . . . 129, 221 . . . 320, 397 . . . 723, 916 . . . 1432, 1575 . . . 1668 |
| Myrth2p4_005177 | 983 | 1506 | 1 . . . 195, 271 . . . 1506 |
| Myrth2p4_005191 | 984 | 1232 | 1 . . . 468, 556 . . . 961, 1096 . . . 1232 |
| Myrth2p4_005222 | 985 | 2622 | 1 . . . 2622 |
| Myrth2p4_005269 | 986 | 1382 | 1 . . . 197, 281 . . . 443, 520 . . . 592, 664 . . . 1382 |
| Myrth2p4_005317 | 987 | 3403 | 1 . . . 309, 389 . . . 2123, 2253 . . . 3403 |
| Myrth2p4_005320 | 988 | 969 | 1 . . . 969 |
| Myrth2p4_005321 | 989 | 972 | 1 . . . 466, 599 . . . 972 |
| Myrth2p4_005328 | 990 | 1194 | 1 . . . 1194 |
| Myrth2p4_005329 | 991 | 1292 | 1 . . . 101, 351 . . . 925, 1040 . . . 1089, 1170 . . . 1204, 1280 . . . 1292 |
| Myrth2p4_005340 | 992 | 1649 | 1 . . . 393, 458 . . . 543, 622 . . . 877, 1005 . . . 1649 |
| Myrth2p4_005343 | 993 | 975 | 1 . . . 55, 139 . . . 480, 611 . . . 748, 827 . . . 975 |
| Myrth2p4_005368 | 994 | 1724 | 1 . . . 294, 497 . . . 886, 1077 . . . 1429, 1493 . . . 1724 |
| Myrth2p4_005452 | 995 | 2892 | 1 . . . 313, 450 . . . 636, 723 . . . 2568, 2638 . . . 2892 |
| Myrth2p4_005454 | 996 | 845 | 1 . . . 440, 511 . . . 607, 675 . . . 845 |
| Myrth2p4_005463 | 997 | 1988 | 1 . . . 129, 199 . . . 302, 401 . . . 1067, 1856 . . . 1905, 1979 . . . 1988 |
| Myrth2p4_005484 | 998 | 1323 | 1 . . . 99, 159 . . . 315, 386 . . . 1323 |
| Myrth2p4_005539 | 999 | 2075 | 1 . . . 1359, 1487 . . . 1545, 1724 . . . 2075 |
| Myrth2p4_005561 | 1000 | 1779 | 1 . . . 392, 450 . . . 1779 |
| Myrth2p4_005590 | 1001 | 1462 | 1 . . . 1144, 1272 . . . 1462 |
| Myrth2p4_005626 | 1002 | 1492 | 1 . . . 594, 730 . . . 883, 978 . . . 1492 |
| Myrth2p4_005639 | 1003 | 1270 | 1 . . . 56, 123 . . . 434, 490 . . . 1270 |
| Myrth2p4_005750 | 1004 | 892 | 1 . . . 55, 156 . . . 512, 606 . . . 892 |
| Myrth2p4_005752 | 1005 | 1246 | 1 . . . 272, 427 . . . 1033, 1172 . . . 1246 |
| Myrth2p4_005753 | 1006 | 1022 | 1 . . . 690, 831 . . . 1022 |
| Myrth2p4_005819 | 1007 | 3131 | 1 . . . 303, 362 . . . 448, 527 . . . 835, 932 . . . 1920, 3032 . . . 3131 |
| Myrth2p4_005822 | 1008 | 1563 | 1 . . . 179, 240 . . . 358, 470 . . . 510, 575 . . . 661, 731 . . . 921, 1029 . . . 1563 |
| Myrth2p4_005854 | 1009 | 778 | 1 . . . 254, 361 . . . 778 |
| Myrth2p4_005856 | 1010 | 1010 | 1 . . . 134, 258 . . . 854, 929 . . . 1010 |
| Myrth2p4_005886 | 1011 | 1379 | 1 . . . 402, 512 . . . 675, 842 . . . 1236, 1330 . . . 1379 |
| Myrth2p4_005920 | 1012 | 1712 | 1 . . . 113, 179 . . . 609, 679 . . . 1270, 1367 . . . 1712 |
| Myrth2p4_005923 | 1013 | 1182 | 1 . . . 154, 221 . . . 344, 409 . . . 514, 590 . . . 769, 832 . . . 1182 |
| Myrth2p4_005937 | 1014 | 1623 | 1 . . . 1413, 1486 . . . 1623 |
| Myrth2p4_005945 | 1015 | 1166 | 1 . . . 267, 363 . . . 953, 1062 . . . 1166 |
| Myrth2p4_005946 | 1016 | 1293 | 1 . . . 284, 378 . . . 445, 532 . . . 955, 1107 . . . 1293 |
| Myrth2p4_005976 | 1017 | 1474 | 1 . . . 195, 261 . . . 307, 377 . . . 443, 499 . . . 556, 643 . . . 884, 982 . . . 1322, |
| | | | 1414 . . . 1474 |
| Myrth2p4_006001 | 1018 | 2433 | 1 . . . 249, 330 . . . 408, 509 . . . 520, 630 . . . 699, 768 . . . 1749, |
| | | | 1857 . . . 1928, 2020 . . . 2433 |
| Myrth2p4_006022 | 1019 | 1265 | 1 . . . 717, 855 . . . 1265 |
| Myrth2p4_006028 | 1020 | 1443 | 1 . . . 558, 781 . . . 1443 |
| Myrth2p4_006058 | 1021 | 967 | 1 . . . 320, 425 . . . 792, 894 . . . 967 |
| Myrth2p4_006119 | 1022 | 1333 | 1 . . . 252, 319 . . . 954, 1088 . . . 1333 |
| Myrth2p4_006140 | 1023 | 1107 | 1 . . . 1107 |
| Myrth2p4_006141 | 1024 | 1434 | 1 . . . 1434 |
| Myrth2p4_006201 | 1025 | 814 | 1 . . . 198, 323 . . . 814 |
| Myrth2p4_006226 | 1026 | 1432 | 1 . . . 149, 231 . . . 400, 490 . . . 728, 874 . . . 1016, 1153 . . . 1432 |
| Myrth2p4_006305 | 1027 | 1746 | 1 . . . 361, 438 . . . 546, 656 . . . 925, 1002 . . . 1621, 1703 . . . 1746 |
| Myrth2p4_006387 | 1028 | 1255 | 1 . . . 88, 153 . . . 1255 |
| Myrth2p4_006397 | 1029 | 2458 | 1 . . . 293, 356 . . . 461, 545 . . . 687, 749 . . . 833, 897 . . . 957, 1017 . . . 1130, |
| | | | 1221 . . . 1232, 1382 . . . 1484, 1589 . . . 2250, 2355 . . . 2458 |
| Myrth2p4_006400 | 1030 | 1242 | 1 . . . 360, 425 . . . 581, 638 . . . 669, 727 . . . 1242 |
| Myrth2p4_006403 | 1031 | 1023 | 1 . . . 1023 |
| Myrth2p4_006408 | 1032 | 2597 | 1 . . . 248, 359 . . . 1232, 1317 . . . 1686, 1819 . . . 2597 |
| Myrth2p4_006434 | 1033 | 1642 | 1 . . . 327, 440 . . . 1642 |
| Myrth2p4_006514 | 1034 | 1662 | 1 . . . 372, 433 . . . 1662 |
| Myrth2p4_006524 | 1035 | 2020 | 1 . . . 51, 144 . . . 1175, 1253 . . . 2020 |
| Myrth2p4_006587 | 1036 | 3143 | 1 . . . 562, 658 . . . 3143 |
| Myrth2p4_006646 | 1037 | 890 | 1 . . . 186, 272 . . . 433, 486 . . . 890 |
| Myrth2p4_006765 | 1038 | 1042 | 1 . . . 460, 672 . . . 1042 |
| Myrth2p4_006772 | 1039 | 836 | 1 . . . 49, 133 . . . 406, 515 . . . 836 |
| Myrth2p4_006795 | 1040 | 1293 | 1 . . . 99, 158 . . . 304, 422 . . . 1111, 1231 . . . 1293 |
| Myrth2p4_006807 | 1041 | 889 | 1 . . . 145, 243 . . . 889 |
| Myrth2p4_006821 | 1042 | 936 | 1 . . . 324, 508 . . . 936 |
| Myrth2p4_006837 | 1043 | 2207 | 1 . . . 192, 256 . . . 311, 380 . . . 550, 627 . . . 797, 901 . . . 1029, 1097 . . . 2207 |
| Myrth2p4_007013 | 1044 | 648 | 1 . . . 209, 273 . . . 648 |
| Myrth2p4_007061 | 1045 | 1350 | 1 . . . 847, 947 . . . 1350 |
| Myrth2p4_007109 | 1046 | 2288 | 1 . . . 262, 315 . . . 2124, 2234 . . . 2288 |
| Myrth2p4_007127 | 1047 | 1074 | 1 . . . 1074 |
| Myrth2p4_007150 | 1048 | 947 | 1 . . . 122, 236 . . . 291 . . . 523 . . . 947 |
| Myrth2p4_007367 | 1049 | 1265 | 1 . . . 159, 260 . . . 605, 895 . . . 1265 |
| Myrth2p4_007409 | 1050 | 775 | 1 . . . 295, 408 . . . 775 |
| Myrth2p4_007425 | 1051 | 1247 | 1 . . . 145, 214 . . . 852, 1000 . . . 1247 |
| Myrth2p4_007444 | 1052 | 2844 | 1 . . . 49, 120 . . . 286, 343 . . . 824, 890 . . . 1229, 1309 . . . 1798, |
| | | | 1868 . . . 2133, 2198 . . . 2291, 2369 . . . 2844 |
| Myrth2p4_007447 | 1053 | 1001 | 1 . . . 281, 398 . . . 1001 |
| Myrth2p4_007461 | 1054 | 1398 | 1 . . . 439, 539 . . . 863, 978 . . . 1398 |
| Myrth2p4_007538 | 1055 | 1748 | 1 . . . 1587, 1716 . . . 1748 |
| Myrth2p4_007539 | 1056 | 1659 | 1 . . . 383, 509 . . . 660, 796 . . . 1218, 1395 . . . 1459, 1576 . . . 1659 |
| Myrth2p4_007540 | 1057 | 412 | 1 . . . 49, 138 . . . 412 |
| Myrth2p4_007556 | 1058 | 1334 | 1 . . . 414, 553 . . . 578, 671 . . . 729, 856 . . . 916, 1049 . . . 1334 |
| Myrth2p4_007648 | 1059 | 2854 | 1 . . . 79, 134 . . . 205, 268 . . . 341, 408 . . . 897, 1065 . . . 2052, |
| | | | 2121 . . . 2188, 2337 . . . 2854 |
| Myrth2p4_007688 | 1060 | 1185 | 1 . . . 993, 1090 . . . 1185 |
| Myrth2p4_007726 | 1061 | 912 | 1 . . . 912 |
| Myrth2p4_007729 | 1062 | 1343 | 1 . . . 168, 225 . . . 554, 612 . . . 1343 |
| Myrth2p4_007771 | 1063 | 1668 | 1 . . . 108, 223 . . . 316, 461 . . . 704, 793 . . . 999, 1104 . . . 1668 |
| Myrth2p4_007781 | 1064 | 1734 | 1 . . . 806, 882 . . . 1077, 1213 . . . 1734 |
| Myrth2p4_007801 | 1065 | 1071 | 1 . . . 1071 |
| Myrth2p4_007815 | 1066 | 1200 | 1 . . . 444, 564 . . . 1004, 1105 . . . 1200 |
| Myrth2p4_007838 | 1067 | 1167 | 1 . . . 181, 292 . . . 371, 443 . . . 492, 572 . . . 616, 771 . . . 1167 |
| Myrth2p4_007849 | 1068 | 1718 | 1 . . . 110, 184 . . . 238, 312 . . . 548, 652 . . . 854, 996 . . . 1137, |
| | | | 1198 . . . 1283, 1403 . . . 1575, 1681 . . . 1718 |
| Myrth2p4_007850 | 1069 | 1609 | 1 . . . 338, 455 . . . 551, 646 . . . 845, 938 . . . 1085, 1168 . . . 1426, |
| | | | 1572 . . . 1609 |
| Myrth2p4_007861 | 1070 | 1877 | 1 . . . 818, 928 . . . 1124, 1193 . . . 1340, 1415 . . . 1877 |
| Myrth2p4_007867 | 1071 | 1410 | 1 . . . 606, 703 . . . 1410 |
| Myrth2p4_007877 | 1072 | 884 | 1 . . . 100, 167 . . . 528, 639 . . . 884 |
| Myrth2p4_007915 | 1073 | 1106 | 1 . . . 155, 313 . . . 930, 1022 . . . 1106 |
| Myrth2p4_007920 | 1074 | 2385 | 1 . . . 255, 313 . . . 498, 1414 . . . 1496, 1564 . . . 2038, 2194 . . . 2385 |
| Myrth2p4_007924 | 1075 | 2637 | 1 . . . 2637 |
| Myrth2p4_007956 | 1076 | 753 | 1 . . . 753 |
| Myrth2p4_007996 | 1077 | 2049 | 1 . . . 622, 701 . . . 2049 |
| Myrth2p4_008028 | 1078 | 833 | 1 . . . 371, 464 . . . 833 |
| Myrth2p4_008123 | 1079 | 1260 | 1 . . . 1260 |
| Myrth2p4_008179 | 1080 | 1099 | 1 . . . 961, 1029 . . . 1099 |
| Myrth2p4_008220 | 1081 | 1215 | 1 . . . 373, 467 . . . 1215 |
| Myrth2p4_008285 | 1082 | 2530 | 1 . . . 148, 224 . . . 661, 729 . . . 2530 |
| Myrth2p4_008298 | 1083 | 832 | 1 . . . 347, 445 . . . 665, 753 . . . 832 |
| Myrth2p4_008299 | 1084 | 2568 | 1 . . . 203, 330 . . . 2568 |
| Myrth2p4_008353 | 1085 | 2398 | 1 . . . 397, 462 . . . 2398 |
| Myrth2p4_008360 | 1086 | 2788 | 1 . . . 476, 603 . . . 835, 942 . . . 2788 |
| Myrth2p4_008429 | 1087 | 1077 | 1 . . . 134, 369 . . . 1077 |
| Myrth2p4_008437 | 1088 | 903 | 1 . . . 903 |
| Myrth2p4_008501 | 1089 | 1776 | 1 . . . 49, 165 . . . 566, 740 . . . 996, 1106 . . . 1178, 1238 . . . 1776 |
| Myrth2p4_008515 | 1090 | 1780 | 1 . . . 92, 167 . . . 642, 738 . . . 1322, 1518 . . . 1780 |
| Myrth2p4_008522 | 1091 | 1443 | 1 . . . 1443 |
| Myrth2p4_008530 | 1092 | 915 | 1 . . . 101, 210 . . . 757, 827 . . . 915 |
| Myrth2p4_008541 | 1093 | 918 | 1 . . . 918 |
| Myrth2p4_008564 | 1094 | 727 | 1 . . . 307, 462 . . . 727 |
| Myrth2p4_008615 | 1095 | 2721 | 1 . . . 2721 |
| Myrth2p4_008650 | 1096 | 246 | 1 . . . 246 |
| Myrth2p4_008756 | 1097 | 1296 | 1 . . . 1296 |
| Myrth2p4_000413 | 1098 | 1746 | 1 . . . 585, 663 . . . 1034, 1108 . . . 1596, 1663 . . . 1746 |
| Myrth2p4_000624 | 1099 | 700 | 1 . . . 93, 267 . . . 415, 538 . . . 700 |
| Myrth2p4_001189 | 1100 | 2148 | 1 . . . 106, 160 . . . 550, 609 . . . 2148 |
| Myrth2p4_001457 | 1101 | 1746 | 1 . . . 1477, 1532 . . . 1746 |
| Myrth2p4_001536 | 1102 | 2030 | 1 . . . 72, 186 . . . 739, 886 . . . 1156, 1232 . . . 1514, 1580 . . . 1814, |
| | | | 1877 . . . 2030 |
| Myrth2p4_001740 | 1103 | 1337 | 1 . . . 1218, 1290 . . . 1337 |
| Myrth2p4_003589 | 1104 | 1593 | 1 . . . 378, 447 . . . 676, 735 . . . 1593 |
| Myrth2p4_003938 | 1105 | 1474 | 1 . . . 257, 466 . . . 1474 |
| Myrth2p4_006092 | 1106 | 828 | 1 . . . 301, 362 . . . 828 |
| Myrth2p4_006213 | 1107 | 1376 | 1 . . . 651, 746 . . . 1055, 1213 . . . 1376 |
| Myrth2p4_008350 | 1108 | 1793 | 1 . . . 348, 452 . . . 889, 972 . . . 1793 |
| MYRTH_1_00009 | 1111 | 1649 | 1 . . . 393, 458 . . . 877, 1005 . . . 1649 |
| MYRTH_1_00020 | 1112 | 1614 | 1 . . . 259, 314 . . . 418, 485 . . . 1614 |
| MYRTH_1_00021 | 1113 | 2445 | 1 . . . 1323, 2275 . . . 2445 |
| MYRTH_1_00032 | 1116 | 2544 | 1 . . . 801, 904 . . . 1891, 1990 . . . 2443, 2511 . . . 2544 |
| MYRTH_1_00037 | 1117 | 815 | 1 . . . 660, 705 . . . 815 |
| MYRTH_1_00069 | 1118 | 1829 | 1 . . . 36, 104 . . . 1270, 1398 . . . 1829 |
| MYRTH_1_00080 | 1119 | 884 | 1 . . . 100, 167 . . . 528, 603 . . . 884 |
| MYRTH_1_00084 | 1120 | 1817 | 1 . . . 620, 721 . . . 792, 912 . . . 1183, 1306 . . . 1817 |
| MYRTH_1_00087 | 1121 | 3029 | 1 . . . 84, 127 . . . 337, 433 . . . 518, 582 . . . 1022, 1141 . . . 1244, |
| | | | 1301 . . . 1597, 1649 . . . 3029 |
| MYRTH_1_00098 | 1122 | 1255 | 1 . . . 88, 153 . . . 460, 581 . . . 1255 |
| MYRTH_2_00218 | 1123 | 929 | 1 . . . 444, 542 . . . 762, 850 . . . 929 |
| MYRTH_2_00583 | 1124 | 3143 | 1 . . . 562, 658 . . . 3143 |
| MYRTH_2_00740 | 1125 | 1290 | 1 . . . 1290 |
| MYRTH_2_01076 | 1127 | 1321 | 1 . . . 107, 174 . . . 485, 541 . . . 1321 |
| MYRTH_2_01077 | 1128 | 1306 | 1 . . . 92, 159 . . . 470, 526 . . . 1306 |
| MYRTH_2_02633 | 1132 | 2398 | 1 . . . 397, 462 . . . 2398 |
| MYRTH_2_04186 | 1134 | 1410 | 1 . . . 606, 703 . . . 1410 |
| MYRTH_2_04244 | 1135 | 1218 | 1 . . . 929, 1002 . . . 1218 |
| MYRTH_3_00003 | 1138 | 1343 | 1 . . . 168, 342 . . . 554, 612 . . . 1343 |
| MYRTH_3_00016 | 1139 | 1839 | 1 . . . 1269, 1819 . . . 1839 |
| MYRTH_3_00086 | 1140 | 1614 | 1 . . . 259, 314 . . . 414, 490 . . . 1614 |
| MYRTH_3_00105 | 1141 | 1101 | 1 . . . 239, 294 . . . 356, 586 . . . 931, 994 . . . 1101 |
| MYRTH_3_00120 | 1142 | 1293 | 1 . . . 81, 155 . . . 304, 422 . . . 1111, 1231 . . . 1293 |
| MYRTH_3_00124 | 1143 | 1208 | 1 . . . 101, 351 . . . 925, 1040 . . . 1089, 1170 . . . 1208 |
| MYRTH_4_05758 | 1145 | 1107 | 1 . . . 1107 |
| MYRTH_4_09820 | 1147 | 929 | 1 . . . 444, 542 . . . 762, 850 . . . 929 |
| Myrth2p4_000387 | 1148 | 2075 | 1 . . . 500, 590 . . . 2075 |
| Myrth2p4_000489 | 1149 | 1882 | 1 . . . 505, 585 . . . 740, 816 . . . 1882 |
| Myrth2p4_001363 | 1150 | 2403 | 1 . . . 2403 |
| Myrth2p4_001546 | 1151 | 2436 | 1 . . . 208, 333 . . . 365, 429 . . . 694, 777 . . . 903, 970 . . . 1434, |
| | | | 1537 . . . 2312, 2407 . . . 2436 |
| Myrth2p4_002267 | 1152 | 2258 | 1 . . . 166, 248 . . . 1162, 1268 . . . 2058, 2214 . . . 2258 |
| Myrth2p4_002365 | 1153 | 2437 | 1 . . . 243, 373 . . . 508, 726 . . . 880, 1037 . . . 1353, 1454 . . . 1629, |
| | | | 1708 . . . 2062, 2146 . . . 2437 |
| Myrth2p4_003086 | 1154 | 2154 | 1 . . . 181, 241 . . . 557, 622 . . . 2022, 2110 . . . 2154 |
| Myrth2p4_004152 | 1155 | 840 | 1 . . . 840 |
| Myrth2p4_004330 | 1156 | 1516 | 1 . . . 204, 280 . . . 709, 840 . . . 1516 |
| Myrth2p4_004961 | 1157 | 1122 | 1 . . . 273, 371 . . . 900, 1044 . . . 1122 |
| Myrth2p4_005807 | 1158 | 1132 | 1 . . . 224, 356 . . . 565, 674 . . . 831, 903 . . . 1132 |
| Myrth2p4_005966 | 1159 | 1806 | 1 . . . 468, 544 . . . 1806 |
| Myrth2p4_006645 | 1160 | 1260 | 1 . . . 1260 |
| Myrth2p4_008594 | 1161 | 1622 | 1 . . . 1047, 1143 . . . 1622 |

TABLE 2C
List of genes of Aureobasidium pullulans with reference to exon boundaries

| | Genomic | | |
|---|---|---|---|
| | sequence | Genomic | |
| | (SEQ | sequence | |
| Gene ID | ID NO:) | length | Exon boundaries (nucleotide positions) and exons |
| Aurpu2p4_000013 | 1774 | 1865 | 1 . . . 920, 1067 . . . 1865 |
| Aurpu2p4_000017 | 1775 | 1820 | 1 . . . 454, 518 . . . 596, 651 . . . 858, 911 . . . 1045, 1099 . . . 1312, |
| | | | 1376 . . . 1681, 1735 . . . 1820 |
| Aurpu2p4_000070 | 1776 | 1376 | 1 . . . 78, 132 . . . 1376 |
| Aurpu2p4_000074 | 1777 | 2572 | 1 . . . 88, 153 . . . 468, 523 . . . 808, 881 . . . 2572 |
| Aurpu2p4_000163 | 1778 | 798 | 1 . . . 215, 276 . . . 798 |
| Aurpu2p4_000184 | 1779 | 1321 | 1 . . . 185, 247 . . . 1139, 1191 . . . 1321 |
| Aurpu2p4_000224 | 1780 | 1215 | 1 . . . 288, 471 . . . 632, 687 . . . 1126, 1185 . . . 1215 |
| Aurpu2p4_000225 | 1781 | 781 | 1 . . . 312, 368 . . . 781 |
| Aurpu2p4_000232 | 1782 | 1110 | 1 . . . 1110 |
| Aurpu2p4_000354 | 1783 | 1638 | 1 . . . 1638 |
| Aurpu2p4_000408 | 1784 | 2361 | 1 . . . 2361 |
| Aurpu2p4_000459 | 1785 | 1362 | 1 . . . 62, 120 . . . 200, 252 . . . 1362 |
| Aurpu2p4_000533 | 1786 | 2403 | 1 . . . 2403 |
| Aurpu2p4_000568 | 1787 | 1215 | 1 . . . 1215 |
| Aurpu2p4_000586 | 1788 | 882 | 1 . . . 882 |
| Aurpu2p4_000590 | 1789 | 2013 | 1 . . . 235, 304 . . . 648, 713 . . . 1024, 1076 . . . 2013 |
| Aurpu2p4_000594 | 1790 | 2027 | 1 . . . 1250, 1298 . . . 2027 |
| Aurpu2p4_000617 | 1791 | 1575 | 1 . . . 1575 |
| Aurpu2p4_000662 | 1792 | 1380 | 1 . . . 279, 329 . . . 1105, 1159 . . . 1380 |
| Aurpu2p4_000692 | 1793 | 1589 | 1 . . . 53, 106 . . . 767, 818 . . . 1406, 1454 . . . 1589 |
| Aurpu2p4_000730 | 1794 | 1727 | 1 . . . 463, 520 . . . 1433, 1488 . . . 1727 |
| Aurpu2p4_000792 | 1795 | 1526 | 1 . . . 299, 351 . . . 534, 583 . . . 646, 779 . . . 810, 954 . . . 1526 |
| Aurpu2p4_000799 | 1796 | 2676 | 1 . . . 2676 |
| Aurpu2p4_000819 | 1797 | 1791 | 1 . . . 329, 389 . . . 548, 625 . . . 1791 |
| Aurpu2p4_000860 | 1798 | 2241 | 1 . . . 304, 353 . . . 2241 |
| Aurpu2p4_000919 | 1799 | 1487 | 1 . . . 609, 661 . . . 926, 976 . . . 1091, 1142 . . . 1174, 1225 . . . 1487 |
| Aurpu2p4_000934 | 1800 | 1381 | 1 . . . 446, 499 . . . 689, 741 . . . 1381 |
| Aurpu2p4_000947 | 1801 | 1896 | 1 . . . 1896 |
| Aurpu2p4_000948 | 1802 | 1863 | 1 . . . 171, 227 . . . 284, 339 . . . 386, 445 . . . 722, 778 . . . 1863 |
| Aurpu2p4_000984 | 1803 | 2609 | 1 . . . 966, 1017 . . . 2609 |
| Aurpu2p4_000995 | 1804 | 1104 | 1 . . . 455, 516 . . . 1104 |
| Aurpu2p4_001037 | 1805 | 1828 | 1 . . . 43, 103 . . . 1070, 1124 . . . 1828 |
| Aurpu2p4_001097 | 1806 | 3426 | 1 . . . 3426 |
| Aurpu2p4_001104 | 1807 | 1469 | 1 . . . 428, 479 . . . 1469 |
| Aurpu2p4_001152 | 1808 | 1435 | 1 . . . 1155, 1214 . . . 1435 |
| Aurpu2p4_001194 | 1809 | 6009 | 1 . . . 6009 |
| Aurpu2p4_001195 | 1810 | 1372 | 1 . . . 189, 239 . . . 651, 761 . . . 1178, 1229 . . . 1372 |
| Aurpu2p4_001256 | 1811 | 802 | 1 . . . 284, 338 . . . 416, 476 . . . 802 |
| Aurpu2p4_001441 | 1812 | 1949 | 1 . . . 361, 418 . . . 1949 |
| Aurpu2p4_001503 | 1813 | 1493 | 1 . . . 133, 185 . . . 503, 557 . . . 1493 |
| Aurpu2p4_001504 | 1814 | 1658 | 1 . . . 292, 350 . . . 517, 571 . . . 719, 773 . . . 868, 942 . . . 1163, 1224 . . . 1658 |
| Aurpu2p4_001512 | 1815 | 1663 | 1 . . . 1581, 1634 . . . 1663 |
| Aurpu2p4_001553 | 1816 | 2249 | 1 . . . 942, 992 . . . 1840, 1896 . . . 2144, 2199 . . . 2249 |
| Aurpu2p4_001599 | 1817 | 1752 | 1 . . . 1752 |
| Aurpu2p4_001600 | 1818 | 1728 | 1 . . . 1728 |
| Aurpu2p4_001633 | 1819 | 1400 | 1 . . . 717, 772 . . . 1049, 1304 . . . 1400 |
| Aurpu2p4_001665 | 1820 | 1821 | 1 . . . 437, 495 . . . 1821 |
| Aurpu2p4_001680 | 1821 | 1270 | 1 . . . 611, 670 . . . 1270 |
| Aurpu2p4_001713 | 1822 | 1140 | 1 . . . 172, 226 . . . 461, 515 . . . 713, 771 . . . 926, 980 . . . 1140 |
| Aurpu2p4_001718 | 1823 | 1215 | 1 . . . 577, 639 . . . 1160, 1211 . . . 1215 |
| Aurpu2p4_001807 | 1824 | 1125 | 1 . . . 1125 |
| Aurpu2p4_001825 | 1825 | 1284 | 1 . . . 242, 299 . . . 416, 472 . . . 1284 |
| Aurpu2p4_001892 | 1826 | 1245 | 1 . . . 1245 |
| Aurpu2p4_001986 | 1827 | 2189 | 1 . . . 589, 646 . . . 2189 |
| Aurpu2p4_002000 | 1828 | 846 | 1 . . . 846 |
| Aurpu2p4_002005 | 1829 | 1173 | 1 . . . 1173 |
| Aurpu2p4_002047 | 1830 | 734 | 1 . . . 185, 245 . . . 734 |
| Aurpu2p4_002086 | 1831 | 1759 | 1 . . . 404, 454 . . . 1759 |
| Aurpu2p4_002155 | 1832 | 850 | 1 . . . 316, 381 . . . 850 |
| Aurpu2p4_002166 | 1833 | 1062 | 1 . . . 1062 |
| Aurpu2p4_002167 | 1834 | 2884 | 1 . . . 269, 322 . . . 1074, 1129 . . . 1237, 1292 . . . 2884 |
| Aurpu2p4_002190 | 1835 | 1310 | 1 . . . 102, 159 . . . 312, 364 . . . 1310 |
| Aurpu2p4_002220 | 1836 | 1478 | 1 . . . 263, 312 . . . 437, 489 . . . 617, 665 . . . 731, 782 . . . 1178, 1231 . . . 1478 |
| Aurpu2p4_002256 | 1837 | 2544 | 1 . . . 178, 234 . . . 932, 985 . . . 1112, 1164 . . . 1747, 1797 . . . 2544 |
| Aurpu2p4_002267 | 1838 | 1185 | 1 . . . 1185 |
| Aurpu2p4_002284 | 1839 | 1497 | 1 . . . 1497 |
| Aurpu2p4_002399 | 1840 | 2713 | 1 . . . 146, 195 . . . 376, 427 . . . 640, 692 . . . 988, 1042 . . . 2713 |
| Aurpu2p4_002518 | 1841 | 1485 | 1 . . . 427, 515 . . . 1485 |
| Aurpu2p4_002522 | 1842 | 1079 | 1 . . . 351, 405 . . . 1079 |
| Aurpu2p4_002533 | 1843 | 2256 | 1 . . . 177, 226 . . . 451, 501 . . . 635, 882 . . . 1744, 1811 . . . 2038, |
| | | | 2098 . . . 2256 |
| Aurpu2p4_002671 | 1844 | 1110 | 1 . . . 1110 |
| Aurpu2p4_002672 | 1845 | 2849 | 1 . . . 118, 168 . . . 263, 317 . . . 373, 430 . . . 615, 701 . . . 733, 785 . . . 874, |
| | | | 923 . . . 1191, 1240 . . . 1732, 1783 . . . 2849 |
| Aurpu2p4_002750 | 1846 | 1170 | 1 . . . 104, 157 . . . 289, 345 . . . 484, 537 . . . 1170 |
| Aurpu2p4_002860 | 1847 | 2220 | 1 . . . 2220 |
| Aurpu2p4_002907 | 1848 | 1674 | 1 . . . 1674 |
| Aurpu2p4_002940 | 1849 | 4560 | 1 . . . 335, 388 . . . 3884, 3938 . . . 3959, 4165 . . . 4342, 4393 . . . 4560 |
| Aurpu2p4_002942 | 1850 | 1209 | 1 . . . 1209 |
| Aurpu2p4_002955 | 1851 | 1686 | 1 . . . 1686 |
| Aurpu2p4_002987 | 1852 | 1216 | 1 . . . 187, 241 . . . 340, 394 . . . 1216 |
| Aurpu2p4_003029 | 1853 | 1020 | 1 . . . 1020 |
| Aurpu2p4_003104 | 1854 | 1737 | 1 . . . 1737 |
| Aurpu2p4_003184 | 1855 | 2442 | 1 . . . 969, 1017 . . . 1371, 1424 . . . 2442 |
| Aurpu2p4_003313 | 1856 | 1809 | 1 . . . 334, 392 . . . 1809 |
| Aurpu2p4_003364 | 1857 | 2784 | 1 . . . 2784 |
| Aurpu2p4_003555 | 1858 | 1440 | 1 . . . 1440 |
| Aurpu2p4_003594 | 1859 | 1575 | 1 . . . 1575 |
| Aurpu2p4_003606 | 1860 | 1092 | 1 . . . 280, 332 . . . 1092 |
| Aurpu2p4_003607 | 1861 | 1306 | 1 . . . 505, 562 . . . 896, 955 . . . 1113, 1166 . . . 1306 |
| Aurpu2p4_003685 | 1862 | 881 | 1 . . . 245, 299 . . . 518, 570 . . . 881 |
| Aurpu2p4_003727 | 1863 | 1468 | 1 . . . 542, 595 . . . 1468 |
| Aurpu2p4_003747 | 1864 | 3552 | 1 . . . 212, 266 . . . 452, 709 . . . 907, 1047 . . . 1236, 1287 . . . 3552 |
| Aurpu2p4_003884 | 1865 | 1113 | 1 . . . 157, 212 . . . 1113 |
| Aurpu2p4_003888 | 1866 | 2763 | 1 . . . 2231, 2283 . . . 2763 |
| Aurpu2p4_003893 | 1867 | 948 | 1 . . . 948 |
| Aurpu2p4_003941 | 1868 | 1336 | 1 . . . 1074, 1128 . . . 1266, 1323 . . . 1336 |
| Aurpu2p4_004107 | 1869 | 2366 | 1 . . . 462, 662 . . . 1591, 1644 . . . 2366 |
| Aurpu2p4_004115 | 1870 | 1038 | 1 . . . 406, 467 . . . 1038 |
| Aurpu2p4_004128 | 1871 | 1434 | 1 . . . 1434 |
| Aurpu2p4_004186 | 1872 | 742 | 1 . . . 261, 320 . . . 742 |
| Aurpu2p4_004265 | 1873 | 2724 | 1 . . . 289, 344 . . . 2724 |
| Aurpu2p4_004286 | 1874 | 2861 | 1 . . . 812, 1268 . . . 1495, 1548 . . . 1575, 1627 . . . 2143, 2197 . . . 2861 |
| Aurpu2p4_004297 | 1875 | 2145 | 1 . . . 1894, 1958 . . . 2145 |
| Aurpu2p4_004347 | 1876 | 1413 | 1 . . . 1413 |
| Aurpu2p4_004477 | 1877 | 2778 | 1 . . . 72, 124 . . . 295, 345 . . . 625, 681 . . . 836, 885 . . . 1884, 1940 . . . 2139, |
| | | | 2193 . . . 2356, 2406 . . . 2778 |
| Aurpu2p4_004489 | 1878 | 2212 | 1 . . . 196, 465 . . . 512, 564 . . . 802, 859 . . . 1040, 1240 . . . 2212 |
| Aurpu2p4_004524 | 1879 | 1011 | 1 . . . 1011 |
| Aurpu2p4_004527 | 1880 | 2151 | 1 . . . 2151 |
| Aurpu2p4_004550 | 1881 | 854 | 1 . . . 452, 509 . . . 854 |
| Aurpu2p4_004694 | 1882 | 1082 | 1 . . . 134, 182 . . . 265, 317 . . . 1082 |
| Aurpu2p4_004762 | 1883 | 1477 | 1 . . . 282, 410 . . . 536, 588 . . . 672, 726 . . . 1335, 1448 . . . 1477 |
| Aurpu2p4_004776 | 1884 | 705 | 1 . . . 705 |
| Aurpu2p4_004801 | 1885 | 2180 | 1 . . . 666, 715 . . . 896, 1229 . . . 1671, 1726 . . . 2180 |
| Aurpu2p4_004899 | 1886 | 1884 | 1 . . . 1884 |
| Aurpu2p4_004916 | 1887 | 1928 | 1 . . . 416, 475 . . . 587, 640 . . . 1928 |
| Aurpu2p4_004926 | 1888 | 1217 | 1 . . . 365, 422 . . . 880, 935 . . . 1217 |
| Aurpu2p4_004937 | 1889 | 2255 | 1 . . . 491, 542 . . . 918, 977 . . . 1678, 1731 . . . 1989, 2048 . . . 2255 |
| Aurpu2p4_004986 | 1890 | 1282 | 1 . . . 158, 209 . . . 518, 569 . . . 1282 |
| Aurpu2p4_005056 | 1891 | 1308 | 1 . . . 1308 |
| Aurpu2p4_005097 | 1892 | 2901 | 1 . . . 423, 483 . . . 717, 770 . . . 2901 |
| Aurpu2p4_005194 | 1893 | 1098 | 1 . . . 1098 |
| Aurpu2p4_005236 | 1894 | 2160 | 1 . . . 399, 454 . . . 2160 |
| Aurpu2p4_005278 | 1895 | 830 | 1 . . . 706, 763 . . . 830 |
| Aurpu2p4_005399 | 1896 | 1036 | 1 . . . 327, 377 . . . 1036 |
| Aurpu2p4_005401 | 1897 | 2397 | 1 . . . 120, 171 . . . 394, 443 . . . 827, 882 . . . 1226, 1283 . . . 1523, |
| | | | 1571 . . . 1734, 1786 . . . 2397 |
| Aurpu2p4_005519 | 1898 | 1026 | 1 . . . 1026 |
| Aurpu2p4_005580 | 1899 | 1854 | 1 . . . 1374, 1630 . . . 1854 |
| Aurpu2p4_005825 | 1900 | 1220 | 1 . . . 413, 475 . . . 1028, 1090 . . . 1220 |
| Aurpu2p4_005865 | 1901 | 1248 | 1 . . . 1248 |
| Aurpu2p4_005914 | 1902 | 2530 | 1 . . . 145, 201 . . . 522, 580 . . . 2530 |
| Aurpu2p4_005929 | 1903 | 1025 | 1 . . . 33, 91 . . . 333, 414 . . . 894, 958 . . . 1025 |
| Aurpu2p4_006113 | 1904 | 1095 | 1 . . . 1095 |
| Aurpu2p4_006128 | 1905 | 884 | 1 . . . 442, 496 . . . 884 |
| Aurpu2p4_006160 | 1906 | 1722 | 1 . . . 1722 |
| Aurpu2p4_006162 | 1907 | 1608 | 1 . . . 1608 |
| Aurpu2p4_006176 | 1908 | 1484 | 1 . . . 445, 513 . . . 1227, 1285 . . . 1396, 1455 . . . 1484 |
| Aurpu2p4_006179 | 1909 | 1392 | 1 . . . 1392 |
| Aurpu2p4_006195 | 1910 | 2233 | 1 . . . 659, 711 . . . 1164, 1231 . . . 1489, 1544 . . . 1708, 1770 . . . 2233 |
| Aurpu2p4_006206 | 1911 | 1725 | 1 . . . 1035, 1093 . . . 1725 |
| Aurpu2p4_006207 | 1912 | 6091 | 1 . . . 203, 255 . . . 455, 508 . . . 4226, 4281 . . . 4787, 4857 . . . 5006, |
| | | | 5064 . . . 5293, 5339 . . . 6091 |
| Aurpu2p4_006222 | 1913 | 1788 | 1 . . . 1788 |
| Aurpu2p4_006237 | 1914 | 773 | 1 . . . 202, 268 . . . 773 |
| Aurpu2p4_006246 | 1915 | 2528 | 1 . . . 172, 222 . . . 320, 373 . . . 2528 |
| Aurpu2p4_006312 | 1916 | 964 | 1 . . . 317, 381 . . . 836, 898 . . . 964 |
| Aurpu2p4_006313 | 1917 | 1052 | 1 . . . 444, 504 . . . 1052 |
| Aurpu2p4_006392 | 1918 | 1655 | 1 . . . 263, 314 . . . 1655 |
| Aurpu2p4_006557 | 1919 | 1451 | 1 . . . 364, 427 . . . 1451 |
| Aurpu2p4_006782 | 1920 | 3618 | 1 . . . 93, 344 . . . 533, 586 . . . 592, 651 . . . 748, 801 . . . 1252, 1300 . . . 1888, |
| | | | 1947 . . . 2106, 2163 . . . 2336, 2390 . . . 2839, 2892 . . . 3618 |
| Aurpu2p4_006900 | 1921 | 2341 | 1 . . . 416, 466 . . . 533, 582 . . . 756, 815 . . . 1135, 1186 . . . 1924, |
| | | | 1976 . . . 2341 |
| Aurpu2p4_006933 | 1922 | 2242 | 1 . . . 278, 515 . . . 1235, 1295 . . . 2242 |
| Aurpu2p4_007070 | 1923 | 1257 | 1 . . . 1257 |
| Aurpu2p4_007082 | 1924 | 1287 | 1 . . . 65, 122 . . . 540, 616 . . . 1082, 1150 . . . 1287 |
| Aurpu2p4_007093 | 1925 | 813 | 1 . . . 257, 308 . . . 455, 509 . . . 679, 733 . . . 813 |
| Aurpu2p4_007113 | 1926 | 2028 | 1 . . . 2028 |
| Aurpu2p4_007124 | 1927 | 777 | 1 . . . 360, 415 . . . 777 |
| Aurpu2p4_007126 | 1928 | 1653 | 1 . . . 263, 321 . . . 1653 |
| Aurpu2p4_007149 | 1929 | 1263 | 1 . . . 1263 |
| Aurpu2p4_007160 | 1930 | 1971 | 1 . . . 1971 |
| Aurpu2p4_007177 | 1931 | 2187 | 1 . . . 2187 |
| Aurpu2p4_007190 | 1932 | 1865 | 1 . . . 289, 348 . . . 1657, 1710 . . . 1865 |
| Aurpu2p4_007196 | 1933 | 1239 | 1 . . . 609, 676 . . . 1239 |
| Aurpu2p4_007206 | 1934 | 1435 | 1 . . . 360, 406 . . . 573, 626 . . . 1435 |
| Aurpu2p4_007220 | 1935 | 1089 | 1 . . . 538, 593 . . . 1089 |
| Aurpu2p4_007270 | 1936 | 2669 | 1 . . . 118, 166 . . . 285, 333 . . . 541, 611 . . . 907, 959 . . . 1064, 1117 . . . 2669 |
| Aurpu2p4_007272 | 1937 | 1961 | 1 . . . 208, 264 . . . 500, 559 . . . 617, 675 . . . 841, 902 . . . 1273, 1325 . . . 1961 |
| Aurpu2p4_007292 | 1938 | 1064 | 1 . . . 71, 134 . . . 1064 |
| Aurpu2p4_007342 | 1939 | 1042 | 1 . . . 77, 133 . . . 1042 |
| Aurpu2p4_007356 | 1940 | 1433 | 1 . . . 723, 777 . . . 1433 |
| Aurpu2p4_007383 | 1941 | 3123 | 1 . . . 3123 |
| Aurpu2p4_007404 | 1942 | 1650 | 1 . . . 1650 |
| Aurpu2p4_007424 | 1943 | 1236 | 1 . . . 1236 |
| Aurpu2p4_007428 | 1944 | 1829 | 1 . . . 14, 430 . . . 927, 976 . . . 1739, 1795 . . . 1829 |
| Aurpu2p4_007429 | 1945 | 1052 | 1 . . . 290, 350 . . . 1052 |
| Aurpu2p4_007455 | 1946 | 1572 | 1 . . . 1572 |
| Aurpu2p4_007488 | 1947 | 1131 | 1 . . . 228, 287 . . . 358, 418 . . . 1131 |
| Aurpu2p4_007493 | 1948 | 1856 | 1 . . . 387, 568 . . . 1515, 1569 . . . 1856 |
| Aurpu2p4_007511 | 1949 | 1152 | 1 . . . 253, 325 . . . 426, 480 . . . 909, 966 . . . 1152 |
| Aurpu2p4_007612 | 1950 | 786 | 1 . . . 278, 330 . . . 531, 589 . . . 786 |
| Aurpu2p4_007614 | 1951 | 1189 | 1 . . . 552, 602 . . . 1189 |
| Aurpu2p4_007621 | 1952 | 1275 | 1 . . . 440, 496 . . . 880, 938 . . . 984, 1035 . . . 1275 |
| Aurpu2p4_007662 | 1953 | 873 | 1 . . . 873 |
| Aurpu2p4_007707 | 1954 | 843 | 1 . . . 132, 184 . . . 409, 461 . . . 843 |
| Aurpu2p4_007805 | 1955 | 2072 | 1 . . . 1612, 1670 . . . 1851, 1911 . . . 2072 |
| Aurpu2p4_007919 | 1956 | 895 | 1 . . . 109, 166 . . . 266, 317 . . . 373, 423 . . . 716, 770 . . . 895 |
| Aurpu2p4_008001 | 1957 | 2647 | 1 . . . 298, 349 . . . 710, 763 . . . 2407, 2460 . . . 2647 |
| Aurpu2p4_008021 | 1958 | 1541 | 1 . . . 1242, 1293 . . . 1541 |
| Aurpu2p4_008140 | 1959 | 1636 | 1 . . . 369, 422 . . . 1636 |
| Aurpu2p4_008212 | 1960 | 1203 | 1 . . . 338, 396 . . . 1203 |
| Aurpu2p4_008231 | 1961 | 2251 | 1 . . . 1600, 1653 . . . 2251 |
| Aurpu2p4_008239 | 1962 | 1672 | 1 . . . 256, 312 . . . 400, 456 . . . 1075, 1128 . . . 1215, 1274 . . . 1672 |
| Aurpu2p4_008255 | 1963 | 880 | 1 . . . 701, 766 . . . 880 |
| Aurpu2p4_008271 | 1964 | 1517 | 1 . . . 153, 213 . . . 1517 |
| Aurpu2p4_008282 | 1965 | 1986 | 1 . . . 217, 356 . . . 1986 |
| Aurpu2p4_008385 | 1966 | 2258 | 1 . . . 121, 188 . . . 871, 922 . . . 1399, 1448 . . . 2258 |
| Aurpu2p4_008412 | 1967 | 3649 | 1 . . . 341, 396 . . . 478, 532 . . . 627, 742 . . . 933, 983 . . . 1180, |
| | | | 1279 . . . 1354, 1406 . . . 1519, 1780 . . . 3649 |
| Aurpu2p4_008485 | 1968 | 1058 | 1 . . . 528, 579 . . . 1058 |
| Aurpu2p4_008495 | 1969 | 2152 | 1 . . . 426, 1304 . . . 1796, 1853 . . . 2023, 2085 . . . 2152 |
| Aurpu2p4_008503 | 1970 | 1875 | 1 . . . 1875 |
| Aurpu2p4_008585 | 1971 | 2640 | 1 . . . 2640 |
| Aurpu2p4_008692 | 1972 | 2039 | 1 . . . 117, 167 . . . 298, 354 . . . 769, 821 . . . 872, 930 . . . 2039 |
| Aurpu2p4_008705 | 1973 | 3036 | 1 . . . 3036 |
| Aurpu2p4_008725 | 1974 | 960 | 1 . . . 960 |
| Aurpu2p4_008775 | 1975 | 3302 | 1 . . . 1356, 1653 . . . 1781, 2223 . . . 3302 |
| Aurpu2p4_008807 | 1976 | 1129 | 1 . . . 310, 362 . . . 479, 532 . . . 1129 |
| Aurpu2p4_008838 | 1977 | 2346 | 1 . . . 818, 1047 . . . 1484, 1539 . . . 2346 |
| Aurpu2p4_008906 | 1978 | 1291 | 1 . . . 13, 64 . . . 791, 846 . . . 952, 1006 . . . 1291 |
| Aurpu2p4_008972 | 1979 | 1941 | 1 . . . 258, 390 . . . 464, 664 . . . 792, 846 . . . 860, 915 . . . 983, 1040 . . . 1050, |
| | | | 1105 . . . 1232, 1430 . . . 1631, 1692 . . . 1941 |
| Aurpu2p4_008980 | 1980 | 3232 | 1 . . . 608, 669 . . . 1379, 1531 . . . 1781, 2047 . . . 2477, 2534 . . . 3232 |
| Aurpu2p4_009032 | 1981 | 2108 | 1 . . . 132, 188 . . . 410, 589 . . . 937, 1042 . . . 1650, 1703 . . . 2108 |
| Aurpu2p4_009051 | 1982 | 2136 | 1 . . . 2136 |
| Aurpu2p4_009071 | 1983 | 1397 | 1 . . . 336, 390 . . . 1397 |
| Aurpu2p4_009125 | 1984 | 2549 | 1 . . . 50, 107 . . . 2549 |
| Aurpu2p4_009223 | 1985 | 2464 | 1 . . . 154, 205 . . . 544, 595 . . . 2464 |
| Aurpu2p4_009233 | 1986 | 1020 | 1 . . . 1020 |
| Aurpu2p4_009300 | 1987 | 2697 | 1 . . . 2697 |
| Aurpu2p4_009394 | 1988 | 1560 | 1 . . . 1560 |
| Aurpu2p4_009401 | 1989 | 684 | 1 . . . 684 |
| Aurpu2p4_009472 | 1990 | 1329 | 1 . . . 1329 |
| Aurpu2p4_009494 | 1991 | 1543 | 1 . . . 392, 442 . . . 495, 547 . . . 1543 |
| Aurpu2p4_009495 | 1992 | 987 | 1 . . . 987 |
| Aurpu2p4_009496 | 1993 | 1746 | 1 . . . 1746 |
| Aurpu2p4_009563 | 1994 | 1059 | 1 . . . 397, 463 . . . 968, 1018 . . . 1059 |
| Aurpu2p4_009597 | 1995 | 1161 | 1 . . . 867, 925 . . . 1161 |
| Aurpu2p4_009603 | 1996 | 800 | 1 . . . 311, 362 . . . 621, 679 . . . 800 |
| Aurpu2p4_009751 | 1997 | 1437 | 1 . . . 452, 512 . . . 1367, 1423 . . . 1437 |
| Aurpu2p4_009762 | 1998 | 1443 | 1 . . . 359, 410 . . . 702, 755 . . . 1443 |
| Aurpu2p4_009775 | 1999 | 938 | 1 . . . 353, 410 . . . 938 |
| Aurpu2p4_009782 | 2000 | 1989 | 1 . . . 417, 464 . . . 1041, 1092 . . . 1680, 1729 . . . 1854, 1909 . . . 1989 |
| Aurpu2p4_009845 | 2001 | 1038 | 1 . . . 1038 |
| Aurpu2p4_009863 | 2002 | 985 | 1 . . . 705, 762 . . . 787, 841 . . . 985 |
| Aurpu2p4_009889 | 2003 | 1692 | 1 . . . 1190, 1245 . . . 1692 |
| Aurpu2p4_009890 | 2004 | 2555 | 1 . . . 528, 578 . . . 869, 919 . . . 2213, 2262 . . . 2555 |
| Aurpu2p4_009910 | 2005 | 2639 | 1 . . . 322, 376 . . . 2639 |
| Aurpu2p4_010058 | 2006 | 1711 | 1 . . . 324, 377 . . . 1711 |
| Aurpu2p4_010070 | 2007 | 2923 | 1 . . . 75, 125 . . . 320, 379 . . . 487, 593 . . . 689, 742 . . . 2032, 2082 . . . 2923 |
| Aurpu2p4_010087 | 2008 | 1783 | 1 . . . 1570, 1629 . . . 1783 |
| Aurpu2p4_010088 | 2009 | 2511 | 1 . . . 2511 |
| Aurpu2p4_010125 | 2010 | 1508 | 1 . . . 102, 162 . . . 1508 |
| Aurpu2p4_010146 | 2011 | 2596 | 1 . . . 1543, 1655 . . . 1871, 1927 . . . 2596 |
| Aurpu2p4_010192 | 2012 | 3218 | 1 . . . 174, 230 . . . 297, 345 . . . 374, 429 . . . 1220, 1316 . . . 3218 |
| Aurpu2p4_010196 | 2013 | 1599 | 1 . . . 477, 535 . . . 1599 |
| Aurpu2p4_010203 | 2014 | 880 | 1 . . . 118, 179 . . . 413, 475 . . . 880 |
| Aurpu2p4_010291 | 2015 | 813 | 1 . . . 528, 580 . . . 813 |
| Aurpu2p4_010300 | 2016 | 1365 | 1 . . . 1365 |
| Aurpu2p4_010313 | 2017 | 1937 | 1 . . . 262, 311 . . . 889, 941 . . . 1335, 1393 . . . 1836, 1890 . . . 1937 |
| Aurpu2p4_010319 | 2018 | 2170 | 1 . . . 226, 279 . . . 535, 582 . . . 1719, 1771 . . . 1950, 2004 . . . 2170 |
| Aurpu2p4_010388 | 2019 | 1489 | 1 . . . 276, 331 . . . 479, 535 . . . 758, 814 . . . 1007, 1071 . . . 1388, |
| | | | 1484 . . . 1489 |
| Aurpu2p4_010455 | 2020 | 1878 | 1 . . . 77, 133 . . . 269, 556 . . . 813, 864 . . . 1189, 1246 . . . 1364, |
| | | | 1422 . . . 1663, 1717 . . . 1791, 1859 . . . 1878 |
| Aurpu2p4_010457 | 2021 | 1581 | 1 . . . 1581 |
| Aurpu2p4_010464 | 2022 | 1873 | 1 . . . 205, 260 . . . 708, 825 . . . 953, 1001 . . . 1873 |
| Aurpu2p4_010466 | 2023 | 731 | 1 . . . 507, 558 . . . 731 |
| Aurpu2p4_010484 | 2024 | 1749 | 1 . . . 292, 346 . . . 1146, 1265 . . . 1551, 1606 . . . 1749 |
| Aurpu2p4_010534 | 2025 | 1023 | 1 . . . 550, 608 . . . 1023 |
| Aurpu2p4_010571 | 2026 | 2226 | 1 . . . 2226 |
| Aurpu2p4_010592 | 2027 | 1352 | 1 . . . 309, 364 . . . 711, 762 . . . 1352 |
| Aurpu2p4_010596 | 2028 | 1058 | 1 . . . 537, 591 . . . 1058 |
| Aurpu2p4_010603 | 2029 | 1298 | 1 . . . 969, 1074 . . . 1298 |
| Aurpu2p4_010618 | 2030 | 1898 | 1 . . . 243, 298 . . . 606, 664 . . . 813, 867 . . . 1898 |
| Aurpu2p4_010680 | 2031 | 1423 | 1 . . . 22, 90 . . . 326, 380 . . . 978, 1046 . . . 1423 |
| Aurpu2p4_010683 | 2032 | 1596 | 1 . . . 275, 345 . . . 393, 446 . . . 665, 721 . . . 1452, 1565 . . . 1596 |
| Aurpu2p4_010701 | 2033 | 1266 | 1 . . . 348, 402 . . . 736, 792 . . . 1266 |
| Aurpu2p4_010884 | 2034 | 1185 | 1 . . . 1185 |
| Aurpu2p4_010891 | 2035 | 2164 | 1 . . . 144, 236 . . . 345, 396 . . . 570, 624 . . . 943, 1003 . . . 2164 |
| Aurpu2p4_010898 | 2036 | 1827 | 1 . . . 1356, 1414 . . . 1827 |
| Aurpu2p4_010982 | 2037 | 1260 | 1 . . . 1260 |
| Aurpu2p4_010999 | 2038 | 1767 | 1 . . . 546, 604 . . . 867, 925 . . . 1767 |
| Aurpu2p4_011049 | 2039 | 1465 | 1 . . . 174, 235 . . . 561, 616 . . . 1081, 1147 . . . 1342, 1402 . . . 1465 |
| Aurpu2p4_011071 | 2040 | 1848 | 1 . . . 208, 262 . . . 662, 714 . . . 764, 817 . . . 1848 |
| Aurpu2p4_011080 | 2041 | 2451 | 1 . . . 127, 179 . . . 648, 699 . . . 756, 809 . . . 2451 |
| Aurpu2p4_011097 | 2042 | 1182 | 1 . . . 1182 |
| Aurpu2p4_011162 | 2043 | 1776 | 1 . . . 1776 |
| Aurpu2p4_000066 | 2044 | 1899 | 1 . . . 1899 |
| Aurpu2p4_000166 | 2045 | 1294 | 1 . . . 54, 106 . . . 519, 569 . . . 1294 |
| Aurpu2p4_000811 | 2046 | 1683 | 1 . . . 138, 187 . . . 1683 |
| Aurpu2p4_001233 | 2047 | 1583 | 1 . . . 901, 1459 . . . 1583 |
| Aurpu2p4_002002 | 2048 | 1050 | 1 . . . 1050 |
| Aurpu2p4_002244 | 2049 | 2043 | 1 . . . 221, 298 . . . 872, 929 . . . 2043 |
| Aurpu2p4_002270 | 2050 | 1277 | 1 . . . 188, 242 . . . 947, 999 . . . 1277 |
| Aurpu2p4_002403 | 2051 | 1857 | 1 . . . 1857 |
| Aurpu2p4_002547 | 2052 | 1042 | 1 . . . 228, 278 . . . 1042 |
| Aurpu2p4_003458 | 2053 | 2144 | 1 . . . 1791, 1848 . . . 2144 |
| Aurpu2p4_003964 | 2054 | 588 | 1 . . . 588 |
| Aurpu2p4_004483 | 2055 | 1073 | 1 . . . 441, 708 . . . 1073 |
| Aurpu2p4_004802 | 2056 | 2161 | 1 . . . 124, 178 . . . 435, 486 . . . 2161 |
| Aurpu2p4_005858 | 2057 | 1242 | 1 . . . 1242 |
| Aurpu2p4_006413 | 2058 | 1557 | 1 . . . 35, 103 . . . 185, 246 . . . 451, 502 . . . 1557 |
| Aurpu2p4_007081 | 2059 | 1865 | 1 . . . 352, 405 . . . 665, 718 . . . 1865 |
| Aurpu2p4_007695 | 2060 | 1434 | 1 . . . 76, 122 . . . 666, 721 . . . 1434 |
| Aurpu2p4_008408 | 2061 | 1127 | 1 . . . 71, 126 . . . 205, 261 . . . 1047, 1100 . . . 1127 |
| Aurpu2p4_008733 | 2062 | 1835 | 1 . . . 723, 810 . . . 1835 |
| Aurpu2p4_009064 | 2063 | 1113 | 1 . . . 1113 |
| Aurpu2p4_009608 | 2064 | 1448 | 1 . . . 553, 605 . . . 1321, 1387 . . . 1448 |
| Aurpu2p4_009911 | 2065 | 1758 | 1 . . . 301, 355 . . . 455, 504 . . . 606, 656 . . . 1758 |
| Aurpu2p4_009938 | 2066 | 435 | 1 . . . 435 |
| Aurpu2p4_010261 | 2067 | 1865 | 1 . . . 873, 927 . . . 1036, 1094 . . . 1865 |
| Aurpu2p4_010853 | 2068 | 1654 | 1 . . . 219, 274 . . . 1503, 1556 . . . 1654 |
| Aurpu2p4_011048 | 2069 | 846 | 1 . . . 846 |
| AURPU_3_00014 | 2107 | 1434 | 1 . . . 449, 509 . . . 1364, 1420 . . . 1434 |
| AURPU_3_00051 | 2111 | 1434 | 1 . . . 76, 203 . . . 666, 721 . . . 1434 |
| AURPU_3_00113 | 2112 | 1206 | 1 . . . 1206 |
| AURPU_3_00118 | 2113 | 1422 | 1 . . . 556, 611 . . . 1422 |
| AURPU_3_00139 | 2114 | 1553 | 1 . . . 524, 861 . . . 1463, 1514 . . . 1553 |
| AURPU_3_00156 | 2115 | 1820 | 1 . . . 454, 518 . . . 596, 651 . . . 825, 911 . . . 1045, 1099 . . . 1285, |
| | | | 1376 . . . 1681, 1735 . . . 1820 |
| AURPU_3_00173 | 2116 | 1541 | 1 . . . 276, 331 . . . 479, 535 . . . 758, 814 . . . 1007, 1071 . . . 1541 |
| AURPU_3_00174 | 2117 | 1398 | 1 . . . 1398 |
| AURPU_3_00209 | 2119 | 5098 | 1 . . . 528, 578 . . . 869, 919 . . . 2213, 2262 . . . 2538, 2593 . . . 2634, |
| | | | 3540 . . . 4596, 4651 . . . 5098 |
| AURPU_3_00307 | 2120 | 3218 | 1 . . . 174, 230 . . . 297, 345 . . . 374, 429 . . . 1117, 1294 . . . 3218 |
| Aurpu2p4_000157 | 2122 | 1723 | 1 . . . 96, 173 . . . 1723 |
| Aurpu2p4_000356 | 2123 | 1035 | 1 . . . 184, 236 . . . 1035 |
| Aurpu2p4_000818 | 2124 | 2028 | 1 . . . 2028 |
| Aurpu2p4_000960 | 2125 | 1847 | 1 . . . 138, 187 . . . 787, 839 . . . 906, 963 . . . 1847 |
| Aurpu2p4_001076 | 2126 | 1797 | 1 . . . 445, 498 . . . 756, 807 . . . 1797 |
| Aurpu2p4_001476 | 2127 | 1280 | 1 . . . 252, 309 . . . 1280 |
| Aurpu2p4_001745 | 2128 | 417 | 1 . . . 184, 241 . . . 326, 382 . . . 417 |
| Aurpu2p4_001987 | 2129 | 2335 | 1 . . . 425, 481 . . . 2335 |
| Aurpu2p4_002339 | 2130 | 1860 | 1 . . . 264, 316 . . . 450, 512 . . . 891, 945 . . . 1860 |
| Aurpu2p4_002490 | 2131 | 2812 | 1 . . . 141, 190 . . . 591, 641 . . . 932, 984 . . . 2812 |
| Aurpu2p4_002528 | 2132 | 1532 | 1 . . . 364, 448 . . . 1532 |
| Aurpu2p4_003052 | 2133 | 1275 | 1 . . . 1275 |
| Aurpu2p4_003108 | 2134 | 2106 | 1 . . . 2106 |
| Aurpu2p4_003243 | 2135 | 7430 | 1 . . . 227, 286 . . . 538, 597 . . . 729, 796 . . . 1037, 1092 . . . 1305, |
| | | | 1362 . . . 1454, 1504 . . . 2180, 2235 . . . 2263, 3199 . . . 3725, |
| | | | 3786 . . . 3809, 3869 . . . 4355, 4434 . . . 4548, 4645 . . . 4686, |
| | | | 4741 . . . 4788, 4848 . . . 5045, 5099 . . . 5146, 5207 . . . 5272, |
| | | | 5447 . . . 5518, 5583 . . . 5624, 5690 . . . 5749, 5864 . . . 5889, |
| | | | 6063 . . . 6093, 6196 . . . 6286, 6343 . . . 6390, 6463 . . . 6492, |
| | | | 6559 . . . 7035, 7090 . . . 7430 |
| Aurpu2p4_003247 | 2136 | 2046 | 1 . . . 316, 365 . . . 2046 |
| Aurpu2p4_003704 | 2137 | 1243 | 1 . . . 226, 283 . . . 822, 885 . . . 1166, 1224 . . . 1243 |
| Aurpu2p4_004187 | 2138 | 2439 | 1 . . . 433, 485 . . . 1291, 1350 . . . 1656, 1713 . . . 2439 |
| Aurpu2p4_004476 | 2139 | 4947 | 1 . . . 189, 239 . . . 267, 320 . . . 570, 620 . . . 3295, 3350 . . . 4947 |
| Aurpu2p4_004865 | 2140 | 909 | 1 . . . 909 |
| Aurpu2p4_005304 | 2141 | 1089 | 1 . . . 1089 |
| Aurpu2p4_005861 | 2142 | 1571 | 1 . . . 279, 330 . . . 1571 |
| Aurpu2p4_005992 | 2143 | 1362 | 1 . . . 1263, 1318 . . . 1362 |
| Aurpu2p4_006091 | 2144 | 1466 | 1 . . . 246, 303 . . . 1466 |
| Aurpu2p4_006277 | 2145 | 1656 | 1 . . . 219, 274 . . . 1656 |
| Aurpu2p4_007520 | 2146 | 1230 | 1 . . . 115, 182 . . . 1001, 1065 . . . 1230 |
| Aurpu2p4_007546 | 2147 | 578 | 1 . . . 115, 166 . . . 578 |
| Aurpu2p4_007951 | 2148 | 2030 | 1 . . . 129, 185 . . . 205, 259 . . . 318, 382 . . . 722, 772 . . . 1063, 1116 . . . 2030 |
| Aurpu2p4_008628 | 2149 | 1632 | 1 . . . 1632 |
| Aurpu2p4_008719 | 2150 | 1026 | 1 . . . 124, 181 . . . 373, 440 . . . 759, 812 . . . 1026 |
| Aurpu2p4_009254 | 2151 | 1936 | 1 . . . 212, 264 . . . 1264, 1329 . . . 1936 |
| Aurpu2p4_009278 | 2152 | 1557 | 1 . . . 1557 |
| Aurpu2p4_009437 | 2153 | 2129 | 1 . . . 47, 100 . . . 838, 891 . . . 2129 |
| Aurpu2p4_009445 | 2154 | 1745 | 1 . . . 225, 278 . . . 559, 693 . . . 1745 |
| Aurpu2p4_010136 | 2155 | 1764 | 1 . . . 110, 165 . . . 1764 |
| Aurpu2p4_010244 | 2156 | 1860 | 1 . . . 1099, 1154 . . . 1310, 1368 . . . 1594, 1652 . . . 1860 |
| Aurpu2p4_010617 | 2157 | 1041 | 1 . . . 1041 |
| Aurpu2p4_010719 | 2158 | 1102 | 1 . . . 52, 111 . . . 563, 622 . . . 787, 991 . . . 1102 |
| Aurpu2p4_010798 | 2159 | 1726 | 1 . . . 240, 290 . . . 634, 683 . . . 1726 |
| Aurpu2p4_010869 | 2160 | 410 | 1 . . . 166, 228 . . . 313, 372 . . . 410 |

The present invention is illustrated in further details by the following non-limiting examples.

### EXAMPLES

### Example 1

### Fermentation of the Organism

### Materials & Methods

In general, for each species, starter mycelium was grown in rich medium (either mycological broth or yeast malt broth (the latter being indicated with YM)) and then washed with water. The starter was then used to inoculate different liquid media or solid substrate and the resulting mycelium was used for RNA extraction and library construction.

Following are the medium recipes and the solid substrates with a referenced source (if available) as well as a table (Table 3) listing the media variations, since in some cases the basic recipes of the referenced source have been altered depending on the species grown. This is then followed by a summary of the specific species as grown in the examples.

### A. Mycological Broth

Per liter: 10 g soytone, 40 g D-glucose, 1 mL Trace Element solution, Double-distilled water;
Adjust pH to 5.0 with hydrochloric acid (HCl) and bring volume to 1 L with double-distilled water.
Trace Element Solution contains 2 mM Iron(II) sulphate heptahydrate (FeSO₄.7H₂O), 1 mM Copper (II) sulphate pentahydrate (CuSO₄.5H₂O), 5 mM Zinc sulphate heptahydrate (ZnSO₄.7H₂O), 10 mM Manganese sulphate monohydrate (MnSO₄.H₂O), 5 mM Cobalt(II) chloride hexahydrate (CoCl₂.6H₂O), 0.5 mM Ammonium molybdate tetrahydrate ((NH₄)₆Mo₇O₂₄.4H₂O), and 95 mM Hydrochloric acid (HCl) dissolved in double-distilled water.

### B. Yeast-Malt Broth (YM)

(Reference: ATCC medium No. 200)
Per liter: 3 g yeast extract, 3 g malt extract, 5 g peptone, 10 g D-glucose, Double-distilled water to 1 L.

### C. Trametes Defined Medium (TDM)

(Reference: Reid and Piace, Effect of Residual lignin type and amount on biological bleaching of kraft pulp by
Trametes versicolor. Applied Environmental Microbiology 60: 1395-1400, 1994.)
Per liter: 10 g D-glucose, 0.75 g L-Asparagine monohydrate, 0.68 g Potassium phosphate monobasic (KH₂PO₄), 0.25 g Magnesium sulphate heptahydrate (MgSO₄.7H₂O), 15 mg Calcium chloride dihydrate (CaCl₂.2H₂O), 100 μg Thiamine hydrochloride, 1 ml Trace Element solution, 0.5 g Tween™ 80, Double distilled water; Adjust pH to 5.5 with 3 M potassium hydroxide and bring volume to 1 L with double-distilled water.

TABLE 3
Variations of TDM media used for library construction

| Varia- | |
|---|---|
| tion | Description |
| TDM-1 | Medium was prepared as in basic recipe described above. |
| TDM-2 | Quantity of asparagine monohydrate was reduced to 0.15 g. |
| TDM-3 | Manganese sulphate monohydrate was omitted from the |
| | medium. |
| TDM-4 | The quantity of manganese sulphate monohydrate was raised |
| | to 0.2 mM final concentration in the medium. |
| TDM-5 | The quantity of copper (II) sulphate pentahydrate was |
| | raised to 20 μM. |
| TDM-6 | Glucose was replaced with 10 g per liter of cellulose |
| | (Solka-Floc, 200FCC) |
| TDM-7 | Glucose was replaced with 10 g per liter of xylan from |
| | birchwood (Sigma Cat. # X-0502) |
| TDM-8 | Glucose was replaced with 10 g per liter of wheat bran¹. |
| TDM-9 | Glucose was replaced with 10 g per liter of citrus pectin |
| | (Sigma Cat. # P-9135). |
| TDM-10 | Tween ™ 80 was omitted from the medium. |
| TDM-11 | The double-distilled water was replaced with whitewater² |
| | collected from peroxide bleaching (which occurs during |
| | the manufacture of fine paper). |
| TDM-12 | The double-distilled water was replaced with whitewater² |
| | collected from newsprint manufacture. |
| TDM-13 | Glucose was replaced with 5 g per liter of ground |
| | hardwood kraft pulp³. |
| TDM-14 | The medium's pH was raised to 7.5. |
| TDM-15 | The strain was incubated at 5° C. above its optimum |
| | growth temperature. |
| TDM-16 | The strain was incubated at 10° C. below its optimum |
| | growth temperature. |
| TDM-17 | One half of the double-distilled water was replaced with |
| | whitewater from newsprint manufacture. Glucose was |
| | omitted. |
| TDM-18 | Potassium phosphate monobasic was replaced with 5 mM |
| | phytic acid from rice (Sigma Cat. # P3168). |
| TDM-19 | Asparagine monohydrate was increased to 4 g per liter. |
| TDM-20 | Asparagine monohydrate was increased to 4 g per liter |
| | and glucose was replaced with 2% fructose. |
| TDM-21 | Asparagine monohydrate was increased to 4 g per liter; |
| | 100 mL of double-distilled water was replaced |
| | with 100 mL kerosene⁴. Glucose was omitted. |
| TDM-22 | Asparagine monohydrate was increased to 4 g per liter; |
| | 100 mL of double-distilled water was replaced with 100 mL |
| | hexadecane (Sigma cat. # H0255). Glucose was omitted. |
| TDM-23 | Asparagine monohydrate was increased to 4 g per liter; |
| | one half of the double-distilled water was replaced with |
| | 25% whitewater from newsprint manufacture plus 25% white |
| | water from peroxide bleaching. Glucose was omitted. |
| TDM-24 | Asparagine monohydrate was increased to 4 g per liter |
| | and the quantity of manganese sulphate monohydrate was |
| | raised to 0.2 mM final concentration in the medium. |
| TDM-25 | Asparagine monohydrate was increased to 4 g per liter |
| | and manganese sulphate monohydrate was omitted from the |
| | medium. |
| TDM-26 | Asparagine monohydrate was increased to 4 g per liter; |
| | and potassium phosphate monobasic was replaced with 5 mM |
| | phytic acid from rice (Sigma Cat. # P3168). |
| TDM-27 | Glucose was replaced with 10 g per liter of olive oil |
| | (Sigma cat. # O1514) |
| TDM-28 | One half of the double-distilled water was replaced with |
| | whitewater from peroxide bleaching. Glucose was omitted. |
| TDM-29 | Glucose was replaced with 10 g per liter of tallow. |
| TDM-30 | Glucose was replaced with 10 g per liter of yellow |
| | grease. |
| TDM-31 | Glucose was replaced with 10 g per liter of defined |
| | lipid (Sigma cat. # L0288). |
| TDM-32 | Glucose was replaced with 50 g per liter of D-xylose. |
| TDM-33 | Glucose was replaced with 20 g per liter of glycerol and |
| | 20 ml per liter of ethanol. |
| TDM-34 | Glucose was reduced to 1 g per liter and 10 g per liter |
| | of bran was added. |
| TDM-35 | Glucose was reduced to 1 g per liter and 10 g per liter |
| | of pectin (Sigma Cat. # P-9135) was added. |
| TDM-36 | Glucose was replaced with 10 g per liter of biodiesel. |
| TDM-37 | Glucose was replaced with 10 g per liter of soy |
| | feedstock. |
| TDM-38 | Glucose was replaced with 10 g per liter of locust bean |
| | gum (Sigma cat # G0753). |
| TDM-39 | One half of double-distilled water was replaced with a |
| | 1:1 ratio of whitewater from newsprint manufacture and |
| | white water from peroxide bleaching. Glucose was |
| | omitted. |
| TDM-40 | The medium's pH was raised to 8.5. |
| TDM-41 | One half of double-distilled water was replaced with |
| | whitewater from peroxide bleaching; plus yeast extract |
| | was added to 1 g per liter. Glucose was omitted. |
| TDM-42 | Glucose was replaced with 5 g per liter of yellow grease |
| | and 5 g per liter of soy feedstock |
| TDM-43 | Glucose was replaced with 20 g per liter of fructose. |
| TDM-44 | Glucose was replaced with 10 g per liter of cellulose |
| | (Solka-Floc, 200FCC) plus 1 g per liter of sophorose. |
| TDM-45 | The medium's pH was raised to 8.84. |

¹Food grade wheat bran sourced from the supermarket was used.
²All Whitewaters were sourced from Quebec paper mills by PAPRICAN on the Applicant's behalf.
³Hardwood kraft pulp was sourced from Quebec paper mills by PAPRICAN on the Applicant's behalf.
⁴Kerosene was sourced from a general hardware store.

### D. Asparagine Salts Medium (AS):

(Reference: Ikeda et al., Laccase and Melanization in Clinically Important Cryptococcus Species Other Than Cryptococcus neoformans Journal of Clinical Microbiology 40: 1214-1218, 2002)
Per liter: 3.0 g D-glucose, 1.0 g L-Asparagine monohydrate, 3.0 g KH2PO4, 0.5 g Mg SO4.7H2O, 1 mg Thiamine.

TABLE 4
Variations of AS media used for library construction

| Varia- | |
|---|---|
| tion | Description |
| AS-1 | Medium was prepared as in basic recipe described above. |
| AS-2 | Glucose was replaced with 10 g per liter of pectin. |
| AS-3 | One half of double-distilled water was replaced with a |
| | 1:1 ratio of whitewater from newsprint manufacture and |
| | white water from peroxide bleaching. Glucose was omitted. |

### E. Solid Substrates Used:

SS-1 5 g Wheat Bran.

SS-2 5 g Wheat bran plus 5 mL defined lipid.

SS-3 5 g Oat bran (food grade, sourced from supermarket).

The Scytalidium thermophilum, Myriococcum thermophilum, and Aureobasidium pullulans strains were each grown according to the methods described above under the following growth conditions: TDM-1, -2, -3, -4, -5, -6, -7, -8, 9, -10, -13, -14, -15, -39; YM, whereby the following optimal growth temperature was used: 25° C.

The strains carrying the recombinant genes were grown according to the methods described above under the following growth conditions: minimal medium as described in Kafer et al., (1977, Adv. Genet. 19:33-131) except that the salt concentrations were raised ten-fold and the glucose concentration was 150 grams per liter, at 30° C.

### Example 2

### Genome Sequencing and Assembly

Genomic DNA was isolated from mycelium when the growth culture had reached the mid log phase. Genomic DNA was sequenced using the Roche 454 Titanium technology (http://www.454.com) to a genome coverage of over 20-fold according to the instructions of the manufacturer. The sequences were assembled using the Newbler and Celera assemblers (http://sourceforge.net/apps/mediawiki/wgs-assembler).

### Example 3

### Building the cDNA Libraries

Total RNA was isolated from fungal cells or mycelia when the growth cultures had reached the late log phase. The mycelia were collected by filtration through Miracloth and washed with water by filtration. The mycelia were padded dry using paper towels, and frozen in liquid nitrogen and stored at −80° C. To extract total RNA, the frozen mycelia or cells were ground to a fine powder in liquid nitrogen using pestle and mortar. Approximately 1-1.5 gram of frozen fungal powder was dissolved in 10 mL of TRIzol® reagent and RNA was extracted according to the manufacturer's protocol (Invitrogen Life Sciences, Cat. #15596-018). Following extraction, the RNA was dissolved at 1-1.5 mg/ml of DEPC-treated water.

The PolyATtract® mRNA Isolation Systems (Promega, Cat. #Z5300) was used to isolate poly(A)+RNA. In general, equal amounts of total RNA extracted from up to ten culture conditions were pooled. One milligram of total RNA was used for isolation of poly(A)+RNA according to the protocol provided by the manufacturer. The purified poly(A)+RNA was dissolved at 200-500 μg/mL of DEPC-treated water.
Five micrograms of poly(A)+RNA were used for the construction of cDNA library. Double-stranded cDNA was synthesized using the ZAP-cDNA® Synthesis Kit (Stratagene, Cat. #200400) according to the manufacturer's protocol with the following modifications. An anchored oligo(dT) linker-primer was used in the first-strand synthesis reaction to force the primer to anneal to the beginning of the poly(A) tail of the mRNA. The anchored oligo(dT) linker-primer has the sequence:

(SEQ ID NO: 2935)
5′-GAGAGAGAGAGAGAGAGAGAACTAGTCTCGAGTTTTTTTTTTTTTTT
TTTVN-3′

where V is A, C, or G and N is A, C, G, or T. A second modification was made by adding trehalose at a final concentration of 0.6 M and betaine at a final concentration of 2 M in the buffer of the first-strand synthesis reaction to promote full-length synthesis. Following synthesis and size fractionation, fractions of double-stranded cDNA with sizes longer than 600 by were pooled. The pooled cDNA was cloned directionally into the plasmid vector BlueScript KS+® (Stratagene) or a modified BlueScript KS+vector that contained Gateway® (Invitrogen) recombination sites. The cDNA library was transformed into E. coli strain XL10-Gold ultracompetent cells (Stratagene, Cat. #Z00315) for propagation.

Bacterial cells carrying cDNA clones were grown on LB agar containing the antibiotic ampicillin for selection of plasmid-borne bacteria and X-gal and IPTG to use the blue/white system to screen for the presence cDNA inserts. The white bacterial colonies, those carrying cDNA inserts, were transferred by a colony-picking robot to 384-well MTP for replication and storage. Clones that were to be analyzed by sequencing were transferred to 96-well deep blocks using liquid-handling robots. The bacteria were cultured at 37° C. with shaking at 150 rpm. After 24 hours of growth, plasmid DNA from the cDNA clones was prepared by alkaline lysis and sequenced from the 5′ end using ABI 3730×1 DNA analyzers (Applied Biosystems). The chromatograms obtained following single-pass sequencing of the cDNA clones were processed using Phred (available at http://www.phrap.org) to assign sequence quality values, Lucy as described in Chou and Holmes (2001, Bioinformatics, 17(12) 1093-1104) to remove vector and low quality sequences, and Phrap (available at http://www.phrap.org/) to assemble overlapping sequences derived from the same gene into contigs.

### Example 4

### Annotations

An in-house automated annotation pipeline was used to predict genes in the assembled genome sequence. The analysis pipeline used in part the ab initio tool Genemark® (http://exon.biology.gatech.edu/) for prediction. It also used the predictor Augustus (http://augustus.gobics.de/) trained on de novo assembled sequences and orthologous sequences for gene finding. Sequence similarity searches against the mycoCLAP® (http://cubique.fungalgenomics.ca/mycoCLAP/) and NCBI non-redundant databases were performed with BLASTX as described in Altschul et al., (1997) (Nucleic Acids Res. 25(17): 3389-3402). Proteins encoding biomass-degrading enzymes possess conserved domains. We used the domains available at the European Bioinformatics Institute (www.ebi.ac.uk/Tools/InterProScan/) to assist in the identification of target enzymes.

Proteins targeted to the extracellular space by the classical secretory pathway possess an N-terminal signal peptide, composed of a central hydrophobic core surrounded by N- and C-terminal hydrophilic regions. We used Phobius (available at http://phobius.cgb.ki.se) and SignalP® version 3 (available at http://www.cbs.dtu.dk/services/SignalP) to recognize the presence of signal peptides encoded by the cDNA clones. The tools TargetP® (available at http://www.cbs.dtu.dk/services/TargetP) and Big-PI Fungal Predictor (available at http://mendel.imp.ac.at/gpi/fungi_server.html) were used to remove sequences that encode proteins which are targeted to the mitochondria or bound to the cell wall. Finally, sequences predicted to encode soluble secreted proteins by these automated tools were analyzed manually. Clones that comprise full-length cDNAs which are predicted to encode soluble secreted proteins were sequenced completely. For genes identified from the genome sequence, oligonucleotide primers specific to the target genes were designed and used to PCR amplified the target genes from double-stranded cDNA or genomic DNA. The PCR amplified products were cloned into an appropriate expression vector for protein production in host cells. The genomic, coding and polypeptide sequences were assigned SEQ ID NOs, annotations, general functions, protein activities, CAZy family classifications, as summarized in Tables 1A-1C. Where appropriate, carbohydrate-binding modules (CBMs) of particular interest for the degradation of biomass were also listed in Tables 1A-1C.

### Example 5

### Assays for Characterization of Polypeptides

Polypeptides of the present invention may be additionally cloned into an expression vector, expressed and characterized (e.g., in sugar release assays) for activity relating to their ability to breakdown and/or process biomass as described in WO/2012/92676, WO/2012/130950, and WO/2012/130964 using appropriate substrates (e.g., acid pre-treated corn stover, hot water treated washed wheat straw, or hot water treated washed corn fiber substrate). Soluble sugars that are released can be analyzed for example by proton NMR.

A number of assays may be used to characterize the polypeptides of the present invention. Selected non-limiting examples of such assays are described and/or referenced below. Of course, other assays not explicitly mentioned or referenced here may also be used, and the expression “can be” used below is intended to reflect this possibility. Furthermore, a person of skill in the art would be able to modify or adapt these and other assays, as necessary, to characterize a particular polypeptide.
Acetylxylan esterase CE5. Polypeptides of the present invention having this activity can be characterized as described in Water et al., Appl Environ Microbiol. (2012), 78(10): 3759-62; or Yang et al., International Journal of Molecular Sciences (2010), 11(12): 5143-5151.
Adhesin protein Mad1. Polypeptides of the present invention having this activity can be characterized for example as described in Wang and St Leger, Eukaryot. Cell (2007), 6(5): 808-816.
Adhesin. Polypeptides of the present invention having this activity (reviewed in Dranginis et al., Microbiology and Molecular Biology Reviews (2007), 71(2): 282-294) can be characterized using techniques well known in the art (e.g. adhesion assays).
Aldose 1-epimerase (mutarotase, aldose mutarotase). Polypeptides of the present invention having this activity can be characterized as described in Timson and Reece, FEBS Letters (2003), 543(1-3):21-24; and Villalobo et al., Exp. Parasitol. (2005) 110(3): 298-302.
Allergen Asp f 15. Polypeptides of the present invention having this activity can be characterized as described in Bowyer et al., Medical Mycology (2007), 45(1): 17-26.
Alpha-arabinofuranosidase. Polypeptides of the present invention having this activity can be characterized for example as described by Poutanen et al. (Appl. Microbiol. Biotechnol. 1988, 28, 425-432) using 5 mM p-nitrophenyl alpha-L-arabinofuranoside as substrates. The reactions may be carried out in 50 mM citrate buffer at pH 6.0, 40° C. with a total reaction time of 30 min. The reaction is stopped by adding 0.5 ml of 1 M sodium carbonate and the liberated p-nitrophenol is measured at 405 nm. Activity is expressed in U/ml. Furthermore, arabionofuranosidases may also be useful in animal feed compositions to increase digestibility. Corn arabinoxylan is heavily di-substituted with arabinose. In order to facilitate the xylan degradation it is advantageous to remove as many as possible of the arabinose substituents. The in vitro degradation of arabinoxylans in a corn based diet supplemented with a polypeptide of the present invention having alpha-arabinofuranosidase activity and a commercial xylanase is studied in an in vitro digestion system, as described in WO/2006/114094.
Alpha-fucosidase. Polypeptides of the present invention having this activity can be characterized for example as described in U.S. Pat. No. 5,637,490; in Zielke et al., J. Lab. Clin. Med. (1972), 79:164; or using commercially available kits (e.g., Alpha-L-Fucosidase (AFU) Assay Kit, Cat. No. BQ082A-EALD, BioSupplyUK).
Alpha-galactosidase. Polypeptides of the present invention having this activity can be characterized for example as described in US patent application publication No. US 2010/0273235 A1. Briefly, a synthetic substrate, 4-Nitrophenyl-α-D-galactoside is used and the release of p-Nitro-phenol is followed at a wavelength of 405 nm in a reaction buffer containing 100 mM sodium phosphate, 50 mM sodium chloride, pH 6.8 at 26° C.
Alpha-glucuronidase GH67. Polypeptides of the present invention having this activity can be characterized for example as described in Lee et al., J Ind Microbiol Biotechnol. (2012), 39(8): 1245-51, or Nagy et al., J. Bacteriol. (2002), 184: 4925-4929.
Aminopeptidase Y. Polypeptides of the present invention having this activity can be characterized for example as described in Yasuhara et al., J. Biol. Chem. (1994) 269(18): 13644-50.
Arabinogalactanase. Polypeptides of the present invention having this activity can be characterized for example as described in Yamamoto and Emi, Methods in Enzymology (1988), 160: 719-725.
Arabinoxylan arabinofuranohydrolase (AXH) GH43. Polypeptides of the present invention having this activity can be characterized for example as described in Yoshida et al., Journal of Bacteriology (2010), 192(20): 5424-5436.
Arabinoxylan arabinofuranosidase GH62. Polypeptides of the present invention having this activity can be characterized for example as described in Sakamoto et al., Applied Microbiology and Biotechnology (2011), 90(1): 137-146.
Aspartic protease. Polypeptides of the present invention having this activity can be characterized for example as described in Tacco et al., Med. Mycol. (2009), 47(8): 845-854; or in Hu et al., Journal of Biomedicine and Biotechnology (2012), 2012:728975.
Aspartic-type endopeptidase. Polypeptides of the present invention having this activity can be characterized for example as described in Tjalsma et al., J. Biol. Chem. (1999), 274: 28191-28197.
Aspergillopepsin-2. Polypeptides of the present invention having this activity can be characterized for example as described in Huang et al., Journal of Biological Chemistry (2000), 275(34): 26607-14.
Avenacinase. Polypeptides of the present invention having this activity can be characterized for example as described in Kwak et al., Phytopathology (2010), 100(5): 404-14; or in Bowyer et al., Science (1995), 267(5196): 371-4.
Beta-galactosidase. Polypeptides of the present invention having this activity can be characterized for example using commercially available kits (e.g., β-Galactosidase Enzyme Assay System with Reporter Lysis Buffer, Cat. No. E2000, Promega).
Beta-glucanase. Polypeptides of the present invention having this activity can be characterized for example as described in US patent application publication number US 2012/0023626 A1; or in U.S. Pat. No. 8,309,338.
Beta-glucosidase. Polypeptides of the present invention having this activity can be characterized for example as described in PCT application publication No. WO/2007/019442; or by using a commercially available kit (e.g., Beta-Glucosidase Assay Kit, Cat. No. KA1611, Abnova Corp).
Beta-glucuronidase GH79. Polypeptides of the present invention having this activity can be characterized for example as described in Eudes et al., Plant Cell Physiology (2008), 49(9): 1331-41; or Michikawa et al., Journal of Biological Chemistry (2012), 287: 14069-14077.
Beta-mannanase. Polypeptides of the present invention having this activity can be characterized for example as described in European patent application No. EP 2261359 A1; or in PCT application publication No. WO2008009673A2.
Beta-mannosidase. Polypeptides of the present invention having this activity can be characterized for example as described in Park et al., N. Biotechnol. (2011), 28(6): 639-48; Duffaud et al., Appl Environ Microbiol. (1997), 63(1): 169-77; or in Fliedrová et al., Protein Expr Purif. (2012), 85(2): 159-64.
Beta-xylosidase. Polypeptides of the present invention having this activity can be characterized for example as described in Wagschal et al., Applied and Environmental Microbiology (2005), 71(9): 5318-5323; or Shao et al., Appl Environ Microbiol. (2011), 77(3): 719-726.
Bifunctional xylanase/deacetylase. Polypeptides of the present invention having this activity can be characterized for example as described in Cepeljnik et al., Folia Microbiol. (2006), 51(4): 263-267; US patent application publication No. US 2012/0028306 A1; U.S. Pat. No. 7,759,102; or PCT application publication No. WO 2006/078256 A2; or Grozinger and Schreiber, Chem Biol. (2002), 9(1): 3-16.
Carbohydrate-binding cytochrome. Polypeptides of the present invention having this activity can be characterized for example as described in Yoshida et al., Appl Environ Microbiol. (2005) 71(8): 4548-4555.
Carboxypeptidase. Polypeptides of the present invention having this activity can be characterized for example as described in US patent application publication No. US 2007/0160711 A1; or in PCT application publication No. WO 1998/014599A1.
Cellobiohydrolase GH6. Polypeptides of the present invention having this activity can be characterized for example as described in Takahashi et al., Applied and Environmental Microbiology (2010), 76(19): 6583-6590.
Cellobiohydrolase GH7. Polypeptides of the present invention having this activity can be characterized for example as described in Segato et al., Biotechnology for Biofuels (2012), 5:21; or Baumann et al., Biotechnol. for Biofuels (2011), 4:45.
Cellobiose dehydrogenase. Polypeptides of the present invention having this activity can be characterized for example as described in Schou et al., Biochem. J. (1998), 330: 565-571; or Baminger et al., J. Microbiol Methods. (1999), 35(3): 253-9.
Chitin deacetylase. Polypeptides of the present invention having this activity can be characterized for example as described in European patent application No. EP 0610320 B1.
Chitinase. Polypeptides of the present invention having this activity can be characterized for example as described in U.S. Pat. No. 7,087,810.
Chitooligosaccharide deacetylase. Polypeptides of the present invention having this activity can be characterized for example as described in John et al., Proc Natl Acad Sci USA (1993), 90(2): 625-9.
Chitotriosidase-1. Polypeptides of the present invention having this activity can be characterized for example as described in U.S. Pat. No. 6,057,142.
Cholinesterase. Polypeptides of the present invention having this activity can be characterized for example as described in Abass Askar et al., Canadian Journal Veterinary Research (2011), 75(4): 261-270; or Cátia et al., PLoS One (2012), 7(3): e33975.
Cutinase. Polypeptides of the present invention having this activity can be characterized for example as described in US patent application publication No. US 2012/0028318 A1; or in Chen et al., J. Biol Chem. (2008), 283(38): 25854-62.
Cytochrome P450. Polypeptides of the present invention having this activity can be characterized for example as using commercially available kits (e.g., P450-Glo™ Assays, Promega); or as described in Walsky and Obach, Drug Metab Dispos. (2004), 32(6): 647-60.
Dehydrogenase. Polypeptides of the present invention having this activity can be characterized for example as described in Mayer and Arnold, J. Biomol. Screen. (2002), 7(2): 135-140.
Endo-1,3(4)-beta-glucanase (laminarinase). Polypeptides of the present invention having this activity can be characterized for example as described in Akiyama et al., J Plant Physiol. (2009), 166(16): 1814-25; or Hua et al., Biosci Biotechnol Biochem. (2011), 75(9): 1807-12.
Endo-1,4-beta-xylanase. Polypeptides of the present invention having this activity can be characterized for example as described in Song et al., Enzyme and Microbial Technology (2013). 52(3): 170-176.
Endo-1,5-alpha-arabinanase. Polypeptides of the present invention having this activity can be characterized for example as described in US patent publication No. US 2012/0270263. More particularly, this assay of arabinase activity is based on colorimetrically determination by measuring the resulting increase in reducing groups using a 3,5-dinitrosalicylic acid reagent. Enzyme activity can be calculated from the relationship between the concentration of reducing groups, as arabinose equivalents, and absorbance at 540 nm. The assay is generally carried out at pH 3.5, but it can be performed at different pH values for the additional characterization and specification of enzymes. Polypeptides of the present invention having this activity can also be characterized for example as described in Hong et al., Biotechnol Lett. (2009), 31(9): 1439-43.
Endo-1,6-beta-glucanase. Polypeptides of the present invention having this activity can be characterized for example as described in Bryant et al., Fungal Genet Biol. (2007), 44(8): 808-17; or in Oyama et al., Biosci Biotechnol Biochem. (2006), 70(7): 1773-5.
Endochitinase. Polypeptides of the present invention having this activity can be characterized for example as described in Wen et al., Biotechnol. Applied Biochem. (2002), 35: 213-219.
Endoglucanase. Polypeptides of the present invention having this activity can be characterized for example as described in U.S. Pat. No. 8,063,267.
Endoglycoceramidase. Polypeptides of the present invention having this activity can be characterized for example as described in U.S. Pat. No. 5,795,765; or US patent application publication No. US 2009/0170155 A1.
Endo-polygalacturonase. Polypeptides of the present invention having this activity can be characterized for example as described in European patent application publication Nos. EP1614748 A1 and EP1114165 A1.
Endo-polygalacturonase. Polypeptides of the present invention having this activity can be characterized for example as described in PCT application publication No. WO 1994/014952 A1; or in European patent application publication No. EP1614748 A1.
Endo-rhamnogalacturonase GH28. Polypeptides of the present invention having this activity can be characterized for example as described in Sprockett et al., Gene (2011), 479(1-2): 29-36; or An et al., Carbohydrate Research (1994), 264(1): 83-96.
Exo-1,3-beta-galactanase GH43. Polypeptides of the present invention having this activity can be characterized for example as described in Ichinose et al., Appl Environ Microbiol. (2006), 72(5): 3515-3523.
Exo-1,3-beta-glucanase. Polypeptides of the present invention having this activity can be characterized for example as described in O'Connell et al., Appl Microbiol Biotechnol. (2011), 89(3): 685-96; or Santos et al., J Bacteria (1979), 139(2): 333-338.
Exo-1,4-beta-xylosidase. Polypeptides of the present invention having this activity can be characterized for example as described in La Grange et al., Applied and Environmental Microbiology (2001), 67(12): 5512-5519.
Exo-arabinanase. Polypeptides of the present invention having this activity can be characterized for example as described in Tatsuji Sakamoto and Thibault, Appl Environ Microbiol. (2001), 67(7): 3319-3321.
Exoglucanase. Polypeptides of the present invention having this activity can be characterized for example as described in Creuzet et al., FEMS Microbiology Letters (1983), 20(3): 347-350; or Kruus et al., Journal of Bacteriology (1995), 177(6): 1641-1644.
Exo-glucosaminidase GH2. Polypeptides of the present invention having this activity can be characterized for example as described in Tanaka et al., Journal of Bacteriology (2003), 185(17): 5175-5181.
Exo-polygalacturonase. Polypeptides of the present invention having this activity can be characterized for example as described in Dong and Wang, BMC Biochem. (2011), 12: 51.
Exo-rhamnogalacturonase GH28. Polypeptides of the present invention having this activity can be characterized for example as described in U.S. Pat. No. 5,811,291.
Expansin. Polypeptides of the present invention having this activity can be characterized for example as described in PCT application publication No. WO 2005/030965 A2; or in U.S. Pat. No. 7,001,743.
Expansin-like protein 1. Polypeptides of the present invention having this activity can be characterized for example as described in Lee et al., Molecules and Cells (2010), 29(4): 379-85.
Feruloyl esterase. Polypeptides of the present invention having this activity can be characterized for example as described in PCT application publication No. WO 2009/076122 A1.
Galactanase GH5. Polypeptides of the present invention having this activity can be characterized for example as described in Ichinose et al., Applied and Environmental Microbiology (2008), 74(8): 2379-2383.
Gamma-glutamyltranspeptidase 2. Polypeptides of the present invention having this activity can be characterized for example as described in Rossi et al., PLoS One (2012), 7(2): e30543.
Glucan 1,3-beta-glucosidase. Polypeptides of the present invention having this activity can be characterized for example as described in Boonvitthya et al., Biotechnol Lett (2012), 34(10): 1937-43.
Glycosidase. Polypeptides of the present invention having this activity can be characterized for example as described in U.S. Pat. No. 8,119,383.
Hephaestin-like protein 1. Polypeptides of the present invention having this activity can be characterized for example as described for oxioreductases.
Hexosaminidase. Polypeptides of the present invention having this activity can be characterized for example as described in Wendeler and Sandhoff, Glycoconj J. (2009), 26(8):945-952.
Hydrophobin. Polypeptides of the present invention having this activity can be characterized for example as described in Bettini et al., Canadian Journal of Microbiology (2012), 58(8): 965-972; or Niu et al., Amino Acids. (2012), 43(2):763-71.
Iron transport multicopper oxidase FET3. Polypeptides of the present invention having this activity can be characterized for example as described in Askwith et al., Cell (1994), 76: 403-10; or De Silva et al., J. Biol. Chem. (1995) 270: 1098-1101.
Laccase. Polypeptides of the present invention having this activity can be characterized for example as described in Dedeyan et al., Appl Environ Microbiol. (2000), 66(3): 925-929.
Laminarinase GH55. Polypeptides of the present invention having this activity can be characterized for example as described in Ishida et al., J Biol Chem. (2009), 284(15): 10100-10109; or Kawai et al., Biotechnol Lett. (2006), 28(6): 365-71.
L-Ascorbate oxidase. Polypeptides of the present invention having this activity can be characterized for example as described in U.S. Pat. Nos. 5,612,208 and 5,180,672.
L-carnitine dehydrogenase. Polypeptides of the present invention having this activity can be characterized for example as described in Aurich et al., Biochim Biophys Acta. (1967), 139(2): 505-7; or U.S. Pat. No. 5,156,966.
Leucine aminopeptidase 1. Polypeptides of the present invention having this activity can be characterized for example as described in Beattie et al., Biochem. J. (1987), 242: 281-283.
Licheninase (beta-D-glucan 4-glucanohydrolase). Polypeptides of the present invention having this activity can be characterized for example as described in Tang et al., J Agric Food Chem. (2012), 60(9): 2354-61.
Lipase. Polypeptides of the present invention having this activity can be characterized for example as described in U.S. Pat. Nos. 7,662,602 and 7,893,232.
L-sorbosone dehydrogenase. Polypeptides of the present invention having this activity can be characterized for example as described in Shinjoh et al., Applied and Environment Microbiology (1995), 61(2): 413-420.
Lysophospholipase. Polypeptides of the present invention having this activity can be characterized for example as described in U.S. Pat. No. 5,965,422.
Metallocarboxypeptidase. Polypeptides of the present invention having this activity can be characterized for example as described in Tayyab et al., J Biosci Bioeng. (2011), 111(3): 259-65; or Song et al., J Biol Chem. (1997), 272(16): 10543-50.
Methylenetetrahydrofolate dehydrogenase [NAD(+)]. Polypeptides of the present invention having this activity can be characterized for example as described in Wohlfarth et al., J Bacteria (1991), 173(4): 1414-1419.
Mixed-link glucanase. Polypeptides of the present invention having this activity can be characterized for example as described in Clark et al., Carbohydr Res. (1978), 61: 457-477.
Multicopper oxidase. Polypeptides of the present invention having this activity can be characterized for example as described in US patent application publication No. US 2012/0094335 A1.
Mutanase. Polypeptides of the present invention having this activity can be characterized for example as described in Pleszczyńska, Biotechnol Lett. (2010), 32(11): 1699-1704; or WO 1998/000528 A1.
N-acetylglucosaminidase GH18. Polypeptides of the present invention having this activity can be characterized for example as described in Murakami et al., Glycobiology (2013), e-pub: February 22, PMID: 23436287; or in US patent application publication No. US20120258089 A1.
NADPH—cytochrome P450 reductase. Polypeptides of the present invention having this activity can be characterized for example as described in Guengerich et al., Nat Protoc. (2009), 4(9): 1245-51.
Non-hemolytic phospholipase C. Polypeptides of the present invention having this activity can be characterized for example as described in Weingart and Hooke, Curr Microbiol. (1999), 38(4): 233-8; Korbsrisate et al., J Clin Microbiol. (1999), 37(11): 3742-5.
Oxidase. Polypeptides of the present invention having this activity can be characterized for example using a number of commercially available kits [e.g., Amplex® Red Galactose/Galactose Oxidase Kit (A22179) and Amplex® Red Glucose/Glucose Oxidase Assay Kit (Molecular Probes/Invitrogen); Cytochrome C Oxidase Assay Kit (Cat. No. CYTOCOX1-1KT; Sigma-Aldrich); Xanthine Oxidase Assay Kit (ab102522, Abcam); Lysyl Oxidase Activity Assay Kit (ab112139, Abcam); Glucose Oxidase Assay Kit (ab138884, Abcam); Monoamine oxidase B (MAOB) Specific Activity Assay Kit (ab109912, Abcam)].
Oxidoreductase. Polypeptides of the present invention having this activity can be characterized for example as described in Hommes et al., Anal Chem. (2013), 85(1): 283-291.
Para-nitrobenzyl esterase. Polypeptides of the present invention having this activity can be characterized for example as described in Moore and Arnold, Nat Biotechnol. (1996), 14(4): 458-67.
Pectate lyase. Polypeptides of the present invention having this activity can be characterized for example as described in Wang et al., BMC Biotechnology (2011), 11: 32.
Pectin methylesterase. Polypeptides of the present invention having this activity can be characterized for example as described in PCT application publication No. WO 1997/031102 A1.
Pectinesterase. Polypeptides of the present invention having this activity can be characterized for example as described in U.S. Pat. No. 5,053,232.
Penicillopepsin. Polypeptides of the present invention having this activity can be characterized for example as described in Cao et al., Protein Sci. (2000), 9(5): 991-1001; or Hofmann et al., Biochemistry. (1984), 14; 23(4): 635-43.
Peroxidase. Polypeptides of the present invention having this activity can be characterized for example using a number of commercially available kits [e.g., Amplex® Red Hydrogen Peroxide/Peroxidase Assay Kit (Molecular Probes/Invitrogen); Peroxidase Activity Assay Kit (Cat. No. K772-100; BioVision); QuantiChrom™ Peroxidase Assay Kit (Cat. No. DPOD-100, BioAssay Systems].
Phospholipase C. Polypeptides of the present invention having this activity can be characterized for example using commercially available kits (Amplex® Red Phosphatidylcholine-Specific Phospholipase C Assay Kit, Molecular Probes/Invitrogen).
Polysaccharide monooxygenase. Polypeptides of the present invention having this activity can be characterized for example as described in Kittl et al., Biotechnol Biofuels. (2012), 5(1):79, Phillips et al., ACS Chem Biol (2011), 6(12): 1399-1406, Wu et al., J. Biol. Chem (2013), 288(18): 12828-39.
Polysaccharide monooxygenases, sometimes referred to functionally as “cellulase-enhancing proteins”, generally belong the enzyme class GH61 and are reported to cleave polysaccharides with the insertion of oxygen.
Protease. Polypeptides of the present invention having this activity can be characterized for example as described in US patent application publication No. US 2005/0010037 A1.
Putative exoglucanase type C (1,4-beta-cellobiohydrolase; beta-glucancellobiohydrolase; exocellobiohydrolase I). Polypeptides of the present invention having this activity can be characterized for example as described in Dai et al., Applied Biochemistry and Biotechnology (1999), 79, Issue 1-3: 689-699.
Rhamnogalacturonan lyase PL4. Polypeptides of the present invention having this activity can be characterized for example as described in Mutter et al., Plant Physiol. (1998), 117: 153-163; or de Vries, Appl. Microbiol Biotechnol. (2003), 61: 10-20.
Rodlet protein. Polypeptides of the present invention having this activity can be characterized for example as described in Yang et al., Biopolymers (2013), 99(1): 84-94.
Serine-type carboxypeptidase F. Polypeptides of the present invention having this activity can be characterized for example as described in U.S. Pat. No. 6,379,913.
Swollenin. Polypeptides of the present invention having this activity can be characterized for example as described in Jäger et al., Biotechnol Biofuels. (2011), 4: 33; or Saloheimo et al., Eur J Biochem. (2002), 269(17): 4202-11.
Tyrosinase. Polypeptides of the present invention having this activity can be characterized for example as described in US patent application publication No. US 2011/0311693 A1.
Unsaturated rhamnogalacturonyl hydrolase YteR. Polypeptides of the present invention having this activity can be characterized for example as described in Itoh et al., Biochem Biophys Res Commun. (2006), 347(4): 1021-9; or Itoh et al., J Mol Biol. (2006), 360(3): 573-85.
Xylan alpha-1,2-glucuronidase. Polypeptides of the present invention having this activity can be characterized for example as described in Ishihara, M. and Shimizu, K., “alpha-(1->2)-Glucuronidase in the enzymatic saccharification of hardwood xylan: Screening of alpha-glucuronidase producing fungi.” Journal Mokuzai Gakkaishi, (1988) 34: 58-64.
Xylanase. Polypeptides of the present invention having this activity can be characterized for example as described in US patent application publication No. US 2012/0028306 A1; U.S. Pat. No. 7,759,102; or PCT application publication No. WO 2006/078256 A2.
Xyloglucanase GH12. Polypeptides of the present invention having this activity can be characterized for example as described in Master et al., Biochem. (2008), 411(1): 161-170.
Xyloglucan-specific endo-beta-1,4-glucanase A. Polypeptides of the present invention having this activity can be characterized for example as described in European patent application publication No. EP0972016 B1; in U.S. Pat. No. 6,077,702; Damásio et al., Biochim Biophys Acta. (2012), 1824(3): 461-7; or Wong et al., Appl Microbiol Biotechnol. (2010), 86(5): 1463-71.
Xylosidase/arabinosidase. Polypeptides of the present invention having this activity can be characterized for example as described in Whitehead and Cotta, Curr Microbiol. (2001), 43(4): 293-8; or Xiong et al., Journal of Experimental Botany (2007), 58(11): 2799-2810.

### Example 6

### General Molecular Biology Procedures

Standard molecular cloning techniques such as DNA isolation, gel electrophoresis, enzymatic restriction modifications of nucleic acids, E. coli transformation, etc., were performed as described by Sambrook et al., 1989, (Molecular cloning: a laboratory manual, 2nd Ed., Cold Spring Harbor Laboratory Press, Cold Spring Harbor, N.Y. and Innes et al. (1990) PCR protocols, a guide to methods and applications, Academic Press, San Diego, edited by Michael A. Innis et al). Primers were prepared by IDT (Integrated DNA Technologies). Sanger DNA sequencing was performed using an Applied Biosystem's 3730×1 DNA Analyzer technology at the Innovation Centre (Génome Québec), McGill University in Montreal.

### Example 7

### Construction of pGBFIN49 Expression Plasmids

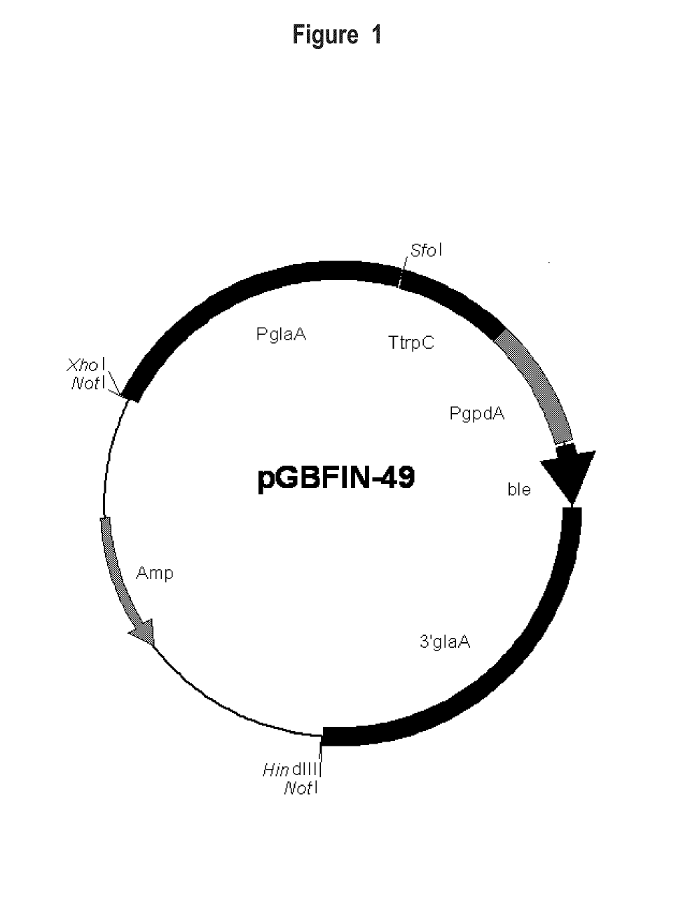

Genes of interest were cloned into the expression vector pGBFIN-49. This vector is a derivative of pGBFIN-41 that contains the A. niger glaA promoter, A. niger TrpC terminator, A. nidulans gpdA promoter, gene encoding the pheomycin resistance gene, A. niger glaA terminator and an E. coli backbone. FIG. 1 represents a schematic map of pGBFIN-49 and the complete nucleotide sequence is presented as SEQ ID NO: 2936. Details of the construction of pGBFIN-49 are as follows:

### 1. TtrpC Terminator PCR Amplification (0.7 kb):

TtrpC terminator was PCR amplified using purified pGBFIN33 plasmid as a template. The following primers and PCR program were used:

(SEQ ID NO: 2937)
Primer-3: 5′-GTCCGTCGCCGTCCTTCAccgccggtccgacg-3′
(SEQ ID NO: 2938)
Primer-4: 5′-GCGGCCGGCGTATTGGGTGttacggagc-3′

Primer-4 is entirely specific to the TtrpC 3′ end. Primer-3 was designed to suit the LIC cloning strategy but also to keep the TtrpC sequence as close to the original sequence. To do so, five adenines were replaced by thymines (underlined).

PCR Master Mix:

| | | |
|---|---|---|
| pGBFIN33 | 1 | μL (5-10 ng) |
| Primer-3 (10 mM) | 1 | μL |
| Primer-4 (10 mM) | 1 | μL |
| dNTPs (2 mM) | 5 | μL |
| HF Buffer (5x) | 10 | μL |
| Phusion DNS pol. | 0.5 | μL |
| Nuclease-free water | 31.5 | μL |
| Total | 50 | μL |

PCR Program:

1×98° C., 2 min; 25×(98° C., 30 sec; 68° C., 30 sec; 72° C., 1 min); 72° C., 7 min.

Reaction conditions: 5 μL of the PCR reaction was separated by electrophoresis on 1.0% agarose gel and the remaining was purified using QIAEX II™ gel Extraction kit (QIAGEN) and resuspended in nuclease-free water.

2. pGBFIN41 Vector PCR Amplification (8.3 kb):

Vector backbone was PCR amplified using pGBFIN41 as a template. Primers were designed outside of the ccdA region (not included in pGBFIN49). The following primers and PCR program were used:

(SEQ ID NO: 2939)
Primer-2: 5′-CACCCAATACGCCGGCCGCgcttccagacagctc-3′
(SEQ ID NO: 2940)
Primer-1C: 5′-GGTGTTTTGTTGCTGGGGAtgaagctcaggctctca
gttgcgtc-3′

Primer-2 contains a pgpdA-specific region and an extra sequence specific to TtrpC 3′ end (also included in Primer-4). Primer-1C was designed to suit the LIC cloning strategy but also to keep PgalA region as close to the original sequence. To do so, three thymines were replaced by adenines (underlined).

PCR Master Mix:

| | | |
|---|---|---|
| pGBFIN41 | 1 | μL (50 ng) |
| Primer-2 (10 mM) | 1 | μL |
| Primer-1C (10 mM) | 1 | μL |
| dNTPs (2 mM) | 5 | μL |
| HF Buffer (5x) | 10 | μL |
| Phusion DNS pol. | 0.5 | μL |
| DMSO | 1 | μL |
| Nuclease-free water | 30.5 | μL |
| Total | 50 | μL |

PCR Program:

1×98° C., 3 min; 10×(98° C., 30 sec; 68° C., 30 sec, 72° C., 5 min); 20×(98° C., 30 sec, 68° C., 30 sec, 72° C., 5 min+10 sec/cycle); 72° C., 10 min.

Reaction Conditions:

5 μL of the PCR reaction was separated on a 0.5% agarose gel and remaining was purified using QIAEX II™ gel Extraction kit (QIAGEN) and resuspended in nuclease-free water.

3. pGBFIN41+TtrpC Overlap-Extension PCR:

Overlap-extension/Long range PCR was performed to: a) fuse the two PCR pieces together; b) add an SfoI restriction site to re-circularize the vector. No primers were used in the overlap-extension stage. Primer-11 and Primer-12 were used for the long range PCR reaction.

(SEQ ID NO: 78)
Primer-11: 5′-CACCGGCGCCGTCCGTCGCCGTCCTTC-3′
(SEQ ID NO: 79)
Primer-12: 5′-ACGGCGCCGGTGTTTTGTTGCTGGGGATG-3′

Primer-11 is specific to the LIC tag located on the TtrpC terminator, while Primer-12 is specific to the LIC tag located on the PglaA region. The SfoI restriction site sequence is underlined above.

A standard PCR master mix was prepared to perform overlap-extension PCR using pGBFIN41 and TtrpC purified PCR products as templates. No primers were added.

Overlap-Extension Master Mix:

| | |
|---|---|
| TtrpC | 1 μL |
| pGBFIN41 | 9 μL |
| Buffer GC (5x) | 10 μL |
| dNTPs (2 mM) | 5 μL |
| Phusion DNA pol. | 0.5 μL |
| Nuclase-free water | 24.5 μL |
| pGBFIN41 | 50 μL |

PCR Program—Overlap (No Primers):

1×98° C., 2 min; 5×(98° C., 15 sec; 58° C., 30 sec; 72° C., 5 min), 5×(98° C., 15 sec; 63° C., 30 sec; 72° C., 5 min), 5×(98° C., 15 sec; 68° C., 30 sec; 72° C., 5 min); 72° C., 10 min.

The overlap-extension PCR product was then, purified on QIAEX II™ column and 5 μL of the purified reaction was used as template DNA for Long range PCR step with Primers-11 and -12.

PCR Master Mix:

| | |
|---|---|
| Overlap product | 5 μL |
| Primer-11 (10 mM) | 1 μL |
| Primer-12 (10 mM) | 1 μL |
| dNTPs (2 mM) | 5 μL |
| HF Buffer (5x) | 10 μL |
| Phusion DNA pol. | 0.5 μL |
| DMSO | 1 μL |
| Nuclease-free water | 26.5 μL |
| pGBFIN41 | 50 μL |

PCR Program—Long Range:

1×98° C., 3 min; 10×(98° C., 30 sec; 68° C., 30 sec; 72° C., 5 min); 20×(98° C., 30 sec; 68° C., 30 sec; 72° C., 5 min+10 sec/cycle); 72° C., 10 min.

Reaction Conditions:

5 μL of the PCR reaction was separated on 0.5% agarose gel and remaining was purified using QIAEX II™ gel Extraction kit and resuspended in nuclease-free water. Then, SfoI digestion was performed and digested product was purified using QIAEX II gel extraction kit follow the procedure as described by the manufacturer.

### 4. Ligation:

100 ng of the purified digested fragment was ligated to itself using 1 μL of T4 DNA Ligase (New England Biolabs, M0202), and incubated at 16° C. overnight. Enzyme inactivation was performed at 65° C. for 10 minutes. Then, 10 μL of ligation product was transformed in DH5 E. coli competent cells and plated on 2xYT agar containing 100 ug/mL ampicillin. DNA extraction was performed on single colonies the next day. Restriction analysis and sequencing were done to confirm the structure.

### Example 8

### Cloning of Scytalidium thermophilum, Myriococcum thermophilum, and Aureobasidium pullulans genes in E. coli

Cloning genes of interest in the pGBFIN-49 expression vector was performed using the Ligation-independent cloning (LIC) method according to Aslanidis, C., de Jong, P. (1990) Nucleic Acids Research Vol. 18 No. 20, 6069-6074.

Coding sequences from genes of interest were amplified by PCR using primers containing LIC tags, which are homologous to Pgla and TrpC sequences in the pGBFIN-49 cloning vector fused to sequences homologous to the coding sequences of the gene of interest, and either genomic DNA or cDNA as template. Primers have the following sequences:

(SEQ ID NO: 2941)
Forward primer: 5′-CCCCAGCAACAAAACACCTCAGCAATG...
15-20 nucleotides specific to each gene to be
cloned
(SEQ ID NO: 2942)
Reverse primer: 5′-GAAGGACGGCGACGGACTTCA...15-20
nucleotides specific to each gene to be cloned

PCR Mix Consists of Following Components:

| | |
|---|---|
| Template (gDNA or cDNA) 1-10 ng/μL | 1 μL |
| 5X Phusion HF Buffer (Finnzymes ™) | 10 μL |
| 2 mM dNTPs | 5 μL |
| LIC primer (F + R) mix 10 mM | 0.5 μL |
| Phusion DNA Polymerase (Finnzymes ™) | 0.5 μL |
| DMSO | 1.5 μL |
| H₂O | 31.5 μL |
| TOTAL | 50 μL |

PCR Amplification was Carried Out with Following Conditions:

3-step protocol

| Cycle step | Temp | Time | | Cycles |
|---|---|---|---|---|
| Initial denaturation | 98° C. | 30 | s | 1 |
| Denaturation | 98° C. | 10 | s | 10 |
| Annealing | 58° C. | 30 | s | |
| Extension | 72° C. | 30 | s | |
| Denaturation | 98° C. | 10 | s | 20 |
| Annealing | 68° C. | 30 | s | |
| Extension | 72° C. | 30 | s | |
| Final extension | 70° C. | 10 | min | 1 |

| | | | |
|---|---|---|---|
| End of PCR storage | 4° C. | hold | 1 |

Following PCR, 90 μL milliQ™ water was added to each sample and the mix was purified using a MultiScreen PCR₉₆ Filter Plate (Millipore) according to manufacturer's instructions. The PCR product was eluted from the filter in 25 μL 10 mM Tris-HCl pH 8.0.

Expression Vector pGBFIN-49 was PCR Amplified Using Primers with Following Sequences:

(SEQ ID NO: 2943)
Forward primer: 5′-GTCCGTCGCCGTCCTTCACCG-3′
(SEQ ID NO: 2944)
Reverse primer: 5′-GGTGTTTTGTTGCTGGGGATGAAGC-3′

### Primers are Located at Either Site of the SfoI Restriction Site

PCR Mix Consists of Following Components:

| | |
|---|---|
| pGBFIN-49 plasmid DNA (10 ng/μL) | 2 μL |
| 5X Phusion HF Buffer (Finnzymes ™) | 20 μL |
| 2 mM dNTPs | 10 μL |
| LIC Primer mix (F + R) 10 mM | 2 μL |
| Phusion DNA Polymerase (Finnzymes ™) | 1.5 μL |
| DMSO | 3 μL |
| H₂O | 61.5 μL |
| TOTAL | 100 μL |

PCR Amplification was Carried Out with Following Conditions:

| | 2-step PCR protocol | | |
|---|---|---|---|
| Cycle step | Temp. | Time | Cycles |
| Initial denaturation | 98° C. | 2 min | 1 |
| Denaturation | 98° C. | 10 s | 35 |
| Annealing + Extension | 68° C. | 4 min + 10 s/cycle | |
| Final extension | 70° C. | 10 min | 1 |
| End of PCR storage | 4° C. | Hold | 1 |

Following PCR, 1 μL of DpnI was added to the PCR mix and digestion was performed overnight at 37° C. Digested PCR product was purified using the Qiaquick™ PCR purification kit (Qiagen) according to manufacturer's instructions.

Obtained PCR fragments were treated with T4 DNA polymerase in the presence of dTTP to create single stranded tails at the ends of the PCR fragments. The single stranded tails of the PCR fragment are complementary to those of the vector, thus permitting non-covalent bi-molecular associations, e.g., circularization between molecules.

The reaction mix of the T4 DNA polymerase treatment of the pGBFIN-49 PCR fragment consisted of the following components:

| | | |
|---|---|---|
| Purified pGBFIN-49 PCR fragment | 600 | ng |
| 10X Neb Buffer 2 | 2 | μL |
| 25 mM dTTP | 2 | μL |
| DTT 100 μM | 0.8 | μL |
| T4 DNA Polymerase 3 U/μL | 1 | μL |
| H₂O | Up to 20 | μL |
| TOTAL | 20 | μL |

The reaction mix of T4 DNA polymerase treatment of the Gene of Interest (GOI) PCR fragment consisted of the following components:

| | |
|---|---|
| Purified GOI PCR | 5 μL |
| 10X NEB Buffer 2 | 2 μL |
| 25 mM dATP | 2 μL |
| DTT 100 μM | 0.8 μL |
| T4 DNA Polymerase 3 U/μL | 1 μL |
| H₂O | 9.2 μL |
| TOTAL | 20 μL |

Reaction Conditions were as Follows:

| Step | Temperature (° C.) | Duration |
|---|---|---|
| Annealing | 22 | 30 min |
| Enzyme inactivation | 75 | 20 min |
| End | 4 | Hold |

Following T4 DNA polymerase treatment, 2 μL of pGBFIN-49 vector and 4 μL of the GOI were mixed and incubated at room temperature allowing annealing of GOI fragment with pGBFIN-49 vector fragment. The bi-molecular forms are used to transform E. coli. Plasmid DNA of resulting transformants was isolated and verified by sequence analyses for correct amplification and cloning of the gene of interest.

### Example 9

### Transformation of Scytalidium thermophilum, Myriococcum Thermophilum, and Aureobasidium pullulans Gene Expression Cassettes into A. niger

As host strain for enzyme production, A. niger GBA307 was used. Construction of A. niger GBA307 is described in WO 2011/009700.

Transformation of A. niger was performed essentially according to the method described by Tilburn, J. et. al. (1983) Gene 26, 205-221 and Kelly, J & Hynes, M. (1985) EMBO J., 4, 475-479 with the following modifications:
Spores were grown for 16-24 hours at 30° C. in a rotary shaker at 250 rpm in Aspergillus minimal medium. Aspergillus minimal medium contains per liter: 6 g NaNO₃; 0.52 g KCl; 1.52 g KH₂PO₄; 1.12 ml 4 M KOH; 0.52 g MgSO₄.7H₂O; 10 g glucose; 1 g casamino acids; 22 mg ZnSO₄.7H₂O; 11 mg H₃BO₃; 5 mg FeSO₄.7H₂O; 1.7 mg CoCl₂.6H₂O; 1.6 mg CuSO₄.5H₂O; 5 mg MnCl₂.2H₂O; 1.5 mg Na₂MoO₄.2H₂O; 50 mg EDTA; 2 mg riboflavin; 2 mg thiamine-HCl; 2 mg nicotinamide; 1 mg pyridoxine-HCl; 0.2 mg panthotenic acid; 4 μg biotin; 10 ml Penicillin (50001 U/mL/Streptomycin (5000 UG/mL) solution (Invitrogen);
Glucanex 200G (Novozymes) was used for the preparation of protoplasts;
After protoplast formation (2-3 hours) 10 mL TB layer (per liter: 109.32 g Sorbitol; 100 mL 1 M Tris-HCl pH 7.5) was pipetted gently on top of the protoplast suspension. After centrifugation for 10 min at 4330×g at 4° C. in a swinging bucket rotor, the protoplasts on the interface were transferred to a fresh tube and washed with STC buffer (1.2 M Sorbitol, 10 mM Tris-HCl pH 7.5, 50 mM CaCl₂). The protoplast suspension was centrifuged for 10 min at 1560×g in a swinging bucket rotor and resuspended in STC-buffer at a concentration of 10⁸ protoplasts/mL;
To 200 μL of the protoplast suspension, 20 μL ATA (0.4 M Aurintricarboxylic acid), the DNA dissolved in 10 μL in TE buffer (10 mM Tris-HCl pH 7.5, 0.1 mM EDTA), 100 μL of a PEG solution (20% PEG 4000 (Merck), 0.8M sorbitol, 10 mM Tris-HCl pH 7.5, 50 mM CaCl₂) was added;
After incubation of the DNA-protoplast suspension for 10 min at room temperature, 1.5 ml PEG solution (60% PEG 4000 (Merck), 10 mM Tris-HCl pH7.5, 50 mM CaCl₂) was added slowly, with repeated mixing of the tubes. After incubation for 20 min at room temperature, suspensions were diluted with 5 ml 1.2 M sorbitol, mixed by inversion and centrifuged for 10 min at 2770×g at room temperature.
The protoplasts were resuspended gently in 1 mL 1.2 M sorbitol and plated onto selective regeneration medium consisting of Aspergillus minimal medium without riboflavin, thiamine.HCl, nicotinamide, pyridoxine, panthotenic acid, biotin, casamino acids and glucose, supplemented with 150 μg/mL Phleomycin (Invitrogen), 0.07 M NaNO₃, 1 M sucrose, solidified with 2% bacteriological agar #1 (Oxoid, England). After incubation for 5-10 days at 30° C., single transformants were isolated on PDA (Potato Dextrose Agar (Difco) supplemented with 150 μg/mL Phleomycin in 96 wells MTP. After 5-7 days growth at 30° C. single transformants were used for MTP fermentation.

### Example 10

### Aspergillus niger Microtiter Plate Fermentation

96 wells microtiter plates (MTP) with sporulated Aspergillus niger strains were used to harvest spores for MTP fermentations. To do this, 100 ?l water was added to each well and after resuspending the mixture, 40 μL of spore suspension was used to inoculate 2 mL A. niger medium (70 g/L glucose.H₂O, 10 g/L yeast extract, 10 g/L (NH₄)₂SO₄, 2 g/L K₂SO₄, 2 g/L KH₂PO₄, 0.5 g/L MgSO₄.7H₂O, 0.5 g/L ZnSO₄.7H₂O, 0.2 g/L CaCl₂, 0.01 g/L MnSO₄.7H₂O, 0.05 g/L FeSO₄.7H₂O, 0.002 Na₂MoO₄.2H₂O, 0.25 g/L Tween™-80, 10 g/L citric acid, 30 g/L MES; pH 5.5 adjusted with 4 M NaOH) in a 24 well MTP. In the MTP fermentations for strains expressing GH61 proteins (e.g., polysaccharide monooxygenases), 30 μM CuSO₄ was included in the media. The MTP's were incubated in a humidity shaker (Infors) at 34° C. at 550 rpm, and 80% humidity for 6 days. Plates were centrifuged and supernatants were harvested.

### Example 11

### Aspergillus niger Shake Flask Fermentation

Approximately 1×10⁸-1×10⁷ spores were inoculated in 20 mL pre-culture medium containing Maltose 30 g/L; Peptone (aus casein) 10 g/L; Yeast extract 5 g/L; KH₂PO₄ 1 g/L; MgSO₄.7H₂O 0.5 g/L; ZnCl₂ 0.03 g/L; CaCl₂ 0.02 g/L; MnSO₄.4H₂O 0.01 g/L; FeSO₄.7H₂O 0.3 g/L; Tween™-80 3 g/L; pH 5.5. After growing overnight at 34° C. in a rotary shaker, 10-15 mL of the growing culture was inoculated in 100 mL main culture containing Glucose.H₂O 70 g/L; Peptone (aus casein) 25 g/L; Yeast extract 12.5 g/L; K₂SO₄ 2 g/L; KH₂PO₄ 1 g/L; MgSO₄.7H₂O 0.5 g/L; ZnCl₂ 0.03 g/L; CaCl₂ 0.02 g/L; MnSO₄.1H₂O 0.009 g/L; FeSO₄.7H₂O 0.003 g/L; pH 5.6.

Note: for GH61 (e.g., polysaccharide monooxygenase) enzymes the culture media were supplemented with 10 μM CuSO₄.

Main cultures were grown until all glucose was consumed as measured with Combur Test N strips (Roche), which was the case mostly after 4-7 days of growth. Culture supernatants were harvested by centrifugation for 10 minutes at 5000×g followed by germ-free filtration of the supernatant over 0.2 μm PES filters (Nalgene).

### Example 12

### Protein Concentration Determination with TCA-Biuret Method

Concentrated protein samples (supernatants) were diluted with water to a concentration between 2 and 8 mg/mL. Bovine serum albumin (BSA) dilutions (0, 1, 2, 5, 8 and 10 mg/mL were made and included as samples to generate a calibration curve. 1 mL of each diluted protein sample was transferred into a 10-mL tube containing 1 mL of a 20% (w/v) trichloro acetic acid solution in water and mixed thoroughly. Subsequently, the tubes were incubated on ice water for one hour and centrifuged for 30 minutes, at 4° C. and 6000 rpm. The supernatant was discarded and pellets were dried by inverting the tubes on a tissue and letting them stand for 30 minutes at room temperature. Next, 4-mL BioQuant Biuret reagent mix was added to the pellet in the tube and the pellet was solubilized upon mixing. Next, 1 mL water was added to the tube, the tube was mixed thoroughly and incubated at room temperature for 30 minutes. The absorption of the mixture was measured at 546 nm with a water sample used as a blank measurement and the protein concentration was calculated via the BSA calibration line.

### Example 13

### Microtiter Plate (MTP) Sugar-Release Activity Assay

For each (hemi-)cellulase assay, the stored samples were analyzed twice according the following procedure 100 μL sample and 100 μL of a (hemi-)cellulase base mix [1.75 mg/g DM TEC-210 or a 3 enzyme mix at a total dosage of 3.5 mg/g DM consisting of 0.5 mg/g DM BG (14% of total protein 3E mix), 1.6 mg/g DM CBHI (47% of total protein 3E mix) and 1.4 mg/g DM CBHII (39% of total protein 3E mix)] was transferred to two suitable vials: one vial containing 800 μL 2.5% (w/w) dry matter of the acid pre-treated corn stover substrate in a 50 mM citrate buffer, buffered at pH 4.5. The other vial consisted of a blank, where the 800 μL 2.5% (w/w) dry matter, acid pre-treated corn stover substrate suspension was replaced by 800 μL 50 mM citrate buffer, buffered at pH 4.5. The assay samples were incubated for 72 hrs at 65° C. After incubation of the assay samples, a fixed volume of an internal standard, maleic acid (20 g/L), EDTA (40 g/L) and DSS (0.5 g/L), was added. After centrifugation, the supernatant of the samples is lyophilized overnight; subsequently 100 μL D₂O is added to the dried residue and lyophilized once more. The dried residue is dissolved in 600 μL of D₂O.

The amount of sugar released, is based on the signal between 4.65-4.61 ppm, relative to DSS, and is determined by means of 1D ¹H NMR operating at a proton frequency of 500 MHz, using a pulseprogram without water suppression, at a temperature of 27° C.

The (hemi)-cellulase enzyme solution may contain residual sugars. Therefore, the results of the assay are corrected for the sugar content measured after incubation of the enzyme solution.

### Example 14

### Sugar-Release Activity Assays: Labscale, Incubation with Shaking

A. niger strains expressing Scytalidium thermophilum, Myriococcum thermophilum, and Aureobasidium pullulans clones were grown in shake flask, as described above (Example 11), in order to obtain greater amounts of material for further testing. The fermentation supernatants (volume between 40 and 80 mL) were concentrated using a 10-kDa spin filter to a volume of approximately 5 mL. Subsequently, the protein concentration in the concentrated supernatant was determined via a TCA-biuret method, as described above in Example 12. The (hemi-)cellulase activity of these protein samples was tested in an assay where the supernatants were spiked on top of an enzyme base mix in the presence of 10% (w/w) acid pretreated corn stover (aCS). ‘To spike’ or ‘spiking of’ a supernatant or an enzyme indicates, in this context, the addition of a supernatant or an enzyme to a (hemi)-cellulase base mix. The feedstock solution was prepared via the dilution of a concentrated feedstock solution with water. Subsequently, the pH was adjusted to pH 4.5 with a 4 M NaOH solution. The proteins were spiked based on dosage; the concentrated supernatant samples were added in a final concentration of 2 mg/gDM to the base enzyme mix (TEC-210 5 mg/gDM) in a total volume of 10 mL at a feedstock concentration of 10% aCS (w/w) in an 30-mL centrifuge bottle (Nalgene Oakridge). All experiments were performed at least in duplicate and were incubated for 72 hours at 65° C. in an oven incubator (Techne HB-1D hybridization oven) while rotating at set-point 3. After incubation, the samples were centrifuged and soluble sugars were analysed by HPLC as described below.

### Example 15

### Soluble Sugar Analysis by HPLC

The sugar content of the samples after enzymatic hydrolysis were analyzed using a High-Performance Liquid Chromatography System (Agilent 1100) equipped with a refection index detector (Agilent 1260 Infinity). The separation of the sugars was achieved by using a 300×7.8 mm Aminex HPX-87P (Bio-Rad cat. no. 125-0098) column; Pre-column: Micro guard Carbo-P (Bio-Rad cat. no. 125-0119); mobile phase was HPLC grade water; flow rate of 0.6 mL/min and a column temperature of 85° C. The injection volume was 10 μL.

The samples were diluted with HPLC grade water to a maximum of 10 g/L glucose and filtered by using 0.2 μm filter (Afridisc LC25 mm syringe filter PVDF membrane). The glucose was identified and quantified according to the retention time, which was compared to the external glucose standard (D-(+)-Glucose Sigma cat. no: G7528) ranging from 0.2; 0.4; 1.0; 2.0 g/L.

### Example 16

### Protein Activity Assays

16.1 Alpha-Arabino(Furano)Sidase Activity Assay

This assay measures the ability of α-arabino(furano)sidases to remove the alpha-L-arabinofuranosyl residues from substituted xylose residues. Single and double substituted oligosaccharides are prepared by incubating wheat arabinoxylan (WAX medium viscosity; 2 mg/mL; Megazyme, Bray, Ireland) in 50 mM acetate buffer pH 4.5 with an appropriate amount of endo-xylanase (Aspergillus Awamori, F J M, Kormelink, Carbohydrate Research, 249 (1993) 355-367) for 48 hours at 50° C. to produce an sufficient amount of arabinoxylo-oligosaccharides. The reaction is stopped by heating the samples at 100° C. for 10 minutes. The samples are centrifuged for 5 minutes at 10,000×g. The supernatant is used for further experiments. Degradation of the arabinoxylan is followed by High Performance Anion Exchange Chromatography (HPAEC).

The enzyme is added to the single and double substituted arabinoxylo-oligosaccharides (endo-xylanase treated WAX) in a dosage of 10 mg protein/g substrate in 50 mM sodium acetate buffer which is then incubated at 65° C. for 24 hours. The reaction is stopped by heating the samples at 100° C. for 10 minutes. The samples are centrifuged for 5 minutes at 10,000×g and 10 times diluted. Release of arabinose from the arabinoxylo-oligosaccharides is analyzed by HPAEC analysis.

The analysis is performed using a Dionex HPLC system equipped with a Dionex CarboPac PA-1 (2 mm ID×250 mm) column in combination with a CarboPac PA guard column (2 mm ID×50 mm) and a Dionex PAD-detector (Dionex Co. Sunnyvale). A flow rate of 0.3 mL/min is used with the following gradient of sodium acetate in 0.1 M NaOH: 0-40 min, 0-400 mM. Each elution is followed by a washing step of 5 min 1000 mM sodium acetate in 0.1 M NaOH and an equilibration step of 15 min 0.1 M NaOH. Arabinose release is quantified by an arabinose standard (Sigma) and compared to a sample where no enzyme was added.

### 16.2 Beta-Xylosidase Activity Assay

This assay measures the release of xylose by the action of beta-xylosidase on xylobiose. Sodium acetate buffer (0.05 M, pH 4.5) is prepared as follows. 4.1 g of anhydrous sodium acetate or 6.8 g of sodium acetate*3H₂O is dissolved in distilled water to a final volume of 1000 mL (Solution A). In a separate flask, 3.0 g (2.86 mL) of glacial acetic acid is mixed with distilled water to make the total volume of 1000 mL (Solution B). The final 0.05 M sodium acetate buffer, pH 4.5, is prepared by mixing Solution A with Solution B until the pH of the resulting solution is equal to 4.5.

Xylobiose was purchased from Sigma and a solution of 100 μg/mL sodium acetate buffer pH 4.5 was prepared. The assay is performed as detailed below.

The enzyme is added to the substrate in a dosage of 10, 5 or 1 mg protein/g substrate, which is then incubated at 62-65° C. for 24 hours. The reaction is stopped by heating the samples for 10 minutes at 100° C. Samples are appropriate diluted and the release of xylose is analyzed by High Performance Anion Exchange Chromatography.

The analysis is performed using a Dionex HPLC system equipped with a Dionex CarboPac PA-1 (2 mm ID×250 mm) column in combination with a CarboPac PA guard column (2 mm ID×50 mm) and a Dionex PAD-detector (Dionex Co. Sunnyvale). A flow rate of 0.3 mL/min is used with the following gradient of sodium acetate in 0.1 M NaOH: 0-20 min, 0-17.8 mM. Each elution is followed by a washing step of 5 min 1000 mM sodium acetate in 0.1 M NaOH and an equilibration step of 15 min 0.1 M NaOH.

In case interfering compounds are present that complicate xylose quantification, the analysis is performed by running isocratic on H₂O for 30 min a gradient (0.5M NaOH is added post-column at 0.1 mL/min for detection) followed by a washing step of 5 min 1000 mM sodium acetate in 0.1 M NaOH and an equilibration step of 15 min H₂O.

Standards of xylose and xylobiose (Sigma) are used for identification and quantification of the substrate and product formed by the enzyme.

### 16.3 Acetyl-Xylan Esterase Activity Assay

Acetyl-xylan esterases are enzymes able to hydrolyze ester linked acetyl groups attached to the xylan backbone, releasing acetic acid. This assay measures the release of acetic acid by the action of acetyl xylan esterase on acid pretreated corn stover (aCS) that contains ester linked acetyl groups.

### Determine the Presence of Acetyl Groups in pCS

The aCS used contains ±284 (±5.5) μg acetic acid/20 mg pCS as determined according to the following method.

About 20 mg of aCS substrate was weighed in a 2 mL reaction tube and placed in an ice-water bath. Then 1 mL of 0.4M NaOH in Millipore water/isopropanol (1:1) was added and the sample was thoroughly mixed. This was incubated on ice for 1 hour. Subsequently, the samples were mixed again and incubated for 2 additional hours at room temperature (mixed once in a while). After this samples were centrifuged for 5 min at 12000 rpm and the supernatant was analyzed for acetic acid content by HPLC.

### Enzyme Incubations

Enzyme incubations were performed in citrate buffer (0.05 M, pH 4.5) which is prepared as follows; 14.7 g of tri-sodium citrate is dissolved in distilled water to a final volume of 1000 mL (Solution A). In a separate flask, 10.5 g citric acid monohydrate is mixed with distilled water to make the total volume of 1000 mL (Solution B). The final 0.05 M sodium citrate buffer, pH 4.5, is prepared by mixing Solution A with Solution B until the pH of the resulting solution is 4.5.

The aCS substrate is solved in citrate buffer to obtain ±20 mg/mL. The enzyme is added to the substrate in a dosage of 1 or 10 mg protein/g substrate, which is then incubated at 60° C. for 24 hours head-over-tail. The reaction is stopped by heating the samples for 10 minutes at 100° C. The release of acetic acid is analyzed by HPLC.

As a blank sample the substrate is treated and incubated in the same way but then without the addition of enzyme.

The analysis is performed using an Ultimate 3000 system (Dionex) equipped with a Shodex RI detector and an Aminex HPX 87H column (7.8 mm ID×300 mm) column (BioRad). A flow rate of 0.6 mL/min is used with 5.0 mM H2SO4 as eluent for 30 minutes at a column temperature of 40° C. Acetic acid was used as a standard to quantify its release from pCS by the enzymes.

### 16.4 Endoxylanase Activity Assay 1

Endoxylanases are enzyme able to hydrolyze β-1,4 bond in the xylan backbone, producing short xylooligosaccharides. This assay measures the release of xylose and xylo-oligosaccharides by the action of xylanases on wheat arabinoxylan oligosaccharides (WAX) (Megazyme, Medium viscosity 29 cSt) and Beech Wood Xylan (Beech) (Sigma).

Sodium acetate buffer (0.05 M, pH 4.5) is prepared as follows; 4.1 g of anhydrous sodium acetate is dissolved in distilled water to a final volume of 1000 mL (Solution A). In a separate flask, 3.0 g (2.86 mL) of glacial acetic acid is mixed with distilled water to make the total volume of 1000 mL (Solution B). The final 0.05 M sodium acetate buffer, pH 4.5, is prepared by mixing Solution A with Solution B until the pH of the resulting solution is 4.5.

The substrates WAX and Beech are solved in sodium acetate buffer to obtain 2.0 mg/mL. The enzyme is added to the substrate in a dosage of 10 mg protein/g substrate which is then incubated at 65° C. for 24 hours. The reaction is stopped by heating the samples for 10 minutes at 100° C. The release of xylose and (arabino)xylan oligosaccharides is analyzed by High Performance Anion Exchange Chromatography.

As a blank sample the substrate is treated and incubated in the same way but then without the addition of enzyme.

The analysis is performed using a Dionex HPLC system equipped with a Dionex CarboPac PA-1 (2 mm ID×250 mm) column in combination with a CarboPac PA guard column (2 mm ID×50 mm) and a Dionex PAD-detector (Dionex Co. Sunnyvale). A flow rate of 0.3 mL/min is used with the following gradient of sodium acetate in 0.1 M NaOH: 0-40 min, 0-400 mM. Each elution is followed by a washing step of 5 min 1000 mM sodium acetate in 0.1 M NaOH and an equilibration step of 15 min 0.1 M NaOH. Standards of xylose, xylobiose, xylotriose and xylotetraose (Sigma) are used to identify and quantify these oligomers released by the action of the enzyme.

### 16.5 Endo-Xylanase Activity Assay 2

Endo-xylanases are enzyme able to hydrolyze beta-1,4 bond in the xylan backbone, producing short xylooligosaccharides. This assay measures the release of xylose and xylo-oligosaccharides by the action of xylanases on wheat arabinoxylan oligosaccharides (WAX) (Megazyme, Medium viscosity 29 cSt).

Sodium acetate buffer (0.05 M, pH 4.5) is prepared as follows: 4.1 g of anhydrous sodium acetate is dissolved in distilled water to a final volume of 1000 mL (Solution A). In a separate flask, 3.0 g (2.86 mL) of glacial acetic acid is mixed with distilled water to make the total volume of 1000 mL (Solution B). The final 0.05 M sodium acetate buffer, pH 4.5, is prepared by mixing Solution A with Solution B until the pH of the resulting solution is 4.5.

The substrate WAX is solved in sodium acetate buffer to obtain 2.0 mg/mL. The enzyme is added to the substrate in a dosage of 1 mg protein/g substrate which is then incubated at 65° C. for 24 hours. During these 24 hours, samples are taken and the reaction is stopped by heating the samples for 10 minutes at 100° C.

The enzyme activity is demonstrated by using a reducing sugars assay (PAHBAH) as detection method.

Reagent A: 5 g of p-Hydroxybenzoic acid hydrazide (PAHBAH) is suspended in 60 mL water, 4.1 mL of concentrated hydrochloric acid is added and the volume is adjusted to 100 mL. Reagent B: 0.5 M sodium hydroxide. Both reagents are stored at room temperature. Working Reagent: 10 mL of Reagent A is added to 40 mL of Reagent B. This solution is prepared freshly every day, and is stored on ice between uses. Using the above reagents, the assay is performed as detailed below.

The assay is conducted in microtiter plate format. After incubation 10 μL of each sample is added to a well and mixed with 150 μL working reagent. These solutions are heated at 70° C. for 30 minutes or for 5 minutes at 90° C. After cooling down, the samples are analyzed by measuring the absorbance at 405 nm. The standard curve is made by treating 10 μL of an appropriate diluted xylose solution the same way as the samples. The reducing-ends formed due to the action of enzyme is expressed as xylose equivalents.

Rasamsonia (Talaromyces) emersonii strain was deposited at CENTRAAL BUREAU VOOR SCHIMMELCULTURES, Uppsalalaan 8, P.O. Box 85167, NL-3508 AD Utrecht, The Netherlands in December 1964 having the Accession Number CBS 393.64.

Other suitable strains can be equally used in the present examples to show the effect and advantages of the invention. For example TEC-101, TEC-147, TEC-192, TEC-201 or TEC-210 are suitable Rasamsonia strains which are described in WO 2011/000949. The “4E mix” or “4E composition” was used containing CBHI, CBHII, EG4 and BG (30 wt %, 25 wt %, 28 wt % and 8 wt %, respectively, as described in WO 2011/098577, wt % on dry matter protein).

Rasamsonia (Talaromyces) emersonii strain TEC-101 (also designated as FBG 101) was deposited at CENTRAAL BUREAU VOOR SCHIMMELCULTURES, Uppsalalaan 8, P.O. Box 85167, NL-3508 AD Utrecht, The Netherlands on 30, Jun. 2010 having the Accession Number CBS 127450.

TEC-210 was fermented according to the inoculation and fermentation procedures described in WO 2011/000949.

The 4E mix (4 enzymes mixture or 4 enzyme mix) containing CBHI, CBHII, GH61 and BG (30%, 25%, 36% and 9%, respectively as described in WO 2011/098577) was used.

3E mix (3 enzymes mixture or 3 enzyme mix) is spiked with a fourth enzyme to form the 4E mix.

### 16.6 Xyloglucanase Activity Assay

Sodium acetate buffer (0.05 M, pH 4.5) is prepared as follows: 4.1 g of anhydrous sodium acetate is dissolved in distilled water to a final volume of 1000 mL (Solution A). In a separate flask, 3.0 g (2.86 mL) of glacial acetic acid is mixed with distilled water to make the total volume of 1000 mL (Solution B). The final 0.05 M sodium acetate buffer, pH 4.5, is prepared by mixing Solution A with Solution B until the pH of the resulting solution is 4.5.

Tamarind xyloglucan is dissolved in sodium acetate buffer to obtain 2.0 mg/mL. The enzyme is added to the substrate in a dosage of 10 mg protein/g substrate, which is then incubated at 60° C. for 24 hours. The reaction is stopped by heating the samples for 10 minutes at 100° C. The formation of lower molecular weight oligosaccharides is analyzed by High Performance size-exclusion Chromatography

As a blank sample, the substrate is treated and incubated in the same way but then without the addition of enzyme.

The analysis is performed using High-performance size-exclusion chromatography (HPSEC) performed on three TSK-gel columns (6.0 mm×15.0 cm per column) in series SuperAW4000, SuperAW3000, SuperAW2500; Tosoh Bioscience), in combination with a PWXguard column (Tosoh Bioscience). Elution is performed at 55° C. with 0.2 M sodium nitrate at 0.6 mL/min. The eluate was monitored using a Shodex RI-101 (Kawasaki) refractive index (RI) detector. Calibration was performed by using pullulans (Associated Polymer Labs Inc., New York, USA) with a molecular weight in the range of 0.18-788 kDa.

### 16.7 Assay Protocol CU1: Colorimetric Assay for Glycosidase or Esterase Activity, Measuring Release of 4-Nitrophenol

Enzyme sample is diluted in 10 mM citrate buffer, pH 5.0, made by dissolving 1.92 g of citric acid in water, adjusting pH to 5.0 with 10 M NaOH and diluting to 1 L. 10 μL of diluted enzyme sample is added to 30 μL of 50 mM acetate-phosphate-borate reaction buffer at appropriate pH (made by dissolving 2.88 mL 99.7% glacial acetic acid, 3.42 mL 85% phosphoric acid, and 3.10 g boric acid in water, adjusting pH with 10 M NaOH and diluting to 1 L) in a PCR plate and preheated to appropriate temperature in a dry bath heater, and reaction is started by addition of 10 μL of preheated 5 mM substrate in water (see Table 5) to buffer and sample. Standards contain 10 μL of 4-nitrophenol (from 0 to 3 mM; 3 mM solution is made by dissolving 139 mg 4-nitrophenol in isopropyl alcohol and diluting 300 μL of resulting 100 mM solution to 10 mL in water) and 40 μL of reaction buffer. Sample blank contains 10 μL of enzyme sample and 40 μL of reaction buffer. Substrate blank contains 10 μL of substrate (see table) and 40 μL of reaction buffer. After appropriate incubation time, 50 μL of [1] for 4-nitrophenyl acetate, 1 M HEPES buffer pH 8 in water; [2] for 4-nitrophenyl butyrate, 250 mM Na2CO3 in water; [3] for all other substrates, 1 M Na2CO3 in water is added. 80 μL is then transferred to a clear microtiter flat-bottomed plate, absorbance is read at 410 nm and compared to the standard curve. One unit is defined as the amount of enzyme that releases one micromole of 4-nitrophenol per minute at the specified pH and temperature. (Adapted from Holmsen et al (1989) Methods in Enzymology, 169, 336-342.)

TABLE 5

| Enzyme activity | Substrate |
|---|---|
| arabinofuranosidase | 4-nitrophenyl alpha-L-arabinofuranoside |
| arabinopyranosidase | 4-nitrophenyl alpha-L-arabinopyranoside |
| beta-galactosidase | 4-nitrophenyl beta-D-galactopyranoside |
| hexosaminidase/N- | 4-nitrophenyl beta-D-glucosaminide |
| acetylglucosaminidase | |
| beta-glucosidase | 4-nitrophenyl beta-D-glucopyranoside |
| beta-mannosidase | 4-nitrophenyl beta-D-mannopyranoside |
| beta-xylosidase | 4-nitrophenyl beta-D-xylopyranoside |
| Acetylesterase | 4-nitrophenyl acetate |
| Cutinase; lipase | 4-nitrophenyl butyrate |

### 16.8 Assay Procedure CU2: Colorimetric Assay for Endo-Glycanase Activity, Measuring Copper (I) Reduced by Polysaccharide Reducing Ends

Enzyme sample is diluted in 10 mM citrate buffer, pH 5.0, made by dissolving 1.92 g of citric acid in water, adjusting pH to 5.0 with 10 M NaOH and diluting to 1 L. 10 μL of diluted sample is added to 30 μL of either [1] 50 mM acetate-phosphate-borate reaction buffer at appropriate pH (made by dissolving 2.88 mL 99.7% glacial acetic acid, 3.42 mL 85% phosphoric acid, and 3.10 g boric acid in water, adjusting pH with 10 M NaOH and diluting to 1 L) or [2] for enzymes that utilize calcium, 50 mM acetate-MOPS-borate reaction buffer at appropriate pH (made by dissolving 2.88 mL 99.7% glacial acetic acid, 10.45 g MOPS, and 3.10 g boric acid in water, adjusting pH with 10 M NaOH and diluting to 1 L) in a PCR plate and preheated to appropriate temperature in a dry bath heater. The reaction is started by addition of 10 μL of preheated 5 mM substrate in water (see Table 6) to buffer and sample. Standards contain 10 μL of 0 to 7.5 mM monosaccharide solution (see Table 6) in water and 40 μL of reaction buffer. Enzyme sample blank contains 10 μL of sample and 40 μL of reaction buffer. Substrate blank contains 10 μL of substrate (see Table 6) and 40 μL of reaction buffer. After appropriate incubation time, 10 μL is removed and added to another PCR plate containing 95 μL of BCA Reagent A (made by dissolving 0.543 g Na2CO3, 0.242 g NaHCO3 and 19 mg disodium 2,2′-bicinchoninate in water and diluting to 1 L) and 95 μL of BCA Reagent B (made by dissolving 12 mg CuSO4 and 13 mg L-Serine in water and diluting to 1 L), sealed and incubated in a dry bath heater for 25 minutes at 80° C. PCR plate is put on ice for 5 minutes, then 160 μL is transferred to a clear microtiter flat-bottomed plate, absorbance is read at 562 nm and compared to the standard curve. One unit is defined as the amount of enzyme that releases one micromole of monosaccharide-equivalent reducing ends per minute at the specified pH and temperature. (Adapted from Fox et al (1991) Anal. Biochem., 195, 93-96.)

TABLE 6

| Enzyme | Substrate | Standard |
|---|---|---|
| Tomatinase | alpha-tomatine | galactose |
| Endomannanase | Beta-Mannan | Mannose |
| Endoglucanase | Carboxymethyl | Glucose |
| | cellulose (1:1 mixture | |
| | of 4M and 7M) | |
| Laminarinase | Laminarin | Glucose |
| Lichenanase | Lichenan | Glucose |
| Endomannanase | Locust bean gum | Mannose |
| Arabinoxylan | Low Viscosity Wheat | Arabinose |
| arabinofuranohydrolase | Arabinoxylan | |
| Endopolygalacturonase | Polygalacturonic acid | Galacturonic acid |
| Sucrase/Alpha-glucosidase/ | Sucrose | Glucose + Fructose |
| Invertase | | (1:1 mixture) |

### 16.9 Assay Procedure CU3: UV Assay for Acetylesterase Activity, Measuring Release of Alpha-Naphthol

Enzyme sample is diluted in 10 mM citrate buffer, pH 5.0, made by dissolving 1.92 g of citric acid in dH₂O, adjusting pH to 5.0 with 10 M NaOH and diluting to 1 L. 20 μL of diluted sample is added to 20 μL of 300 mM acetate-phosphate-borate reaction buffer at appropriate pH (made by dissolving 17.28 mL 99.7% glacial acetic acid, 20.52 mL 85% phosphoric acid, and 18.6 g boric acid in water, adjusting pH with 10 M NaOH and diluting to 1 L) in a clear microtiter plate and preheated to appropriate temperature in the plate reader. The reaction is started by addition of 160 μL 0.5 mM alpha-naphthyl acetate substrate solution in water (prepared by diluting 46.55 mg of a-Naphthyl acetate in 1 mL of acetone and then transferring to 499 mL of water), preheated to assay temperature in a dry block heater, to the buffer and enzyme sample. Standards contain 180 μL of 0 to 0.1 mM alpha-naphthol in water and 20 μL of reaction buffer. Blank contains 20 μL of reaction buffer, 20 μL of water and 160 μL of substrate solution. Absorbance is continuously monitored at 303 nm and compared to that of the standards. One unit is the amount of enzyme that produces one micromole of alpha-naphthol per minute under the specified conditions. (Adapted from Yuorno et al. (1981), Anal. Biochem. 115, 188-193)

### 16.10 Assay Procedure CU4: Polarimetric Assay for Aldose 1-Epimerase Activity, Measuring the Rate Increase of the Mutarotation of Alpha-D-Glucose

5 mM phosphate reaction buffer (prepared by dissolving 342 μL 85% phosphoric acid in water, adjusting to pH 5.0 with 1 M NaOH and diluting to 1 L) is preheated to 40° C. A Perkin-Elmer 341 polarimeter (USA) with sodium/halogen and mercury lamps preheated to 40° C. and is blanked by measuring the optical rotation of polarized 578 nm light by 5 mL reaction buffer. 36 mg of alpha-D-Glucose is dissolved in 10 mL of reaction buffer, then 60 μL of undiluted enzyme is added to 4.94 mL of the resulting solution and optical rotation is immediately measured in the polarimeter. Readings are recorded at 40° C. every minute until equilibrium is reached. One unit is the amount of enzyme that converts one micromole of alpha-D-glucose to beta-D-glucose (calculated by determining the reaction's first-order rate constant less that of the blank) in one minute. (Adapted from Bailey et al. (1975), Methods in Enzymology 41, 471-484).

### 16.11 Assay Procedure CU5: Colorimetric Assay Measuring Acid Release by the Absorbance of a pH Indicator

Reaction buffer is 2.5 mM MOPS, pH 7.2 (0.52 g MOPS dissolved in water, pH adjusted with 1 M NaOH and diluted to 1 L) or 2.5 mM acetate, pH 5.3 (144 μL glacial acetic acid dissolved in water, pH adjusted with 1 M NaOH and diluted to 1 L). Substrate stock solution is made by dissolving 111.1 mg of ethyl ferulate and 70 mg of 4-nitrophenol or 350 mg of bromocresol green in isopropyl alcohol. Substrate working solution is made by diluting substrate stock solution 1:10 with reaction buffer: pH 7.2 reaction buffer is used for substrate stock solution containing 4-nitrophenol, pH 5.3 for stock containing bromocresol green. Enzyme is thoroughly buffer exchanged into reaction buffer before use in the assay. Enzyme and substrate working solution are preheated to the appropriate temperature; 100 μL substrate working solution is added to a microtiter plate, and 20 μL of enzyme solution is added. The change in absorbance at 410 nm (pH 7.2) or 600 nm (pH 5.3) is determined. The pH of the solution is calculated by comparing the absorbance to that of the blank, and the amount of acid released is calculated. One unit is defined as the amount of enzyme that produces one micromole of ferulic acid per minute. (Adapted from Ramirez et al. (2008), Appl Biochem Biotechnol 151, 711-723.)

### 16.12 Assay Procedure CU6: UV Assay of Lyase Activity, Measuring Formation of Unsaturated Bonds

Enzyme sample is diluted in 50 mM acetate-mops-borate reaction buffer at appropriate pH (made by dissolving 2.88 mL 99.7% glacial acetic acid, 10.45 g MOPS, 3.10 g boric acid and 1.11 g calcium chloride in water, adjusting pH with 10 M NaOH and diluting to 1 L) and left to equilibrate for 30 minutes at room temperature. Reaction buffer is mixed in a 1:1 ratio with substrate solution (1% polygalacturonic acid in water or 0.75% Rhamnogalacturonan I from potato in water) and preheated to reaction temperature in a dry bath heater (if reaction temperature is greater than plate reader maximum temperature) or in a microtiter plate in plate reader. Reaction is started by addition of 10 μL of diluted enzyme sample to 240 μL of reaction buffer/substrate in UV-transparent microtiter flat-bottomed plate. Blank contains 10 μL of reaction buffer added to 240 μL of reaction buffer/substrate solution. Absorbance at 235 nm is continuously monitored, and the molar absorptivity coefficient of unsaturated galacturonic acid is used to determine activity. One unit is the amount of enzyme that releases one micromole of unsaturated galacturonic acid equivalents per minute under the specified conditions. Adapted from Hansen et al. (2001) J. AOAC International, 84, 1851-1854)

### 16.13 Assay Procedure CU7: Fluorescence Assay, Measuring Release of 4-Methylumbelliferone

Enzyme sample is diluted in 10 mM citrate buffer, pH 5.0, made by dissolving 1.92 g of citric acid in water, adjusting pH to 5.0 with 10 M NaOH and diluting to 1 L. 10 μL of diluted sample is added to 30 μL of 50 mM acetate-phosphate-borate reaction buffer at appropriate pH (made by dissolving 2.88 mL 99.7% glacial acetic acid, 3.42 mL 85% phosphoric acid, and 3.10 g boric acid in water, adjusting pH with 10 M NaOH and diluting to 1 L) in a PCR plate and preheated to appropriate temperature in a dry bath heater. The reaction is started by addition of 10 μL of preheated 1 mM substrate in water (made by diluting 5.0 mg of 4-methylumbelliferyl cellobioside or 4-methylumbelliferyl lactoside in 10 mL water) to buffer and sample. Standards contain 10 μL of 4-methylumbelliferone (from 0 to 50 uM; 19.8 mg of 4-methylumbelliferone sodium salt is dissolved in 100 mL methanol and resulting solution is diluted 20× in water) and 40 μL of reaction buffer. Enzyme sample blank contains 10 μL of enzyme sample and 40 μL of reaction buffer. Substrate blank contains 10 μL of substrate and 40 μL of reaction buffer. After appropriate incubation time, 20 μL is removed and added to a black microtiter plate containing 180 μL of glycine/carbonate buffer, pH 10.7 (made by dissolving 10 g glycine and 8.8 g sodium carbonate in water, adjusting pH with 10 M NaOH and diluting to 1 L). The fluorescence of the wells is measured at 355 nm excitation, 460 nm emission and compared to the standard curve. One unit is defined as the amount of enzyme that releases one micromole of 4-methylumbelliferone per minute. (Adapted from van Tilbeurgh et al. (1988), Methods in Enzymology 160: 45-59.)

### 16.14 Assay Procedure CUB: Spectrophotometric Assay of Acetylxylanesterase Activity, Measuring Release of Acetic Acid

Enzyme sample is diluted in 10 mM citrate buffer, pH 5.0, made by dissolving 1.92 g of citric acid in dH₂O, adjusting pH to 5.0 with 10 M NaOH and diluting to 1 L. 40 μL of 1% acetylated xylan from birchwood are added to 40 μL of 50 mM phosphate reaction buffer (prepared by dissolving 3.42 mL of 85& phosphoric acid in water, adjusting pH to 6.0 with 10 M NaOH and diluting to 1 L) in the wells of a 96-well PCR plate and preheated to the appropriate temperature in a dry block heater. The reaction is started by adding 20 μL of diluted sample to the wells containing substrate and reaction buffer. Standards contain 20 μL of 0 mg/mL to 1 mg/mL acetic acid in water, and 80 ul reaction buffer. Sample blank contains 20 μL of diluted enzyme sample, 40 μL of reaction buffer and 40 μL of water. Substrate blank contains 40 μL of substrate and 60 μL of reaction buffer. After appropriate incubation time, the plate is heated to 90° C. for 5 minutes and centrifuged 10 minutes at 1500×g. The amount of acetic acid in the supernatant is then determined with the K-ACETAK kit by Megazyme; one unit is defined as the amount of enzyme required to release one micromole of acetic acid per minute under the specified conditions. (Adapted from Johnson et al. (1988), Methods in Enzymology 160, 551-560 and K-ACETAK assay kit procedure by Megazyme (Ireland)).

### 16.15 Assay Procedure CU9: Gas Chromatographic Assay of Methylesterase Activity, Measuring Release of Methanol

Reaction buffer is 50 mM phosphate, pH 6.6, made by dissolving 3.42 mL 85% phosphoric acid in water, adjusting pH with 10 M NaOH and diluting to 1 L. Enzyme sample is diluted in buffer and preheated to reaction temperature. Substrate solution, 1% esterified pectin in water, is preheated to reaction temperature; reaction is started by adding 100 μL of diluted enzyme to 900 μL of substrate solution. Standards contain 100 μL methanol (0 to 100 mM in water) and 900 μL of substrate solution. After appropriate incubation time, samples are mixed and aliquot is injected into a gas chromatograph; peak areas of samples are compared to that of standards. One unit is amount of enzyme that produces one micromole of methanol per minute. (Adapted from Bartolome et al. (1972), J. Agric. Food Chem. 20 (2), 262-266.)

### 16.16 Assay Procedure CU10: Colorimetric Assay of Cellobiose Dehydrogenase, Measuring Reduction of DCIP

Enzyme sample is diluted in 10 mM citrate buffer, pH 5.0, made by dissolving 1.92 g of citric acid in water, adjusting pH to 5.0 with 10 M NaOH and diluting to 1 L. 10 μL of diluted enzyme sample is added to 10 μL of 48 mM sodium fluoride (made by dissolving 2 mg NaF in 10 mL water), 10 μL of 3.6 mM 2,6-dichloroindophenol (DCIP, made by dissolving 9.6 mg in 10 mL water) and 80 μL of 50 mM acetate-phosphate-borate reaction buffer at appropriate pH (made by dissolving 2.88 mL 99.7% glacial acetic acid, 3.42 mL 85% phosphoric acid, and 3.10 g boric acid in water, adjusting pH with 10 M NaOH and diluting to 1 L) in a clear microtiter flat-bottomed plate and preheated to the appropriate temperature in a dry bath heater. Reaction is started by addition of 120 μL of 360 mM lactose (made by dissolving 1.23 g lactose in 100 mL water). Blank contains 10 μL sample, 10 μL 48 mM NaF, 10 μL 3.6 mM DCIP, 80 μL reaction buffer and 120 μL water. Absorbance at 520 nm is continuously monitored and compared to the molar absorptivity coefficient of DCIP. One unit is the amount of enzyme that reduces one micromole of DCIP per minute under the specified assay conditions. (Adapted from Baminger et al. (2001), Appl Environ Microbiol, 67(4), 1766-1774.)

### 16.17 Assay Procedure CU11: Colorimetric Assay of Aldolactonase, Measuring Glucono-Delta-Lactone

Enzyme sample is diluted in 50 mM acetate reaction buffer, pH 5 (made by dissolving 2.88 mL 99.7% glacial acetic acid in water, adjusting pH with 10 M NaOH and diluting to 1 L) and preheated to 37° C. 50 mL of 4.0 M hydroxylamine hydrochloride (made by dissolving 27.6 g hydroxylamine hydrochloride in water and diluting to 100 mL) is mixed with 50 mL of 3.0 M sodium hydroxide (made by dissolving 12 g of sodium hydroxide and diluting to 100 mL); the resulting alkaline hydroxylamine solution is used within the next 3 hours. 0.239 g of glucono-delta-lactone are dissolved in 100 mL reaction buffer that has been preheated to 37° C., and 125 μL of the resulting 13.4 mM substrate solution is immediately pipetted to a clear flat-bottomed microtiter plate. The reaction is started by addition of 15 μL diluted sample to substrate solution. Standards contain 80-125 μL of substrate solution, with the volume made up to 140 μL with reaction buffer. After 10 minutes incubation, 28 μL alkaline hydroxylamine solution is added, then 14 μL 4 M HCl is added (made by diluting concentrated HCl threefold in water), then 14 μL of 0.5 M FeCl₃ (made by dissolving 8.1 g FeCl₃ in water and diluting to 100 mL) is added. Absorbances are read at 540 nm and compared to the standard curve. One unit is the amount of enzyme that removes one micromole of glucono-delta-lactone per minute. (Adapted from Hestrin et al. (1949), J. Biol. Chem. 180, 249-261.)

### 16.18 Activity-Temperature Profiles

Temperature optima are determined by first determining the range of enzyme concentration that reproducibly displays initial velocity kinetics at 40° C. in the appropriate assay. Enzyme is then diluted to an amount within this range, divided into aliquots, and, where possible, each aliquot is assayed simultaneously at the different temperatures (e.g., when reaction is incubated in a dry bath heater, then transferred to a plate reader for endpoint measurement). Where simultaneous measurements at different temperatures are impossible (e.g., when reaction is incubated in a plate reader for continuous measurement) activities are measured in sequence at different temperatures.

### Example 17

### Identification of Genes that Encode Secreted Proteins

Genes (and polypeptides) from the organisms Scytalidium thermophilum (Scyth), Myriococcum thermophilum (Myrth), and Aureobasidium pullulans (Aurpu) were identified that, based on curation (described above, see Example 4), encoded a secreted protein. A list of these genes and polypeptides is shown in Tables 1A-1C.

### Example 18

### Improvement of Thermophilic Cellulase Mixture by Various Proteins in an MTP Activity Assay Using aCS as Substrate

(Hemi-)cellulosic proteins of interest were cloned and expressed in A. niger as described above in Examples 8-10. Supernatants of protein MTP fermentations were added to a TEC-210 cellulase enzyme base mix as described above (Example 13), and acid pretreated corn stover (aCS) was used as the substrate. Several proteins demonstrated increased sugar release, as seem below in Table 7.

TABLE 7
Effect of various proteins spiked on TEC-210 using aCS substrate
in MTP assay

| Target ID | SEQ ID NOs: | Glucose (AU) |
|---|---|---|
| TEC only | — | 32.6 |
| Scyth2p4_010825 | 231, 516, 801 | 36.8 |
| Scyth2p4_008294 | 153, 438, 723 | 37.0 |
| Scyth2p4_006005 | 110, 395, 680 | 37.0 |
| MYRTH_3_00099 | 1054, 1360, 1666 | 37.0 |
| Myrth2p4_006397 | 1029, 1335, 1641 | 37.1 |
| MYRTH_2_03760 | 897, 1203, 1509 | 43.3 |
| AURPU_3_00017 | 1775, 2162, 2549 | 36.8 |
| AURPU_3_00429 | 1814, 2201, 2588 | 37.0 |
| AURPU_3_00353 | 1848, 2235, 2622 | 37.3 |

In a second set of experiments with acid pretreated corn stover (aCS) as the substrate, supernatants of a different set of protein fermentations were added to TEC-210 as described above. Several proteins demonstrated increased sugar release, as shown below in Table 8.

TABLE 8
Effect a different set of various proteins spiked on TEC-210 using
aCS substrate

| Target ID | SEQ ID NO: | Glucose (AU) |
|---|---|---|
| TEC only | — | 52.6 |
| Scyth2p4_009823 | 201, 486, 771 | 56.5 |
| SCYTH_1_02579 | 258, 543, 828 | 57.1 |
| SCYTH_1_00740 | 12, 297, 582 | 57.7 |
| Myrth2p4_008437 | 1088, 1394, 1700 | 57.7 |
| Myrth2p4_003274 | 931, 1237, 1543 | 58.3 |
| Myrth2p4_006213 | 1107, 1413, 1719 | 70.5 |
| AURPU_3_00208 | 2118, 2505, 2892 | 60.7 |
| Aurpu2p4_008503 | 1970, 2357, 2744 | 61.5 |
| Aurpu2p4_006782 | 1920, 2307, 2694 | 62.3 |

In a third set of experiments with aCS as the substrate, supernatant of GH61 MTP fermentations was added to a 3 enzyme cellulase base mixes, as described above. Spiking showed increased sugar release, as shown below in Table 9.

TABLE 9
Effect of various GH61 enzymes spiked
on 3 enzyme mix using aCS substrate

| Target ID | SEQ ID NOs: | Glucose (AU) |
|---|---|---|
| 3 enzyme mix | — | 21.9 |
| SCYTH_1_00672 | 104, 389, 674 | 26.6 |
| SCYTH_1_05851 | 157, 442, 727 | 26.8 |
| Scyth2p4_002689 | 50, 335, 620 | 27.6 |
| MYRTH_2_03236 | 857, 1163, 1469 | 26.8 |
| MYRTH_2_01413 | 953, 1259, 1565 | 27.1 |
| MYRTH_2_03391 | 950, 1256, 1562 | 27.1 |
| AURPU_3_00402 | 2001, 2388, 2775 | 24.0 |
| AURPU_3_00407 | 2010, 2397, 2784 | 24.7 |
| AURPU_3_00395 | 1904, 2291, 2678 | 26.3 |

In another set of experiments with acid pretreated corn stover (aCS) as the substrate, the supernatants of one protein MTP fermentations was added to TEC-210 as described above. This protein showed increased sugar release, as shown below in Table 10.

TABLE 10
Effect of the AURPU_3_00184 protein
spiked on TEC-210 using aCS substrate

| Target ID | SEQ ID NOs: | Glucose (AU) |
|---|---|---|
| TEC only | — | 25.1 |
| AURPU_3_00184 | 1902, 2289, 2676 | 28.9 |

### Example 19

### Improvement of Thermophilic Cellulase Mixture by Various Scytalidium thermophilum Proteins in an Activity Assay at Labscale Including Mixing

Scytalidium thermophilum proteins were cloned and expressed in A. niger as described above (Examples 8-10). Concentrated supernatants from shake flask fermentations were used in sugar release activity assays as described above (Example 14), using 10% aCS NREL as feedstock. In one set of experiments, supernatant of the Scytalidium thermophilum protein Scyth2p4_{—}009442 was spiked based on protein dosage on top of a TEC-210 base mix, as described above. The protein showed increased sugar release, as shown below in Table 11.

TABLE 11
Effect of a Scytalidium thermophilum protein spiked based
on protein dosage on TEC-210 using 10% DM aCS substrate

Glucose

| Target ID | SEQ ID NOs: | Average (AU) | stdev |
|---|---|---|---|
| TEC only | — | 31.4 | 0.07 |
| Scyth2p4_009442 | 178, 463, 748 | 32.5 | 0.09 |

### Example 20

### Improvement of Thermophilic Cellulase Mixture by Various Aureobasidium pullulans Proteins in an Activity Assay at Labscale Including Mixing

The cellulase enhancing activity of various Aureobasidium pullulans beta-galactosidase (BG) proteins were further analyzed. The supernatant of the A. niger expressing shake flask fermentations were concentrated and spiked in a dosage of 0.45 mg/gDM on top of a base activity of a three enzyme base mix (4.55 mg/gDM composed of: CBHI at 1.25 g/gDM, CBHII at 1.5 mg/gDM and GH61 at 1.8 mg/gDM) at a feedstock concentration of 10% (w/w) aCS, as described above (Example 14). As a negative control, the 3 enzyme base mix was also tested. All experiments were performed at least in duplicate and were incubated for 72 hours at 65° C. in an oven incubator (Techne HB-1D hybridization oven) while rotating at set-point 3. After incubation, the samples were centrifuged and soluble sugars were analysed by HPLC as described above (Example 15). Addition of the Aureobasidium pullulans BG proteins yielded increased sugar release, as shown below in Table 12.

TABLE 12
Effect of Aureobasidium pullulans BG proteins
spiked on top of a 3E mix using aCS substrate

| Protein ID | SEQ ID NOs: | Glucose (g/L) |
|---|---|---|
| 3 enzyme mix | — | 6.7 |
| Aurpu2p4_006782 | 1920, 2307, 2694 | 26.1 |
| AURPU_3_00208 | 2118, 2505, 2892 | 26.8 |

### Example 21

### Improvement of Thermophilic Cellulase Mixture by Various Proteins in an Activity Assay at Labscale Including Mixing

The cellulase enhancing activity of various GH61 proteins were further analyzed. The supernatant of the A. niger expressing Scyth2p4_{—}002220, MYRTH_{—}2_{—}04272, and MYRTH_{—}2_{—}01413 shake flask fermentations were concentrated and spiked in a dosage of 1.8 mg/gDM on top of a base activity of a three enzyme base mix (3.2 mg/gDM composed of: BG at 0.45 g/gDM, CBHI at 1.5 mg/gDM and CBHII at 1.25 mg/gDM) at a feedstock concentration of 10% (w/w) aCS, as described above (Example 14). As a negative control, the 3 enzyme base mix was also tested. All experiments were performed at least in duplicate and were incubated for 72 hours at 65° C. in an oven incubator (Techne HB-1D hybridization oven) while rotating at set-point 3. After incubation, the samples were centrifuged and soluble sugars were analysed by HPLC as described above (Example 15). Addition of these GH61 proteins yielded increased sugar release, as shown below in Table 13.

TABLE 13
Effect of various GH61 proteins spiked
on top of a 3E mix using aCS substrate

| Target ID | SEQ ID NOs: | Glucose (g/L) |
|---|---|---|
| 3 enzyme mix | — | 29.7 |
| Scyth2p4_002220 | 42, 327, 612 | 32.5 |
| MYRTH_2_04272 | 1040, 1346, 1652 | 32.1 |
| MYRTH_2_01413 | 953, 1259, 1565 | 32.4 |

In another experiment, the cellulase enhancing activity of Scytalidium thermophilum CBHII protein SCYTH_{—}1_{—}03721 was further analyzed. The SCYTH_{—}1_{—}03721 gene was cloned and expressed in A. niger as described above (Examples 8-10). The supernatant of an A. niger expressing SCYTH_{—}1_{—}03721 shake flask fermentation was concentrated and spiked in a dosage of 1.5 mg/gDM on top of a base activity of a three enzyme base mix (3.5 mg/gDM composed of: BG at 0.45 g/gDM, CBHI at 1.25 mg/gDM and GH61 at 1.8 mg/gDM) at a feedstock concentration of 10% (w/w) aCS, as described above (Example 14). As a negative control, the 3 enzyme base mix was also tested. All experiments were performed at least in duplicate and were incubated for 72 hours at 65° C. in an oven incubator (Techne HB-1D hybridization oven) while rotating at set-point 3. After incubation, the samples were centrifuged and soluble sugars were analysed by HPLC as described above (Example 15). Addition of the SCYTH_{—}1_{—}03721 protein yielded increased sugar release, as shown below in Table 14.

TABLE 14
Effect of CBHII SCYTH_1_03721 protein spiked
on top of a 3E mix using aCS substrate

| Target ID | SEQ ID NOs: | Glucose (g/L) |
|---|---|---|
| 3 enzyme mix | — | 28.1 |
| SCYTH_1_03721 | 129, 414, 699 | 32.5 |

In another experiment, the cellulase enhancing activity of another Myriococcum thermophilum GH61 protein was further analysed. The supernatant of the A. niger expressing MYRTH_{—}2_{—}03760 shake flask fermentation was concentrated and spiked in a dosage of 1.8 mg/gDM on top of a base activity of a three enzyme base mix (3.2 mg/gDM composed of: BG at 0.45 g/gDM, CBHI at 1.5 mg/gDM and CBHII at 1.25 mg/gDM) at a feedstock concentration of 10% (w/w) aCS, as described above (Example 14). As a negative control, the 3 enzyme base mix was also tested. All experiments were performed at least in duplicate and were incubated for 72 hours at 65° C. in an oven incubator (Techne HB-1D hybridization oven) while rotating at set-point 3. After incubation, the samples were centrifuged and soluble sugars were analysed by HPLC as described above (Example 15). Addition of this Myriococcum thermophilum GH61 protein yielded increased sugar release, as shown below in Table 15.

TABLE 15
Effect of GH61 protein MYRTH_2_03760 spiked
on top of a 3E mix using aCS substrate

| Target ID | SEQ ID NOs: | Glucose (g/L) |
|---|---|---|
| 3 enzyme mix | — | 17.8 |
| MYRTH_2_03760 | 897, 1203, 1509 | 19.1 |

In another experiment, the cellulose-enhancing activity of Myriococcum thermophilum CBHI protein MYRTH2p4_{—}003203 was further analyzed. The MYRTH2p4_{—}003203 gene was cloned and expressed in A. niger as described above (Examples 8-10). The supernatant of an A. niger expressing MYRTH2p4_{—}003203 shake flask fermentation was concentrated and spiked in a dosage of 1.25 mg/gDM on top of a base activity of a three enzyme base mix (3.75 mg/gDM composed of: BG at 0.45 g/gDM, CBHII at 1.5 mg/gDM and GH61 at 1.8 mg/gDM) at a feedstock concentration of 10% (w/w) aCS, as described above (Example 14). As a negative control, the 3 enzyme base mix was also tested. All experiments were performed at least in duplicate and were incubated for 72 hours at 65° C. in an oven incubator (Techne HB-1D hybridization oven) while rotating at set-point 3. After incubation, the samples were centrifuged and soluble sugars were analysed by HPLC as described above (Example 15).

Addition of this Myriococcum thermophilum CBHI protein yielded increased sugar release, as shown below in Table 16.

TABLE 16
Effect of CBHI MYRTH2p4_003203 protein
spiked on top of a 3E mix using aCS substrate

| Target ID | SEQ ID NOs: | Glucose (g/L) |
|---|---|---|
| 3 enzyme mix | — | 19.2 |
| MYRTH2p4_003203 | 930, 1237, 1543 | 22.1 |

In another experiment, the cellulase enhancing activity of Myriococcum thermophilum beta-galactosidase (BG) protein MYRTH_{—}1_{—}00021 was further analyzed. The supernatant of an A. niger expressing MYRTH_{—}1_{—}00021 shake flask fermentation was concentrated and spiked in a dosage of 0.45 mg/gDM on top of a base activity of a three enzyme base mix (4.55 mg/gDM composed of: CBHI at 1.25 g/gDM, CBHII at 1.5 mg/gDM and GH61 at 1.8 mg/gDM) at a feedstock concentration of 10% (w/w) aCS, as described above (Example 14). As a negative control, the 3 enzyme base mix was also tested. All experiments were performed at least in duplicate and were incubated for 72 hours at 65° C. in an oven incubator (Techne HB-1D hybridization oven) while rotating at set-point 3. After incubation, the samples were centrifuged and soluble sugars were analysed by HPLC as described above (Example 15).

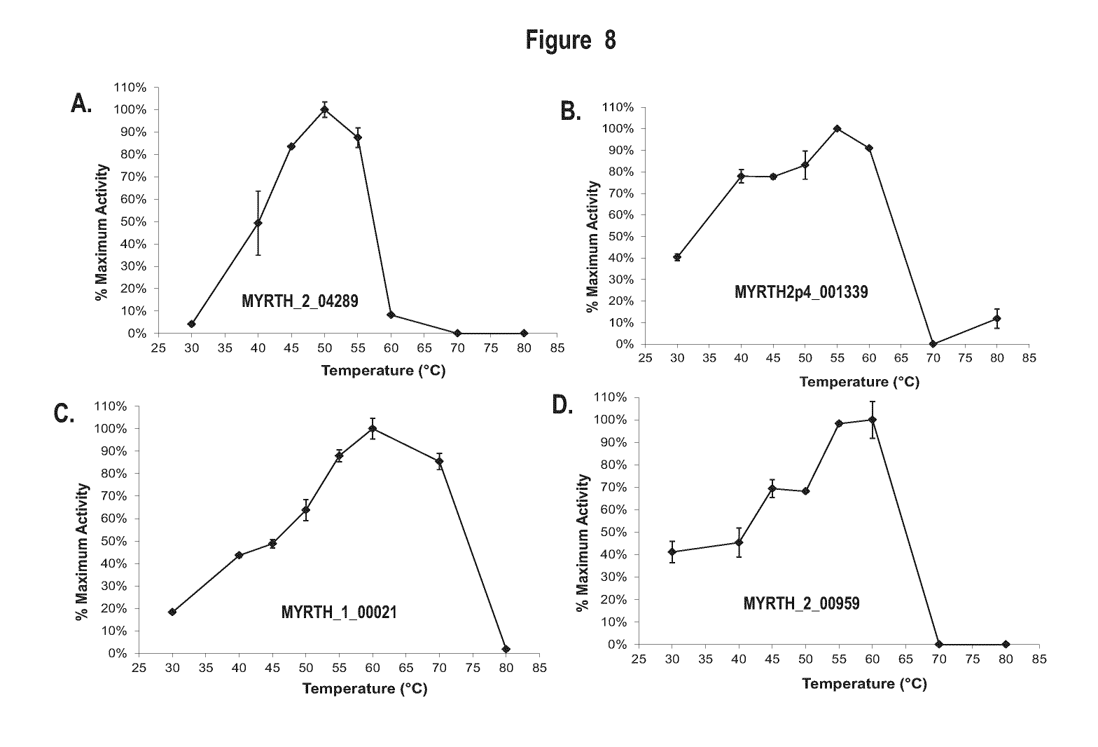

Addition of this Myriococcum thermophilum BG protein yielded increased sugar release, as shown below in Table 17 and in FIG. 8.

TABLE 17
Effect of beta-galactosidase MYRTH_1_00021 protein
spiked on top of a 3E mix using aCS substrate

| Target ID | SEQ ID NOs: | Glucose (g/L) |
|---|---|---|
| 3 enzyme mix | — | 13.8 |
| MYRTH_1_00021 | 1113, 1419, 1725 | 16.6 |

### Example 22

### Identification of Thermophilic Various Arabino(Furano)Sidases

The arabino(furano)sidase activity of various enzymes was further analysed, as described above (Example 16.1). The supernatant of A. niger shake flask fermentations were concentrated and assayed for arabinose release from wheat arabinoxylan, which was pre-digested by an endo-xylanase, after incubation for 24 hours at pH 4.5 and 65° C. Three enzymes showed increased arabinose release as shown below in Table 18.

TABLE 18
Effect of various proteins on pre-
digested wheat arabinoxylan substrate

| | | μg/mL | % arabinose |
|---|---|---|---|
| Target ID | SEQ ID NOs: | arabinose | release of max |
| no enzyme | — | 4.5 | 0 |
| SCYTH_1_01777 | 36, 321, 606 | 7.6 | 0.4 |
| SCYTH_1_01831 | 191, 476, 761 | 23.4 | 2.7 |
| MYRTH_1_00007 | 1015, 1321, 1627 | 10.6 | 0.9 |
| MYRTH_3_00127 | 1144, 1450, 1756 | 161.4 | 22 |
| MYRTH_1_00002 | 1109, 1415, 1721 | 5.3 | 0.1 |
| MYRTH_2_00959 | 1126, 1432, 1738 | 149.0 | 20.6 |
| AURPU_3_00341 | 1976, 2363, 2750 | 7.8 | 0.5 |
| AURPU_3_00342 | 1824, 2211, 2598 | 7.3 | 0.4 |
| AURPU_3_00410 | 1992, 2379, 2766 | 11.7 | 1 |
| AURPU_3_00333 | 1993, 2380, 2767 | 5.6 | 0.2 |

### Example 23

### Identification of Thermophilic Beta-Xylosidases

The beta-xylosidase activity of various enzymes was further analyzed. The supernatants of the A. niger shake flask fermentations were concentrated and assayed in different dosages for xylose release from xylobiose after incubation for 24 hours at pH 4.5 and 65° C. as described above (Example 16.2). Several enzymes showed significant xylose release from xylobiose as shown below in Table 19.

TABLE 19
Effect of various enzymes on release of xylose from xylobiose

| | | Concen- | % xylose release |
|---|---|---|---|
| | | tration | from xylobiose (% |
| Target ID | SEQ ID NOs: | (w/w) | from max possible) |
| Scyth2p4_001371 | 19, 304, 589 | 0.1% | 6 |
| | | 1% | 22 |
| MYRTH_1_00003 | 1138, 1444, 1750 | 0.1% | 0 |
| | | 1% | 1 |
| MYRTH_2_01280 | 1131, 1437, 1743 | 0.1% | 0 |
| | | 1% | 4 |
| MYRTH2p4_001496 | 894, 1200, 1506 | 0.5% | 9 |
| MYRTH_2_00959 | 1126, 1433, 1739 | 0.5% | 1 |
| AURPU_3_00184 | 1902, 2289, 2676 | 0.1% | 57 |
| | | 1% | 100 |

### Example 24

### Identification of Thermophilic Scytalidium thermophilum Acetyl-Xylan Esterase

The acetyl-xylan esterase activity of Scytalidium thermophilum SCYTH_{—}2_{—}07393 was further analyzed. The supernatant of this Scytalidium thermophilum A. niger shake flask fermentation was concentrated and assayed for acetic acid release from acid pretreated corn stover as described above (Example 16.3). The enzymes was identified as active acetyl xylan esterase because it was able to release acetic acid from the substrate as is shown in Table 20.

TABLE 20
Effect of SCYTH_2_07393 (SEQ ID NOs: 262, 547, 832) enzyme
on release of acetic acid from pretreated corn stover

| SCYTH_2_07393 | Acetic acid (μg/mL) |
|---|---|
| no enzyme | 63 |
| 0.1% (w/w) | 131 |
| 1% (w/w) | 152 |

### Example 25

### Characterization Various Thermophilic Endoxylanases

The endoxylanase activity of SCYTH_{—}1_{—}09019, SCYTH_{—}1_{—}09441, SCYTH_{—}1_{—}01114, MYRTH_{—}2_{—}03560, AURPU_{—}3_{—}00013, and AURPU_{—}3_{—}00019 proteins was further analyzed. The supernatant of the A. niger shake flask fermentations were concentrated and assayed for endoxylanase activity on wheat arabinoxylan oligosaccharides and beech wood xylan as described above in endoxylanase activity assay 1 (Example 16.4). The proteins were able to release xylose and xylose oligomers release from the two substrates after incubation for 24 hours with 1% (w/w) enzyme dose at pH 4.5 and 65° C. as is shown in Table 21.

TABLE 21
Effect of various proteins on release of xylose and xylose oligomers
from Beech wood xylan and Wheat arabinoxylan

| | | | Amount released (μg/mg substrate) | | | |
|---|---|---|---|---|---|---|
| | 1% (w/w) E/S | SEQ ID NOs: | xylose | xylobiose | xylotriose | xylotetraose |
| Beech wood | no enzyme | — | 1.4 | 0.3 | 0.0 | 0.2 |
| xylan | SCYTH_1_09019 | 260, 545, 830 | 63.0 | 373.0 | 10.7 | 0.5 |
| | SCYTH_1_09441 | 155, 440, 725 | 24.4 | 129.9 | 140.6 | 30.2 |
| | SCYTH_1_01114 | 218, 503, 788 | 29.3 | 180.4 | 162.5 | 20.5 |
| | MYRTH_2_03560 | 1022, 1328, 1634 | 7.0 | 383.9 | 7.8 | 0.8 |
| | AURPU_3_00013 | 1924, 2311, 2698 | 84.6 | 337.1 | 53.4 | 0.5 |
| ara | no enzyme | — | 0.5 | 0.0 | 0.0 | 0.1 |
| bin | SCYTH_1_09019 | 260, 545, 830 | 43.6 | 75.3 | 1.9 | 0.0 |
| | SCYTH_1_09441 | 155, 440, 725 | 5.7 | 14.3 | 7.3 | 2.0 |
| | SCYTH_1_01114 | 218, 503, 788 | 8.0 | 18.4 | 8.3 | 1.4 |
| | MYRTH_2_03560 | 1022, 1328, 1634 | 4.1 | 73.4 | 2.3 | 0.0 |
| | AURPU_3_00013 | 1924, 2311, 2698 | 55.8 | 87.0 | 1.0 | 0.0 |
| | AURPU_3_00019 | 1925, 2312, 2699 | 1.7 | 4.3 | 5.7 | 2.6 |

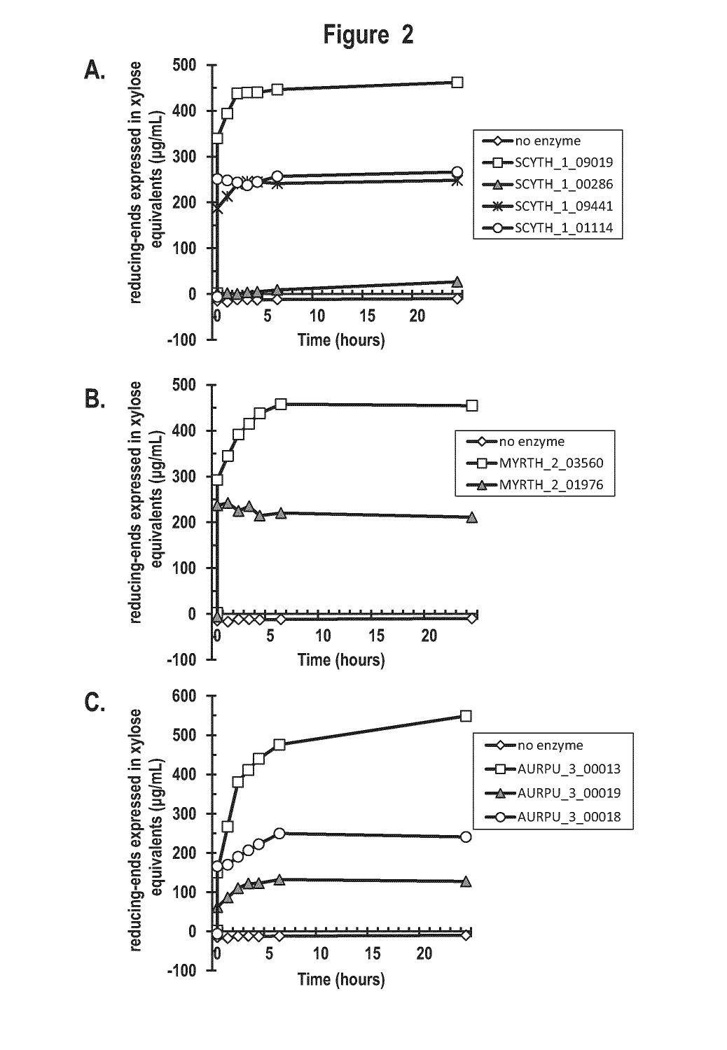

In a second set of experiments, the endoxylanase activity of the proteins SCYTH_{—}1_{—}09019, SCYTH_{—}1_{—}00286, SCYTH_{—}1_{—}09441, SCYTH_{—}1_{—}01114, MYRTH_{—}2_{—}03560, MYRTH_{—}2_{—}01976, AURPU_{—}3_{—}00013, AURPU_{—}3_{—}00019, AURPU_{—}3_{—}00018 was further analyzed as described above in endoxylanase activity assay 2 (Example 16.5). The supernatant of the A. niger shake flask fermentations were concentrated and assayed for endoxylanase activity by measuring reducing-end formation expressed as xylose equivalents after incubation of the enzymes at 0.1% (w/w) dose on wheat arabinoxylan during 24 hours at 65° C. and pH 4.5. The enzymes were able to release reducing sugars from the substrates, as shown in Table 22 and in FIG. 2, where panels A, B and C correspond to proteins from Scytalidium thermophilum, Myriococcum thermophilum, and Aureobasidium pullulans, respectively.

TABLE 22
Effect of various proteins on the release of reducing sugars
(reported as xylose equivalents) from Wheat arabinoxylan

| | | reducing-ends expressed in xylose equivalents (μg/mL) | | | | | | | |
|---|---|---|---|---|---|---|---|---|---|
| Target ID | SEQ ID NOs: | t = 0 h | t = 0.5 h | t = 1 h | t = 2 h | t = 3 h | t = 4 h | t = 6 h | t = 24 h |
| no enzyme | — | −6.0 | −14.5 | −17.0 | −11.9 | −11.7 | −12.6 | −11.8 | −9.9 |
| SCYTH_1_09019 | 260, 545, 830 | 2.3 | 340.0 | 394.0 | 438.0 | 440.1 | 440.2 | 446.4 | 462.1 |
| SCYTH_1_00286 | 237, 522, 807 | 2.3 | 0.8 | 1.9 | 0.4 | 3.6 | 5.0 | 9.1 | 26.7 |
| SCYTH_1_09441 | 155, 440, 725 | −6.0 | 187.1 | 213.4 | 240.1 | 245.8 | 244.7 | 241.4 | 248.4 |
| SCYTH_1_01114 | 218, 503, 788 | −6.0 | 251.3 | 248.2 | 243.7 | 237.6 | 244.8 | 257.0 | 266.4 |
| MYRTH_2_03560 | 1022, 1328, 1634 | 2.3 | 292.5 | 344.9 | 391.8 | 415.2 | 437.7 | 457.9 | 454.6 |
| MYRTH_2_01976 | 972, 1278, 1584 | −6.0 | 236.9 | 242.3 | 224.9 | 235.1 | 214.4 | 220.3 | 211.2 |
| AURPU_3_00013 | 1924, 2311, 2698 | 2.3 | 149.8 | 267.0 | 380.5 | 411.1 | 439.5 | 475.6 | 548.5 |
| AURPU_3_00019 | 1925, 2312, 2699 | −6.0 | 62.0 | 86.3 | 110.2 | 122.1 | 122.8 | 131.9 | 127.4 |
| AURPU_3_00018 | 2108, 2495, 2882 | −6.0 | 165.9 | 169.9 | 190.1 | 206.6 | 222.1 | 249.5 | 240.6 |

### Example 26

### Characterization of Thermophilic Aureobasidium pullulans Xyloglucanase

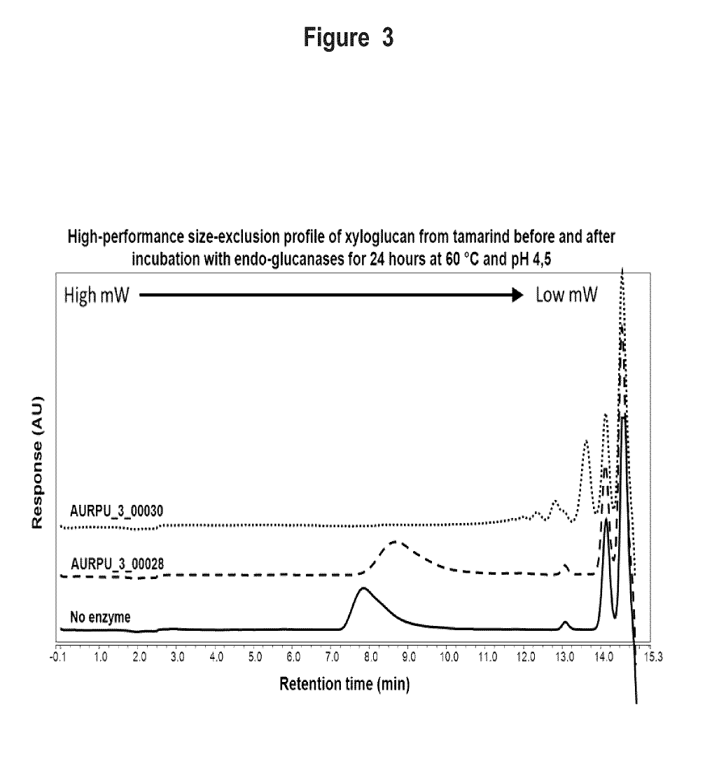

The xyloglucanase activity of AURPU_{—}3_{—}00030 (SEQ ID NOs: 1778, 2165, 2552) and AURPU_{—}3_{—}00028 (SEQ ID NOs: 1947, 2334, 2721) proteins were further analyzed. The supernatant of these two Aureobasidium pullulan A. niger shake flask fermentations were concentrated and assayed for xyloglucanase activity on Tamarind xyloglucan as described above (Example 16.6). Both enzymes were identified as active xyloglucanase because they were able to release low molecular weight oligosaccharides, as shown in FIG. 3.

### Example 27

### Further Characterization of Expressed Enzymes from Scytalidium thermophilum

The Scytalidium thermophilum proteins SCYTH_{—}2_{—}07268, SCYTH_{—}2_{—}07393, SCYTH_{—}1_{—}00740, SCYTH_{—}1_{—}03721, SCYTH_{—}1_{—}03688, SCYTH_{—}1_{—}01623, Scyth2p4_{—}005037, and SCYTH_{—}2_{—}07965 were further characterized using the assay protocols and assay conditions indicated in the table below.

| | SEQ ID | | | Assay | Activity | Fold increase |
|---|---|---|---|---|---|---|
| Target ID | NOs: | Assay Protocol | Substrate | Conditions | (U/mL)^{‡} | over control* |
| SCYTH_2_07268 | 174, 459, | CU5 | Ethyl ferulate, 4 mM | pH 5.3, | 7 | na |
| | 744 | (Example 16.11) | | 40° C., | | |
| | | | | 30 min | | |
| SCYTH_2_07393 | 262, 547, | CU8 | acetylated xylan from | pH 5, | 3.1 | na |
| | 832 | (Example 16.14) | beechwood 0.4% | 40° C., | | |
| | | | | 15 min | | |
| SCYTH_1_00740 | 12, 297, | CU1 | 4-nitrophenyl beta-D- | pH 5, | 3.3 | na |
| | 582 | (Example 16.7) | glucosaminide, 1 mM | 40° C., | | |
| | | | | 30 min | | |
| SCYTH_1_03721 | 129, 414, | CU7 | 4-methylumbelliferyl | pH 5, | 0.002 | na |
| | 699 | (Example 16.13) | beta-D-lactoside, 0.2 mM | 40° C., | | |
| | | | | 30 min | | |
| SCYTH_1_03688 | 259, 544, | CU1 | 4-nitrophenyl alpha- | pH 5, | 1.3 | 65 |
| | 829 | (Example 16.7) | L-arabinofuranoside, | 40° C., | | |
| | | | 1 mM | 30 min | | |
| SCYTH_1_01623 | 160, 445, | CU7 | 4-methylumbelliferyl | pH 5, | 0.0066 | 6.6 |
| | 730 | (Example 16.13) | beta-D-cellobioside, | 40° C., | | |
| | | | 0.2 mM | 30 min | | |
| Scyth2p4_005037 | 86, 371, | CU6 | Polygalacturonic | pH 8, | 0.43 | na |
| | 656 | (Example 16.12) | acid, 0.9% | 40° C., | | |
| | | | | initial rate | | |
| SCYTH_2_07965 | 125, 410, | CU6 | Rhamnogalacturonan | pH 6, | 1.1 | na |
| | 695 | (Example 16.12) | I, 0.7% | 40° C., | | |
| | | | | initial rate | | |

*na, not applicable as control exhibited no detectable activity. Control is an equal volume of supernatant from a vector-only transformant
^{‡}U, micromole product formed per minute under the indicated assay conditions

### Example 28

### Further Characterization of Expressed Enzymes from Myriococcum thermophilum

The Myriococcum thermophilum proteins Myrth2p4_{—}003495, Myrth2p4_{—}005155, Myrth2p4_{—}007061, MYRTH_{—}2_{—}01934, MYRTH2p4_{—}001537, MYRTH2p4_{—}005923, MYRTH2p4_{—}003942, MYRTH_{—}1_{—}00080, MYRTH_{—}4_{—}09372, MYRTH2p4_{—}001451, MYRTH_{—}4_{—}09820, Myrth2p4_{—}003941, MYRTH_{—}1_{—}00024, MYRTH2p4_{—}002293, MYRTH_{—}3_{—}00003, MYRTH_{—}3_{—}00097, MYRTH_{—}4_{—}06111, Myrth2p4_{—}001304, Myrth2p4_{—}000359, Myrth2p4_{—}007801, MYRTH2p4_{—}003203, and Myrth2p4_{—}006226 were further characterized using the assay protocols and assay conditions indicated in the table below.

| | | | | | | Fold |
|---|---|---|---|---|---|---|
| | | | | | | increase |
| | SEQ ID | | | Assay | Activity | over |
| Target ID | NOs: | Assay Protocol | Substrate | Conditions | (U/mL)^{‡} | control* |
| Myrth2p4_003495 | 934, 1240, | CU11 | glucono-delta-lactone | pH 5, | 55 | 12.2 |
| | 1546 | (Example 16.17) | 12 mM | 37° C., | | |
| | | | | 30 min | | |
| Myrth2p4_005155 | 982, 1288, | CU4 | alpha-D-Glucose, | pH 5, | 83 | na |
| | 1594 | (Example 16.12) | 10 umol/mL | 40° C., | | |
| | | | | continuous | | |
| Myrth2p4_007061 | 1045, 1351, | CU4 | alpha-D-Glucose, | pH 5, | 96 | na |
| | 1657 | (Example 16.10) | 10 umol/mL | 40° C., | | |
| | | | | continuous | | |
| MYRTH_2_01934 | 980, 1286, | CU3 | alpha-naphthyl acetate, | pH 5, | 11.8 | na |
| | 1592 | (Example 16.9) | 0.4 mM | 30° C., | | |
| | | | | continuous | | |
| MYRTH2p4_001537 | 895, 1201, | CU8 | acetylated xylan from | pH 5, | 2.7 | na |
| | 1507 | (Example 16.14) | beechwood 0.4% | 40° C., | | |
| | | | | 15 min | | |
| MYRTH2p4_005923 | 1013, 1319, | CU8 | acetylated xylan from | pH 5, | 3.5 | na |
| | 1625 | (Example 16.14) | beechwood 0.4% | 40° C., | | |
| | | | | 15 min | | |
| MYRTH2p4_003942 | 944, 1250, | CU9 | esterified pectin, 1% | pH 8, | 34 | na |
| | 1556 | (Example 16.15) | | 40° C., | | |
| | | | | 15 min | | |
| MYRTH_1_00080 | 1119, 1425, | CU1 | 4-nitrophenyl acetate, 1 mM | pH 5, | 0.2 | 5 |
| | 1731 | (Example 16.7) | | 30° C., | | |
| | | | | 30 min | | |
| MYRTH_4_09372 | 1146, 1452, | CU2 | Xylan from beechwood | pH 5, | 17.3 | 58 |
| | 1758 | (Example 16.8) | 0.2% | 40° C., | | |
| | | | | 30 min | | |
| MYRTH2p4_001451 | 889, 1195, | CU2 | Xylan from beechwood | pH 5, | 12.7 | 42 |
| | 1501 | (Example 16.8) | 0.2% | 40° C., | | |
| | | | | 30 min | | |
| MYRTH_4_09820 | 1147, 1453 | CU2 | Carboxymethylcellulose, | pH 5, | 3 | 10 |
| | 1759 | (Example 16.8) | 0.2% | 40° C., | | |
| | | | | 30 min | | |
| Myrth2p4_003941 | 943, 1249, | CU2 | Laminarin, 0.2% | pH 5, | 2.2 | 220 |
| | 1555 | (Example 16.8) | | 40° C., | | |
| | | | | 30 min | | |
| MYRTH_1_00024 | 943, 1249, | CU2 | Lichenan, 0.2% | pH 5, | 1.05 | 11.7 |
| | 1555 | (Example 16.8) | | 40° C., | | |
| | | | | 30 min | | |
| MYRTH2p4_002293 | 908, 1214, | CU2 | Carboxymenthylcellulose, | pH 5, | 4.1 | 13.7 |
| | 1520 | (Example 16.8) | 0.2% | 40° C., | | |
| | | | | 30 min | | |
| MYRTH_3_00003 | 1138, 1444, | CU2 | Locust bean gum, 0.2% | pH 5, | 1.6 | 1600 |
| | 1750 | (Example 16.8) | | 40° C., | | |
| | | | | 30 min | | |
| MYRTH_3_00097 | 974, 1280, | CU2 | Carboxymethylcellulose, | pH 5, | 2 | 6.7 |
| | 1586 | (Example 16.8) | 0.2% | 40° C., | | |
| | | | | 30 min | | |
| MYRTH_4_06111 | 1071, 1377, | CU2 | Carboxymethylcellulose, | pH 5, | 16 | 53 |
| | 1683 | (Example 16.8) | 0.2% | 40° C., | | |
| | | | | 30 min | | |
| Myrth2p4_001304 | 876, 1182, | CU10 | Lactose, 180 mM | pH 5, | 0.37 | na |
| | 1488 | (Example 16.16) | | 40° C., | | |
| | | | | continuous | | |
| Myrth2p4_000359 | 858, 1164, | CU10 | Lactose, 180 mM | pH 5, | 0.43 | na |
| | 1470 | (Example 16.16) | | 40° C., | | |
| | | | | continuous | | |
| Myrth2p4_007801 | 1065, 1371, | CU6 | Polygalacturonic acid, | pH 8, | 1.1 | na |
| | 1677 | (Example 16.12) | 0.9% | 40° C., | | |
| | | | | continuous | | |
| MYRTH2p4_003203 | 930, 1236, | CU7 | 4-methylumbelliferyl | pH 5, | 0.03 | 30 |
| | 1542 | (Example 16.13) | beta-D-cellobioside | 40° C., | | |
| | | | | 30 min | | |
| Myrth2p4_006226 | 1026, 1332, | CU6 | Polygalacturonic acid | pH 8, | 10 | na |
| | 1638 | (Example 16.12) | 0.9% | 40° C., | | |
| | | | | continuous | | |

*na, not applicable as control exhibited no detectable activity. Control is an equal volume of supernatant from a vector-only transformant
^{‡}U, micromole product formed per minute under the indicated assay conditions

### Example 29

### Further Characterization of Expressed Enzymes from Aureobasidium pullulans

The Aureobasidium pullulans proteins Aurpu2p4_{—}002220, Aurpu2p4_{—}008140, Aurpu2p4_{—}010203, Aurpu2p4_{—}009597, Aurpu2p4_{—}009401, AURPU_{—}3_{—}00030, AURPU_{—}3_{—}00153, AURPU_{—}3_{—}00155, AURPU_{—}3_{—}00166, AURPU_{—}3_{—}00175, AURPU_{—}3_{—}00177, AURPU_{—}3_{—}00191, AURPU_{—}3_{—}00241, AURPU_{—}3_{—}00284, AURPU_{—}3_{—}00296, AURPU_{—}3_{—}00035, and Aurpu2p4_{—}011071, were further characterized using the assay protocols and assay conditions indicated in the table below.

| | | | | | | Fold |
|---|---|---|---|---|---|---|
| | | | | | | increase |
| | SEQ ID | Assay | | Assay | Activity | over |
| Target ID | NOs: | Protocol | Substrate | Conditions | (U/ml)^{‡} | control* |
| Aurpu2p4_002220 | 1836, 2223, | CU4 | alpha-D-Glucose, 10 umol/mL | pH 5, 40° C., | 39 | na |
| | 2610 | (Example 16.10) | | continuous | | |
| Aurpu2p4_008140 | 1959, 2346, | CU5 | Ethyl ferulate, 4 mM | pH 5.3, | 121 | na |
| | 2733 | (Example 16.11) | | 40° C., 30 min | | |
| Aurpu2p4_010203 | 2014, 2401, | CU1 | 4-nitrophenyl acetate, 1 mM | pH 5, 30° C., | 0.17 | 4.3 |
| | 2788 | (Example 16.7) | | 30 min | | |
| Aurpu2p4_009597 | 1995, 2382, | CU3 | alpha-naphthyl acetate, 0.4 mM | pH 5, 30° C., | 48 | na |
| | 2769 | (Example 16.9) | | continuous | | |
| Aurpu2p4_009401 | 1989, 2376, | CU1 | 4-nitrophenyl butyrate, 1 mM | pH 7, 30° C., | 1.2 | na |
| | 2763 | (Example 16.7) | | 30 min | | |
| AURPU_3_00030 | 1778, 2165, | CU2 | xyloglucan from tamarind, | pH 5, 40° C., | 7.6 | 109 |
| | 2552 | (Example 16.8) | 0.08% | 30 min | | |
| AURPU_3_00153 | 1987, 2374, | CU1 | 4-nitrophenyl alpha-L- | pH 5, 40° C., | 0.51 | na |
| | 2761 | (Example 16.7) | arabinopyranoside, 1 mM | 30 min | | |
| AURPU_3_00155 | 1952, 2339, | CU2 | polygalacturonic acid, 0.1% | pH 5, 40° C., | 157 | 390 |
| | 2726 | (Example 16.8) | | 30 min | | |
| AURPU_3_00166 | 1893, 2280, | CU2 | polygalacturonic acid, 0.1% | pH 5, 40° C., | 4.1 | 10.3 |
| | 2667 | (Example 16.8) | | 30 min | | |
| AURPU_3_00175 | 1978, 2365, | CU2 | polygalacturonic acid, 0.1% | pH 5, 40° C., | 12 | 30 |
| | 2752 | (Example 16.8) | | 30 min | | |
| AURPU_3_00177 | 1934, 2321, | CU2 | polygalacturonic acid, 0.1% | pH 5, 40° C., | 2.6 | 6.5 |
| | 2708 | (Example 16.8) | | 30 min | | |
| AURPU_3_00191 | 1873, 2260, | CU1 | 4-nitrophenyl beta-D- | pH 5, 40° C., | 20.6 | 412 |
| | 2647 | (Example 16.7) | glucopyranoside, 1 mM | 30 min | | |
| AURPU_3_00241 | 1936, 2323, | CU1 | 4-nitrophenyl beta-D- | pH 5, 40° C., | 89.8 | 1800 |
| | 2710 | (Example 16.7) | glucopyranoside, 1 mM | 30 min | | |
| AURPU_3_00284 | 1801, 2188, | CU2 | sucrose, 0.2% | pH 5, 40° C., | 10.4 | 210 |
| | 2575 | (Example 16.8) | | 30 min | | |
| AURPU_3_00296 | 1847, 2234, | CU2 | sucrose, 0.2% | pH 5, 40° C., | 12.6 | 250 |
| | 2621 | (Example 16.8) | | 30 min | | |
| AURPU_3_00035 | 1931, 2318, | CU1 | 4-nitrophenyl alpha-L- | pH 5, 40° C., | 53 | na |
| | 2705 | (Example 16.7) | arabinopyranoside, 1 mM | 30 min | | |
| Aurpu2p4_011071 | 2040, 2427, | CU6 | Rhamnogalacturonan I, 0.7% | pH 6, 40° C., | 0.86 | na |
| | 2814 | (Example 16.12) | | continuous | | |

*na, not applicable as control exhibited no detectable activity. Control is an equal volume of supernatant from a vector-only transformant
^{‡}U, micromole product formed per minute under the indicated assay conditions

### Example 30

### Determination of Activity-Temperature Profiles

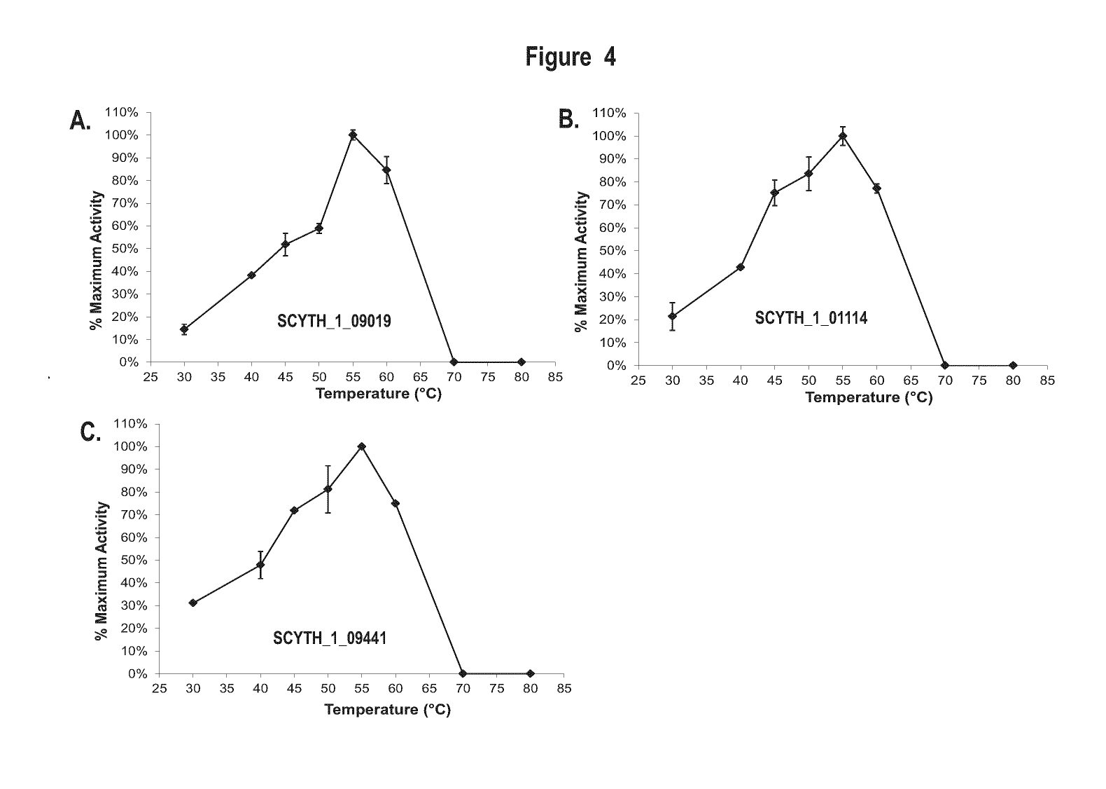

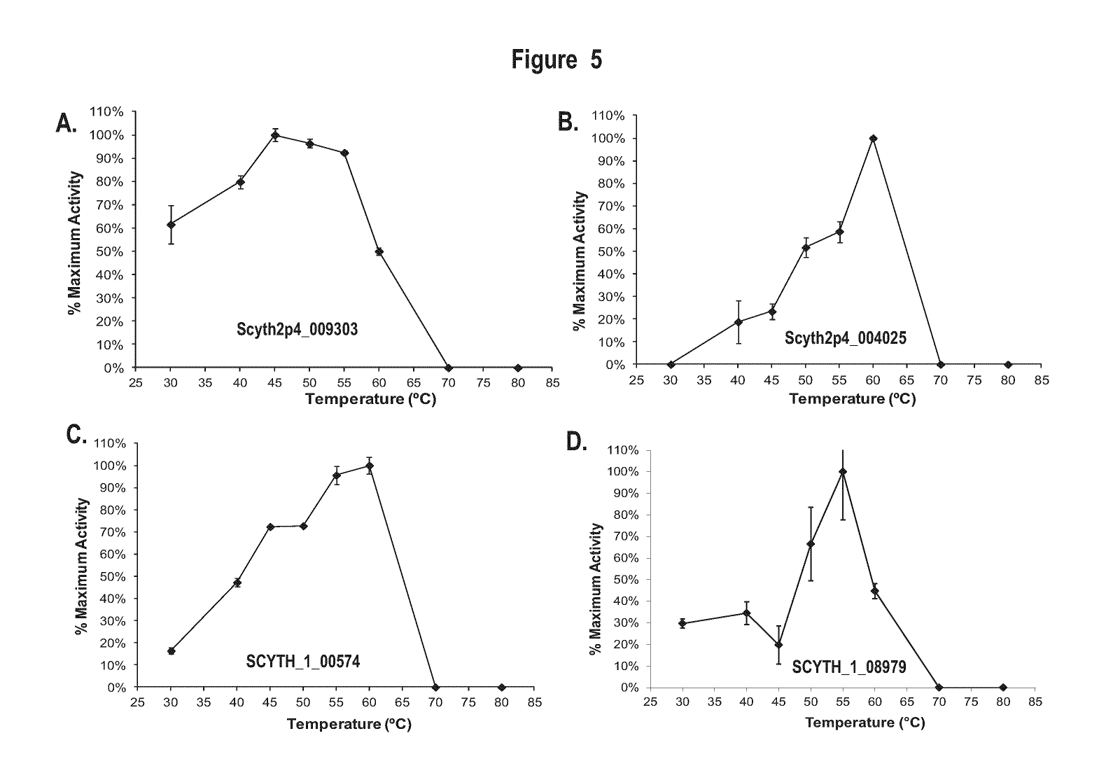

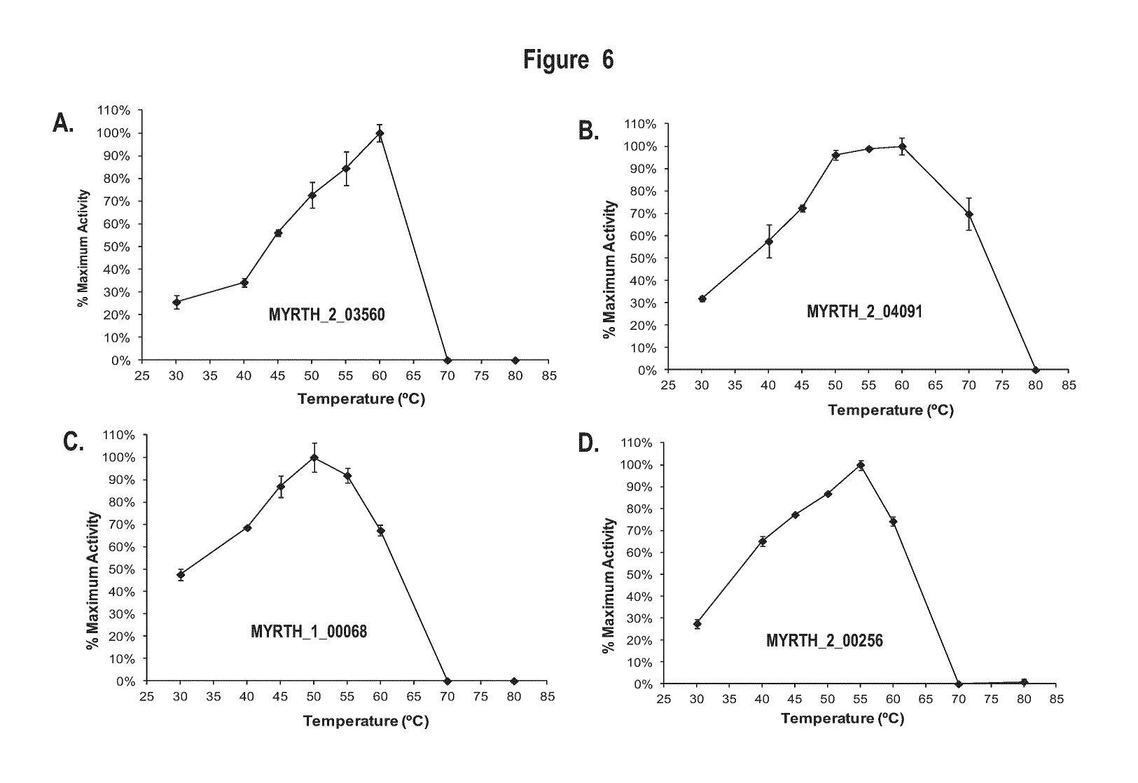

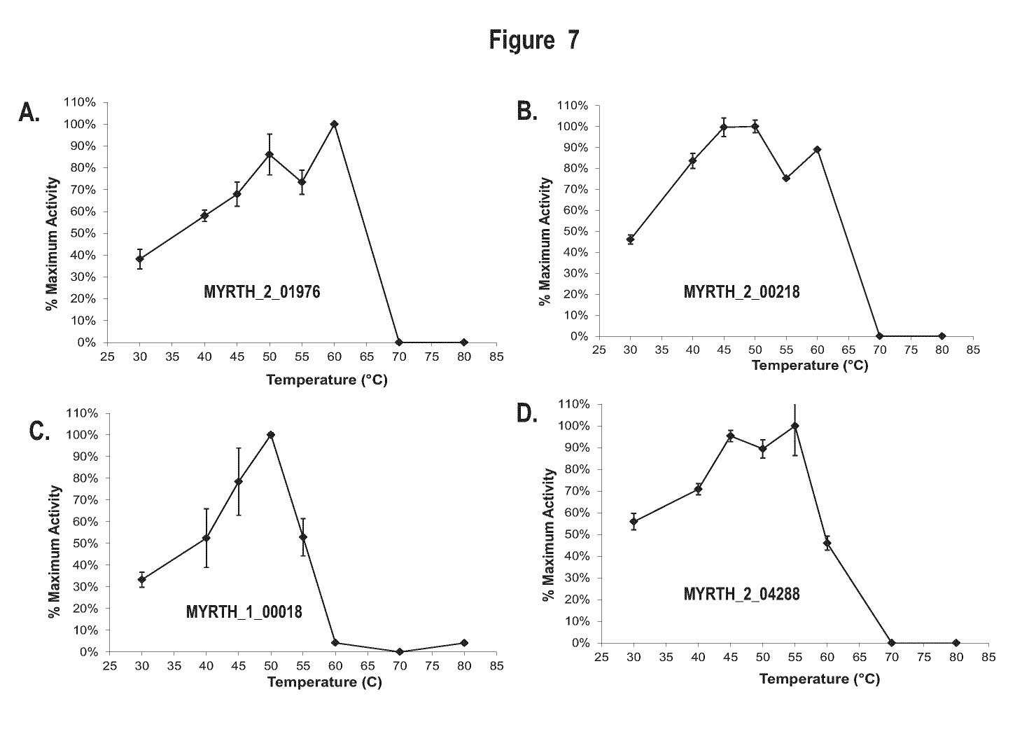

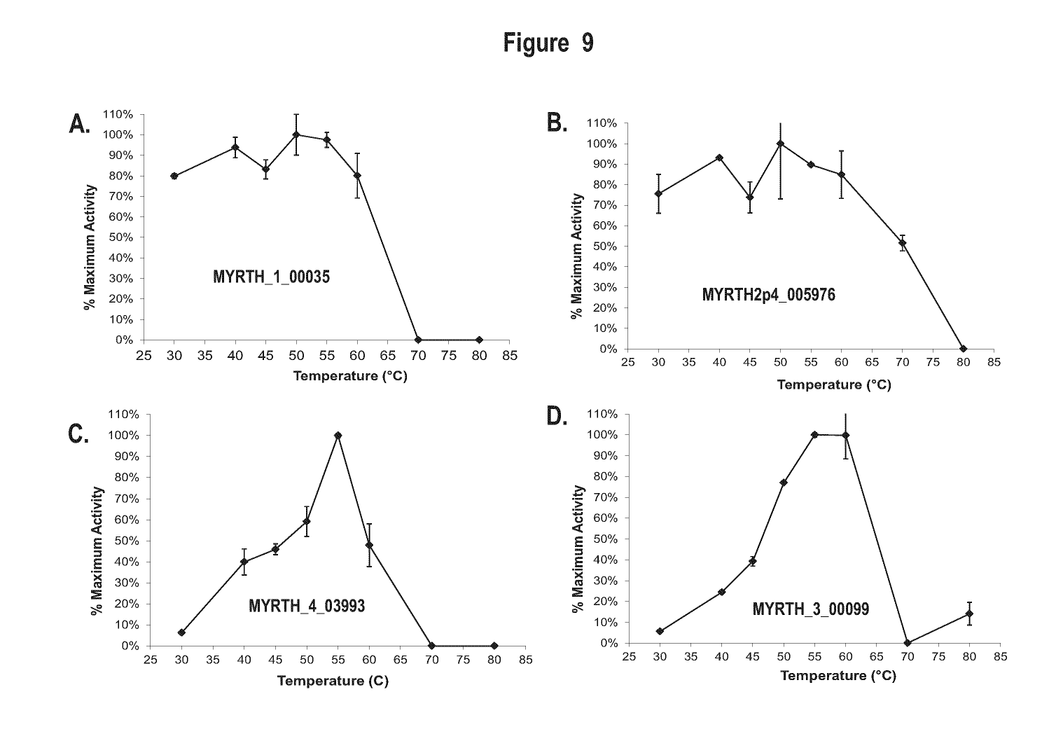

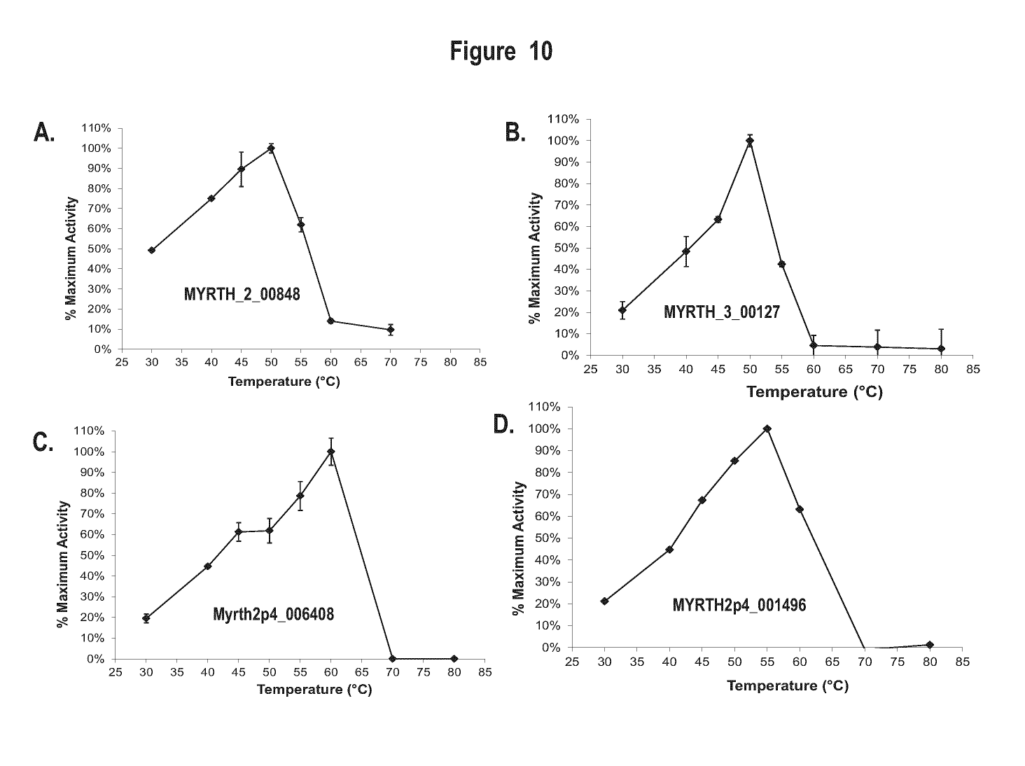

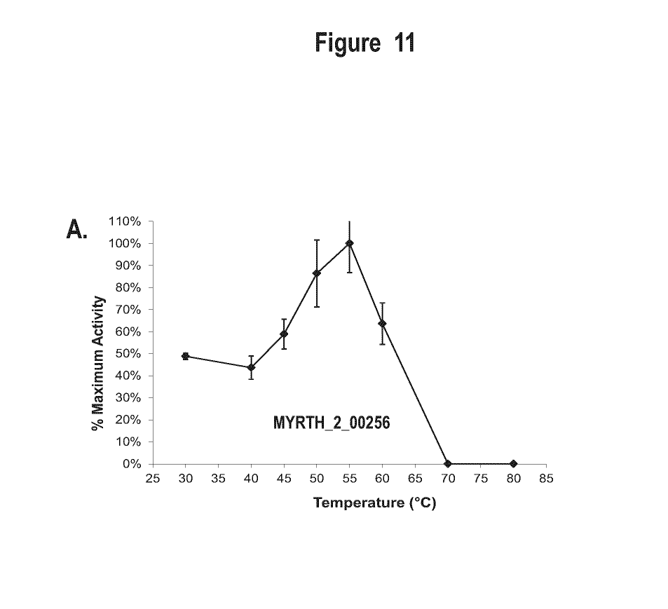

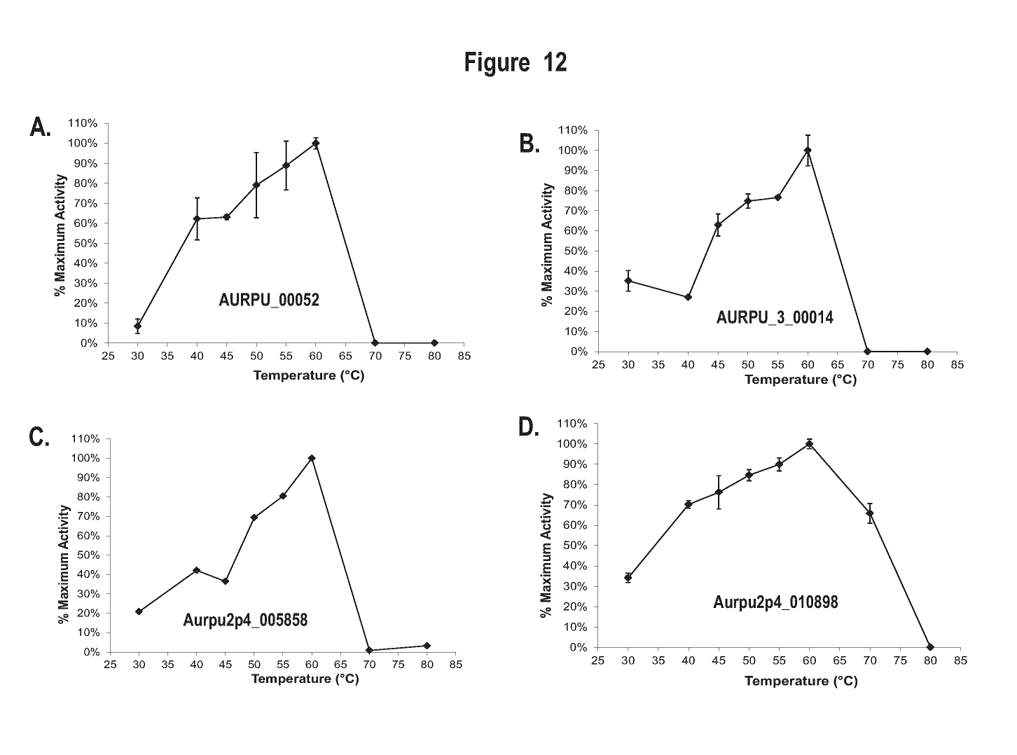

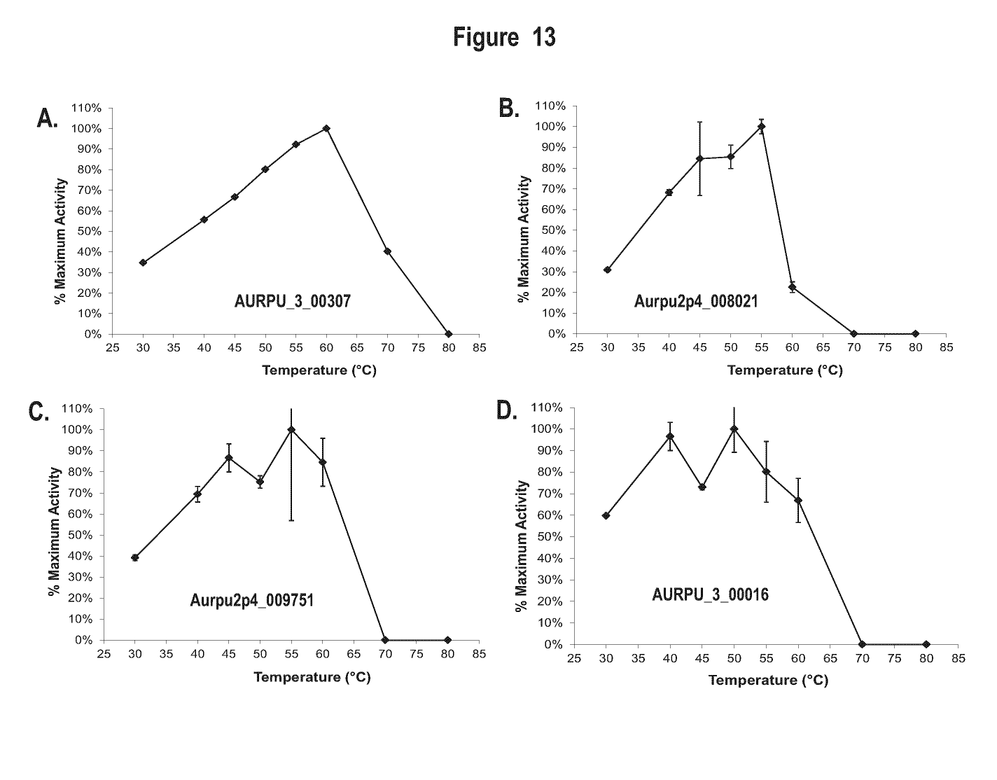

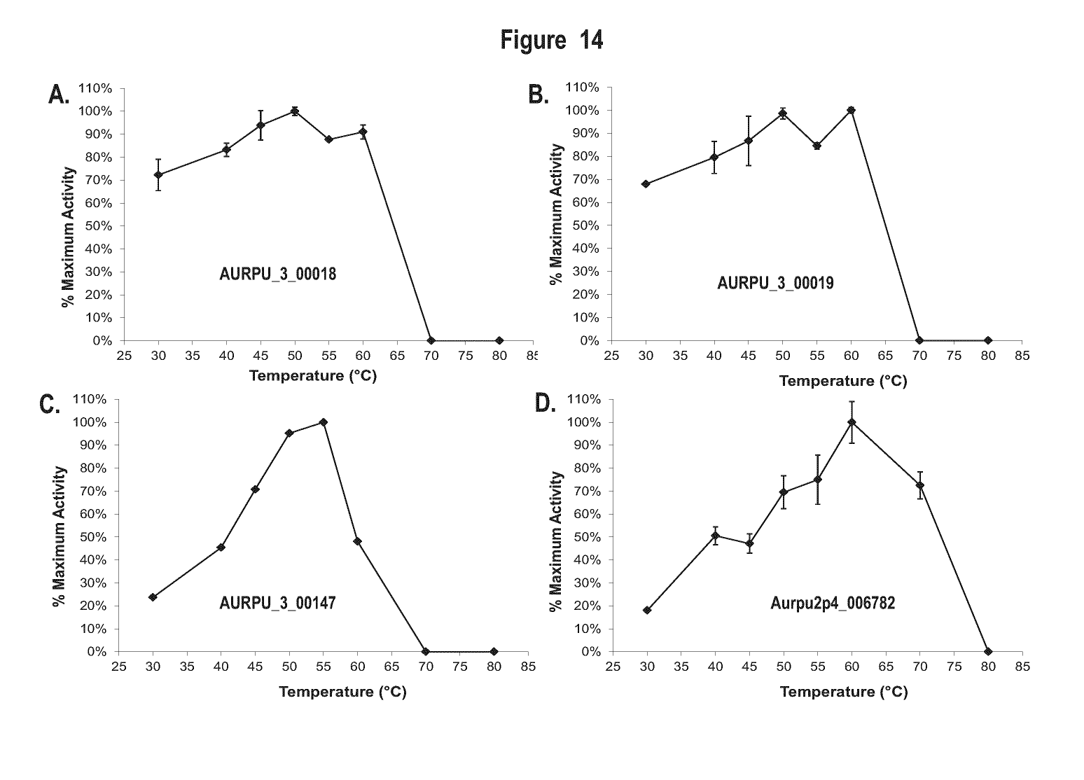

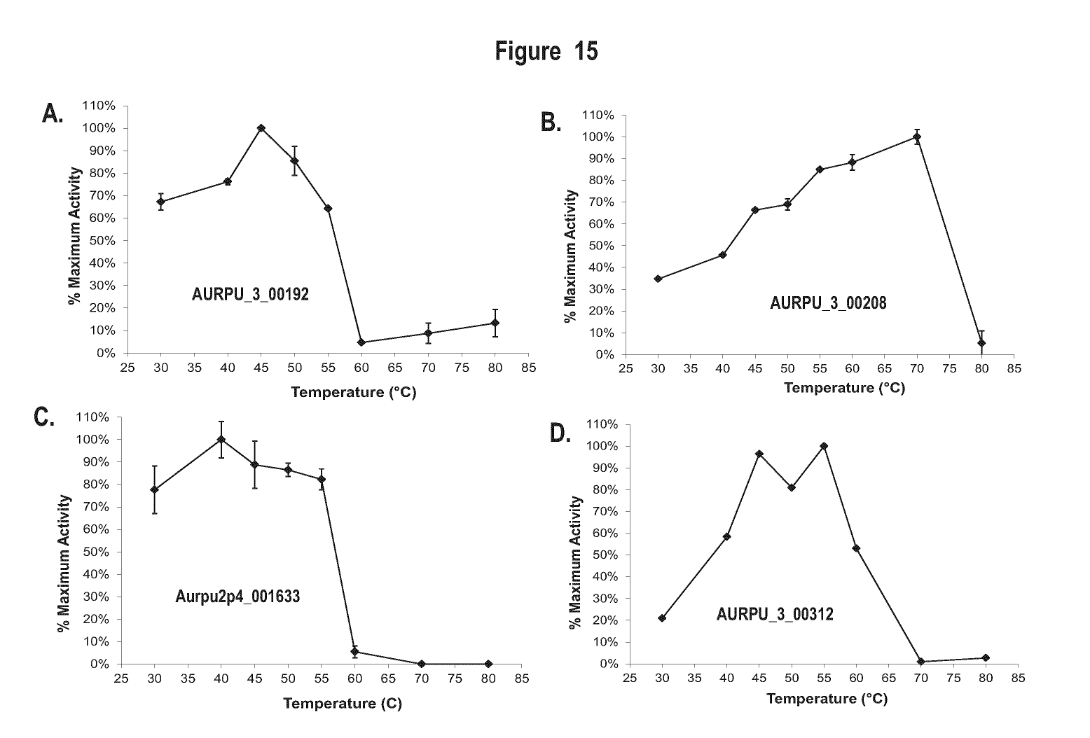

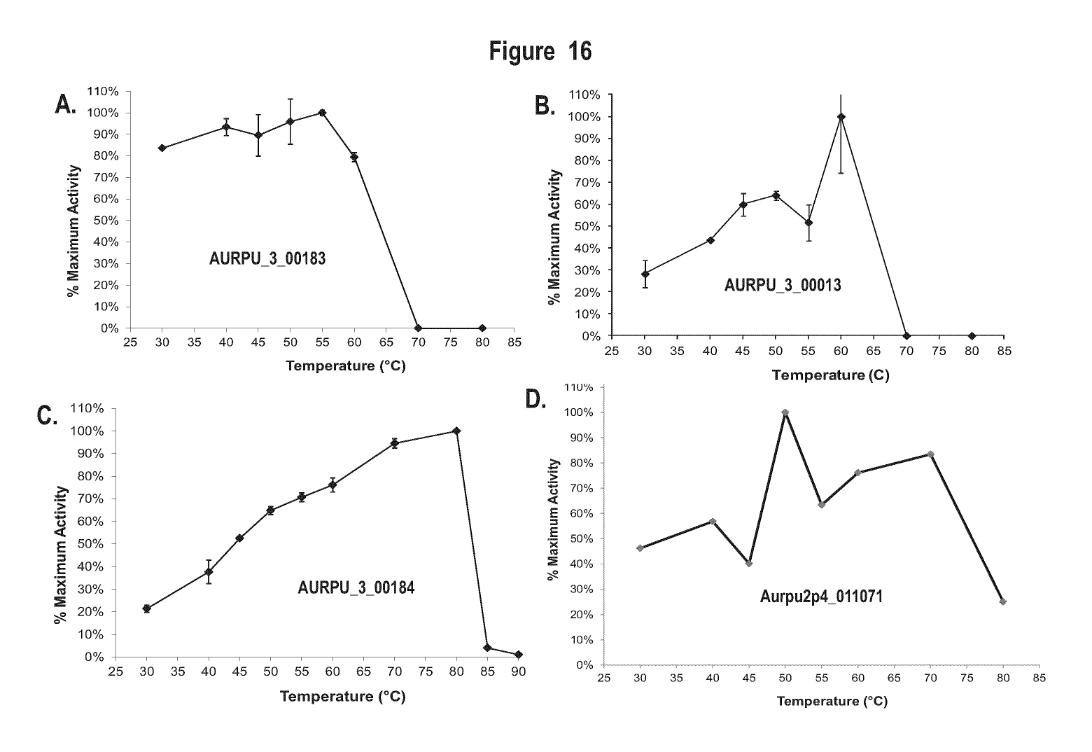

The activity-temperature profiles were determined for various proteins of the present invention according to the protocol in Example 16.18. Results for are shown in FIGS. 4-16 for various proteins from Scytalidium thermophilum, Myriococcum thermophilum, and Aureobasidium pullulans, using the Assay Protocols and Assay Conditions indicated below in Tables 23-25.

TABLE 23
Activity-temperature profiles for various Scytalidium thermophilum proteins

| | | | | |
|---|---|---|---|---|
| | | SEQ ID | Assay | |
| Figure | Protein | NOs: | protocol | Assay conditions |

| | | | |
|---|---|---|---|
| 4A | SCYTH_1_09019 | CU2 | 0.2% xylan from beechwood, |
| | | (Example 16.8) | pH 6.0, 30 min |
| 4B | SCYTH_1_01114 | CU2 | 0.2% xylan from beechwood, |
| | | (Example 16.8) | pH 5.5, 30 min |
| 4C | SCYTH_1_09441 | CU2 | 0.2% xylan from beechwood, |
| | | (Example 16.8) | pH 5.5, 30 min |
| 5A | Scyth2p4_009303 | CU1 | 1 mM pNP-alpha-L-Arabinofuranoside, |
| | | (Example 16.7) | pH 5.0, 30 min |
| 5B | Scyth2p4_004025 | CU2 | 0.1% wheat arabinoxylan, |
| | | (Example 16.8) | low viscosity, pH 5.0, 30 min. |
| 5C | SCYTH_1_00574 | CU2 | 0.2% carboxymethylcellulose, |
| | | (Example 16.8) | pH 6.0, 30 min |
| 5D | SCYTH_1_08979 | CU7 | 0.2 mM 4-methylumbelliferyl- |
| | | (Example 16.13) | cellobioside, pH 5.0, 30 min |

TABLE 24
Activity-temperature profiles for various Myriococcum thermophilum proteins

| | | | | |
|---|---|---|---|---|
| | | SEQ ID | | |
| Figure | Protein | NOs: | Assay protocol | Assay conditions |

| | | | |
|---|---|---|---|
| 6A | MYRTH_2_03560 | CU2 | 0.2% beechwood xylan, |
| | | (Example 16.8) | pH 4.0, 30 min |
| 6B | MYRTH_2_04091 | CU2 | 0.2% beechwood xylan |
| | | (Example 16.8) | pH 7.0, 30 min |
| 6C | MYRTH_1_00068 | CU2 | 0.2% beechwood xylan |
| | | (Example 16.8) | pH 4.0, 30 min |
| 6D | MYRTH_2_00256 | CU7 | 4-methylumbelliferyl- |
| | | (Example 16.13) | cellobioside, pH 5.5, 30 min |
| 7A | MYRTH_2_01976 | CU2 | 0.2% beechwood xylan |
| | | (Example 16.8) | pH 6.0, 30 min |
| 7B | MYRTH_2_00218 | CU2 | 0.2% carboxymethylcellulose |
| | | (Example 16.8) | (7M + 4M), pH 5, 30 min |
| 7C | MYRTH_1_00018 | CU1 | 1 mM 4-nitrophenyl beta-D- |
| | | (Example 16.7) | galactopyranoside, pH 4.0, 30 min |
| 7D | MYRTH_2_04288 | CU2 | 0.08% locust bean gum, |
| | | (Example 16.8) | pH 5.0, 30 min |
| 8A | MYRTH_2_04289 | CU2 | 0.08% locust bean gum, |
| | | (Example 16.8) | pH 5.0, 30 min |
| 8B | MYRTH2p4_001339 | CU1 | 1 mM pNP-beta-glucopyranoside, |
| | | (Example 16.7) | pH 5.0, 30 min |
| 8C | MYRTH_1_00021 | CU1 | 1 mM pNP beta-glucopyranoside, |
| | | (Example 16.7) | pH 5.5, 30 min |
| 8D | MYRTH_2_00959 | CU2 | 0.1% low viscosity wheat |
| | | (Example 16.8) | arabinoxylan, pH 6.0, 30 min |
| 9A | MYRTH_1_00035 | CU2 | 0.08% locust bean gum, |
| | | (Example 16.8) | pH 6.0, 30 min |
| 9B | MYRTH2p4_005976 | CU2 | 0.2% carboxymethylcellulose, |
| | | (Example 16.8) | pH 5.5, 30 min |
| 9C | MYRTH_4_03993 | CU7 | 4-methylumbelliferyl-cellobioside, |
| | | (Example 16.13) | pH 4.5, 30 min |
| 9D | MYRTH_3_00099 | CU7 | 4-methylumbelliferyl-lactoside, |
| | | (Example 16.13) | pH 4.0, 30 min |
| 10A | MYRTH_2_00848 | CU1 | 1 mM p-nitrophenyl-alpha-L- |
| | | (Example 16.7) | arabinopyranoside, pH 5.0, 30 min |
| 10B | MYRTH_3_00127 | CU2 | 0.1% wheat arabinoxylan, low |
| | | (Example 16.8) | viscosity, pH 4.5, 30 min |
| 10C | Myrth2p4_006408 | CU2 | 0.08% xyloglucan, |
| | | (Example 16.8) | pH 6.0, 30 min |
| 10D | MYRTH2p4_001496 | CU1 | 1 mM p-nitrophenyl-beta-D- |
| | | (Example 16.7) | Xylopyranoside, pH 4.5, 30 min |
| 11A | MYRTH_2_00256 | CU7 | 0.2 mM 4-methylumbelliferyl- |
| | | (Example 16.13) | cellobioside, pH 5.0, 30 min |

TABLE 25
Activity-temperature profiles for various Aureobasidium pullulans proteins

| | | | | |
|---|---|---|---|---|
| | | SEQ ID | | |
| Figure | Protein | NOs: | Assay protocol | Assay conditions |

| | | | |
|---|---|---|---|
| 12A | AURPU_00052 | CU2 | 0.2% xylan from beechwood, |
| | | (Example 16.8) | pH 4.5, 30 min |
| 12B | AURPU_3_00014 | CU2 | 0.2% xylan from beechwood, |
| | | (Example 16.8) | pH 4.0, 30 min |
| 12C | Aurpu2p4_005858 | CU1 | 5 mM pNP-beta-D Glucopyranoside, |
| | | (Example 16.7) | pH 5.0, 30 min |
| 12D | Aurpu2p4_010898 | CU1 | 5 mM pNP-N-acetyl-beta-D- |
| | | (Example 16.7) | glucosaminide, pH 4.0, 30 min |
| 13A | AURPU_3_00307 | CU1 | (1 mM pNP-Beta-Galactopyranoside, |
| | | (Example 16.7) | pH 4.0, 30 min) GH20 |
| 13B | Aurpu2p4_008021 | CU2 | (0.2% Beta-mannan, |
| | | (Example 16.8) | pH 5, 30 min) GH5 |
| 13C | Aurpu2p4_009751 | CU2 | (0.2% xylan from beechwood, |
| | | (Example 16.8) | pH 4.0, 30 min) GH10 |
| 13D | AURPU_3_00016 | CU2 | 0.2% alpha-tomatine, |
| | | (Example 16.8) | pH 4.0, 30 min |
| 14A | AURPU_3_00018 | CU2 | (0.2% xylan from beechwood |
| | | (Example 16.8) | pH 3.5, 30 min) GH 11 |
| 14B | AURPU_3_00019 | CU2 | (0.2% xylan from beechwood |
| | | (Example 16.8) | pH 3.5, 30 min) GH 11 |
| 14C | AURPU_3_00147 | CU1 | (1 mM 4-nitrophenyl beta-D- |
| | | (Example 16.7) | mannopyranoside, pH 3.0, 30 min) GH2 |
| 14D | Aurpu2p4_006782 | CU1 | (1 mM pNP-β-D Glucopyranoside, |
| | | (Example 16.7) | 30 min, pH 4.0) GH 3 |
| 15A | AURPU_3_00192 | CU1 | (1 mM pNP-beta-Glucopyranoside, |
| | | (Example 16.7) | pH 4.0, 30 min) GH3 |
| 15B | AURPU_3_00208 | CU1 | (1 mM pNP-beta-Glucopyranoside, |
| | | (Example 16.7) | pH 4.0, 30 min) GH3 |
| 15C | Aurpu2p4_001633 | CU2 | (0.2% carboxymethylcellulose, |
| | | (Example 16.8) | pH 5.5, 30 min) GH5 |
| 15D | AURPU_ 3_00312 | CU1 | (1 mM pNP-beta-Glucopyranoside, |
| | | (Example 16.7) | pH 5.0, 30 min) GH5 |
| 16A | AURPU_3_00183 | CU2 | 0.08% locust bean gum, |
| | | (Example 16.8) | pH 4.5, 30 min |
| 16B | AURPU_3_00013 | CU2 | 0.2% xylan from beechwood, |
| | | (Example 16.8) | pH 6.0, 30 min |
| 16C | AURPU_3_00184 | CU1 | 1 mM p-nitrophenyl-beta-d- |
| | | (Example 16.7) | xylopyranoside, pH 5.0, 30 min |
| 16D | Aurpu2p4_011071 | CU6 | 0.7% Rhamnogalacturonan I from |
| | | (Example 16.12) | potato, pH 5.0, initial rate |

Although the present invention has been described hereinabove by way of specific embodiments thereof, it can be modified, without departing from the spirit and nature of the subject invention as defined in the appended claims.

1. An isolated polypeptide which is:
(a) a polypeptide comprising the amino acid sequence of any one of SEQ ID NOs: 571-855, 1468-1773, or 2548-2934;
(b) a polypeptide comprising an amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% amino acid sequence identity to the polypeptide defined in (a);
(c) a polypeptide comprising an amino acid sequence encoded by the nucleic acid sequence of any one of SEQ ID NOs: 286-570, 1162-1467, or 2161-2547;
(d) a polypeptide comprising an amino acid sequence encoded by any one of the exonic nucleic acid sequences corresponding to the positions as defined in Tables 2A-2C;
(e) a polypeptide comprising an amino acid sequence encoded by a polynucleotide molecule that hybridizes under medium-high stringency conditions, high stringency conditions, or very high stringency conditions with the full-length complement of a polynucleotide molecule comprising the nucleic acid sequence defined in (c) or (d);
(f) a polypeptide comprising an amino acid sequence encoded by a polynucleotide molecule having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% nucleic acid sequence identity to a polynucleotide comprising the nucleic acid sequence defined in (c) or (d);
(g) a functional variant of the polypeptide defined in (a) comprising a substitution, deletion, and/or insertion at one or more residues; or
(h) a functional fragment of the polypeptide of any one of (a) to (g).

2. The isolated polypeptide of claim 1, wherein said polypeptide has a corresponding function and/or protein activity according to Tables 1A-1C.

3. The isolated polypeptide of claim 1 or 2 comprising or consisting of the amino acid sequence of any one of SEQ ID NOs: 571-855, 1468-1773, or 2548-2934.

4. The isolated polypeptide of any one of claims 1 to 3, wherein said polypeptide is a recombinant polypeptide.

5. The isolated polypeptide of any one of claims 1 to 4 obtainable from a fungus.

6. The isolated polypeptide of any one of claims 1 to 5, wherein said fungus is from the genus Scytalidium, Myriococcum, or Aureobasidium.

7. The isolated polypeptide of any one of claims 1 to 6, wherein said fungus is Scytalidium thermophilum, Myriococcum thermophilum, or Aureobasidium pullulans.

8. An antibody that specifically binds to the isolated polypeptide of any one of claims 1 to 7.

9. An isolated polynucleotide molecule encoding the polypeptide of any one of claims 1 to 7.

10. An isolated polynucleotide molecule which is:
(a) a polynucleotide molecule comprising a nucleic acid sequence encoding the polypeptide of any one of SEQ ID NOs: 571-855, 1468-1773, or 2548-2934;
(b) a polynucleotide molecule comprising the nucleic acid sequence of any one of SEQ ID NOs: 1-285, 856-1161, or 1774-2160;
(c) a polynucleotide molecule comprising the nucleic acid sequence of any one of SEQ ID NOs: 286-570, 1162-1467, or 2161-2547;
(d) a polynucleotide molecule comprising any one of the exonic nucleic acid sequences corresponding to the positions as defined in Tables 2A-2C;
(e) a polynucleotide molecule comprising a nucleic acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% nucleic acid sequence identity to any one of the polynucleotide molecules defined in (a) to (d); or
(f) a polynucleotide molecule that hybridizes under medium-high stringency conditions, high stringency conditions, or very high stringency conditions with the full-length complement of any one of the polynucleotide molecules defined in (a) to (e).

11. The isolated polynucleotide molecule of claim 9 or 10 obtainable from a fungus.

12. The isolated polynucleotide molecule of claim 11, wherein said fungus is from the genus Scytalidium, Myriococcum, or Aureobasidium.

13. The isolated polynucleotide molecule of claim 12, wherein said fungus is Scytalidium thermophilum, Myriococcum thermophilum, or Aureobasidium pullulans.

14. A vector comprising a polynucleotide molecule as defined in any one of claims 9 to 13.

15. The vector of claim 14 further comprising a regulatory sequence operatively linked to said polynucleotide molecule for expression of same in a suitable host cell.

16. The vector of claim 15, wherein said suitable host cell is a bacterial cell.

17. The vector of claim 15, wherein said suitable host cell is a fungal cell.

18. The vector of claim 17, wherein said fungal cell is a filamentous fungal cell.

19. A recombinant host cell comprising the polynucleotide molecule as defined in any one of claims 9 to 13, or a vector as defined in any one of claims 14 to 18.

20. The recombinant host cell of claim 19, wherein said cell is a bacterial cell.

21. The recombinant host cell of claim 19, wherein said cell is a fungal cell.

22. The recombinant host cell of claim 21, wherein said fungal cell is a filamentous fungal cell.

23. A polypeptide obtainable by expressing the polynucleotide molecule of any one of claims 9 to 13, or the vector of any one of claims 14 to 18 in a suitable host cell.

24. A composition comprising the polypeptide of any one of claim 1 to 7 or 23, or the recombinant host cell of any one of claims 19 to 22.

25. The composition of claim 24 further comprising a suitable carrier.

26. The composition of claim 24 or 25 further comprising a substrate of said polypeptide.

27. The composition of claim 26, wherein said substrate is biomass.

28. A method for producing the polypeptide of any one of claim 1 to 7 or 23, said method comprising:
(a) culturing a strain comprising the polynucleotide molecule of any one of claims 9 to 13 or the vector of any one of claims 14 to 18 under conditions conducive for the production of said polypeptide; and
(b) recovering said polypeptide.

29. The method of claim 28, wherein said strain is a bacterial strain.

30. The method of claim 28, wherein said strain is a fungal strain.

31. The method of claim 30, wherein said fungal strain is a filamentous fungal strain.

32. A method for producing the polypeptide of any one of claim 1 to 7 or 23, said method comprising:
(a) culturing the recombinant host cell of any one of claims 19 to 22 under conditions conducive for the production of said polypeptide; and
(b) recovering said polypeptide.

33. A method for preparing a food product, said method comprising incorporating the polypeptide of any one of claim 1 to 7 or 23 during preparation of said food product.

34. The method of claim 33, wherein said food product is a bakery product.

35. Use of the polypeptide of any one of claim 1 to 7 or 23 for the preparation or processing of a food product.

36. The use of claim 33, wherein said food product is a bakery product.

37. The polypeptide of any one of claim 1 to 7 or 23 for use in the preparation or processing of a food product.

38. The polypeptide of claim 37, wherein said food product is a bakery product.

39. Use of the polypeptide of any one of claim 1 to 7 or 23 for the preparation of animal feed.

40. Use of the polypeptide of any one of claim 1 to 7 or 23 for increasing digestion or absorption of animal feed.

41. The use of claim 39 or 40, wherein said animal feed is a cereal-based feed.

42. The polypeptide of any one of claim 1 to 7 or 23 for the preparation of animal feed, or for increasing digestion or absorption of animal feed.

43. The polypeptide of claim 42, wherein said animal feed is a cereal-based feed.

44. Use of the polypeptide of any one of claim 1 to 7 or 23 for the production or processing of kraft pulp or paper.

45. The use of claim 44, wherein said processing comprises prebleaching.

46. The use of claim 44, wherein said processing comprises de-inking.

47. The polypeptide of any one of claim 1 to 7 or 23 for the production or processing of kraft pulp or paper.

48. The polypeptide of claim 47, wherein said processing comprises prebleaching or de-inking.

49. Use of the polypeptide of any one of claim 1 to 7 or 23 for processing lignin.

50. The polypeptide of any one of claim 1 to 7 or 23 for processing lignin.

51. Use of the polypeptide of any one of claim 1 to 7 or 23 for producing ethanol.

52. The polypeptide of any one of claim 1 to 7 or 23 for producing ethanol.

53. The use of any one of claims 35, 36, 40, 41, 44 to 46, 49 and 51 in conjunction with cellulose or a cellulase.

54. Use of the polypeptide of any one of claim 1 to 7 or 23 for treating textiles or dyed textiles.

55. The polypeptide of any one of claim 1 to 7 or 23 for treating textiles or dyed textiles.

56. Use of the polypeptide of any one of claim 1 to 7 or 23 for degrading biomass or pretreated biomass.

57. The polypeptide of any one of claim 1 to 7 or 23 for degrading biomass or pretreated biomass.
